SEQUENCE LISTING (112 sequences)

SEQ ID NO: 1 - protein, 154 aa, Artificial Sequence
  Description of Artificial Sequence Synthetic peptide
METDTLLLWVLLLWVPGSTGDGSMSDKIIHLTDDSFDTDVLKADGAILVDFWAEWCGPNSGGGGMIWEAWSCYACGTSGPCKMIAPILDEIADEYQGKLTVAKLNIDQNPGTAPKYGIRGIPTLLLFKNGEVAATKVGALSKGQLKEFLDANLA

SEQ ID NO: 2 - protein, 26 aa, Artificial Sequence
  Description of Artificial Sequence Synthetic peptide
YRRAAVPPSPSLSRHSSPHQSEDEEE

SEQ ID NO: 3 - protein, 27 aa, Artificial Sequence
  Description of Artificial Sequence Synthetic peptide
KYRRAAVPPSPSLSRHSSPHQSEDEEE

SEQ ID NO: 4 - protein, 24 aa, Artificial Sequence
  Description of Artificial Sequence Synthetic peptide
CGGSYLDSGIHSGATTTAPSLSGK

SEQ ID NO: 5 - DNA, 21 bp, Artificial Sequence
  Description of Artificial Sequence Synthetic primer
tgaagggaagaagcgatcctt

SEQ ID NO: 6 - DNA, 17 bp, Artificial Sequence
  Description of Artificial Sequence Synthetic primer
ggccggaagcaccatct

SEQ ID NO: 7 - DNA, 22 bp, Artificial Sequence
  Description of Artificial Sequence Synthetic primer
ggagcctggacacacagtacag

SEQ ID NO: 8 - DNA, 22 bp, Artificial Sequence
  Description of Artificial Sequence Synthetic primer
tgcatgttgaaaggttcctgaa

SEQ ID NO: 9 - DNA, 23 bp, Artificial Sequence
  Description of Artificial Sequence Synthetic primer
agttcctgggcctatctgatctc

SEQ ID NO: 10 - DNA, 18 bp, Artificial Sequence
  Description of Artificial Sequence Synthetic primer
cggcccaatcctgttcaa

SEQ ID NO: 11 - DNA, 21 bp, Artificial Sequence
  Description of Artificial Sequence Synthetic primer
tgttggcagggaaaatgttga

SEQ ID NO: 12 - DNA, 22 bp, Artificial Sequence
  Description of Artificial Sequence Synthetic primer
ggtgcaagatattggtggcttt

SEQ ID NO: 13 - DNA, 20 bp, Artificial Sequence
  Description of Artificial Sequence Synthetic primer
agtcggctgtttgggttgag

SEQ ID NO: 14 - DNA, 18 bp, Artificial Sequence
  Description of Artificial Sequence Synthetic primer
ttcccgcgatgagtgctt

SEQ ID NO: 15 - DNA, 24 bp, Artificial Sequence
  Description of Artificial Sequence Synthetic primer
tgcggaacagtgaaatgtgtataa

SEQ ID NO: 16 - DNA, 20 bp, Artificial Sequence
  Description of Artificial Sequence Synthetic primer
tggagccacccttacaggat

SEQ ID NO: 17 - DNA, 24 bp, Artificial Sequence
  Description of Artificial Sequence Synthetic primer
agaaatgtactctgctttgctgaa

SEQ ID NO: 18 - DNA, 23 bp, Artificial Sequence
  Description of Artificial Sequence Synthetic primer
ggtgaccttgtaagtgtgccttt

SEQ ID NO: 19 - DNA, 22 bp, Artificial Sequence
  Description of Artificial Sequence Synthetic primer
ccctccaaggcttgagtaaaag

SEQ ID NO: 20 - DNA, 22 bp, Artificial Sequence
  Description of Artificial Sequence Synthetic primer
cctccctttgtcatcatgtgaa

SEQ ID NO: 21 - DNA, 24 bp, Artificial Sequence
  Description of Artificial Sequence Synthetic primer
tcatatatgtggaagcctctggaa

SEQ ID NO: 22 - DNA, 22 bp, Artificial Sequence
  Description of Artificial Sequence Synthetic primer
gcccgacttcacagtctacatg

SEQ ID NO: 23 - DNA, 22 bp, Artificial Sequence
  Description of Artificial Sequence Synthetic primer
ggagcaaggacagtggagaatc

SEQ ID NO: 24 - DNA, 19 bp, Artificial Sequence
  Description of Artificial Sequence Synthetic primer
cgggcgtttctttccatgt

SEQ ID NO: 25 - DNA, 22 bp, Artificial Sequence
  Description of Artificial Sequence Synthetic primer
gagaatgggaagccgaacatac

SEQ ID NO: 26 - DNA, 21 bp, Artificial Sequence
  Description of Artificial Sequence Synthetic primer
cccctctatgaccggaatcac

SEQ ID NO: 27 - DNA, 23 bp, Artificial Sequence
  Description of Artificial Sequence Synthetic primer
aggctgttggaatttacgcataa

SEQ ID NO: 28 - DNA, 18 bp, Artificial Sequence
  Description of Artificial Sequence Synthetic primer
tggcttcggtctgatgca

SEQ ID NO: 29 - DNA, 20 bp, Artificial Sequence
  Description of Artificial Sequence Synthetic primer
tctgcggcgatgtcactatg

SEQ ID NO: 30 - DNA, 19 bp, Artificial Sequence
  Description of Artificial Sequence Synthetic primer
cctcgggctcaggttccta

SEQ ID NO: 31 - DNA, 23 bp, Artificial Sequence
  Description of Artificial Sequence Synthetic primer
catttcatgcatctcccctgatc

SEQ ID NO: 32 - DNA, 23 bp, Artificial Sequence
  Description of Artificial Sequence Synthetic primer
ccccaagtaacggagaaagaaga

SEQ ID NO: 33 - DNA, 21 bp, Artificial Sequence
  Description of Artificial Sequence Synthetic primer
ccactcgaacctcaccacaga

SEQ ID NO: 34 - DNA, 24 bp, Artificial Sequence
  Description of Artificial Sequence Synthetic primer
gacttgtcaaaactatgcaagcaa

SEQ ID NO: 35 - DNA, 21 bp, Artificial Sequence
  Description of Artificial Sequence Synthetic primer
accagcccgtcttctctctct

SEQ ID NO: 36 - DNA, 21 bp, Artificial Sequence
  Description of Artificial Sequence Synthetic primer
tgttctgtgtcctggcactga

SEQ ID NO: 37 - DNA, 21 bp, Artificial Sequence
  Description of Artificial Sequence Synthetic primer
ggacactttcttgcttgccaa

SEQ ID NO: 38 - DNA, 20 bp, Artificial Sequence
  Description of Artificial Sequence Synthetic primer
aagccatgcaaccagaccat

SEQ ID NO: 39 - DNA, 20 bp, Artificial Sequence
  Description of Artificial Sequence Synthetic primer
tccgaaccagtagctcctaa

SEQ ID NO: 40 - DNA, 20 bp, Artificial Sequence
  Description of Artificial Sequence Synthetic primer
ccctccacaaggcttcaaga

SEQ ID NO: 41 - DNA, 20 bp, Artificial Sequence
  Description of Artificial Sequence Synthetic primer
ggagatccgcagtctttgga

SEQ ID NO: 42 - DNA, 20 bp, Artificial Sequence
  Description of Artificial Sequence Synthetic primer
tggctgtcgtcagggaaatc

SEQ ID NO: 43 - DNA, 21 bp, Artificial Sequence
  Description of Artificial Sequence Synthetic primer
gcttgcgacccacgtagtaga

SEQ ID NO: 44 - DNA, 20 bp, Artificial Sequence
  Description of Artificial Sequence Synthetic primer
caccctcctgttgcctctga

SEQ ID NO: 45 - DNA, 20 bp, Artificial Sequence
  Description of Artificial Sequence Synthetic primer
cctgatgcaggacagaccaa

SEQ ID NO: 46 - DNA, 19 bp, Artificial Sequence
  Description of Artificial Sequence Synthetic primer
cgcctcggaagacctcagt

SEQ ID NO: 47 - DNA, 20 bp, Artificial Sequence
  Description of Artificial Sequence Synthetic primer
ggcactgaacaagccaacaa

SEQ ID NO: 48 - DNA, 17 bp, Artificial Sequence
  Description of Artificial Sequence Synthetic primer
tcgctgcgtccctctca

SEQ ID NO: 49 - DNA, 21 bp, Artificial Sequence
  Description of Artificial Sequence Synthetic primer
acagctggcttgctagaggaa

SEQ ID NO: 50 - DNA, 21 bp, Artificial Sequence
  Description of Artificial Sequence Synthetic primer
atttggcctggtgctcattaa

SEQ ID NO: 51 - DNA, 23 bp, Artificial Sequence
  Description of Artificial Sequence Synthetic primer
agatttgctgtagctgcgaagtc

SEQ ID NO: 52 - DNA, 22 bp, Artificial Sequence
  Description of Artificial Sequence Synthetic primer
gcaagtggtatgtggccttctg

SEQ ID NO: 53 - DNA, 20 bp, Artificial Sequence
  Description of Artificial Sequence Synthetic primer
gggctgtaggcactgagcaa

SEQ ID NO: 54 - DNA, 23 bp, Artificial Sequence
  Description of Artificial Sequence Synthetic primer
tccatctcttcatgttcccagaa

SEQ ID NO: 55 - DNA, 21 bp, Artificial Sequence
  Description of Artificial Sequence Synthetic primer
agcacatgcataggcggtgta

SEQ ID NO: 56 - DNA, 20 bp, Artificial Sequence
  Description of Artificial Sequence Synthetic primer
ggacagtggcacaggtgaca

SEQ ID NO: 57 - DNA, 21 bp, Artificial Sequence
  Description of Artificial Sequence Synthetic primer
gcaggactctcgtggtgttca

SEQ ID NO: 58 - DNA, 18 bp, Artificial Sequence
  Description of Artificial Sequence Synthetic primer
gccggaccagatccagaa

SEQ ID NO: 59 - DNA, 21 bp, Artificial Sequence
  Description of Artificial Sequence Synthetic primer
cctgagaggagagcgtcattg

SEQ ID NO: 60 - DNA, 20 bp, Artificial Sequence
  Description of Artificial Sequence Synthetic primer
gcccaattgcagttgagtga

SEQ ID NO: 61 - DNA, 21 bp, Artificial Sequence
  Description of Artificial Sequence Synthetic primer
tttcccgttcttcagcatttg

SEQ ID NO: 62 - DNA, 22 bp, Artificial Sequence
  Description of Artificial Sequence Synthetic primer
cggatatagtgtggcctttgtg

SEQ ID NO: 63 - DNA, 23 bp, Artificial Sequence
  Description of Artificial Sequence Synthetic primer
catgcttgggtcagtcaatattg

SEQ ID NO: 64 - DNA, 20 bp, Artificial Sequence
  Description of Artificial Sequence Synthetic primer
cccaggtgagtctgctccat

SEQ ID NO: 65 - DNA, 20 bp, Artificial Sequence
  Description of Artificial Sequence Synthetic primer
gccctctgttgccagaattc

SEQ ID NO: 66 - DNA, 25 bp, Artificial Sequence
  Description of Artificial Sequence Synthetic primer
aagaggagtggccaaaagatagact

SEQ ID NO: 67 - DNA, 19 bp, Artificial Sequence
  Description of Artificial Sequence Synthetic primer
gcgaagactgtcccatcca

SEQ ID NO: 68 - DNA, 21 bp, Artificial Sequence
  Description of Artificial Sequence Synthetic primer
gttgtcagaagccagcgttca

SEQ ID NO: 69 - DNA, 23 bp, Artificial Sequence
  Description of Artificial Sequence Synthetic primer
caatctcttctgggctgatcttc

SEQ ID NO: 70 - DNA, 24 bp, Artificial Sequence
  Description of Artificial Sequence Synthetic primer
tggctatgtcagctcctaaagtca

SEQ ID NO: 71 - DNA, 21 bp, Artificial Sequence
  Description of Artificial Sequence Synthetic primer
cagcaccaggaagggtacaga

SEQ ID NO: 72 - DNA, 25 bp, Artificial Sequence
  Description of Artificial Sequence Synthetic primer
tttgacccttgagctgacataagaa

SEQ ID NO: 73 - DNA, 24 bp, Artificial Sequence
  Description of Artificial Sequence Synthetic primer
cagatttatccccattttcatcct

SEQ ID NO: 74 - DNA, 22 bp, Artificial Sequence
  Description of Artificial Sequence Synthetic primer
gtccccaggagactcttcagaa

SEQ ID NO: 75 - DNA, 19 bp, Artificial Sequence
  Description of Artificial Sequence Synthetic primer
acttggtgggcagcagatg

SEQ ID NO: 76 - DNA, 20 bp, Artificial Sequence
  Description of Artificial Sequence Synthetic primer
ggtagaccctcgctggaaca

SEQ ID NO: 77 - DNA, 19 bp, Artificial Sequence
  Description of Artificial Sequence Synthetic probe
acgccaccaccggcccact

SEQ ID NO: 78 - DNA, 21 bp, Artificial Sequence
  Description of Artificial Sequence Synthetic probe
caactcccacgcccagccgtt

SEQ ID NO: 79 - DNA, 21 bp, Artificial Sequence
  Description of Artificial Sequence Synthetic probe
accaacacaacccgggcgctt

SEQ ID NO: 80 - DNA, 29 bp, Artificial Sequence
  Description of Artificial Sequence Synthetic probe
agtgtcgtcgtttctttctgctggtcaga

SEQ ID NO: 81 - DNA, 23 bp, Artificial Sequence
  Description of Artificial Sequence Synthetic probe
tcccctcttgctgctgctccctc

SEQ ID NO: 82 - DNA, 21 bp, Artificial Sequence
  Description of Artificial Sequence Synthetic probe
cccctgccgcagacacttgga

SEQ ID NO: 83 - DNA, 19 bp, Artificial Sequence
  Description of Artificial Sequence Synthetic probe
ttcagccgccgccatcagc

SEQ ID NO: 84 - DNA, 21 bp, Artificial Sequence
  Description of Artificial Sequence Synthetic probe
agcgccatgcccactcccttc

SEQ ID NO: 85 - DNA, 24 bp, Artificial Sequence
  Description of Artificial Sequence Synthetic probe
cccgaggaaatgaccatgctctgg

SEQ ID NO: 86 - DNA, 27 bp, Artificial Sequence
  Description of Artificial Sequence Synthetic probe
tgctcttctctgtgacccagtccatcc

SEQ ID NO: 87 - DNA, 19 bp, Artificial Sequence
  Description of Artificial Sequence Synthetic probe
acgacccttgccgcgggac

SEQ ID NO: 88 - DNA, 21 bp, Artificial Sequence
  Description of Artificial Sequence Synthetic probe
aggctgtcccaggcagcacca

SEQ ID NO: 89 - DNA, 23 bp, Artificial Sequence
  Description of Artificial Sequence Synthetic probe
aggccctcagcctcactccctgg

SEQ ID NO: 90 - DNA, 22 bp, Artificial Sequence
  Description of Artificial Sequence Synthetic probe
tctgagaacaccctgcccggct

SEQ ID NO: 91 - DNA, 22 bp, Artificial Sequence
  Description of Artificial Sequence Synthetic probe
tcgttgactgcccaaggctgcc

SEQ ID NO: 92 - DNA, 19 bp, Artificial Sequence
  Description of Artificial Sequence Synthetic probe
ttcccacgcgtcgaacgcc

SEQ ID NO: 93 - DNA, 23 bp, Artificial Sequence
  Description of Artificial Sequence Synthetic probe
ccagcctcccaaggagacccaga

SEQ ID NO: 94 - DNA, 21 bp, Artificial Sequence
  Description of Artificial Sequence Synthetic probe
ccgacgtgatgcccacgatga

SEQ ID NO: 95 - DNA, 22 bp, Artificial Sequence
  Description of Artificial Sequence Synthetic probe
ccaaccccagcctgaccagcaa

SEQ ID NO: 96 - DNA, 21 bp, Artificial Sequence
  Description of Artificial Sequence Synthetic probe
ttctggcccacccatggctca

SEQ ID NO: 97 - DNA, 24 bp, Artificial Sequence
  Description of Artificial Sequence Synthetic probe
cacagttccacccgcctcacattg

SEQ ID NO: 98 - DNA, 15 bp, Artificial Sequence
  Description of Artificial Sequence Synthetic probe
catccagcagctcgt

SEQ ID NO: 99 - DNA, 16 bp, Artificial Sequence
  Description of Artificial Sequence Synthetic probe
agcagactgcatagat

SEQ ID NO: 100 - DNA, 23 bp, Artificial Sequence
  Description of Artificial Sequence Synthetic probe
ccagaggaagacgtgcccatccg

SEQ ID NO: 101 - DNA, 20 bp, Artificial Sequence
  Description of Artificial Sequence Synthetic probe
agcgtcctgcaaaccgtgca

SEQ ID NO: 102 - DNA, 16 bp, Artificial Sequence
  Description of Artificial Sequence Synthetic probe
ccctatccaaaggaag

SEQ ID NO: 103 - DNA, 22 bp, Artificial Sequence
  Description of Artificial Sequence Synthetic probe
cacgtggtcctgcccttgtcga

SEQ ID NO: 104 - DNA, 25 bp, Artificial Sequence
  Description of Artificial Sequence Synthetic probe
cagactgctctgatggcaccgtctg

SEQ ID NO: 105 - DNA, 23 bp, Artificial Sequence
  Description of Artificial Sequence Synthetic probe
caggcaggctctccatcaaggca

SEQ ID NO: 106 - DNA, 28 bp, Artificial Sequence
  Description of Artificial Sequence Synthetic probe
tgttggtcaccaggtgcctttcaaattt

SEQ ID NO: 107 - DNA, 21 bp, Artificial Sequence
  Description of Artificial Sequence Synthetic probe
tgccgcctcacctgcccttgt

SEQ ID NO: 108 - DNA, 15 bp, Artificial Sequence
  Description of Artificial Sequence Synthetic probe
ccagcatgagatcca

SEQ ID NO: 109 - DNA, 16 bp, Artificial Sequence
  Description of Artificial Sequence Synthetic probe
tgaaagcatcatcttc

SEQ ID NO: 110 - DNA, 16 bp, Artificial Sequence
  Description of Artificial Sequence Synthetic probe
aggcaacacactgtca

SEQ ID NO: 111 - DNA, 16 bp, Artificial Sequence
  Description of Artificial Sequence Synthetic probe
agcacaacccaggatg

SEQ ID NO: 112 - DNA, 15 bp, Artificial Sequence
  Description of Artificial Sequence Synthetic probe
tgctgtgacaatgac

(19) United States
(12) Patent Application Publication
(10) Pub. No.: US 2006/0252045 A1
(43) Pub. Date: Nov. 9, 2006

(54) Methods and materials for identifying agents which modulate bone remodeling and agents identified thereby

(76) Inventors: Moitreyee Chatterjee-Kishore, Westford, MA (US); John A. Robinson, Downingtown, PA (US); Bheem M. Bhat, West Chester, PA (US); Frederick James Bex III, Newtown Square, PA (US)

Correspondence Address: DRINKER BIDDLE & REATH (DC), 1500 K STREET, N.W., SUITE 1100, WASHINGTON, DC, 20005-1209, US

(21) Appl. No.: 10/559,426

(22) PCT Filed: Jun. 7, 2004
(86) PCT No.: PCT/US04/17951

§ 371 Date: Apr. 24, 2006

(30) Foreign Application Priority Data
Jun. 6, 2003 (US) 60476164
Sep. 10, 2003 (US) 60501398

(51) Int. Cl. C12Q 1/68; A61K 31/404

(52) U.S. Cl. 435/6; 514/414

(57) ABSTRACT

The invention discloses compositions, compounds, apparatuses and methods of using them to study bone mineralization and identify agents that regulate bone mineralization. Methods of using bone mineralization gene profiles and signatures for compound screening and research are also disclosed. Reagents for modulating bone mineralization are provided for both therapeutic and research usage.

This application is related to U.S. Provisional No. 60/476,164, filed Jun. 6, 2003 and U.S. Provisional No. 60/501,398, filed Sep. 10, 2003, and the contents of which are herein incorporated in their entirety for all purposes. All cited patents and publications referred to in this application are herein incorporated by reference in their entirety for all purposes.

### BACKGROUND OF THE INVENTION

Bone disorders that involve bone mineral loss are a large contributor to health care costs and poor health in the aging population in the United States. Osteoporosis is the leading condition resulting in the large healthcare costs.

Bone mineral loss results from an imbalance in bone remodeling homeostasis and maintenance of normal serum calcium levels. Serum calcium depends on the interplay of intestinal calcium absorption, renal excretion and skeletal mobilization or uptake of calcium. Although serum calcium represents less than 1% of total body calcium, the serum level is extremely important for maintenance of normal cellular functions.

Serum calcium regulates and is regulated by three major hormones. Parathyroid hormone (PTH) and 1,25-dihydroxyvitamin D are the major regulators of calcium and bone homeostasis. PTH acts on the kidney to increase calcium reabsorption, phosphate excretion and 1,25-dihydroxyvitamin D production. PTH increases bone resorption. 1,25-dihydroxyvitamin D is a potent stimulator of bone resorption and an even more potent stimulator of intestinal calcium (and phosphate) absorption. 1,25-dihydroxyvitamin D is also necessary for bone mineralization. The third hormone involved in serum calcium regulation is calicitonin. Calcitonin modulates calcium homeostasis to a lesser extent than PTH and 1,25-dihydroxyvitamin D.

A number of feedback loops operate to control the level of serum calcium and the two major homeostatic hormones. A calcium-sensing receptor, identified in parathyroid and kidney cells, but also found in other tissues that senses extracellular calcium, plays a critical role in calcium homeostasis. Low serum calcium levels stimulate 1,25-dihydroxyvitamin D synthesis directly through stimulation of PTH release (and synthesis). To prevent an elevated level of serum calcium, a second set of feedback loops operate to decrease PTH and 1,25 dihydroxyvitamin D levels. These feedback loops maintain serum calcium within a narrow physiological range, regardless of the amount of calcium consumed by the individual.

In addition to calcium homeostasis and hormonal control of calcium, bone mineralization is also greatly influenced by cellular bone remodeling. Bone consists of extracellular matrix (largely mineralized), collagen and cells. Collagen fibers are of type I and comprise 90% of the total protein in bone. Within the collagen fibers are spindle or plate-shaped crystals of hydroxyapatite, [3Ca₃(PO₄)₂].(OH)₂. These spindle or plate shaped crystals are the calcium-phosphate containing compound derived from the serum calcium and phosphate. Hydroxyapatite is also found on the “ground substance”. The ground substance is composed primarily of glycoproteins and proteoglycans. These highly anionic complexes have a high ion binding capacity and therefore are believed to play an important role in calcification.

In addition to collagen, there are several cellular players that play an enormous role in bone remodeling and mineralization. The principal cells in bone are osteoclasts and osteoblasts (which also include bone-lining cells and osteocytes). Osteoclasts are the cells responsible for resorption of the bone and are derived from haematopoietic stem cells. Osteoblasts are derived from local mesenchymal cells and are directly responsible for bone formation. Osteoblasts are indirectly responsible for regulating osteoclastic bone resorption via paracrine factors.

Bone is continually undergoing renewal; this is called bone remodeling. In a normal adult, new bone is laid down by osteoblasts. New bone production is equally matched by osteoclast cell bone resorption. Most of the bone turnover occurs on bone surfaces, especially at endosteal surfaces. The rate of remodeling differs in different locations due to physical loading on a particular bone, proximity to a synovial joint or the presence of hematopoietic rather than fatty tissue in the marrow, and even the type of bone. Trabecular bone remodels 3-10 times more rapidly than cortical bone.

Remodeling follows an ordered sequence referred to as the basic multicellular unit of bone turnover or bone remodeling unit (BMU). In this cycle, bone resorption is initiated by the recruitment of osteoclasts, which act on matrix exposed by proteinases derived from bone lining cells. A resorptive pit (i.e., Howship's lacuna) is created by the osteoclasts. The pit results from the release of lysosomal enzymes from the osteoclasts into the pockets, which result in matrix resorption. This resorptive phase is then followed by a bone formation phase where osteoblasts fill the lacuna with osteoid. The osteoid is then mineralized with hydroxyapatite to form new bone matrix. It is the uncoupling of this remodeling cycle which can result in a detrimental net bone change that is observed in osteoporosis and other bone mineralization disorders.

Loss of bone mineral has no clinical effect itself, unless a fracture occurs. Common sites of fracture due to osteoporosis or bone mineralization loss disorders include fractures of the spine, wrist, hip or pelvis after minor trauma. Fractures can also manifest in loss of anterior height (i.e., wedge fractures), loss of midvertebral height (i.e., codfish vertebrae) or loss of anterior, middle and posterior height (i.e., compression or crush fractures). Other diseases that include bone loss include osteomalacia and Rickets.

Increased bone creation can also cause fractures. Paget's disease is a condition in which localized areas of bone show increased bone turnover due to overactive osteoclasts. The increased remodeling results in potential limb deformity, bone pain and increased fracture risk.

Currently, methods of preventing or inhibiting bone loss include exercise, a daily dietary calcium intake of 800-1200 mg/day in women, and avoidance of corticosteroids, which deleteriously affect calcium metabolism (e.g., inhibits osteoblastic bone formation). Vitamin D supplementation may be recommended when there is an indication of calcium malabsorption. In women, estrogen replacement therapy is also a common treatment, as it reduces osteoclastogenesis by decreasing production of cytokines such as IL-1 and RANK. Finally, bisphosphonates are an effective means of treating bone loss. These compounds act by inhibiting osteoclast function. However, no treatment exists that enhances bone mineralization, and the existing treatments are not greatly effective at inhibiting bone loss in affected populations. Most treatments only slow the progression of bone loss, but affected individuals will continue, despite treatment, to lose bone mass density.

In view of the complexity of serum calcium homeostasis and bone remodeling homeostasis, the feedback mechanisms that control them, and the current treatments available for treating bone disorders, additional methods of treating bone remodeling disorders are needed. Methods for screening agents, which modulate bone remodeling and mineralization are also needed.

### SUMMARY OF THE INVENTION

This invention is directed towards providing new reagents, which modulate bone remodeling and/or mineralization. The invention further provides for new research tools that can screen for compounds and compositions that modulate bone remodeling and/or mineralization based on the newly elucidated pathway which modulates bone remodeling, the Wnt pathway.

One aspect of the invention is directed to a gene expression profile of bone cells subjected to bone load, and wherein bone load has been modulated by a Wnt pathway modulator. The gene expression profile encompasses any two or more genes of any of Tables 1-5 or 12 or any of the genes and proteins derived there from involved in the pathway model of FIG. 16. Preferably, the Wnt pathway modulator is an agonist of the Wnt pathway. More preferably, the agonist is a GSK-3 inhibitor or a Wnt 3A, Wnt 3A mimetic, or Wnt 3A agonist. Other preferred modulators are discussed herein. Preferable GSK-3 inhibitors include lithium chloride or other lithium salt, a maleimide, a muscarinic agonist, an aloisine, a hymeninidisine or an inidirubin. The preferred maleimide is 3-(2,4-dichlorophenyl)-4-(1-methyl-1H-indol-3-yl)-1H-pyrrole-2,5-dione or 3-(3-chloro-4-hydroxyphenylamino)-4-(2-nitrophenyl)-1Hpyrrole-2,5-dione.

In another aspect of the invention, the gene profiles are derived from cultured cells, and preferably bone cells. Preferable bone cells are osteoblasts, osteoclasts, osteocytes, preosteoblasts, osteoprogenitor cells, or mesenchymal stem cells, or any combination of these cells.

Another object of the invention provides a method of identifying Wnt pathway modulating agents and thereby modulate bone remodeling comprising the steps of:
(A) obtaining a gene expression profile of bone cells exposed to a candidate agent; and
(B) comparing the gene expression profile of step (A) with a preferred gene expression profile thereby determining whether the Wnt pathway was modulated.

In yet another aspect of the invention, the gene expression profiles can be from cultured cells or cells obtained from animals (in vivo). The cells are preferably bone cells or stem cells, such as osteoblasts, osteoclasts, osteocytes, or mesenchymal cells. The profiles obtained include data from mechanically loaded cells or unloaded cells. Additional profiles can be prepared from cells expressing an LRP5 mutation (HBM cells) that yields a high bone mass phenotype.

It is a further object of the invention to provide a method of preparing a bone loading gene expression profile comprising the steps of:
(A) obtaining a gene expression profile of a bone cell population which is not exposed to mechanical stress and a gene expression profile of a bone cell population which is exposed to mechanical stress; and
(B) comparing the gene expression profile without mechanical stress with the gene expression profile with exposure to mechanical stress thereby obtaining a bone loading gene expression profile.
   This method can further comprise the steps of:
(C) obtaining a gene expression profile of a bone cell population to which a Wnt pathway modulator and mechanical stress have been administered;
(D) comparing the gene expression profile of step (C) with the gene expression profiles of steps (A) and (B) thereby obtaining an augmented bone loading gene expression profile.
   This method preferably uses osteoclasts, osteoblasts or other bone cells.

In a further aspect of the invention, a modulator of the above method is a Wnt pathway agonist or antagonist. Preferable agonists include Dkk antagonists (preferably Dkk1 antagonists), Wnt 3A agonists or mimetics (as well as Wnt 3A) GSK-3 antagonists, LRP5 agonists, LRP6 agonists, β-catenin agonists.

Another object of the invention provides for a method of screening agents that enhance bone remodeling due to mechanical load comprising the steps of: determining effect of a candidate agent on the load response of a cultured bone cell by comparing data sets from a gene expression profile generated in the absence of the candidate agent and in the presence of the candidate agent. Preferably such screening tools and methods comprise reference compounds (controls). Positive controls include for example GSK-3 inhibitors, and parathyroid hormone and. Other reference samples would be evident from the disclosure.

The agents identified by the above method can be used to treat such conditions and diseases as osteoporosis, a bone fracture, chondrodystrophies, a drug-induced bone disorder, high bone turnover, hypercalcemia, hyperostosis, osteoarthritis, osteomyelitis, and Paget's disease. Preferred bone fractures include but are not limited to hip fracture, Colle's fracture, or a vertebral crush fracture. Preferred drug-induced disorders include but are not limited to glucocorticoid induced osteoporosis, heparin-induced osteoporosis, an aluminum hydroxide induced osteomalacia, anticonvulsant induced osteomalacia, or glutethimide induced osteomalacia.

In yet another aspect, the invention relates to a composition comprising a plurality of probes, which correspond to genes of a bone loading gene expression profile. The plurality of probes preferably comprises probes that bind to nucleic acid sequences of connexin 43, COX-2, eNOS, SFRP1, Jun and Fos or any of the genes listed in Tables 1-5, 11 or 12.

Another aspect of the invention contemplates modulating bone mineralization in a cell using a reagent that produces one of the above bone load or mechanical load expression profiles. Preferred reagents are GSK-3 antagonists, such as, but not limited to a maleimide, a muscarinic agonist, an aloisine, a hymeninidisine or an inidirubin. Also preferred are Wnt 3A, its mimetics or functional variants thereof, and Wnt 3A agonists.

These reagents, in another aspect, can be combined with already approved therapies. For example, agonists of the Wnt pathway can be combined with existing bone mineralization modulating agents such as but not limited to parathyroid hormone, estrogen, vitamin D, a vitamin D analog, a selective estrogen receptor modulator, a glucocorticoid, a calcium preparation or a bisphosphonate.

In another object of the invention provides for a composition comprising a plurality of reagents (e.g., immunoglobulins or other protein-binding ligands) which recognize bind to two or more proteins encoded by the genes of Tables 1-5, 11 or 12. Preferable proteins recognized and bound by these reagents are two or more proteins are eNOS, connexin 43, SFRP1, cyclin D1, Wnt10B, Jun, Fos, and COX-2.

Another aspect of the invention provides for a composition for studying bone load modulation comprising (A) a substrate; and (B) a plurality of bone cell lysate two or more lysates from (i) cells without mechanical stress, (ii) cells with mechanical stress, (iii) HBM cells without mechanical stress, (iv) HBM cells with mechanical stress, and (v) any of the prior cells with a Wnt pathway modulator.

These compositions can then be utilized to screen reagents that bind to the proteins.

Another object of the invention contemplates a method of determining whether a compound or a composition enhances the effect of bone load on bone cell activity/function and/or mineralization comprising
(A) administering the compound or the composition to a cell line;
(B) administering thereafter a mechanical stimulus to the cell line;
(C) obtaining a cell lysate from the cell line;
(D) contacting the cell lysate to a solid substrate (e.g., plate, slide, bead, and the like) under suitable conditions to allow binding of proteins in the cell lysate to the solid substrate; and
(E) determining whether the compound or the composition enhances the effect of bone load on bone cell activity/function and/or mineralization by comparing the pattern obtained from step (D) with an expression pattern obtained from a cell lysate of cells to which mechanical load stimulus only was administered.

### BRIEF DESCRIPTION OF THE DRAWINGS

FIG. 1. FIG. 1A shows a dose dependent activation of TCF-signal by a GSK-3 inhibitor in HEK-293A cells. The graph shows that between 30 μM and 60 μM concentration of iGSK-3 activates transfected TCF-reporter, and hence Wnt signaling in 293A cells. FIG. 1B shows a comparison of dose dependent activation of TCF-signal by GSK-3 inhibitor in HEK-293A cells and U2OS bone cells. The data indicates that in addition to 293A cells, iGSK-3 inhibitor activates TCF-signal in U2OS bone cells. U2OS cells are more responsive that 293A cells to iGSK-3 mediated TCF-signal activation. The TCF-induction starts at lower dose (10 μM) than in 293A cells and peaks at 30 μM unlike 293A cells.

FIG. 2. GSK-3 inhibitor can be used to release Dkk1 mediated inhibition of TCF-signal in U2OS cells. As demonstrated, Wnt1 and Wnt3A activates TCF-signal about 10-15× over control. Addition of Dkk1 inhibit Wnt mediated TCF signal. GSK-3 inhibitor can reverse the inhibition. This demonstrates that this and other GSK-3 inhibitors can be used as controls or active agents in Dkk1-antagonist reporter assays. Other Wnt antagonists can be calibrated by using GSK-3 inhibitors.

FIG. 3. Effects of local administration of iGSK-3 on mouse calvarial thickness. H&E stained transverse section of parietal bone from mouse treated 18 days after administration of a local iGSK-3 injection. The local anabolic effect of 1 mg/kg/d iGSK-3 on the right hemicalvarium is evident.

FIG. 4. Local Effect of iGSK-3 on mouse calvariae thickness represented by percent change from the non-injected side of the calvariae. Quantification of calvarial bone thickness in mice treated with human PTH (hPTH), iGSK-3, and vehicle (50% DMSO containing 2% Tween 80 and 0.5% methylcellulose). Human PTH (1-34) at 20 μg/kg/day, served as a positive control and produced a significant increase in calvarial thickness. A significant increase in calvarial thickness was observed on the right hemicalvarium injected with iGSK-3 for 18 d when compared to the left non-injected hemicalvarium of the same animal (11.8%, p<0.005).

FIG. 5. Local Effect of 18 day iGSK-3 treatment on calvarial thickness compared to vehicle treated calvaria. Quantification of calvarial bone thickness in mice treated with hPTH, iGSK-3, and vehicle (50% DMSO containing 2% Tween 80 and 0.5% methylcellulose). Human PTH (1-34) at 20 μg/kg/day, served as a positive control and produced a significant increase in calvarial thickness. An increase (6%) in calvarial thickness was observed on the right hemicalvarium injected with iGSK-3 for 18 d when compared with vehicle alone.

FIG. 6. Local effect of 7 day PTH 1-34 and iGSK-3 treatment on calvarial thickness compared to vehicle treated calvaria (upper panel). Quantification of calvarial bone thickness in mice treated for 7 days with hPTH, iGSK-3, and using a different vehicle (i.e., 10% DMSO containing 2% Tween 80 with 0.5% methylcellulose) there was a statistically significant 10% increase in calvarial thickness compared to vehicle control treated calvaria (lower panel).

FIG. 7. The effects of iGSK-3 on endogenous alkaline phosphatase activity (ALPase) and β-catenin protein expression on mouse calvariae. The effect of iGSK-3 on calvarial bone was assessed by ALPase enzyme histochemical staining and β-catenin expression by immunohistochemistry. ALPase activity was markedly enhanced in osteoblasts following either iGSK-3 or PTH administration (upper panel). Immunohistochemistry of calvaria injected with iGSK-3 revealed strong β-catenin expression in osteoblastic cells lining the periosteum. In contrast, PTH had no effect on levels of β-catenin expression (bottom).

FIG. 8. Effects of strain on gene response of an expanded list of genes in MC3T3 cells immediately following load. Cyclin D1, Connexin 43, SFRP1, Wnt 10B, COX-2 and eNOS gene expression is induced, as well as Frizzled 2, Fos and Jun expression with the application of load. There was minimal induction of WISP2 gene expression following 5 hr of load.

FIG. 9. Effect of load alone on activation of the β-catenin pathway with iGSK-3 and load in combination with iGSK-3. The data demonstrate that load alone induced the expression of each of the genes (except WISP2) compared to non-loaded controls. The GSK-3 inhibitor (5 μM) alone induced the expression of Frizzled 2 and WISP2, but had no effect on Connexin 43, Cyclin D1, Wnt 10B, SFRP1, COX-2, eNOS, Fos or Jun. However, treatment of the MC3T3 cells with 5 μM GSK-3 inhibitor in the presence of load caused a synergistic induction of gene expression for each of the target genes.

FIG. 10. Dose dependent effects of iGSK-3 on Wnt target gene expression in the presence of load. The data demonstrate that load alone induced the expression of each of the genes compared to non-loaded controls. The GSK-3 inhibitor alone had no effect on gene expression for the genes listed at any concentration (data not shown). However, treatment of the MC3T3 cells with increasing concentrations (0.05-20 μM) of the GSK-3 inhibitor in the presence of load caused a dose-dependent synergistic induction of gene expression for each of the target genes.

FIG. 11. In vivo loading effects on calcein labeling. Female mice were loaded with 6 N of force while the male mice were loaded with 7 N. A robust bone formation response was observed as demonstrated by the increased calcein labeled surface in the tibia of both non-transgenic and HBM transgenic and in both sexes of loaded mice compared to non-loaded controls.

FIG. 12. TaqMan® data showing expression of COX-2, PTGS and eNOS in unloaded and loaded tibiae from non-TG and LRP5 G 171V TG mice. Load induced increase of mRNA levels for all three genes was higher in LRP5 G171V TG mice than in non-TG mice.

FIG. 13. FIG. 13A depicts TaqMan® data showing expression of Wnt related and Wnt target genes in non-TG and LRP5 G 171V TG (HBM TG) mice at 4 hr post load. Load induces an increase in transcription of β-catenin target genes in both non-TG and LRP5 G171V TG mice. However, this induction is more significant in the LRP5 G171V TG mice. FIG. 13B depicts TaqMan® data showing expression of Wnt related and Wnt target genes in non-TG and LRP5 G 171 V TG (HBM TG) mice at 24 hr post-load.

FIG. 14. TAQMAN® data showing expression of RANKL and OPG, at 4 and 24 hr post load, in non-TG and G 171V LRP5 TG (HBM TG) mice. RANKL gene transcription is not induced significantly in either non-TG or LRP5 G171V TG mice. OPG gene transcription is induced only in the LRP5 G171V TG mice and not in the non-TG mice.

FIG. 15. Effects of inhibiting COX-2 expression on load induced gene expression. One hour prior to loading (3,400 με strain for 5 hrs), the COX-2 inhibitor, NS-398, was added to the cells at various concentrations (1-60 μM). The COX-2 inhibitor was demonstrated to block the induction of Connexin 43, Cyclin D1, Wnt 10b, SFRP1 and COX-2 gene expression induced by load, while having no effect on Frizzled 2, eNOS, Fos and Jun. These data demonstrate that COX-2 expression plays an important role in mediating the response of Wnt target gene expression upon application of a load stimulus.

FIG. 16. Model describing the involvement of LRP5 in the activation of the Wnt/β-catenin pathway.

FIG. 17. Natural Wnt Ligand (Wnt 3A) Synergistically Induces β-catenin Target Gene Expression.

### DETAILED DESCRIPTION OF THE INVENTION

The methods, compositions and assays disclosed herein are for identification and analysis of compounds and compositions and their use to treat bone mineralization disorders and diseases. Such disorders and diseases include but are not limited to a bone development disorder, a bone fracture (e.g., fractures of the spine, hip, wrist or pelvis, wedge fractures, compression and crush fractures), age related loss of bone, a chondrodystrophy (e.g., achondroplasia, thanatophoric dysplasia, Jackson-Weiss syndromes with mutations in FGFR-2, and Pfeiffer syndrome with mutations in FGFR-1), a drug-induced bone disorder (e.g., glucocorticoid induced bone loss), high bone turnover, hypercalcemia, hyperostosis, osteomyelitis, osteoporosis, osteopetrosis, loss of midvertebral, anterior, middle, or posterior height, Paget's disease, or any of the other disorders and diseases discussed herein.

1. Definitions and Abbreviations

1.1 Definitions

By “subject” is meant to any animal. Preferred animals include avians, fish, mammals and rodents. Other categories of animals include domesticated animals or agricultural animals (e.g., poultry such as chickens, turkeys, ducks, and quail as well as pigs, sheep, goats, cattle, buffalo and the like). Preferred mammals include equines, porcines, ovines, caprines, bovines, and primates, with the preferred primate being humans.

By “agent” or “reagent” is meant to include a compound or composition that preferably modulates the Wnt pathway or a member thereof.

By a “reference compound” is meant to include a compound which modulates the Wnt pathway and more preferably both the Wnt pathway and bone remodeling that can serve as a control. Reference compounds include but are not limited to parathyroid hormone (PTH) and GSK inhibitors.

By “modulate” or “regulate” is meant the ability to alter by either up-regulating or down-regulating the activity of a protein, nucleic acid encoding a protein, a pathway (e.g., the Wnt pathway), a protein within a pathway and the like.

By “bone cell modulation” is meant to include modulation of bone density and/or bone mineralization. Modulation of bone cells can be determined in vitro by assessing changes in bone mineralization, alkaline phosphatase induction or induction of osteoblasts. In vivo, bone modulation can be assessed by any of the same methods studied in vitro as well as studying changes in bone mass density by bone scans or changes in Wnt pathway activity by staining tissue samples for β-catenin or other marker for bone modulation discussed herein.

The terms “force”, “load”, “stress” and “strain” are used interchangeably herein and are relate to the principles of force which in mechanics is any action that tends to maintain or alter the position of a body or to distort it and this term is used interchangeably with load in this document. Force as a measure per unit area is defined as “stress” and is also referred to in this document as “mechanical stress” and can be classified as compressive, tensile or shear depending on how the forces (load) are applied. Specifically, compressive stresses are developed if loads are applied so that the material becomes shorter, whereas tensile stresses are developed when the material is stretched. Shear stresses are developed when one region of a material slides relative to an adjacent region. The result of stress is defined as deformation and the percentage of the relative deformation or change in length is termed “strain”. If for example a material is stretched to 101% of its original length it has a strain of 0.01 or 1%. Since strain has no units it is either reported as relative deformation where a strain of 0.01 is equal to 1% deformation or in terms of microstrain where 10,000 microstrain is equal to 0.01 strain or 1% deformation (Turner et al., Bone, 14: 595-608 (1993)).

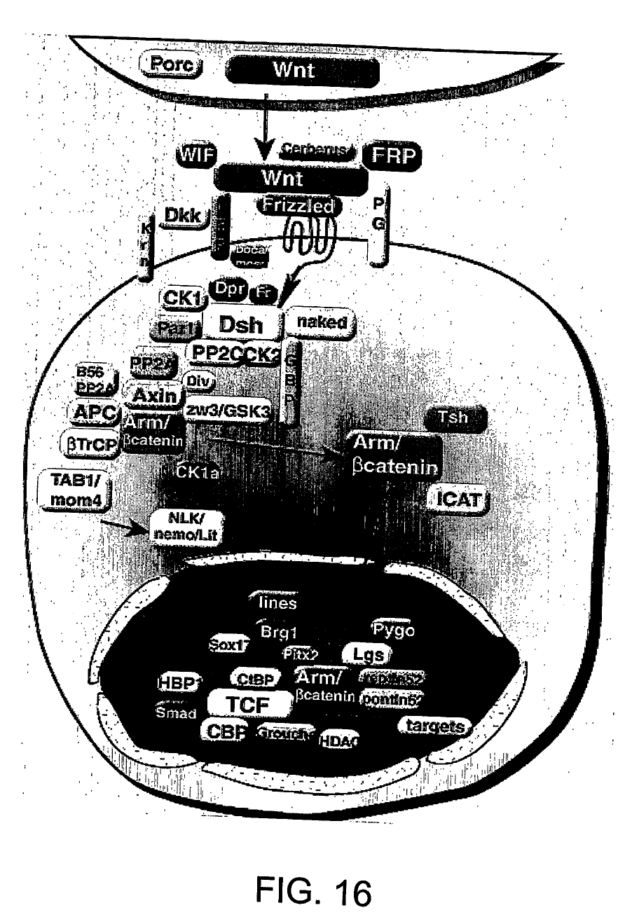

By “Wnt pathway” is meant to include any of the proteins downstream or upstream of Wnt protein activity (refer to FIG. 16). For example, this could include LRP5, LRP6, Dkk, GSK-3, Wnt10B, Wnt6, Wnt3 (e.g., Wnt 3A), Wnt1 or any of the other proteins discussed herein, and the genes that encode these proteins. Discussion of the Wnt pathway also is meant to include all of the pathways downstream of Wnt which are involved in bone remodeling, such as the LRP5 or HBM pathways, the Dkk pathway, the β-catenin pathway, the MAPKAPK2 pathway, the OPG/RANK pathway, and the like.

By “GSK inhibitor” is meant any agent which inhibits GSK activity. These can include non-selective GSK inhibitors, such as LiCl or other lithium salts, as well as selective GSK inhibitors. Preferred GSK inhibitors are GSK-3 inhibitors. More preferred GSK inhibitors are GSK-3 isoform specific inhibitors, such as GSK-3β or GSK-3α inhibitors. Additional inhibitors include but are not limited to monoclonal or polyclonal antibodies or immunogenically active fragments thereof, peptide aptamers, a GSK binding protein, an antisense molecule to a GSK nucleic acid, an RNA interference molecule, a morpholino oligonucleotide, a peptide nucleic acid (PNA), a ribozyme, and a peptide.

By “Dkk1 antagonist” is meant to include but not limited to monoclonal or polyclonal antibodies or immunogenically active fragments thereof, peptide aptamers, a GSK binding protein, an antisense molecule to a GSK nucleic acid, an RNA interference molecule, a morpholino oligonucleotide, a peptide nucleic acid (PNA), a ribozyme, and a peptide the inhibit Dkk1 activity in the Wnt pathway.

By “Wnt 3A agonist” is meant to include reagents which upregulate Wnt 3A synthesis and/or activity. By “Wnt 3A mimetic” is meant a molecule that mimics Wnt3A activity, preferably in a manner to that seen in Example 9. By “Wnt 3A variant” would include any functional variant which when administered with load can enhance activation with a Wnt/β-catenin response.

By “bone disorder” and “bone disease” is meant to include disorders wherein bone mineralization homeostasis has been adversely disrupted in the subject. Adverse disruption can be in the form of increased bone mineralization and decreased bone mineralization. Bone disorders include any of the disorders discussed herein. Preferable bone disorders include loss of bone mass or loss of bone mineralization homeostasis. For examples, preferable bone disorders and diseases include but are not limited to osteoporosis, bone fractures, chondrodystrophies, a drug-induced bone disorder, high bone turnover, hypercalcemia, hyperostosis, osteoarthritis, osteomyelitis and Paget's disease. Preferred fractures include but are not limited to hip fractures, Colle's fracture or a vertebral crush fracture. Preferred drug-induced disorders include but are not limited to glucocorticoid induced osteoporosis, heparin-induced osteoporosis, an aluminum hydroxide induced osteomalacia, anticonvulsant induced osteomalacia or glutethimide induced osteomalacia.

By “bone cell” is meant to include cells from tissue culture (“cultured cell”) or cells obtained from bone tissue. Such cells include but are not limited to osteoblasts, preosteoblasts, osteoprogenitor cells, osteoclasts, osteocytes, mesenchymal stem cells or any combination thereof. By bone tissue would mean to include a combination of these cells, as may be obtained from a bone biopsy.

By “bone remodeling” is meant the process of bone growth and turnover. By “bone remodeling agent” is meant a compound or a composition that modulates bone remodeling. Preferably the agent enhances bone remodeling such that bone mineralization is enhanced and bone resorption is inhibited. Thus, such agents may also include “bone mineralization modulators”. Bone remodeling can be studied both in vivo and in vitro.

By “bone mineralization” is meant the process hydroxyapatite formation in bone. Reagents which modulated bone mineralization are contemplated herein wherein the amount of hydroxyapatite forming in bone is modulated. For example, a bone mineralization agonist would be one that enhances the amount of hydroxyapatite formation in a subject in need thereof. Bone remodeling can be studied both in vivo and in vitro.

By “LRP5 pathway” and “IBM pathway” is meant any proteins/genes including LRP5 or the HBM mutant and proteins downstream of LRP5 or the HBM mutant involved in signaling relative to bone remodeling. Preferred agents of the invention are agonists of the LRP5 pathway that would be useful in treating a bone loss related disorder. Also contemplated are agents that are agonists of the related LRP6 pathway. Because of the great similarity between LRP5 and LRP6, all mention of LRP5 and HBM modulation are also contemplated with respect to LRP6.

By “HBM” is meant to include high bone mass, as well as the phenotype associated with the HBM1 kindred. In human LRP5, there is a mutation of G171V that produces the phenotype observed in the HBM1 kindred. Any mutation at this site however is contemplated in the human LRP5 gene or in any mammalian LRP5 gene or the equivalent site in the beta propellers of LRP6.

By “HBM phenotype” is meant to include all mutations that result in a phenotype such as that observed with the HBM1 kindred. The mutations can be at residue 171 of human LRP5 or at other sites in LRP5 or similar sites in LRP6 which induce high bone mass when expressed in an animal.

By “β-catenin pathway” is meant any proteins/genes including β-catenin and proteins downstream of β-catenin involved in signaling relative to bone remodeling. Preferred agents of the invention are those that activate the β-catenin pathway (i.e., β-catenin agonists).

By “MAPKAPK2 pathway” is meant any proteins/genes including MAPKAPK2 and proteins downstream of MAPKAPK2 involved in signaling relative to bone remodeling.

By “OPG/RANKL pathway” is meant any proteins/genes including OPG/RANKL and proteins downstream of OPG and RANKL involved in signaling relative to bone remodeling.

By “Dkk pathway” is meant to include any proteins/genes involved in Dkk-1 and LRP5 and/or LRP6 interaction that is part of the Wnt pathway. Dkk-1 inhibits LRP5 activity. Thus for bone loss disorders, Dkk-1 antagonists are preferred.

A “protein” means a polymer of amino acid residues linked together by peptide bonds. The term, as used herein, refers to proteins, polypeptides, and peptides of any size, structure, or function. Typically, however, a protein will be at least six amino acids long. Preferably, if the protein is a short peptide, it will be at least about 10 amino acid residues long. A “protein” also includes naturally occurring, recombinant, or synthetic proteins. Use of the term may also be referring to a protein fragment. A protein may be a single molecule or may be a multi-molecular complex. The term protein may also apply to amino acid polymers in which one or more amino acid residues are an artificial chemical analogue of a corresponding naturally occurring amino acid. An amino acid polymer in which one or more amino acid residues is an “unnatural” amino acid, not corresponding to any naturally occurring amino acid, is also encompassed by the use of the term “protein”. Preferably the proteins possess biological activity with respect to bone remodeling and/or bone mineralization.

A “fragment of a protein” or “protein fragment” means a protein/polypeptide, which is a portion of another protein. For instance, fragments of proteins may be polypeptides obtained by digesting full-length protein isolated from cultured cells. A fragment of a protein will typically comprise at least six amino acids. More typically, the fragment will comprise at least ten amino acids. Preferably, the fragment comprises at least about 16 amino acids. Such protein fragments preferably have biological activity. Such biological activity preferably is the modulation of the Wnt pathway, which results in modulation of bone mineralization.

By “immunoglobulin” is meant to include an antibody, and antibody fragment, and recombinant proteins that are a portion of an antibody. The use of the term “antibody” means an immunoglobulin, whether natural, or wholly or partially synthetically produced. All derivatives thereof that maintain specific binding ability to an antigen are also included in the term. The term also covers any protein having a binding domain, which is homologous or largely homologous to an immunoglobulin binding domain. These proteins may be derived from natural sources, or partly or wholly synthetically produced. An antibody may be monoclonal or polyclonal. The antibody may be a member of any immunoglobulin class, including any of the human classes: IgG, IgM, IgA, IgD, and IgE, as well as subclasses (e.g., IgG1, IgG2). Derivatives of the IgG class, however, are preferred in the present invention.

The term “antibody fragment” refers to any derivative of an antibody, which is less than full-length. Preferably, the antibody fragment retains at least a significant portion of the full-length antibody's specific binding ability. Examples of antibody fragments include, but are not limited to, Fab, Fab′, F(ab′)₂, scFv, Fv, dsFv diabody, and Fd fragments. The antibody fragment may be produced by any means. For instance, the antibody fragment may be enzymatically or chemically produced by fragmentation of an intact antibody, or it may be recombinantly produced from a gene encoding the partial antibody sequence. Alternatively, the antibody fragment may be wholly or partially synthetically produced. The antibody fragment may optionally be a single chain antibody fragment. Alternatively, the fragment may comprise multiple chains, which are linked together, for instance, by disulfide linkages. The fragment may also optionally be a multimolecular complex. A functional antibody fragment will typically comprise at least about 50 amino acids and more typically will comprise at least about 200 amino acids, or any length in between these values.

“Single-chain Fvs” (“scFvs”) are recombinant antibody fragments consisting of only the variable light chain (V_{L}) and variable heavy chain (V_{H}) covalently connected to one another by a polypeptide linker. Either V_{L} or V_{H} may be the NH₂-terminal domain. The polypeptide linker may be of variable length and composition so long as the two variable domains are bridged without serious steric interference. Typically, the linkers are comprised primarily of stretches of glycine and serine residues with some glutamic acid or lysine residues interspersed for solubility.

“Diabodies” are dimeric scFvs. The components of diabodies typically have shorter peptide linkers than most scFvs, and they show a preference for associating as dimers.

An “Fv” fragment is an antibody fragment that consists of one V_{H} and one V_{L} domain held together by non-covalent interactions. The term “dsFv” is used herein to refer to an Fv with an engineered intermolecular disulfide bond to stabilize the V_{H}-V_{L} pair.

A “F(ab′)₂” fragment is an antibody fragment essentially equivalent to that obtained from immunoglobulins (typically IgG) by digestion with the enzyme pepsin at pH 4.0-4.5. The fragment may also be recombinantly produced.

A “Fab” fragment is an antibody fragment essentially equivalent to that obtained by reduction of the disulfide bridge or bridges joining the two heavy chain pieces in the F(ab′)₂ fragment. The Fab′ fragment may also be recombinantly produced.

A “Fab” fragment is an antibody fragment essentially equivalent to that obtained by digestion of immunoglobulins (typically IgG) with the enzyme papain. The Fab fragment may also be recombinantly produced. The heavy chain segment of the Fab fragment is the Fd piece.

The term “protein-capture agent” means a molecule or a multi-molecular complex, which can bind a protein to itself. Protein-capture agents preferably bind their binding partners in a substantially specific manner. Protein-capture agents with a dissociation constant (K_{D}) of less than about 10⁻⁶ are preferred. Antibodies or antibody fragments are highly suitable as protein-capture agents. Antigens may also serve as protein-capture agents, since they are capable of binding antibodies. A receptor that binds a protein ligand is another example of a possible protein-capture agent. Protein-capture agents are understood not to be limited to agents, which only interact with their binding partners through non-covalent interactions. Protein-capture agents may also optionally become covalently attached to the proteins, which they bind. For instance, the protein-capture agent may be photo-crosslinked to its binding partner following binding.

The term “binding partner” means a protein that is bound by a particular protein-capture agent, preferably in a substantially specific manner. In some cases, the binding partner may be the protein normally bound in vivo by a protein that is a protein-capture agent. In other embodiments, however, the binding partner may be the protein or peptide on which the protein-capture agent was selected (through in vitro or in vivo selection) or raised (as in the case of antibodies). A binding partner may be shared by more than one protein-capture agent. For instance, a binding partner that is bound by a variety of polyclonal antibodies may bear a number of different epitopes. One protein-capture agent may also bind to a multitude of binding partners (for instance, if the binding partners share the same epitope).

“Conditions suitable for protein binding” means those conditions (in terms of salt concentration, pH, detergent, protein concentration, temperature, etc.) which allow for binding to occur between a protein and its binding partner in solution. Preferably, the conditions are not so lenient that a significant amount of non-specific protein binding occurs.

An “array” is an arrangement of entities in a pattern on a substrate. Although the pattern is often a two-dimensional pattern, the pattern may also be a three-dimensional pattern for a greater application of the material to the array substrate.

The term “substrate” refers to the bulk, underlying, and core material of the arrays of the invention. The substrate is the material to which nucleic acids, antibodies, immunoglobulins and other compounds are affixed.

The terms “micromachining” and “microfabrication” both refer to any number of techniques that are useful in the generation of microstructures (structures with feature sizes of sub-millimeter scale). Such technologies include, but are not limited to, laser ablation, electrodeposition, physical and chemical vapor deposition, photolithography, and wet chemical and dry etching. Related technologies such as injection molding and LIGA (e.g., X-ray lithography, electrodeposition, and molding) are also included. Most of these techniques were originally developed for use in semiconductors, microelectronics, and Micro-ElectroMechanical Systems (MEMS) but are applicable to the present invention as well.

The term “coating” means a layer that is either naturally or synthetically formed on or applied to the surface of the substrate. For instance, exposure of a substrate, such as silicon, to air results in oxidation of the exposed surface. In the case of a substrate made of silicon, a silicon oxide coating is formed on the surface upon exposure to air. In other instances, the coating is not derived from the substrate and may be placed upon the surface via mechanical, physical, electrical, or chemical means. An example of this type of coating would be a metal coating that is applied to a silicon or polymer substrate or a silicon nitride coating that is applied to a silicon substrate. Although a coating may be of any thickness, typically the coating has a thickness smaller than that of the substrate.

An “interlayer” is an additional coating or layer that is positioned between the first coating and the substrate. Multiple interlayers may optionally be used together. The primary purpose of a typical interlayer is to aid adhesion between the first coating and the substrate. For example, titanium or chromium interlayers are utilized to adhere a gold coating to a silicon or glass surface. However, other possible functions of an interlayer are also anticipated. For instance, some interlayers may perform a role in the detection system of the array (such as a semiconductor or metal layer between a nonconductive substrate and a nonconductive coating).

An “affinity tag” is a functional moiety capable of directly or indirectly immobilizing a polypeptide onto an exposed functionality of the organic thinfilm. Preferably, the affinity tag enables the site-specific immobilization and thus enhances orientation of the polypeptide or nucleic acid onto the organic thinfilm. In some cases, the affinity tag may be a simple chemical functional group. Other possibilities include nucleic acids, amino acids, poly(amino acid) tags, or full-length proteins. Still other possibilities include carbohydrates and nucleic acids. For instance, the affinity tag may be a polynucleotide that hybridizes to another polynucleotide serving as a functional group on the organic thinfilm or another polynucleotide serving as an adaptor. The affinity tag may also be a synthetic chemical moiety. If the organic thinfilm of each of the patches comprises a lipid bilayer or monolayer, then a membrane anchor is a suitable affinity tag. The affinity tag may be covalently or noncovalently attached to the protein. For instance, if the affinity tag is covalently attached to the polypeptide, it may be attached via chemical conjugation or as a fusion protein. The affinity tag may also be attached to the protein via a cleavable linkage. Alternatively, the affinity tag may not be directly in contact with the polypeptide. The affinity tag may instead be separated from the protein by an adaptor. The affinity tag may immobilize the protein to the organic thinfilm either through non-covalent interactions or through a covalent linkage.

An “adaptor”, for purposes of this invention, is any entity that links an affinity tag to the immobilized protein of a patch of the array. The adaptor may be, but need not necessarily be, a discrete molecule that is non-covalently attached to both the affinity tag and the protein. The adaptor can instead be covalently attached to the affinity tag or the protein or both (via chemical conjugation or as a fusion protein, for instance). Proteins such as full-length proteins, polypeptides, or peptides are typical adaptors. Other possible adaptors include carbohydrates and nucleic acids.

The term “fusion protein” refers to a protein composed of two or more polypeptides that, although typically unjoined in their native state, are joined by their respective amino and carboxyl termini through a peptide linkage to form a single continuous polypeptide. It is understood that the two or more polypeptide components can either be directly joined or indirectly joined through a peptide linker/spacer.

The term “normal physiological condition” means conditions that are typical inside a living organism or a cell. While it is recognized that some organs or organisms provide extreme conditions, the intra-organismal and intra-cellular environment normally varies around pH 7 (i.e., from pH 6.5 to pH 7.5), contains water as the predominant solvent, and exists at a temperature above 0° C. and below 50° C. It will be recognized that the concentration of various salts depends on the organ, organism, cell, or cellular compartment used as a reference. Normal physiological condition may further encompass both loaded and unloaded states in bone tissue and bone cells.

“Proteomics” means the study of or the characterization of either the proteome or some fraction of the proteome. The “proteome” is the total collection of the intracellular proteins of a cell or population of cells and the proteins secreted by the cell or population of cells. This characterization most typically includes measurements of the presence, and usually quantity, of the proteins that have been expressed by a cell. The function, structural characteristics (such as post translational modification), and location within the cell of the proteins may also be studied. “Functional proteomics” refers to the study of the functional characteristics, activity level, and structural characteristics of the protein expression products of a cell or population of cells.

1.2 Abbreviations
ACP5 acid phosphatase 5
Akt-3 protein kinase B (PKB) or RAC-PK
AlPASE alkaline phosphatase
AP1 adaptor-related protein 1
AP1B1 adaptor protein complex AP-1, beta 1 subunit
AXIN axin
b.i.d. bis in die (twice daily)
BGN bone specific biglycan
BMP1 bone morphogenetic protein 1
BMP4 bone morphogenetic protein 4
BMU bone remodeling unit
BSA bovine serum albumin
BTG2 B-cell translocation gene 2, anti-proliferative
CBFB core binding factor beta
CCND1 cyclin D1
CCND3 cyclin D3
CCNI cyclin I
CELSR2 cadherin EGF LAG seven-pass G-type receptor 2
CHUK/IKK alpha conserved helix-loop-helix ubiquitous kinase, IkB kinase alpha
CK1 alpha casein kinase 1, alpha 1
CKB creatine kinase, brain
CNK1 connector enhancer of KSR-like
Col1A1 collagen, type 1, alpha 1
Col3A1 collagen, type 3, alpha 1
Col6A3 collagen, type VI, alpha 3
Connx43 Connexin 43
COX-2 cyclooxygenase-2
CRABP2 cellular retinoic acid binding protein II
CSF1R colony stimulating factor 1 receptor
CSPG2 chondroitin sulphate proteoglycan
CTGF connective tissue growth factor
CTSK cathepsin K
CX3CR1 chemokine (C-X3-C) receptor 1
Cyclin D1 see also CCND1
DELTEX deltex homolog 2 (Drosophila), see EphB2
DMSO dimethyl sulphoxide
DVL1 disheveled, dsh homolog (Drosophila)
EDTA ethylenediaminetetra acetic acid
EGTA ethylene glycol-O—O′-bis(2-amino-ethyl)-N,N,N′N′-tetraacetic acid
EPHB2 connector enhancer of KSR-like (Drosophila kinase suppressor of ras)
EPHB6 Eph receptor B6
ERBB3 GRO1 oncogene
ERK also known as mitogen activated protein kinase p44/42 (MAPK)
FAP fibroblast activation protein, alpha
FBLN1 fibulin 1
FBS fetal bovine serum
FGF-2 Fibroblast growth factor 2 (basic)
FGF-7 Fibroblast growth factor 7 (keratinocyte growth factor)
FOS FBJ murine osteosarcoma viral oncogene homolog
FOSL1 Fos-like antigen 1
Frizzled2 Frizzled (Drosophila) homolog 2, also called FZD2
FZD2 Frizzled (Drosophila) homolog 2
G171V glycine to valine mutation at position 171 of human LRP5
GADD45A growth arrest and DNA-damage inducible, alpha
GADD45B growth arrest and DNA-damage inducible 45, beta
GADD45G growth arrest and DNA-damage inducible 45, gamma
GAS6 growth arrest-specific 6
GJA1 gap junction membrane channel protein alpha 1 (also known as Connexin 43)
GJB3 gap junction membrane channel protein beta 3
GSK-3 glycogen synthase kinase-3
GSK-3α glycogen synthase kinase-3, alpha isoform
GSK-3β glycogen synthase kinase-3, beta isoform
iGSK GSK inhibitor
iGSK-3 GSK-3 inhibitor
HBM high bone mass
HERPUD1 homocysteine-inducible, endoplasmic reticulum stress-inducible, ubiquitin0like domain member 1
HRT hormone replacement therapy
i.m. intramuscular
i.v. intravenous
IDB2 inhibitor of DNA binding 2
IDB3 inhibitor of DNA binding 3
IGF2 insulin-like growth factor 2 (somatomedin A)
IGF2R insulin-like growth factor 2 receptor
IGFBP6 insulin-like growth factor binding protein 6
IL-1 interleukin-1
IL1R1 interleukin-1 receptor, type I
IL1RL1 interleukin 1 receptor-like 1
IL4RA interleukins 4 receptor, alpha
IL-6 interleukin-6
ITGA5 integrin alpha 5 (fibronectin receptor alpha)
ITGB5 integrin, beta
ITGBL1 integrin, beta-like 1
JNK c-jun amino kinase pathway
JUN v-jun avian sarcoma virus 17 oncogene homolog
JUND1 Jun proto-oncogene related gene d1
LBD ligand binding domain of LRP5
LDLR low density lipoprotein receptor
LOX lysyl oxidase
LRP5 low density lipoprotein receptor-related protein 5
LRP6 low density lipoprotein receptor-related protein 6
LSP1 lymphocyte-specific protein 1
LUM lumican
MAPK mitogen activated protein kinase (p42,44) (ERK)
MAPKAPK2 mitogen-activated protein kinase-activated protein kinase 2, also called MK2
MCC mutated in colorectal cancers
MDSC mesenchyme derived stem cells
MET met proto-oncogene (hepatocyte growth factor receptor)
MMP-14 matrix metalloproteinase 14
MMP-9 matrix metalloproteinase 9
MSX1 homeo box, msh-like 1
MYBL1 v-myb myeloblastosis viral oncogene homolog (avian)-like 1
MYC v-myc avian myelocytomatosis viral oncogene homolog
MYCS Myc-like oncogene, s-myc protein
NCAM1 neural cell adhesion molecule 1
NFATC1 nuclear factor of activated T-cells, cytoplasmic 1
NFKB1 nuclear factor of kappa light chain gene enhancer in B-cells 1, p105
Non-TG non-transgenic
NOS3 nitric oxide synthase 3 (NOS3), also known as eNOS
NR4A1 nuclear receptor subfamily 4, group A, member 1
OGN osteoglycin
OPG osteoprotegerin
OSMR oncostatin M receptor
P.O. per os (by mouth)
PCOLCE procollagen c-proteinase enhancer protein
PDGFA Cluster Incl. M29464: Platelet derived growth factor alpha
PDGFRA platelet-derived growth factor receptor alpha polypeptide
PKA protein kinase A
PKC protein kinase C
PLAT tissue-type plasminogen activator, t-PA
PRDC-PENDING protein related to DAC and Cerberus
PTGIS prostaglandin synthase
PTGS1 prostaglandin-endoperoxide synthase 1, also called COX-1
PTGS2 prostaglandin-endoperoxide synthase 2 (prostaglandin G/H synthase or cyclooxygenase 2) or COX-2
PTH parathyroid hormone
q.d. quaque die (every day)
q.h. quaque hora (e.g., q24, q6h)
q.o.d. quaque altera die (every other day)
RAMP3 receptor (calicitonin) activity modifying protein 3
RANK receptor activator of NF-kB
RANKL receptor activator of NF-kB ligand
RNAi RNA interference
RUNX1 runt related transcription factor 1
RUNX2/CBFA1 runt related transcription factor 2
s.c. subcutaneous
S100A10 calcium binding protein similar to calpactin
SDC1 syndecan 1
SDF1 stromal derived factor 1
SERM selective estrogen receptor modulator
SERPINE1 serine (or cysteine) proteinase inhibitor, clade E (nexin, plasminogen activator inhibitor type 1), member 1
SFRP1 secreted frizzled-related protein 1
SFRP4 secreted frizzled-related protein 4
shRNA small hairpin RNA
siRNA short interfering RNAs
SPARC sparc/osteonectin
SPARCL1 SPARC-like 1 (mast9, hevin)
SPP1 secreted phosphoprotein 1
SPR surface plasmon resonance
STAT1 signal trandsducer and activator of transcription 1
STAT3 RIKEN cDNA 1110034C02 gene
TANK TRAF family member-associated Nf-kappa B activator
TG transgenic
TGFB1 transforming growth factor, beta 1
TGFB1 transforming growth factor, beta receptor II
THBD thrombomodulin
THBS1 thrombospondin 1
TIEG TGFB inducible early gene
TIMP1 tissue inhibitor of metalloproteinase
TIMP2 tissue inhibitor of metalloproteinase 2
TIMP3 tissue inhibitor of metalloproteinase 3
TNF tumor necrosis factor
TNFRSF10B tumor necrosis factor receptor superfamily, member 10b
TNFRSF11B tumor necrosis factor receptor superfamily, member 11b (osteoprotegerin)
TNFSF11 tumor necrosis factor (ligand) superfamily, member 11 (see RANKL)
TOB1 transducer of ErbB-2.1
TRAF3 TNF receptor-associated factor 3
TUNEL terminal deoxynucleotidyl transferase dUTP nick end labeling
UNK_D83402 prostaglandin 12 (prostacyclin) synthase
VCAM1 vascular cell adhesion molecule 1
VEH vehicle
WIF Wnt inhibitory factor
WISP1 WNT1 inducible pathway protein 1
WISP2 WNT1 inducible signaling pathway protein 2
wk week
Wnt wingless-type MMTV integration site
Wnt 3A wingless-type MMTV integration site family member 3A
Wnt6 wingless-type MMTV integration site family member 6
Wnt10B wingless-type MMTV integration site family member 10B
   2. Bone Load Gene Expression Profile

One novel aspect of the invention is the elucidation that the Wnt pathway is involved in bone mineralization homeostasis and that by modulating this pathway, mineralization can also be modulated. Using both in vivo and in vitro assays, a gene expression profile of bone load was elucidated. Most typically a gene expression profile (i.e., the identification of which genes are up- and down-regulated), and more particularly a gene signature profile (i.e., the quantities of genes' transcripts up-regulated and down-regulated relative to each other) was developed for a wide variety of genes directly or indirectly associated with activation of the Wnt signaling pathway.

Performing the gene expression analysis as disclosed herein (see additional section below as well as the examples), it was discovered that numerous genes are up-regulated in response to bone load and enhancement of bone load, most especially including COX-2, eNOS, Connexin 43, Fos, Jun and SFRP1 (additional genes are listed in the tables below). It was further determined that β-catenin is an essential component in the canonical Wnt pathway. Upon activation of this pathway, β-catenin is no longer phosphorylated. The unphosphorylated form of β-catenin accumulates in the cytoplasm and translocates into the nucleus. Once in the nucleus, β-catenin can then relieve inhibitors of targeted transcription factors, including TCF and LEF, and in turn activate transcription.

Signaling pathway agonists (i.e., Wnt pathway agonists) include but are not limited to GSK inhibitors. Additional signaling pathway inhibitors include but are not limited to Wnt 3A, Wnt 3A mimetics, Wnt 3A agonists, PKC inhibitors (e.g., SQ22536), PKA inhibitors (e.g., H89, Calbiochem), MEK1/2 inhibitors (e.g., U0126, PD98059 of Calbiochem), P38 MAPK inhibitors (e.g., SB203580, Calbiochem), JNK inhibitors (SP-600125 of Calbiochem), MAPKAP2 inhibitors (Calbiochem Cat. No. 3850880), calcium mobilization inhibitors (e.g., TMB-8 hydrochloride), G-protein coupled signaling inhibitors (e.g., pertussis toxin), nitric oxide synthase inhibitors (e.g., L-NAME), and COX-2 inhibitors (e.g., NS-398, indomethacin).

Thus, the agonists and antagonists discussed above can be used both as research tools to study (1) the Wnt pathway, (2) Wnt pathway signaling as related to bone homeostasis, (3) Wnt pathway regulation with respect to bone homeostasis, (4) contribution of other signaling pathways in conjunction with the Wnt pathway signaling, (5) bone load response and gene expression profiles of bone load both in vivo and in vitro, (6) and bone homeostasis and modulation thereof. The reagents can be used, for example, to identify new bone anabolic gene targets; they can also be used to treat subjects in need of bone homeostasis modulation. For example, Wnt pathway agonists can be used to treat bone loss, and Wnt pathway antagonists can be used to treat disorders with elevated bone mineralization, such as is seen in osteopetrosis.

2.1 Gene Expression Profiling

Gene expression profiling is performed by analyzing transcription of genes into RNA. A preferred method of doing this is via real-time PCR and TaqMan® methodology. Real-time PCR offers a rapid and reproducible method of preparing a transcriptional profile and gene transcriptional signature in response to a stimulus, especially at time points immediately after the stimuli. This method therefore is particularly useful for analyzing bone cell response to bone load. The signal detected is in direct proportion to the amount of PCR product in a reaction. By recording the amount of fluorescence emission at each cycle, it is possible to monitor the PCR reaction during the exponential phase of PCR, wherein the first significant increase in the PCR product correlates to the initial amount of target template.

Real-time PCR and the use of TaqMan® technology therefore also allows the analysis of multiple targets on the same plate, as long as all the primer sets utilize the same thermal cycling parameters. Consequently analysis of a plurality of genes, such as the genes that have been shown to be up- and down-regulated in response to bone stress stimuli, can be assessed. Methods of using real-time PCR are disclosed herein and in the examples. Additional methods would be known to the skilled artisan. See, for example, RAPID CYCLE REAL-TIME PCR: METHODS AND APPLICATION (S. Meuer et al., eds., Springer Verlag 2001) and RAPID CYCLE REAL-TIME PCR—METHODS AND APPLICATIONS (W. Dietmaier et al., eds., Springer Verlag 2002).

Although real-time PCR is a preferred method of performing gene expression profiling, other methods of RNA analysis and quantification can also be employed. Additional means for analyzing RNA expression are known in the art and including eTAG (ACLARA Biosciences), Northern blot analysis, S1 nuclease analysis, RNase protection assays and Western blot (viewing changes at the protein level). Methods for doing these assays are known in the art. See for example, USING ANTIBODIES: A LABORATORY MANUAL, Harlow, Ed and Lane, David (Cold Spring Harbor Press, 1999); Sambrook et al., MOLECULAR CLONING: A LABORATORY MANUAL (2nd Ed. Cold Spring Harbor Laboratory Press, 1989); and Maniatis et al., MOLECULAR CLONING, A LABORATORY MANUAL, (Cold Spring Harbor Laboratory, Cold Spring Harbor, N.Y. 1982).

Gene expression profiling can be performed on cells grown in culture for in vitro analysis of bone loading, as well as in vivo analysis of transcription in cells obtained from bone tissue. Methods of administering bone stimuli for both in vivo and in vitro analysis is discussed further below. Briefly, gene expression profiles and signatures were obtained for unloaded cells, cells to which load has been administered, cells to which agents which modulate the Wnt pathway have administered, HBM cells at rest and to which have been administered load, and from cells from the prior categories from either HBM transgenic (TG) or normal animals. The compilation of gene expression profiles obtained from each population of cells has provided both single gene profile and gene signature sets by which agent screening can be preformed, as well as an optimized set gene expression profile, which provides a set of up and down regulated genes that is the same set of genes which is found to be up- and down-regulated in response to bone stimulus in nature.

Bone gene expression profiles were obtained for the following set of parameters:
(1) in vitro cell cultures absent load,
(2) in vitro cell cultures subjected to a load stimulus,
(3) in vitro cell cultures subjected to a load stimulus after administration of a compound that modulates Wnt pathway activity,
(4) cells obtained from HBM animals subjected to load,
(5) cells obtained from HBM TG animals subjected to load animals AND a compound that modulates the Wnt pathway,
(6) cells obtained from non-TG animals subjected to load,
(7) cells obtained from non-TG animals subjected to load and a Wnt pathway modulator, and
(8) cells obtained from either TG or non-TG animals not subject to load.
   Based on the data obtained for each set of cells, gene expression profiles (i.e., an indication of the genes that are up- and down-regulated) and gene expression signatures (i.e., the degree of up regulation and down regulation of gene expression as compared to resting state) was obtained. Prom that data, a core set of genes was obtained which constitutes genes that are always up- or down-regulated in response to bone load.

The tables below break down the gene expression profiles obtained for each of the parameters above.

TABLE 1
HBM Gene Expression Profile

| | | Observed Effect of HBM |
|---|---|---|
| Gene | Pathway | Genotype on Gene Expression |
| ACP5 | HBM | Up-regulated in HBM cells |
| Col1A1 | HBM | No significant affect |
| Connexin 43 | Wnt | No significant affect |
| CTSK | HBM | Up-regulated in HBM cells |
| Cyclin D1 | Wnt | No significant affect |
| ENOS | Load Sensor | No significant affect |
| Frizzled 2 | Wnt | No significant affect |
| GADD45A | HBM | Down-regulated in HBM cells |
| IGF2 | HBM | Down-regulated in HBM cells |
| IGFBP6 | HBM | Up-regulated in HBM cells |
| IL-6 | Load Sensor | Down-regulated in HBM cells |
| IL-8 | Stress & Osteoclast | Down-regulated in HBM cells |
| | Function | |
| MK2 | Stress & Osteoclast | Down-regulated in HBM cells |
| | Function | |
| OPG | Stress & Osteoclast | No significant affect |
| | Function | |
| Osteonectin | HBM | No significant affect |
| PTGS2 | Load Sensor | No significant affect |
| RANKL | Stress & Osteoclast | No significant affect |
| | Function | |
| SFRP1 | Wnt | Up-regulated in HBM cells |
| SFRP4 | Wnt | Up-regulated in HBM cells |
| TGFβ | HBM | Up-regulated in HBM cells |
| TIMP3 | HBM | Up-regulated in HBM cells |
| WISP2 | Wnt | Up-regulated in HBM cells |
| Wnt10B | Wnt | Up-regulated in HBM cells |

By “stress and osteoclast function” in Table 1 is meant a gene that is a stress responsive gene as well as a gene that is required for osteoclastogenesis and function. By “load sensor” as used in Table 1, is meant a gene known in the literature to respond to mechanical load. By “HBM signature” as used for Table 1 and throughout the application is meant to include a set of genes that is differentially expressed in cell lines expressing the HBM mutation or in affected individuals of the human HBM1 kindred.

TABLE 2
Effect of Load on Gene Expression In vivo
Comparing HBM TG and Non-TG Animals

| Gene | Pathway | Effect of Load on Gene Expression |
|---|---|---|
| ACP5 | HBM | Up-regulated equally in the males and is |
| | | more significantly induced in female |
| | | HBM-TG |
| Col1A1 | HBM | No significant change in either |
| Connexin 43 | Wnt | Up-regulated; More significant in HBM-TG |
| CTSK | HBM | Up-regulated in both animals equally |
| Cyclin D1 | Wnt | Up-regulated; More significant in HBM-TG |
| ENOS | Load Sensor | Up-regulated; More significant in HBM-TG |
| Frizzled 2 | Wnt | Up-regulated; More significant in HBM-TG |
| GADD45A | HBM | Down-regulated in both animals |
| IGF2 | HBM | Up-regulated in both male animals |
| IGFBP6 | HBM | Up-regulated; More significant in HBM-TG |
| IL-6 | Load Sensor | Up-regulated; More significant in HBM-TG |
| IL-8 | Stress & | Up-regulated; More significant in HBM-TG |
| | Osteoclast | |
| | Function | |
| LRP5 | — | No significant change in either |
| MK2 | Stress & | Up-regulated in non-TG animals only |
| | Osteoclast | |
| | Function | |
| OPG | Stress & | Up-regulated in HGM-TG animals only |
| | Osteoclast | |
| | Function | |
| Osteonectin | HBM | Up-regulated; More significant in HBM-TG |
| PTGS | Load Sensor | Up-regulated; More significant in HBM-TG |
| RANKL | Stress & | No significant change in either |
| | Osteoclast | |
| | Function | |
| SFRP1 | Wnt | Up-regulated; More significant in HBM-TG |
| SFRP4 | Wnt | Up-regulated; More significant in HBM-TG |
| TGFβ | HBM | No significant change in either |
| TIMP3 | HBM | No significant change in either |
| WISP2 | Wnt | Up-regulated; More significant in HBM-TG |
| Wnt10B | Wnt | Up-regulated; More significant in HBM-TG |

TABLE 3
Effect of Load on Gene Expression In vitro

| | | MC3T3 Cell |
|---|---|---|
| | | Response to |
| | | Gravitational |
| Gene | Gene type | Load |
| AP1B1 | Stress regulated gene | Up-regulated |
| AXIN | Wnt pathway component | Up-regulated |
| BMP1 | Observed to be induced by iGSK-3 | Up-regulated |
| CBFB | Osteoblast function | Up-regulated |
| CCND1 | Wnt target gene | Up-regulated |
| CCND3 | Cell cycle | Up-regulated |
| CELSR2 | G-type receptor | Up-regulated |
| CHUK/IKK alpha | Facilitates β-catenin nuclear | Up-regulated |
| | translocation | |
| CK1 alpha | Wnt pathway component | Up-regulated |
| CKB | Kinase | Up-regulated |
| CRABP2 | Osteoblast differentiation | Up-regulated |
| CSF1R | Osteoclastogenesis | Up-regulated |
| CTGF | Growth factor | Up-regulated |
| DVL1 | Wnt signaling intermediate | Up-regulated |
| EPHB6 | Wnt target gene | Up-regulated |
| FOSL1 | Stress regulated gene | Up-regulated |
| GADD45B | Cell cycle | Up-regulated |
| GADD45G | Cell cycle | Up-regulated |
| GJA1 | Wnt target gene | Up-regulated |
| GJB3 | Wnt target gene | Up-regulated |
| HERPUD1 | Wnt target gene | Up-regulated |
| IGFBP6 | IGF binding protein | Up-regulated |
| IL1R1 | IL-1 mediated signaling, | Up-regulated |
| | inflammation | |
| IL1RL1 | IL-1 mediated signaling, | Up-regulated |
| | Inflammation | |
| IL4RA | Inflammation | Up-regulated |
| ITGA5 | Integrin signaling | Up-regulated |
| JUN | Stress regulated gene | Up-regulated |
| JUND1 | Stress regulated gene | Up-regulated |
| LDLR | Lipoprotein receptor | Up-regulated |
| LOX | Lysyl oxidase | Up-regulated |
| MAPKAPK2 | Kinase in stress regulated signaling | Up-regulated |
| MSX1 | Wnt target gene | Up-regulated |
| MYCS | Wnt target gene | Up-regulated |
| NCAM1 | Wnt target gene | Up-regulated |
| NFATC1 | Inflammation | Up-regulated |
| NFKB1 | Inflammation, proliferation | Up-regulated |
| PDGFA | Growth factor, osteoblast | Up-regulated |
| | development | |
| PRDC-PENDING | Cereberus like protein | Up-regulated |
| PTGS1 | Inflammation | Up-regulated |
| PTGS2 | Wnt target gene | Up-regulated |
| RAMP3 | Calcium signaling | Up-regulated |
| RUNX | Osteoblast function | Up-regulated |
| RUNX2/CBFA1 | Osteoblast function | Up-regulated |
| SDC1 | Proteoglycan required for Wnt | Up-regulated |
| | signaling | |
| SERPINE1 | Protease | Up-regulated |
| SPARCL1 | Osteoblast function | Up-regulated |
| STAT3 | Proliferation and cell growth | Up-regulated |
| TANK | Inflammation, NF-kB signaling | Up-regulated |
| TGFB1 | TGF beta signaling gene | Up-regulated |
| THBD | Endothelial cell function | Up-regulated |
| TIEG | TGF beta signaling gene | Up-regulated |
| TIMP1 | Matrix metalloproteinase | Up-regulated |
| TIMP3 | Matrix metalloproteinase | Up-regulated |
| TNFRSF11B/OPG | Wnt target gene | Up-regulated |
| TRAF3 | NF-kB signaling | Up-regulated |
| WISP1 | Wnt target gene | Up-regulated |

The above listed genes were modulated in response to application of gravitational load to cultured MC3T3 cells.

TABLE 4
The Effects of Load Using the FlexerCell in the Presence and Absence of iGSK-3; a
Wnt/β-catenin Pathway Activator

| Treatment/GENE | CCND1 | CXN43 | SFRP1 | Wnt10b | eNOS | COX-2 | FOS |
|---|---|---|---|---|---|---|---|
| No iGSK/No load | 1.00 | 1.00 | 1.00 | 1.00 | 1.00 | 1.00 | 1.00 |
| No iGSK + load | 3.64 | 3.39 | 3.05 | 2.76 | 2.35 | 2.48 | 3.20 |
| iGSK 0.05 μM + load | 3.80 | 4.27 | 3.04 | 3.70 | 2.54 | 2.56 | 3.66 |
| iGSK 0.2 μM + load | 4.39 | 4.42 | 3.36 | 3.53 | 2.65 | 2.74 | 3.76 |
| iGSK 1 μM + load | 5.17 | 4.76 | 3.59 | 3.69 | 3.06 | 3.16 | 4.51** |
| iGSK 5 μM + load | 6.93* | 5.38 | 5.41* | 4.40* | 4.33* | 6.50** | 6.20** |
| iGSK 20 μM + load | 7.13** | 7.72** | 10.00** | 6.95** | 5.95** | 8.17** | 10.77** |

*indicates a near 2 fold induction over load.
**indicates equal to or >2 fold induction over fold.

For additional genes that get up and down regulated, see the Examples and other Tables provided herein.

3. Methods of Studying Bone Loading In Vivo

3.1 Bone Studies

To understand the mechanism underlying the anabolic nature of the HBM mutations, HBM transgenic (TG) mice were subjected to in vivo mechanical loading to look for changes in gene expression as compared to their non-transgenic (non-TG) control littermates. This was performed by obtaining tibias or calvaria from the animals to which bone load stimuli has been administered, but other suitable bones can be used, including but not limited to ulnas, femurs and vertebrae. RNA was obtained from the HBM TG and non-TG littermate mice after load stimuli was administered. RNA was then extracted from the calvaria or tibias (or other bones) and compared between the animals (i.e., HBM TG and non-TG animals at rest and after load stimuli).

It was observed that the HBM mice had significantly greater Wnt pathway gene response than their non-TG littermate controls. From this observation, it was concluded that the HBM mutation causes the bone to be more sensitive to mechanical loading. One signature set of genes produced in response to a load stimuli in vivo comprises up-regulation of connexin 43, osteonectin, osteoprotegerin, eNOS, COX-2, prostacyclin synthase (PTGS), interleukins-6 (IL-6), cyclin D1, Wnt 10B, SFRP1 and SFRP4. Additional genes also were up-regulated as discussed in greater detail below and in the examples.

Methods of inducing bone load stimuli include the four-point load system discussed in the Examples. Additional in vivo methods of administering load are know in the art (e.g., three-point load system) and can also be used as would be known to the artisan of ordinary skill.

With the above expression profile obtained in the HBM TG mice or with any combination of the additional genes discussed herein, agents can be screened in the non-TG and HBM TG animals to ascertain whether a particular agent enhances activation of the Wnt pathway and thereby bone mineralization. Several positive controls for studying agents which enhance mineralization include PTH, and a GSK-3 inhibitor which enhances mineralization via activation of the Wnt pathway, 3-(3-chloro-4-hydroxyphenylamino)-4-(2-nitrophenyl)-1-H-pyrrole-2,5-dione. Other GSK-3 inhibitors described herein can also be used as positive controls.

In addition to gene expression profiles and signatures obtained from animals subjected to load stimuli and/or Wnt pathway modulation, animals can also be studied for changes in bone pathology as a result of load and/or Wnt pathway modulation. For example, changes in calvaria thickness (or thickness changes in other bones) and protein expression of any of the RNAs or proteins listed in any of the Tables herein as being up- or down-regulated in response to bone load stimulus alone or in combination with one or more compounds that modulate bone remodeling.

Bone calvaria analysis can be performed for example by administering a test agent to an animal in an amount of about 0.01 mg/kg/day to about 100 mg/kg/day. More preferably, the agent is provided in an amount of 0.1 mg/kg/day to about 50 mg/kg/day. For example animals can be administered the agent at about 0.5, 10 and 50 mg/kg/day. Typically, animals are done in batches of 6 mice per group (total of 72 mice in a study) and studied 5, 15 and 30 days post administration. Parathyroid hormone (PTH) can be used as a positive control, as can the GSK-3 inhibitor, 3-(3-chloro-4-hydroxyphenylamino)-4-(2-nitrophenyl)-1Hpyrrole-2,5-dione. After bone load stimulus in the presence and absence of these reagents, differences in calvaria size can be measured.

Other methods of studying pathological changes to bone would be evident to one of skill in the art. These pathological changes in bone can then be compared to the gene expression and gene signature profiles obtained both in vivo and in vitro and the data further correlated. As previously discussed, the gene expression profiles can be obtained by any of the) methods discussed herein or as would be evident to one of ordinary skill.

Although any of the genes in the discussed above can be assayed for modulated activity in response to a bone load stimulus, preferred genes for evaluation include but are not limited to SFRP1, TIMP3, GJA1, CTSK, Col1A1, CCND1, TIMP2, GADD45A, WISP2, FZD2, SFRP4, IGFBP6, LRP5, LRP6, IL6, IGF2, SPARC, MAPKAPK2, TNF, TNFRSF11B, TNFSF11, PTGS2 (COX-2), eNOS, GRO1 and Wnt10B. See also the genes that are listed in any of the Tables herein as being up- or down-regulated in response to bone load stimulus alone or in combination with one or more compounds that modulate bone remodeling.

4. Methods of Studying Bone Loading In Vitro

One aspect of the invention is the study of the effect of bone load in vitro and means by which the benefits of bone load (i.e., increased bone mineralization) can be enhanced. Studying bone load enhancement can be done both in vivo (as discussed above) and in vitro. Preferably bone load enhancement is first performed in vitro followed then with in vivo experiments, such as those discussed above.

Consequently, one aspect of the invention involves placing cells under conditions, which simulate load stimuli. There are several methods available for placing strain on cell cultures to mimic the bone load response observed in vivo. These methods include but are not limited to fluid shear, hydrostatic compression, uniaxial stretch, biaxial stretch, gravitational loading and load induced using a Flexercell® or equivalent system.

4.1 Bone Load Stimuli

Preferred genes which are modulated by a bone load stimuli, such as those provided by any of the above methods, include but are not limited to SFRP1, connexin 43, CCND1, Wnt10b, Jun, Fos, PTGS2 (COX-2) and eNOS. Additional genes that can be monitored for increases in their activity (e.g., increased mRNA transcripts and protein) as reflected in many of the Tables herein. At least six genes that have been shown to be consistently up-regulated in response to bone load (i.e., Jun, Fos, eNOS, SFRP1, COX-2 and Connexin 43) are also enhanced by the addition of an agent which activates the Wnt pathway. Other genes, such as Wnt2, are not enhanced by the addition of reagents that activated the Wnt pathway (e.g., GSK-3 inhibitors and Wnt 3A and its agonists, mimetics, and variants) and only respond to bone load.

4.1.1 Fluid Shear Stimulus

One method of inducing bone load is by fluid shear. Fluid shear involves a cone plate viscometer that generates continuous laminar shear by a stirring mechanism. Alternatively, a flow loop apparatus can produce such shear in a parallel flow culture chamber. The latter method and apparatus is exemplified by the Streamer system produced by Flexcell International Corporation. The flow loop apparatus also is known to produce a reproducible and consistent stimulus. The only drawbacks are that the end points are typically short-lived and whether these changes impact the function of differentiated osteoblasts (Basso et al., Bone 30(2): 347-51 (2002)).

4.1.2 Hydrostatic Compression Stimulus

A second method of inducing bone load is use of hydrostatic compression. Hydrostatic compression utilizes compressed air to generate a continuous or intermittent force that is believed to localize the force specifically to regions where the cells interact with the extracellular matrix protein/adhesion proteins.

4.1.3 Uniaxial Stretch Stimulus

A third means of inducing bone load in vitro is use of a uniaxial stretch stimulus. The uniaxial stretch method utilizes stretch force in one direction. The method involves growing cells in a tissue culture on a treated strip of polystyrene film or other film, which is fixed to a flexible layer of silicone. The layer of silicone is further attached to two metal bars. The metal bars can be manipulated relative to each other using an electromagnet or some other moving means. This method does not create any fluid shear. The lack of fluid shear makes this method less preferred, because interstitial fluid flow may play a larger role in bone remodeling than mechanical stretch. Accordingly, this method may not fully mimic what occurs in vivo despite the reproducible and consistent stimulus produced (Basso et al., Bone 30(2): 347-51 (2002)).

4.1.4 Biaxial Stretch Stimulus

Biaxial stretch is essentially the Flexercell® system discussed herein. This method utilized a collagen coated silastic membrane upon which the cells are grown. The plates are then placed in a special tray, which is attached to a vacuum pump. The vacuum pump stretches and relaxes the membrane, by stretching or otherwise distorting the cell membrane. Additionally, any media or fluid movement will further add fluid shear.

4.1.5 Gravitational Load Stimulus

Gravitational loading is another method by which bone load can be induced in vitro. Essentially, force is placed on the cells causing the cells to flatten. For additional details, see for example, Hatton et al., J. Bone & Min. Res. 18(1): 58-66 (2003); and Fitzgerald et al., Exp. Cell. Res. 228: 168-71 (1996). Specifically, the cells are grown on plates or cover slips and then are exposed to increasing G forces.

4.1.6 Flexercell® Stimulus

One preferred method for assessing reagent-based enhancement of the Wnt pathway and bone mineralization is using the Flexercell® system, a biaxial stretch stimulus. Briefly, bone cells (e.g., MC3T3 cells) are exposed to 3,400 με. Loads of about 50 με to about 5,000 με (and any value in between) can be used as well for mechanical load stimuli. Any stimulus in this range mimics physiological bone load stimuli. Stimuli above 5,000 με result in pathophysiological loads and therefore are not preferred. The cells also can be exposed to a Wnt pathway modulator (e.g., a GSK inhibitor) prior to exposure to biaxial stretch.

The genes up-regulated by the administration of the load alone or with a GSK-3 inhibitor include, but are not limited to COX-2, eNOS, connexin 43, and SFRP1. The expression profile obtained in vitro from the Flexercell® studies mimics the in vivo loading gene expression profile (i.e., RNA analysis performed on cells from HBM TG mice tibia wherein the mice were subjected to bone load using a four-point system). Thus, this mechanical load assay, or the use of other mechanical load means with the variety of cell lines disclosed herein, can be used to identify small molecules, peptides, immunoglobulins, and the like that modulate, and preferably activate, the canonical Wnt pathway and which mimic the HBM phenotype.

The in vitro methods of inducing mechanical stress stimuli on cells can also be used to study cell proliferation and apoptosis, which is relevant to bone remodeling and the need for osteoblast and osteoclast proliferation and osteoclast resorption. For example, HBM and unaffected osteoblastic cells can be seeded into bioflex 6 well plates and cultured for 2-3 days in growth media containing 10% FBS until the cells are about 60% confluent. Twenty-four hours prior to mechanical loading, the media is replaced with 1 mL of basal media containing about 2 to about 4% FBS. The cells are then subjected to about 50 to about 5,000 με of load for about 1 to about 5 hours.

Following load, the cells are cultured for an additional period of time. Subsequently, cell number and proliferation can be assessed using a number of commercial assays or assays known in the art, including but not limited to [³H]-thymidine incorporation, 5-bromo-2′-deoxyuridine (BrdU) incorporation, 3-(4,5 dimethylthiazol-2-yl)-5-(3-carboxymethoxyphenyl)-2-(4-sulfophenyl)-2H-trazolium alts (MTS) assay, TUNEL assay (i.e., terminal deoxynucleotidyltransferase dUTP nick end labeling) or Annexin V assay.

Additional Wnt pathway agonists include other GSK-3 inhibitor compounds as discussed herein, natural Wnt pathway ligands, synthetic ligands, small molecules as well as known antagonists cerebrus, SFRP and WIF (Wnt Inhibitory Factor) can be analyzed using the in vitro bone load methods described above for their ability to enhance bone load. Known Wnt pathway activators include Wnt1 and Wnt3A, small molecule Wnt mimetics, peptide aptamers that interact with LRP5 and activate Wnt signaling. Preferred peptide aptamers include:

| | Aptamer Sequence |
|---|---|
| Aptamer | (Amino to Carboxy Terminus) |
| 262 | METDTLLLWVLLLWVPGSTGDGSMSDKIIHLTDDSFDTDVLK |
| | ADGAILVDFWAEWCGPNSGGGGMIWEAWSCYACGTSGPCKMI |
| | APILDEIADEYQGKLTVAKLNIDQNPGTAPKYGIRGIPTLLL |
| | FKNGEVAATKVGALSKGQLKEFLDANLA |

In another embodiment, Wnt antagonists can be screened or used to treat individuals wherein bone demineralization (e.g., osteopetrosis) is needed. Wnt antagonists include but are not limited to Dkk1 antagonists.

4.2 Cell Cultures

The cells to which in vitro loading experiments can be performed include but are not limited to the following human cell lines: U2OS cells (ATCC), MG-63 cells (ATCC), SAOS-2 cells (ATCC), HOS-TE85 cells (ATCC), HOB03CE6 cells (Wyeth), HOBO1C1 preosteocytes (Wyeth), and human primary osteoblasts. Additionally, cells can be cultured from any mammalian system. Preferred animal lines for study include rat and mouse bone cells. For example, mouse bone cells, which can be used with any of the above methods include but are not limited to MC3T3 cells (ATCC) as discussed in the examples and primary osteoblasts or any cell line analogous to the human cell lines above. Rat cells that can be used with any of the disclosed methods of inducing stress in vitro include but are not limited to UMR-106 cells (ATCC), ROS17/2.8 cells and primary osteoblasts or any cell line analogous to the human cell lines above. Methods of culturing the cells would be known to the skilled artisan. See, e.g., IAN FRESHNEY, CULTURE OF ANIMAL CELLS—A MANUAL OF BASIC TECHNIQUE (4^{th} ed., Wiley-Liss, New York, 2000).

In another aspect of the invention cells can be taken from bones and can include osteoblasts, osteoclasts and osteocytes as well as progenitor and stem cells. Preferred osteoblasts and their progenitor and stem cells include mature osteoblasts, preosteoblasts (mature and immature), and mesenchymal stem cells (also referred to as mesenchyme-derived stem cells, MDSC).

In another aspect of the invention, human cell lines obtained from HBM and unaffected individuals can be used in conjunction with the bone load methodologies discussed herein. These cell lines can be used to investigate the gene inductions identified from the in vivo loading experiments performed on HBM and non-transgenic mice.

4.3 TCF Luciferase Assays

A TCF-luciferase assay system can also be used to monitor Wnt signaling activity. Constructs for the TCF-luciferase assays can be prepared as would be known in the art. For example, Wnt pathway proteins such as LRP5, LRP6 and HBM amongst others, can be expressed in pcDNA3.1, using Kozak and signal sequences to target peptides for secretion.

Once constructs have been prepared, cells such as osteoblasts and HEK293 cells are seeded in well plates and transfected with construct DNA, CMV β-galactosidase plasmid DNA, and TCF-luciferase reporter DNA. The cells are then lysed and assayed for β-galactosidase and luciferase activity to determine whether Wnt pathway interacting proteins, or other molecules such as antibodies affect Wnt signaling. Additional detail is provided in the examples below regarding methods of using TCF-luciferase constructs.

In another embodiment, the Flexercell® mechanical cell loading system (or any of the in vitro means of inducing load on cells) can be used in combination with the TCF-luciferase reporter system, or other reporter systems, to measure the effects of mechanical loading on the Wnt pathway. Such experiments can be performed as follows. For these experiments, MC3T3 cells (or another equivalent cell discussed herein) are plated as described above and cultured for three days or until confluent. The media is then changed to either serum free media containing BSA or low serum media (1% FBS) containing αMEM. The cells on this low or serum free media are then incubated for another 24 hours. About one hour prior to mechanical load, one plate is pre-treated with a dose of a Wnt pathway modulator (e.g., GSK inhibitor, natural Wnt ligand including but not limited to Wnt 1 and Wnt 3A), while another plate is untreated. Following pretreatment with any Wnt mimetic ligands, small molecules, etc., the cells are then subjected to mechanical load (e.g., 3,400 με) for about 5 hr as previously described. RNA is harvested from the loaded and the unloaded control samples immediately following load and 24 hours post-load using the Qiagen mini kit, as discussed above.

Real-time PCR can then be performed on the load signature set genes (or any suitable RNA assay as would be known in the art) at each time point to observe the changes in gene expression with treatment. Alternatively, the RNA can be analyzed using other methods known to the skilled artisan or as discussed herein.

5. Arrays

One method of utilizing the gene profiles and signatures of Wnt pathway involvement in bone remodeling and modulation thereof is in the form of preparing nucleic acid and protein arrays. These arrays can then be utilized to farther study the Wnt pathway and its involvement in bone remodeling. These arrays can also be used to screen for agents that modulate bone remodeling through the Wnt pathway.

5.1 Nucleic Acid Arrays

Nucleic acid arrays would be prepared as is known to one skilled in the art. Methods of preparing and utilizing such arrays are described in, for example, P. Baldi et al., DNA MICROARRAYS AND GENE EXPRESSION: FROM EXPERIMENTS TO DATA ANALYSIS AND MODELING (Cambridge University Press 2002); and DNA MICROARRAYS: A MOLECULAR CLONING MANUAL (David Bowtell and Joseph Sambrook, eds., Cold Spring Harbor Laboratory, 2002).

Preferred nucleic acid arrays would contain nucleic acids corresponding to members of the Wnt signaling pathway of any of the genes in Tables 1-5 or FIG. 16. For example, such arrays would contain 2, 3, 4, 5, 6, 7, 8, 9, 10, 15, 20, 25, 30, 35, 40, 45, 50 or more (or any integer value inbetween) of the genes involved in bone modeling. Such genes include any of the modulated genes listed in any of the tables, in the examples or are part of the pathways depicted in FIG. 16. These nucleic acids are exemplary of nucleic acids associated with a bone loading response.

In another embodiment, arrays can be prepared which include the Wnt pathway bone remodeling genes and genes involved in for example serum calcium modulation, osteoclast apoptosis, osteoblast proliferation, and the like.

TABLE 5
List of Genes for Development of High Bone
Mass Microarray or Protein/Antibody Array

| GENE | DESCRIPTION | WHERE EXPRESSED |
|---|---|---|
| ACP5 | acid phosphatase 5, tartrate resistant | Bone and colon |
| | | cancer |
| CCND1 | cyclin D1 (PRAD1: parathyroid | HBM Bone |
| | adenomatosis 1) | |
| CNK1 | v-erb-b2 erythroblastic leukemia viral | Bone and colon |
| | oncogene homolog 3 (avian) | cancer |
| COL1A1 | collagen, type I, alpha 1 | HBM Bone |
| COL6A3 | collagen, type VI, alpha 3 | HBM Bone |
| CTGF | connective tissue growth factor | HBM Bone |
| CTSK | cathepsin K (pycnodysostosis) | HBM Bone |
| CX3CR1 | chemokine (C—X3—C) receptor 1 | Inflammation in |
| | | bone |
| DELTEX | deltex homolog 2 (Drosophila), EphB2 | Bone and colon |
| | | cancer |
| EPHB2 | connector enhancer of KSR-like | Bone and colon |
| | (Drosophila kinase suppressor of ras) | cancer |
| ERBB3 | GRO1 oncogene (melanoma growth | Bone and colon |
| | stimulating activity, alpha) | cancer |
| FAP | fibroblast activation protein, alpha | Bone and colon |
| | | cancer |
| FBLN1 | fibulin 1 | HBM Bone |
| FGF-2 | fibroblast growth factor 2 (basic) | Inflammation in |
| | | bone |
| FGF-7 | fibroblast growth factor 7 (keratinocyte | Inflammation in |
| | growth factor) | bone |
| FOS | fos FBJ murine osteosarcoma viral | Bone and colon |
| | oncogene homolog | cancer/load |
| | | sensing gene |
| FZD2 | frizzled (Drosophila) homolog 2 | HBM Bone |
| GADD45A | growth arrest and DNA-damage- | HBM Bone |
| | inducible, alpha | |
| GAS6 | growth arrest-specific 6 | HBM Bone |
| GJA1 | gap junction protein, alpha 1, 43 kD | HBM Bone |
| | (connexin 43) | |
| IGF2 | insulin-like growth factor 2 | Inflammation in |
| | (somatomedin A) | bone |
| IGF2R | insulin-like growth factor 2 receptor | Inflammation in |
| | | bone |
| IGFBP6 | insulin-like growth factor binding | HBM Bone |
| | protein 6 | |
| IL-6 | interleukin 6 (interferon, beta 2) | Inflammation in |
| | | bone |
| ITGB5 | integrin, beta 5 | HBM Bone |
| ITGBL1 | integrin, beta-like 1 (with EGF-like | HBM Bone |
| | repeat domains) | |
| JUN | jun avian sarcoma virus 17 oncogene | Bone and colon |
| | homolog | cancer/load |
| | | sensing gene |
| LOX | lysyl oxidase | HBM Bone |
| LRP5 | low density lipoprotein receptor-related | HBM Bone |
| | protein 5 | |
| LRP6 | low density lipoprotein receptor-related | HBM Bone |
| | protein 6 | |
| LSP1 | lymphocyte-specific protein 1 | Inflammation in |
| | | bone |
| MAPKAPK2 | mitogen-activated protein kinase- | Osteoclast activity |
| | activated protein kinase 2 | |
| MCC | mutated in colorectal cancers | Bone and colon |
| | | cancer |
| MET | met proto-oncogene (hepatocyte growth | HBM Bone |
| | factor receptor) | |
| MYBL1 | v-myb myeloblastosis viral oncogene | HBM Bone |
| | homolog (avian)-like 1 | |
| MYC | v-myc avian myelocytomatosis viral | Bone and colon |
| | oncogene homolog | cancer |
| Enos | nitric oxide synthase 3 (endothelial cell) | Load responsive |
| | | genes |
| OSMR | oncostatin M receptor | HBM Bone |
| PDGFRA | platelet-derived growth factor receptor, | HBM Bone |
| | alpha polypeptide | |
| PTGS2/COX-2 | prostaglandin-endoperoxide synthase 2 | Load responsive |
| | (prostaglandin G/H synthase and | genes |
| | cyclooxygenase) | |
| SFRP1 | secreted frizzled-related protein 1 | HBM Bone |
| SFRP4 | secreted frizzled-related protein 4 | HBM Bone |
| SPARC | sparc/osteonectin, cwcv and kazal-like | Inflammation in |
| | domains proteoglycan (testican) | bone |
| STAT1 | signal transducer and activator of | Inflammation in |
| | transcription 1, 91 kD | bone |
| TGFBR2 | transforming growth factor, beta | Inflammation in |
| | receptor II (70-80 kD) | bone |
| THBS1 | thrombospondin 1 | HBM Bone |
| TIMP2 | tissue inhibitor of metalloproteinase 2 | HBM Bone |
| TIMP3 | tissue inhibitor of metalloproteinase 3 | HBM Bone |
| | (Sorsby fundus dystrophy, | |
| | pseudoinflammatory) | |
| TNF | tumor necrosis factor (TNF superfamily, | Osteoclast activity |
| | member 2) | |
| TNFRSF10B | tumor necrosis factor receptor | Inflammation in |
| | superfamily, member 10b | bone |
| TNFRSF11B/OPG | tumor necrosis factor receptor | Osteoclast activity |
| | superfamily, member 11b | |
| | (osteoprotegerin) | |
| TNFSF11/RANKL | tumor necrosis factor (ligand) | Osteoclast activity |
| | superfamily, member 11 | |
| UNK_D83402 | prostaglandin I2 (prostacyclin) synthase | HBM Bone |
| VCAM1 | Vascular cell adhesion molecule 1 | Inflammation in |
| | | bone |
| WISP2 | WNT1 inducible signaling pathway | HBM Bone |
| | protein 2 | |
| WNT10B | wingless-type MMTV integration site | Bone and colon |
| | family, member 10B | cancer |
| WNT6 | wingless-type MMTV integration site | HBM Bone |
| | family, member 6 | |

Preferably, the nucleic acid arrays would contain two or more sequences corresponding to genes observed to express in “HBM Bone”. Such arrays could comprise at least 2, 3, 4, 5, 10, 15, 20, 25, 30 or more (and any integer value in between) of the sequences that are up- or down-regulated in response to bone load listed in the tables, examples or FIG. 16. Similarly protein/antibody arrays can be prepared that are high bone mass specific that comprise proteins, peptides, and/or immunoglobulins that bind to at least 2, 3, 4, 5, 10, 15, 20, 25, 30 or more (and any integer value in between) of the proteins listed in Table 5 or to any of the proteins involved in any of the pathways discussed herein.

5.1.2 DNA Microarray Construction

Frequently, it is desirable to amplify the nucleic acid sample prior to hybridization. Suitable amplification methods include, but are not limited to polymerase chain reaction (PCR) (Innis, et al., PCR PROTOCOLS. A GUIDE TO METHODS AND APPLICATION. ACADEMIC PRESS, Inc. San Diego, (1990)), ligase chain reaction (LCR) (see Wu et al., Genomics, 4: 560 (1989); Landegren et al., Science, 241: 1077 (1988); and Barringer et al., Gene, 89: 117 (1990)), transcription amplification (Kwoh et al., Proc. Natl. Acad. Sci. USA 86: 1173 (1989)), and self-sustained sequence replication (Guatelli et al., Proc. Nat. Acad. Sci. USA, 87: 1874 (1990)).

In a preferred embodiment, the hybridized nucleic acids are detected by detecting one or more labels attached to the sample nucleic acids. The labels may be incorporated by any of a number of means well known in the art. However, preferably the label is simultaneously incorporated during the amplification step in the preparation of the sample nucleic acids. Thus, for example, polymerase chain reaction (PCR) with labeled primers or labeled nucleotides will provide a labeled amplification product. In a preferred embodiment, transcription amplification, as described above, using a labeled nucleotide (e.g., fluorescein-labeled UTP and/or CTP) incorporates a label into the transcribed nucleic acids.

Alternatively, a label may be added directly to the original nucleic acid sample (e.g., mRNA, polyA mRNA, cDNA, and the like) or to the amplification product after the amplification is completed. Means of attaching labels to nucleic acids are well known to those of skill in the art and include, for example nick translation or end-labeling (e.g., with a labeled RNA) by the addition of a kinase to the reaction mixture containing the nucleic acid and subsequent attachment (ligation) of a nucleic acid linker joining the sample nucleic acid to a label (e.g., a fluorophore).

Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, Texas red, rhodamine, green fluorescent protein, and the like), radiolabels (e.g., ³H, ₁₂₅I, ³⁵S, ¹⁴C, or ³²P), enzymes (e.g., horse radish peroxidase (HRP), alkaline phosphatase and others commonly used in an ELISA), and calorimetric labels such as colloidal gold or colored glass or plastic (e.g., polystyrene, polypropylene, latex, and the like) beads. Patents teaching the use of such labels include U.S. Pat. Nos. 3,817,837; 3,850,752; 3,939,350; 3,996,345; 4,277,437; 4,275,149; and 4,366,241.

The reference sequences derived from other the genes, such as, for example, COX-2, can vary widely from a full-length genome, to an individual chromosome, episome, gene, component of a gene, such as an exon or regulatory sequences, to a few nucleotides. A reference sequence of between about 2, 5, 10, 20, 50, 100, 500, 1000, 5,000 or 10,000, 20,000 or 100,000 nucleotides (and any integer value in between) is common. Sometimes only particular regions of a sequence are of interest.

The methods of this invention employ oligonucleotide arrays, which comprise probes exhibiting complementarity to one or more selected reference sequences whose sequence is known (e.g., eNOS, COX-2, Jun, Fox, Connexin 43, SFRP or any of the other genes discussed herein). Typically, these arrays are immobilized in a high density array (“DNA on chip”) on a solid surface, as described for example in U.S. Pat. No. 5,143,854 and PCT patent publication Nos. WO 90/15070, WO 92/10092 and WO 95/11995, each of which is incorporated herein by reference.

Various strategies are available to order and display the oligonucleotide probe arrays on the chip and thereby maximize the hybridization pattern and sequence information derivable regarding the target nucleic acid. Exemplary display and ordering strategies are described in PCT No. WO 94/12305, incorporated herein by reference. For the purposes of fuller description, a brief description of the basic strategy is described below.

The basic tiling strategy provides an array of immobilized probes for analysis of target sequences showing a high degree of sequence identity to one or more selected reference sequences. The strategy is illustrated for an array that is subdivided into four probe sets, although it will be apparent that satisfactory results are obtained from one probe set (i.e., a probe set complementary to the reference sequence as described earlier).

A first probe set comprises a plurality of probes exhibiting perfect complementarity with a selected reference sequence. The perfect complementarity usually exists throughout the length of the probe. However, probes having a segment or segments of perfect complementarity that is/are flanked by leading or trailing sequences lacking complementarity to the reference sequence can also be used. Within a segment of complementarity, each probe in the first probe set has at least one interrogation position that corresponds to a nucleotide in the reference sequence. That is, the interrogation position is aligned with the corresponding nucleotide in the reference sequence, when the probe and reference sequence are aligned to maximize complementarity between the two. If a probe has more than one interrogation position, each corresponds with a respective nucleotide in the reference sequence. The identity of an interrogation position and corresponding nucleotide in a particular probe in the first probe set cannot be determined simply by inspection of the probe in the first set. As will become apparent, an interrogation position and corresponding nucleotide is defined by the comparative structures of probes in the first probe set and corresponding probes from additional probe sets.

In principle, a probe could have an interrogation position at each position in the segment complementary to the reference sequence. Sometimes, interrogation positions provide more accurate data when located away from the ends of a segment of complementarity. Thus, typically a probe having a segment of complementarity of length “x” does not contain more than “x−2” interrogation positions. Since probes are typically 9-21 nucleotides, and usually all of a probe is complementary, a probe typically has 1-19 interrogation positions. Often the probes contain a single interrogation position, at or near the center of probe.

For each probe in the first set, there are, for purposes of the present illustration, up to three corresponding probes from three additional probe sets. Thus, there are four probes corresponding to each nucleotide of interest in the reference sequence. Each of the four corresponding probes has an interrogation position aligned with that nucleotide of interest. Usually, the probes from the three additional probe sets are identical to the corresponding probe from the first probe set with one exception. The exception is that at least one (and often only one) interrogation position, which occurs in the same position in each of the four corresponding probes from the four probe sets, is occupied by a different nucleotide in the four probe sets. For example, for an adenine (A) nucleotide in the reference sequence, the corresponding probe from the first probe set has its interrogation position occupied by a thymine (T), and the corresponding probes from the additional three probe sets have their respective interrogation positions occupied by adenine (A), cytosine (C), or guanine (G), a different nucleotide in each probe. Of course, if a probe from the first probe set comprises trailing or flanking sequences lacking complementarity to the reference sequences, these sequences need not be present in corresponding probes from the three additional sets. Likewise corresponding probes from the three additional sets can contain leading or trailing sequences outside the segment of complementarity that are not present in the corresponding probe from the first probe set. Occasionally, the probes from the additional three-probe set are identical (with the exception of interrogation position(s)) to a contiguous subsequence of the full complementary segment of the corresponding probe from the first probe set. In this case, the subsequence includes the interrogation position and usually differs from the full-length probe only in the omission of one or both terminal nucleotides from the termini of a segment of complementarity. That is, if a probe from the first probe set has a segment of complementarity of length “n”, corresponding probes from the other sets will usually include a subsequence of the segment of at least length “n−2”. Thus, the subsequence is usually at least 3, 4, 7, 9, 15, 21, or 25 nucleotides long (and any by the omission of a 3′ base complementary to the reference sequence and the acquisition of a 5′ base complementary to the reference sequence.

The number of probes on the chip can be quite large (e.g., 10⁵-10⁶). However, often only a relatively small proportion (i.e., less than about 50%, 25%, 10%, 5% or 1%) of the total number of probes of a given length are selected to pursue a particular tiling strategy, in this case a tiling strategy that would reflect bone load gene expression profiles and bone load gene enhancement expression profiles. For example, a complete set of octomer probes comprises 65,536 probes; thus, an array of the invention typically has fewer than 32,768 octomer probes. A complete array of decamer probes comprises 1,048,576 probes; thus, an array of the invention typically has fewer than about 500,000 decamer probes. Often arrays have a lower limit of 25, 50 or 100 probes and as many probes as 10⁴, 10⁵, 10⁶, 10⁷, 10⁸, 10⁹, 10¹⁰, etc. probes. The arrays can have other components besides the probes such as linkers attaching the probes to a support.

Some advantages of using only a proportion of all possible probes of a given length include: (i) each position in the array is highly informative, whether or not hybridization occurs; (ii) nonspecific hybridization is minimized; (iii) it is straightforward to correlate hybridization differences with sequence differences, particularly with reference to the hybridization pattern of a known standard; and (iv) the ability to address each probe independently during synthesis, using high resolution photolithography, allows the array to be designed and optimized for any sequence. For example the length of any probe can be varied independently of the others.

Although the array of probes is usually laid down in rows and columns as described above, such a physical arrangement of probes on the chip is not essential. Provided that the spatial location of each probe in an array is known, the data from the probes can be collected and processed to yield the sequence of a target irrespective of the physical arrangement of the probes on a chip. In processing the data, the hybridization signals from the respective probes can be reasserted into any conceptual array desired for subsequent data reduction whatever the physical arrangement of probes on the chip.

A range of lengths of probes can be employed in the chips. As noted above, a probe may consist exclusively of complementary segments, or may have one or more complementary segments juxtaposed by flanking, trailing and/or intervening segments. In the latter situation, the total length of complementary segment(s) is more important than the length of the probe. In functional terms, the complementary segment(s) of the first probe set should be sufficiently long to allow the probe to hybridize detectably more strongly to a reference sequence compared with a variant of the reference including a single base mutation at the nucleotide corresponding to the interrogation position of the probe. Similarly, the complementary segment(s) in corresponding probes from additional probe sets should be sufficiently long to allow a probe to hybridize detectably more strongly to a variant of the reference sequence having a single nucleotide substitution at the interrogation position relative to the reference sequence. A probe usually has a single complementary segment having a length of at least 3 nucleotides, and more usually at least 5, 6, 7, 8, 9, 10, 11, 12, 13, 14, 15, 16, 17, 18, 19, 20, 21, 22, 23, 24, 25 or 30 or more bases exhibiting perfect complementarity (other than possibly at the interrogation position(s) depending on the probe set) to the reference sequence.

In some chips, all probes are the same length. Other chips employ different groups of probe sets, in which case the probes are of the same size within a group, but differ between different groups. For example, some chips have one group comprising four sets of probes as described above in which all the probes are 15-mers, together with a second group comprising four sets of probes in which all of the probes are 20-mers. Of course, additional groups of probes can be added. Thus, some chips contain, e.g., four groups of probes having sizes of 15-mers, 20-mers, 26-mers and 30-mers. Other chips have different size probes within the same group of four probes. In these chips, the probes in the first set can vary in length independently of each other. Probes in the other sets are usually the same length as the probe occupying the same column from the first set. However, occasionally different lengths of probes can be included at the same column position in the four lanes. The different length probes are included to equalize hybridization signals from probes depending on the hybridization stability of the oligonucleotide probe at the pH, temperature, and ionic conditions of the reaction.

The length of a probe can be important in distinguishing between a perfectly matched probe and probes showing a single-base mismatch with the target sequence. The discrimination is usually greater for short probes. Shorter probes are usually also less susceptible to formation of secondary structures. However, the absolute amount of target sequence bound, and hence the signal, is greater for larger probes. The probe length representing the optimum compromise between these competing considerations may vary depending on, e.g., the GC content of a particular region of the target DNA sequence, secondary structure, synthesis efficiency and cross-hybridization. In some regions of the target, depending on hybridization conditions, short probes (e.g., 11-mers) may provide information that is inaccessible from longer probes (e.g., 19-mers) and vice versa. Maximum sequence information can be achieved by including several groups of different sized probes on the chip as noted above. However, for many regions of the target sequence, such a strategy provides redundant information in that the same sequence is read multiple times from the different groups of probes. Equivalent information can be obtained from a single group of different sized probes in which the sizes are selected to maximize readable sequence at particular regions of the target sequence.

5.2 Protein Arrays

The two major types or protein arrays are primary phase arrays (i.e., antibodies, antibody fragments, immunoglobulins or peptides are affixed to a substrate) and reverse phase arrays (i.e., cell lysate is affixed to a substrate and then subsequently screened with, for example, antibodies). These protein arrays can be utilized to rapidly screen for agents that modulate the Wnt pathway, agents which enhance Wnt pathway activity, bone protein expression in response to different stimuli, determination of additional proteins expressed in bone in response to different stimuli and the like.

5.2.1 Primary Phase Array

One preferred method is a primary phase protein array comprising one or more (and preferably more than one) antibody, antibody fragment, immunoglobulin which recognizes and binds to a protein of the genes listed in any of the Tables, or peptide which recognizes and binds to a protein of the genes listed in any of the Tables. Therefore, in one aspect, an array is contemplated wherein there is 1, 2, 3, 4, 5, 6, 7, 8, 9, 10 or more antibodies, immunogenic fragments thereof or immunoglobulin polypeptides with immunogenic activity to a protein/polypeptide of interest, or other peptide which can recognize and bind to a protein/polypeptide of interest or any combination thereof adhered to a suitable substrate. Cell lysates are then placed in contact with the primary phase array under suitable conditions and detection of antibodies to which a ligand are bound are determined by methods known in the art. See, e.g., MacBeath, Nat. Genet. Suppl. 32: 526-32 (2002).

Primary phase arrays (also known as protein-detecting microarrays) can comprise many different affinity reagents arrayed at high spatial density on a solid support. Each agent captures its target protein or polypeptide from a complex mixture, such as serum, cell culture fluid or a cell lysate. The capture proteins are then subsequently detected and quantified. The primary phase arrays can come in the form of a sandwich array (i.e., capture immunoglobulins are peptides immobilized on the solid support, and bound proteins are detected using second labeled detection antibodies) or antigen capture arrays (i.e., proteins are similarly captured by immobilized antibodies but the captured proteins are detected directly usually by chemically labeling the complex mixture of proteins before applying them to the array). For discussion, see MacBeath, (2002) and the references cited therein.

In a preferred embodiment, the protein immobilized on each patch is an antibody or antibody fragment. The antibodies or antibody fragments of the array may optionally be single-chain Fvs (scFvs), Fab fragments, Fab′ fragments, F(ab′)₂ fragments, Fv fragments, dsFvs diabodies, Fd fragments, full-length, antigen-specific polyclonal antibodies, or full-length monoclonal antibodies. In a preferred embodiment, the immobilized proteins on the patches of the array are monoclonal antibodies, Fab fragments, or scFvs.

The antibodies or antibody fragments are ones that recognize and bind to any of the proteins (1) up- or down-regulated in response to bone load, (2) Wnt pathway proteins, (3) Wnt pathway proteins that are up- or down-regulated in response to addition of Wnt pathway agonists or antagonists, (4) proteins expressed in response to bone load stimuli and/or agonist/antagonist stimuli in HBM TG animals or HBM cell lines or (5) any proteins listed in the tables discussing up- and down-regulated genes/proteins. More preferably, the antibodies or fragments thereof are ones that recognize proteins that are up-regulated or down-regulated in response to enhanced Wnt pathway activity. Antibodies to down-regulated proteins preferably can either detect the presence of the protein down-regulated or can detect, for example, differences in phosphorlylation patterns and thereby active state of the protein (e.g., phosphorylation pattern of GSK-3).

Preferably these immunoglobulin arrays comprise immunoglobulins that recognize 1, 2, 3, 4, 5, 6, 7, 8, 9, 10, 15, 20, 25, 30, 35, 40, 45, 50, and 100 or more (any integer value inbetween) proteins which are up- or down-regulated under the various conditions described herein (e.g., application of load, an agent which enhances load, and the like). Thus, such arrays may comprise 1, 2, 3, 4, 5, 6, 7, 8, 9, 10, 15 or more immunoglobulins that recognize each of the proteins being detected from the cell lysates, cell culture liquid or serum, or cell fractions (e.g., nuclear versus cytoplasmic fractions). Antibodies or fragments thereof, immunoglobulins, or protein recognizing peptides or other moieties as discussed herein optimally recognize or bind to any of the proteins mentioned in the gene expression profiles or gene expression signatures discussed herein. The antibodies can be spotted onto the array substrate using poly-L-lysine or other linker agent. See for details Sreekumar et al., Cancer Res. 61: 7585-93 (2001). Antibody microarrays are known in the art. See for example Silzel et al., (Clin. Chem. 44: 2036-43 (1998)) wherein a sandwich microarray style was used.

Antibody and peptide arrays typically are prepared using inkjet printer technology, wherein the printer spots the monoclonal antibodies on to a substrate forming spots of a specified amount (e.g., 200 μM). Alternatively, antibody arrays can be prepared in a 3×3 pattern using a 96-well polystyrene microtiter plate to monitor the production of protein in cells. For additional methods of spotting arrays, see e.g., Moody et al., Biotechniques 31: 186-194 (2001); Huang et al., Anal. Biochem. 294: 55-62 (2001); Wiese et al., Clin. Chem. 47: 1451-7 (2001); Jenison et al., Clin. Chem. 47: 1894-1900 (2001); Tam et al., J. Immunol. Methods 261: 157-165 (2002); and Schweitzer et al., Nature Biotechnol. 20: 359-65 (2002).

5.2.2 Reverse Phase Array

In another aspect, use of a reverse phase array (also known as a direct array) is contemplated, wherein lysates of bone cells are adhered to a suitable cell surface and then screened for the presence or absence of proteins using immunoglobulins or other agents conjugated to a detectable tag. The bone cells can be from cell cultures or from mice such as transgenic mice expressing HBM, human LRP5, human LRP6, combined knock-out and knock-ins of same animal genes of LRP5 and LRP6 (both alone and in combination) or the non-TG litter mates. Other cells lines may be transiently transfected cell lines which have been transfected with a nucleic acid which expresses the HBM protein, LRP5, LRP6, or other Wnt pathway proteins. The reverse phase lysate arrays are miniaturized dot-blots of lysate on a substrate capable of being screened. The number of spots per substrate will vary depending on manner in which the lysate is to be screened. For additional discussion, see for example Sreekumar et al., Cancer Res. 61: 7585-93 (2001).

Once the lysate is affixed to the substrate it can be screened with a detectable ligand, such as an antibody, an RNA (if the protein is known to bind RNA), a DNA (if the protein is known to bind DNA), a peptide (which is known to interact with the protein), another protein, and the like, wherein each of these moieties can have a detectable label attached.

In another aspect of the invention, combination of lysates from the above types of cells can be placed on the array substrate. For example, lysates from animals to which bone load stimuli and/or Wnt pathway modulators have been administered can be combined with lysates from cell cultures. The cell culture lysates can be of cells to which mechanical load has been administered, or not. It can be of cell cultures to which Wnt pathway modulators and load have been administered or any combination of cell lysates. Such arrays can be used for rapid screening of the proteins expressed in response to load and/or compound candidates that modulate the Wnt pathway and thereby bone remodeling.

5.2.3 Apparatus for Protein Arrays

For either style of array, a detectable label such as a radioisotope, chromophore, fluorophore, or chemiluminescent species, can be attached to the detection moiety (e.g., secondary detection antibody, peptide, and the like). The detection moiety is then incubated with the microchip under suitable conditions to allow binding to the primary antibody or antigen.

After the excess probe protein is washed away, the chip surface is analyzed for signal from the label. Detection of a signal indicates interaction of the labeled protein with one or more unique members of the protein library. The identity of proteins that are able to bind to the probe protein or other probe moiety can then be determined from the location of the spots on the chip (if using a primary array) or by the detectable label and associated antibody if using a reverse phase array. Other methods can be used to detect protein-protein, protein-ligand, or protein-nucleic acid interactions. For example, when the solid surface used to form the protein array is a gold layer, surface plasmon resonance (SPR) can be used to detect mass changes at the surface. When gold surfaces are employed, the reactive moiety on the oligonucleotide capture probe is a thiol group (rather than an amino group) and the gold surface need not be functionalized to achieve capture probe attachment. Mass spectrometry (especially, MALDI-TOF) can also be used to analyze species bound to unique members of the protein library.

In another embodiment, the present invention also provides a protein-coated substrate (e.g., antibody coated substrate) comprising a plurality of patches arranged in discrete, known regions on a substrate (if using a primary array), where each of the patches comprises an immobilized protein with a different, known sequence and where each of the patches is separated from neighboring patches by from about 50 nm to about 500 μm. In a preferred embodiment, the protein-coated substrate comprises 9 or more patches.

Biosensors, micromachined devices, and medical devices that contain the protein-coated substrate comprising a plurality of patches arranged in discrete, known regions on a substrate, where each of the patches comprises an immobilized protein with a different, known sequence and where each of the patches is separated from neighboring patches by from about 50 nm to about 500 μm are also contemplated.

Alternatively, the different patches can be designated regions of lysates to be screened using different antibodies, with each patch being one of each of the different cell lysates (e.g., control, in vivo samples, in vitro samples, bone load, bone load with known Wnt pathway agonist, and the like) of interest to be screened. Thus a patch could have a cell lysate of 1, 2, 3, 4, 5, 6, 7, 8, 9, 10 or more different sets of experiments, with multiple patches per array substrate.

In one embodiment, the array of proteins comprises a plurality of patches, preferably 9 or more, arranged in discrete known regions on a substrate, wherein each of the patches comprises an immobilized protein with a different, known sequence and wherein each of the patches is separated from neighboring patches by from about 50 nm to about 500 μm. In a preferred embodiment, the patches are separated from neighboring patches from about 200 nm to about 500 μm.

In some versions of the array, the diameter of each of the patches is proportional to the distance separating the patches. Therefore, the area of each patch may be from about 100 nm² to about 40,000 μm². Each patch preferably has an area from about 1 μm² to about 10,000 μm².

In one embodiment of the array, the array comprises 9 or more patches within a total area of about 1 cm². In preferred embodiments of the array, the array comprises 100 or more patches within a total area of 1 cm². In another embodiment, the array comprises 10³ or more patches within a total area of 1 cm².

In one embodiment of the array, the protein immobilized on one patch differs from the protein immobilized on a second patch of the same array. For example, an antibody to one phosphorylated form of GSK-3 next to an antibody to a different phosphorylated form of GSK-3 (if using a primary protein array).

In an alternative embodiment of the invention array, the proteins on different patches are identical. These can serve as useful control regions.

The substrate of the array may be either organic or inorganic, biological or non-biological, or any combination of these materials. In one embodiment, the substrate is transparent or translucent. The portion of the surface of the substrate on which the patches reside is preferably flat and firm or semi-firm.

Numerous materials are suitable for use as a substrate in the array embodiment of the invention. For instance, the substrate of the invention array can comprise a material selected from a group consisting of silicon, silica, quartz, glass, controlled pore glass, carbon, alumina, titanium dioxide, germanium, silicon nitride, zeolites, and gallium arsenide. Many metals such as gold, platinum, aluminum copper, titanium, and their alloys are also options for array substrates. In addition, many ceramics and polymers may also be used as substrates. Polymers which may be used as substrates include, but are not limited to, the following: polystyrene; poly(tetra)fluorethylene; (poly)vinylidenedifluoride; polycarbonate; polymethylmethacrylate; polyvinylethylene; polyethyleneimine; poly(etherether)ketone; polyoxymethylene (POM); polyvinylphenol; polylactides; polymethacrylimide (PM); polyalkenesulfone (PAS); polyhydroxyethylmethacrylate; polydimethylsiloxane; polyacrylamide; polyimide; co-block-polymers; and Eupergit™, Photoresists, polymerized Langmuir-Blodgett films, and LIGA structures may also serve as substrates in the present invention. The preferred substrates for the array comprise silicon, silica, glass, or a polymer.

In a preferred embodiment of the invention array, the patches further comprise a monolayer on the surface of the substrate and the proteins of the patches are immobilized on the monolayer. The monolayer is preferably a self-assembling monolayer. This monolayer may optionally comprise molecules of the formula X—R—Y, wherein R is a spacer, X is a functional group that binds R to the surface, and Y is a functional group for binding proteins onto the monolayer.

A variety of chemical moieties may function as monolayers in the array. However, three major classes of monolayer formation are preferably used to expose high densities of bioreactive omega-functionalities on the patches of the array: (i) alkylsiloxane monolayers (“silanes”) on hydroxylated surfaces; (ii) alkylthiol/dialkyldisulfide monolayers on noble metals (preferably Au(111)); and (iii) alkyl monolayer formation on oxide-free passivated. One of ordinary skill in the art will recognize that many possible moieties may be substituted for X, R, and/or Y, dependent primarily upon the choice of substrate, coating, and affinity tag. Many examples of monolayers are described in Ulman, AN INTRODUCTION TO ULTRATHIN ORGANIC FILMS: FROM LANGMUIR-BLODGETT TO SELF ASSEMBLY (Academic Press, 1991).

Deposition or formation of the coating (if present) on the substrate is done prior to the formation of patches of bioreactive monolayers thereon. Monolayer-compatible surface patches may optionally be fabricated using photolithography, micromolding (PCT Publication WO 96/29629), wet chemical etching, or any combination of these. Bio-reactive monolayers are then formed on the patches. Alternatively, arrays of bioreactive-monolayer-functionalized surface patches can be created by microstamping (see e.g., U.S. Pat. Nos. 5,512,131 and 5,731,152) or microcontact printing (μCP) (see e.g., PCT Publication WO 96/29629). Subsequent immobilization of biomolecules results in two-dimensional protein arrays. Inkjet chemical dispensers provide another option for patterning monolayer X—R—Y molecules or components thereof to nanometer or micrometer scale sites on the surface of the substrate or coating (see e.g., Lemmo et al., Anal Chem. 69: 543-551 (1997)).

Diffusion boundaries between the patches may be integrated as topographic patterns or surface functionalities with orthogonal wetting behavior. For instance, walls of substrate material or photoresist may be used to separate some of the patches from some of the others or all of the patches from each other. In a preferred embodiment, the patches are separated from each other by surfaces free of monolayers of the form X—R—Y. Alternatively, non-bioreactive monolayers with different wettability may be used to separate patches from one another.

In another preferred embodiment of the invention, the proteins immobilized to each patch of the array are protein-capture agents.

In an alternative embodiment of the invention array, the proteins on different patches are identical.

For additional information of how protein arrays can be prepared, see e.g., U.S. Pat. Nos. 6,475,808; 6,537,749; 6,495,314; 6,406,921 and 6,406,840. See also, PROTEINS AND PROTEOMICS: A LABORATORY MANUAL (Richard J. Simpson, ed., Cold Spring Harbor Laboratory Press 2002).

7. Agents which Modulate Bone Density

Agents which modulate bone density via the canonical Wnt pathway include but are not limited to small compounds, interfering RNAs, antisense nucleic acids, polypeptides, aptamers, immunoglobulins, and protein mimetics. These compounds can be used as research reagents to further analyze bone load responses and enhancement thereof, as well as means of modulating bone density in a subject. Preferably these compounds are used to activate the Wnt pathway, thereby enhancing bone mineralization in a subject in need thereof, such as an individual with osteoporosis.

7.1 Small Compounds

Small compounds can be used as controls to develop gene expression profiles for studying bone load. The small compounds can also be used to treat bone mineralization disorders involving the Wnt pathway. The small compounds can be used to modulate β-catenin, GSK-3, Wnt (e.g. Wnt 3A), LRP5 (or LRP6) and any of the proteins that are expressed in response to bone load or in the Wnt pathway.

7.1.1 GSK-3 Inhibitors

Glycogen synthase kinase-3 (GSK-3) is a multifunctional serine/threonine kinase found in all eukaryotes. When GSK-3 was first identified, it was shown to phosphorylate the enzyme glycogen synthase, thereby inactivating it. The activity of GSK-3 is modulated by the degree by which GSK-3 is phosphorylated. Reduced phosphorylation results in increased GSK-3 activity. Today, GSK-3 has been implicated in the development of diabetes, Alzheimer's disease, bipolar disorder and cancer. GSK-3 has also been indicated to be an important mediator of hypoxia-induced apoptosis via activation of the mitochondrial death pathway (Loberg et al., J. Biol. Chem. 277(44): 41667-73 (2002).

GSK-3 is modulated by phosphoinositide 3-kinase, the kinase responsible for phosphorylating GSK-3 and thereby inactivating the protein.

A well known GSK-3 inhibitor is LiCl. However, LiCl is not selective, regulating many proteins not just GSK-3 and therefore is less preferred. Selective GSK inhibitors and agonists are preferred that modulate GSK protein activity and not other proteins. More preferred are GSK inhibitors or agonists that are selective for GSK-3 and not other GSK proteins. Most preferred, are GSK inhibitors or agonists that can distinguish (are selective between) for a specific GSK-3 isoform (i.e., GSK-3α or GSK-3β). Selective GSK-3 inhibitors include aloisine A, amiloride (an inhibitor of Na⁺, H⁺ antiporters), and maleimide compounds.

Aloisine A is highly selective for CDK1/cyclin B, CDK2/cycline A-E, CDK25/p25 and both GSK-3 isoforms. It appears to act by interacting with the ATP-binding pocket and inhibits cell proliferation (Mettey et al., J. Med. Chem. 46(2): 222-36 (2003)).

In particular, the compounds of the subject invention include a series of pyrazolo[3,4-b]pyrid[az]ines that have been identified that are potent inhibitors of GSK-3. These pyrazolo[3,4-b]pyrid[az]ines are of the following formula: 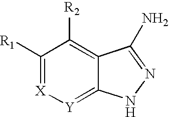
Automated ligand dockeing of the pyridazine derivatives into a GSK-3α homology model suggested an interaction with the ATP binding site.

Also contemplated for use herein are maleimide derivatives as described in WO 00/38675 (SmithKline Beecham), incorporated by reference in its entirety.

As taught in WO 00/38675, published Patents and Patent Applications, EP 470490 (Roche), WO 93/18766 (Wellcome), WO 93/18765 (Wellcome), EP 397060 (Goedecke), WO 98/11105 (Astra), WO 98/11103 (Astra), WO 98/11102 (Astra), WO 98/04552 (Roche), WO 98/04551 (Roche), DE 4243321 (Goedecke), DE 4005970 (Boehringer), DE 3914764 (Goedecke), WO 96/04906 (Wellcome), WO 95/07910 (Wellcome), DE 4217964 (Goedecke), U.S. Pat. No. 5,856,517 (Roche), U.S. Pat. No. 5,891,901 (Roche), and WO 99/42100 (Sagami) (which patents and patent applications are hereinafter also referred to as the “Publications of Group (IA)”) disclose certain bisindole maleimides, indole aryl maleimides, and indolocarbazoles (hereinafter also referred to as the “Compounds of Group (IA)”) and methods for their preparation.

Published Patents and Patent Applications EP 328026 (Roche), EP 384349 (Roche), EP 540956 (Roche), and DE 4005969 (Boehringer) (which patents and patent applications are hereinafter also referred to as the “Publications of Group (IB)”) disclose certain bisindole maleimides, indole aryl maleimides, and indolocarbazoles (hereinafter also referred to as the “Compounds of Group (IB)”) and methods for their preparation.

Published Patent Application EP 508792 (Schering) (which patent application is hereinafter also referred to as the “Publication of Group (IC)”) discloses certain maleimide derivatives (hereinafter also referred to as the “Compounds of Group (IC)”) and methods for their preparation.

The group of publications consisting of the “Publications of Group (IA)”, the “Publications of Group (IB)”, and the “Publications of Group (IC)” is hereinafter referred to as the “Publications of Group (I)”.

The group of compounds consisting of the “Compounds of Group (IA)”, the “Compounds of Group (B)”, and the “Compounds of Group (IC)” is hereinafter referred to as the “Compounds of Group (I)”.

Published Patents and Patent Applications WO 95/17182 (Lilly), WO 95/35294 (Lilly), EP 624586 (Roche), EP 657458 (Lilly), EP 776899 (Lilly), EP 805158 (Lilly), U.S. Pat. No. 5,491,242 (Lilly), U.S. Pat. No. 5,541,347 (Lilly), U.S. Pat. No. 5,545,636 (Lilly), U.S. Pat. No. 5,552,396 (Lilly), U.S. Pat. No. 5,624,949 (Lilly), U.S. Pat. No. 5,710,145 (Lilly), U.S. Pat. No. 5,721,272 (Lilly), WO 97/18809 (Lilly), and WO 98/07693 (Lilly) (which patents and patent applications are hereinafter also referred to as the “Publications of Group (II)”) disclose certain compounds (hereinafter also referred to as the “Compounds of Group (II)”) which are selective Protein Kinase C (PKC) beta 1 and PKC beta 2 inhibitors which are stated to be useful in the treatment of conditions associated with diabetes mellitus and complications thereof.

Hers et al., FEBS Letters 460 (1999) 433-436 disclose certain bisindolylinaleimides as inhibitors of GSK-3.

The disclosures of the “Publications of Group (I)” and the “Publications of Group (II)” are incorporated herein by reference.

A series of certain bisindole maleimides, indole aryl maleimides, and indolocarbazoles are particularly potent and selective inhibitors of GSK-3. These compounds are indicated to be useful for the treatment and/or prophylaxis of conditions associated with a need for the inhibition of GSK-3.

Accordingly, in one aspect, the malimide derivatives for use herein are compounds selected from the “Compounds of Group (I)”. A suitable compound selected from the “Compounds of Group (I)” is a compound of formula (I) as respectively defined in EP 470490, WO 93/18766, WO 93/18765, EP 397060, WO 98/11105, WO 98/11103, WO 98/11102, WO 98/04552, WO 98/04551, DE 4243321, DE 4005970, DE 3914764, WO 96/04906, WO 95/07910, DE 4217964, U.S. Pat. No. 5,856,517, U.S. Pat. No. 5,891,901, WO 99/42100, EP 328026, EP 384349, EP 540956, DE 4005969, or EP 508792 (the Publications of Group (I))”.

In particular, a compound selected from the “Compounds of Group (I)” includes a compound selected from those compounds specifically disclosed as examples in the “Publications of Group (I)”.

An example of a compound selected from the “Compounds of Group (I)” is a compound selected from those disclosed in the “Publications of Group (IA)” or the “Publications of Group (B)”, and is of formula (A): 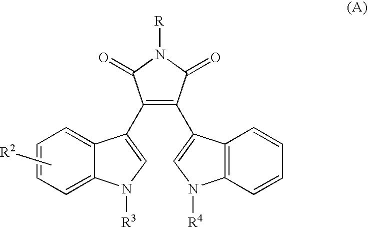
wherein
R is hydrogen;
R² is hydrogen, 5-On-Pr, 5-Ph, 5-CO₂Me or 5-NO₂;
R³ is Me or (CH₂)₃OH, and;
R⁴ is Me, n-Pr, —(CH₂)₃X wherein X is selected from CN, NH₂, CO₂H, CONH₂, or OH.

A further example of a compound selected from the “Compounds of Group (I)” is a compound selected from those disclosed in the “Publications of Group (IB)” and is of formula (B): 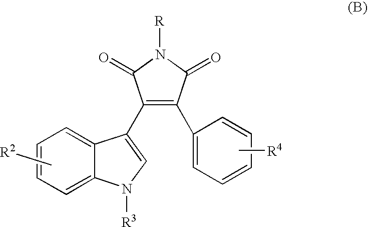
wherein
R is hydrogen;
R² is hydrogen;
R³ is Me or a group —(CH₂)₃Y wherein Y is NH₂ or OH, and;
R⁴ is 2-Cl or 2,4-di-Cl.

Yet a further example of a compound selected from the “Compounds of Group (I)” is a compound selected from those disclosed in the “Publications of Group (IC)” and is 9,10,11,12-tetrahydro-10-carboxy-9,12,-epoxy-1H-diindolo[1,2,3-fg:3′,2′,1′-kl]pyrrolo[3,4-i]benzodiazocine-1,3(2H)-dione (formula (C)).

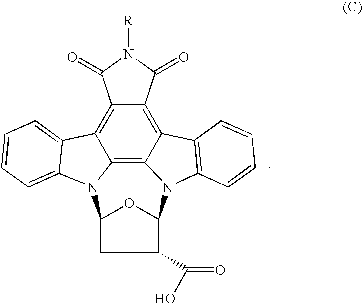

A suitable compound selected from the “Compounds of Group (II)” is a compound of formula (I) as defined in WO 95/17182, WO 95/35294, EP 624586, EP 657458, EP 776899, EP 805158, U.S. Pat. No. 5,491,242, U.S. Pat. No. 5,541,347, U.S. Pat. No. 5,545,636, U.S. Pat. No. 5,552,396, U.S. Pat. No. 5,624,949, U.S. Pat. No. 5,710,145, U.S. Pat. No. 5,721,272, WO 97/18809, or WO 98/07693 (the “Publications of Group (II)”)

In particular, a compound selected from the “Compounds of Group (II)” includes a compound selected from those compounds specifically disclosed as examples in the “Publications of Group (II)”.

Examples of compounds of formula (A) include those on the list below (hereinafter referred to as “List A”):
3,4-bis(1-methyl-3-indolyl)pyrrole-2,5-dione;
3-(1-methyl-3-indolyl)-4-(1-propyl-3-indolyl)pyrrole-2,5-dione;
3-(1-methyl-3-indolyl)-4-(1-[3-cyanopropyl]-3-indolyl)pyrrole-2,5-dione;
3-(1-methyl-3-indolyl)-4-(1-[3-aminopropyl]-3-indolyl)pyrrole-2,5-dione;
3-(1-methyl-3-indolyl)-4-(1-[3-carbamoylpropyl]-3-indolyl)pyrrole-2,5-dione;
3-(1-methyl-5-propyloxy-3-indolyl)-4-(1-[3-aminopropyl]-3-indolyl)pyrrole-2,5-dione;
3-(1-methyl-5-phenyl-3-indolyl)-4-(1-[3-hydroxypropyl]-3-indolyl)pyrrole-2,5-dione;
3-(1-methyl-5-phenyl-3-indolyl)-4-(1-[3-aminopropyl]-3-indolyl)pyrrole-2,5-dione;
3-(1-methyl-5-methoxycarbonyl-3-indolyl)-4-(1-[3-hydroxypropyl]-3-indolyl)pyrrole-2,5-dione;
3-(1-methyl-5-nitro-3-indolyl)-4-(1-[3-hydroxypropyl]-3-indolyl)pyrrole-2,5-dione; and
3-(1-[3-hydroxypropyl]-5-nitro-3-indolyl)-4-(1-methyl-3-indolyl)pyrrole-2,5-dione;
   or a pharmaceutically acceptable derivative thereof.

Examples of compounds of formula (B) include those on the list below (hereinafter referred to as “List B”):
3-(1-methyl-3-indolyl)-4-(2-chlorophenyl)pyrrole-2,5-dione;
3-(1-methyl-3-indolyl)-4-(2,4-dichlorophenyl)pyrrole-2,5-dione;
3-(1-[3-hydroxypropyl)-3-indolyl)-4-(2-chlorophenyl)pyrrole-2,5-dione; and
3-(1-[3-aminopropyl-3-indolyl)-4-(2-chlorophenyl)pyrrole-2,5-dione;
   or a pharmaceutically acceptable derivative thereof.

The example compound of formula (C) is:
10,11,12-tetrahydro-10-carboxy-9,12,-epoxy-1H-diindolo[1,2,3-fg:3′,2′,1′-kl]pyrrolo[3,4-i]benzodiazocine-1,3(2H)-dione, or a pharmaceutically acceptable derivative thereof.

Suitably, a compound selected from the “Compounds of Group (I)” is a compound selected from those disclosed in the “Publications of Group IA)” or the “Publications of Group (IB)” and is of formula (A) as hereinbefore defined.

Suitably, a compound selected from the “Compounds of Group (I)” is a compound selected from those disclosed in the “Publications of Group (IC)” and is of formula (C) as hereinbefore defined.

Favourably, a compound selected from the “Compounds of Group (I)” is a compound of formula (A) selected from “List A”.

Favourably, a compound selected from the “Compounds of Group (I)” is 10,11,12-tetrahydro-10-carboxy-9,12,-epoxy-1H-diindolo[1,2,3-fg:3′,2′,1′-kl]pyrrolo[3,4-i]benzodiazocine-1,3 (2H)-dione or a pharmaceutically acceptable derivative thereof.

Preferably, a compound selected from the “Compounds of Group (I)” is a compound selected from those disclosed in the “Publications of Group (B)” and is of formula (B) as hereinbefore defined.

More preferably, a compound selected from the “Compounds of Group (I)” is a compound of formula (B) selected from “List B”.

Most preferably, a compound selected from the “Compounds of Group (I)” is 3-(1-methyl-3-indolyl)-4-(2,4-dichlorophenyl)pyrrole-2,5-dione.

Certain of the “Compounds of Group (I)” and the “Compounds of Group (II)” may contain at least one chiral atom and/or may contain multiple bonds and hence may exist in one or more stereoisomeric forms.

The present invention encompasses all of the isomeric forms of the “Compounds of Group (I)” and the “Compounds of Group (II)” including enantiomers and geometric isomers whether as individual isomers or as mixtures of isomers, including racemic modifications.

The present invention also includes the pharmacologically active derivatives of the “Compounds of Group (I)” and the “Compounds of Group (II)” as described in the “Publications of Group (I)” and the “Publications of Group (II)” respectively.

Suitable pharmacologically active derivatives of the compounds of the invention include salts and solvates as described in the “Publications of Group (I)” and the “Publications of Group (II)”.

Suitable pharmaceutically acceptable derivatives of the “Compounds of Group (I)” and the “Compounds of Group (II)” include pharmaceutically acceptable salts and pharmaceutically acceptable solvates.

Also contemplated for use herein are maleimide derivatives as described in WO 00/21927 (SmithKline Beecham), incorporated by reference in its entirety.

WO 00/21927 discloses compounds of the following formula (I): 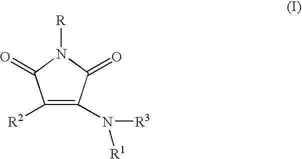
or a pharmaceutically acceptable derivative thereof, wherein:

R is hydrogen, alkyl, aryl, or aralkyl;

R¹ is hydrogen, alkyl, aralkyl, hydroxyalkyl or alkoxyalkyl;

R² is substituted or unsubstituted aryl or substituted or unsubstituted heterocyclyl;

R³ is hydrogen, substituted or unsubstituted alkyl, cycloalkyl, alkoxyalkyl, substituted or unsubstituted aryl, substituted or unsubstituted heterocyclyl or aralkyl wherein the aryl moiety is substituted or unsubstituted; or,

R¹ and R³ together with the nitrogen to which they are attached form a single or fused, optionally substituted, saturated or unsaturated heterocylic ring.

Suitably, R is hydrogen, C₁₋₆ alkyl, such as methyl or ethyl, or R is phenyl or benzyl.

Preferably, R is hydrogen.

Suitably, R¹ is hydrogen, C₁₋₆ alkyl, such as methyl, ethyl, or R¹ is hydroxyethyl or methoxyethyl.

Preferably, R¹ is hydrogen.

When R² is substituted or unsubstituted aryl, examples of aryl groups include phenyl and naphthyl.

When R² is substituted or unsubstituted heterocyclyl, examples of heterocyclyl groups include indolyl, benzofuranyl, thienyl and benzothienyl.

When R² is substituted phenyl, suitable substituents include up to three groups independently selected from halo, C₁₋₆ alkoxy, nitro, perfluoroC₁₋₆ alkyl, benzoyl, C₁₋₆alkoxycarbonyl, C₁₋₆alkylsulphonyl, hydroxy, —O(CH₂)_{w}O—, where w is 1 to 4, phenoxy, benzyloxy, C₁₋₆alkoxy C₁₋₆alkyl, perfluoroC₁₋₆alkoxy, C₁₋₆alkylS—, perfluoroC₁₋₆alkylS—, (diC₁₋₆alkyl)N—, amino, C₁₋₆alkylcarbonylamino, substituted or unsubstituted ureido, phenylcarbonylamino, benzylcarbonylamino, styrylcarbonylamino, (diC₁₋₆alkoxy)(phenyl)C—, C₁₋₆alkyl, and phenyl. Suitable substituents for ureido include fluorophenyl, phenylC₁₋₆alkyl-, cyclohexyl, C₁₋₆alkenyl, C₁₋₆alkyl, and C₁₋₆alkoxyphenyl.

When R² is substituted indolyl, suitable substituents include C₁₋₆alkyl.

When R² is substituted benzothienyl, suitable substituents include C₁₋₆alkyl.

Suitably, R² is substituted or unsubstituted phenyl.

Favourably, R² is phenyl substituted with 4-Cl; 3-Cl; 2-Cl; 2,4-di-Cl; 3,4-di-Cl; 3,5-di-Cl; 2,6-di-Cl; 2-F-6-Cl; 2-F; 3-F; 4-F; 2,3-di-F; 2,5-di-F; 2,6-di-F; 3,4-di-F; 3,5-di-F; 2,3,5-tri-F; 3,4,5-tri-F; 2-Br; 3-Br; 4-Br; 2-I; 4-I; 3-Cl-4-OMe; 3-NO₂-4-Cl; 2-OMe-5-Br; 2-NO₂; 3-NO₂; 4-NO₂; 2-CF₃; 3-CF₃; 4-CF₃; 3,5-di-CF₃; 4-PhC(O)—; 4-MeO(O)C—; 4-MeSO₂—; 4-OH; 2-OMe; 3-OMe; 4-OMe; 2,4-di-OMe; 2,5-di-OMe; 3,4-di-OMe; 3,4-OCH₂O—; 3,4,5-tri-OMe; 3-NO₂-4-OMe; 4-OnBu; 2-OEt; 2-OPh; 3-OPh; 4-OPh; 2-OCH₂Ph; 4-OCH₂Ph; 4-(MeOCH₂); 2-OCF₃; 4-OCF₃; 4-SMe; 3-SCF₃; 4-NMe₂; 3-NH₂; 3-(NHC(O)Me); 3-[NHC(O)NH(3-F-Ph)]; 3-[NHC(O)NH(CH₂)₂Ph]; 3-[NHC(O)NHCyclohexyl]; 3-[NHC(O)NHCH₂CH═CH₂]; 3-[NHC(O)Ph]; 3-[NHC(O)CH₂Ph]; 3-[trans-NHC(O)CH═CHPh]; 3-[NHC(O)nPr]; 3-[NHC(O)NHEt]; 3-[NHC(O)NH(3-OMe-Ph)]; 4-[C(OMe)₂Ph]; 2-Me; 3-Me; 4-Me; 4-iPr; 2,5-di-Me; 3,5-di-Me, 4-Ph, 2,3-[(—CH₂═CH₂—)], or 3,4-[(—CH₂═CH₂—)].

When R³ is alkyl, examples include methyl and ethyl.

When R³ is cycloalkyl, examples include cyclohexyl.

When R³ is alkoxyalkyl, examples include methoxyethyl.

When R³ is aralkyl, examples include benzyl and phenylethyl.

When R³ is substituted or unsubstituted aryl, examples include fluorenyl, phenyl, and dibenzofuryl.

When R³ is substituted or unsubstituted heterocyclyl, examples include thienyl, oxazolyl, benzoxazolyl, pyridyl, and pyrimidinyl.

When R¹ and R³ together with the nitrogen atom to which they are attached form a fused heterocyclic ring, which ring may be unsubstituted or substituted, examples include indolinyl, indolyl, oxindolyl, benzoxazolinonyl, tetrahydroquinolinyl, tetrahydroisoquinolinyl, benzimidazolyl, benzazepinyl, isoindolin-2-yl, and 1,3,3-trimethyl-6-azabicyclo[3,2,1]oct-6-yl.

When R¹ and R³ together with the nitrogen atom to which they are attached form a single heterocyclic ring, which ring may be unsubstituted or substituted, examples include 1-phenyl-1,3,8-triazaspiro-[4,5]-decan-4-one-8-yl, piperazinyl, pyrrolidinyl, piperidinyl, morpholinyl, thiomorpholinyl, and a pyridinium ring.

When R³ is substituted phenyl, suitable substituents include up to three groups independently selected from substituted or unsubstituted C₁₋₆alkyl, phenyl, benzyl, substituted or unsubstituted C₁₋₆alkylS—, halo, hydroxy, substituted or unsubstituted C₁₋₆alkoxy, substituted or unsubstituted phenoxy, indolyl, naphthyl, carboxy, C₁₋₆alkoxycarbonyl, benzyloxy, pentafluorophenoxy, nitro, N-substituted or unsubstituted carbamoyl, substituted or unsubstituted C₁₋₆ alkylcarbonyl, benzoyl, cyano, perfluoroC₁₋₆ alkylSO₂—, C₁₋₆alkylNHSO₂—, oxazolyl, C₁₋₆ alkylcarbonylpiperazinyl, substituted or unsubstituted phenylS—, C₁₋₆ alkylpiperazinyl-, cyclohexyl, adamantyl, trityl, substituted or unsubstituted C₁₋₆ alkenyl, perfluoroC₁₋₆alkyl, perfluoroC₁₋₆ alkoxy, perfluoroC₁₋₆ alkylS—, aminosulphonyl, alkylaminosulphonyl, dialkylaminosulphonyl, arylaminosulphonyl, morpholino, (diC₁₋₆alkyl)amino, C₁₋₆alkylCONH-(diC₁₆alkoxy)phenyl(CH₂)ₙNHC(O)CH(phenyl)S—, where n is 1 to 6, and C₁₋₆alkylCON(C₁₋₆alkyl)-, thiazolidinedionylC₁₋₆alkyl, phenylCH(OH)—, substituted or unsubstituted piperazinylC₁₋₆alkoxy, substituted or unsubstituted benzoylamino; or —[CH═CH—C(O)O]—, —[(CH═CH)₂]—, —[(CH₂)ₓN(C₁₋₆alkylcarbonyl)]-, —(CH₂)ₓ—, —SCH═N—, —SC(C₁₋₆alkyl)═N—, —OCF₂O—, —CH═N—NH—, —CH═CH—NH—, —OC(NHC₁₋₆alkyl)═N—, —OC(O)NH—, —C(O)NC₁₋₆alkylC(O)—, —[CH═CH—CH═N]—, —[CH═C(C₁₋₆alkylcarbonyl)O]—, —C(O)NHC(O)—, —[(CH₂)ₓC(O)]—, —N═N—NH—, —N═C(C₁₋₆alkyl)O—, —O(CH₂)ₓO—, (CH₂)ₓSO₂(CH₂)_{y}—, —N(C₁₋₆ alkylcarbonyl)(CH₂)ₓ—, where x and y are independently 1 to 4, pyrimidin-2-yloxy, phenylamino, N-[pyrimidin-2-yl]-N—[C₁₋₆alkyl]amino, C₁₋₆alkylsulphonylamino, and 1,2,3-thiadiazolyl.

Suitable substituents for C₁₋₆alkyl include hydroxy, carboxy, unsubstituted or N-substituted carbamoyl, N-morpholinylcarbonyl, C₁₋₆alkylaminocarbonyl, fluoro, cyano, C₁₋₆alkyl, C₁₋₆alkoxycarbonylamino, amino, C₁₋₆alkylcarbonylamino, benzoylamino, phenylaminocarbonylamino, C₁₋₆alkoxycarbonyl, phosphono, mono- or bis C₁₋₆alkylphosphonate, C₁₋₆alkylaminosulphonyl, and C₁₋₆alkylcarbonylaminoC₁₋₆alkylaminoCO—.

Suitable substituents for C₁₋₆alkylS— include carboxy, C₁₋₆alkoxycarbonyl, C₁₋₆alkoxyC₁₋₆alkylaminocarbonyl, unsubstituted or N-substituted carbamoyl, and fluoro.

Suitable substituents for C₁₋₆ alkoxy include C₁₋₆ alkoxy, phenyl, carboxy, C₁₋₆alkoxycarbonyl, unsubstituted or N-substituted carbamoyl, and phenyl.

Suitable substituents for carbamoyl include C₁₋₆alkyl, and C₁₋₆alkoxyC₁₋₆ alkyl.

Suitable substituents for C₁₋₆alkylcarbonyl include carboxy, and C₁₋₆ alkoxycarbonyl.

Suitable substituents for phenylS— include chloro, nitro, carboxy, C₁₋₆ alkylaminocarbonyl, unsubstituted or N-substituted carbamoyl, and C₁₋₆ alkoxycarbonyl.

Suitable substituents for C₁₋₆alkenyl include (diC₁₋₆alkyl)aminocarbonyl, carboxy, C₁₋₆alkoxycarbonyl, carbamoyl, and phenyl.

Suitable substituents for piperazinylC₁₋₆alkoxy include methyl.

Suitable substituents for phenoxy include chloro.

Suitable substituents for benzoylamino include hydroxy.

When R³ is substituted benzofuryl, suitable substituents include C₁₋₆ alkylcarbonyl.

When R³ is substituted thienyl, suitable substituents include C₁₋₆ alkylcarbonyl.

When R³ is substituted oxazolyl, suitable substituents include C₁₋₆alkyl

When R³ is substituted benzoxazolyl, suitable substituents include halo.

When R³ is substituted pyridyl, suitable substituents include up to three substituents independently selected from C₁₋₆alkyl, C₁₋₆alkoxy, and halo.

Suitably, R³ is substituted or unsubstituted phenyl.

Favourably, R³ is phenyl substituted with 2-Me; 2-Et 2-iPr; 2-CH₂OH 2-Ph; 2-CH₂Ph; 2-SMe; 2-F; 2-Cl; 2-OH; 2-OMe; 2-OPh; 2-Me-5-F; 2-Me-3-Cl; 2-Me-4-Cl; 2-Me-5-Cl; 2-Me-3-Br; 2,3-di-Me; 2,4-di-Me; 2-Me-4-OH; 2-Me-4-OMe; 2-Me-5-CH₂OH; 2,4,6-tri-Me; 2-(2-indolyl); (1-naphthyl); 2-Me-5-COOH; 2-Me-5-COOMe; 2-OH-5-COOH; 2-[O(CH₂)₂OMe]-5-[(CH₂)₂—,COOH]; 2-[SCH(Ph)CONH(CH₂)₂(3,4-di-OMePh)]; 3-Me; 3-Et; 3-CH₂OH; 3-CH₂OH-6-Me; 3-CH₂OH-4-OMe; 3-(CH₂NMe₂)-4-OMe; 3-[CH₂COOH]; 3-[CH₂COOMe]; 3-[CH₂CONH₂]; 3-[CH₂CONHMe]; 3-[CH₂-(thiazolidine-2,4-dion-5-yl)]; 3-SMe; 3-F; 3-Cl; 3-Br; 3-I; 3-CF₃; 3-OH; 3-OMe; 3-OCH₂Ph; 3-OiPr; 3-OPh; 3-O-pentafluorophenyl; 3-(OCH₂CO₂H); 3-(OCH₂CO₂Me); 3-(OCH₂CO₂Et); 3-NO₂; 3-CO₂H; 3-CO₂Me; 3-CONH₂; 3-CONHMe; 3-CONHCH₂CH₂OMe; 3-COMe; 3-COPh; 3-(COCH₂CH₂CO₂H); 3-(COCH₂CH₂CO₂Me); 3-CN; 3-SO₂CF₃; 3-SO₂NH-nBu; 3-(5-oxazolyl); 3-[4-methylpiperazin-1-yl]-4-OMe; 3-[O-pyrimidin-2-yl)]; 3-OH-4-OMe; 3,4-di-OMe; 3,5-di-OMe; 3,4-di-Me; 3,5-di-Me; 3-[trans-CH═CHCONMe₂]-4-Cl; 3-F-4-Me; 3-Cl-4-Me; 3-Br-4-Me; 3,5-di-F; 3,4-di-Cl; 3,5-di-Cl; 3,5-di-Br; 3-Cl-4-Br; 3-Cl-4-I; 3-Cl-4-OH; 3-Br-4-OH; 3-F-4-OMe; 3-Cl-4-OMe; 3-Cl-4-SMe; 3-Br-4-Cl; 3-Br-4-OCF₃; 3-Br-5-CF₃; 3,5-di-Cl-4-OH; 3,5-di-Br-4-OH; 3,5-di-Cl-4-Me; 3,5-di-Br-4-Me; 3-[CH₂CH(Me)CO₂H]; 3-CO₂H-4-Cl; 3-CO₂Me-4-Cl; 3-CO₂H-4-OH; 3-CONH₂-4-Me; 3-NO₂-4-OH; 3-CO₂H-4-SPh; 3-CO₂H-4-[S-(2-CO₂H-Ph)]; 3-CO₂H-4-[S-(2-CONHMe-Ph)]; 3-CO₂Et-4-[S-(2-CO₂Et-Ph)]; 3-CO₂H-4-[S-(3-CO₂H-Ph)]; 3-CO₂Me-4-[S-(4-Cl-Ph)]; 4-[N(Me)(Pyrimidin-2-yl)]; 4-Me; 4-nBu; 4-tBu; 4-cyclohexyl; 4-adamantyl; 4-CPh₃; 4-CH₂CN; 4-CH(OH)Me; 4-CH(OMe)Me; 4-CH₂OH; 4-CH₂NHC(O)ₜ-Bu; 4-CH₂NH₂; 4-CH₂NHCOMe; 4-CH₂NHCOPh; 4-CH₂NHCONHPh; 4-CH₂CO₂H; 4-CH₂CO₂Me; 4-[CH₂P(O)(OH)₂]; 4-[CH₂P(O)(OEt)₂]; 4-[CH₂SO₂NHMe]; 4-(CH₂)₂OH; 4-(CH₂)₂NH₂; 4-(CH₂)₂NHCOPh; 4-(CH₂)₂NHC(O)Ot-Bu; 4-[(CH₂)₂CO₂H]; 4-[(CH₂)₂CO₂Me]; 4-[(CH₂)₂CH₂CONH₂); 4-[CH₂CH₂CONH(CH₂)₆NHCOMe]; 4-[(CH₂)₃C0₂H]; 4-[(CH₂)₃CO₂Me]; 4-[CH═CH₂]; 4-(CH═CHCO₂H); 4-(CH═CHCO₂Et); 4-(CH═CHCONH₂); 4-(CH═CHPh); 4-(CH═CH(4-OHPh)); 4-[1,2,3-thiadiazol-4-yl]; 4-[OCH₂-(1-methylpiperazin-4-yl)]; 4-[4-methylpiperazin-1-yl]; 4-CF₃; 4-SMe; 4-(SCH₂CO₂H); 4-(SCH₂CO₂Me); 4-[SCH₂CONH(CH₂)₂OMe]; 4-SCF₃; 4-[S-(4-NO₂-Ph)]; 4-[S-(2-CO₂H-Ph)]; 4-[S-(3-CO₂H-Ph)]; 4-SO₂NH₂; 4-F; 4-Cl; 4-Br; 4-I; 4-OH; 4-OMe; 4-OnBu; 4-OPh; 4-[O-(4-Cl-Ph)]; 4-OCH₂Ph; 4-OCH₂CO₂Me; 4-COPh; 4-COMe; 4-CONH₂; 4-CO₂H; 4-CN; 4-NO₂; 4-morpholinyl; 4-[CH₂CO-morpholin-1-yl)]; 4-[CH₂CONH(CH₂)₂OMe]; 4-[(CH₂)₂CONH(CH₂)₆NHC(O)Ot-Bu]; 4-[(CH₂)₂CONH(CH₂)₆NH₂]; 4-[(CH₂)₂CONH(CH₂)₆NH-biotinyl]; 4-NMe₂; 4-NHCOMe; 4-N(Me)COMe; 2,3-di-F; 4-[NHCO(Ph-2-OH)], 4-(phenylamino); 4-methylsulphonylamino, 2,4-di-F; 2,5-di-F; 2-OMe-3-F; 3-CH₂OMe; 3-CH(OH)Ph; 3,4-di-F; 3-CO₂H-4-CH₂CO₂H; 3-CO₂H-4-[S-(2-CO₂Et)Ph]; 3-CO₂Et-4-[S-(4-CO₂H)Ph]; 3-CONHMe-4-[S-(2-CONHMe)-Ph]; 3-[4-(dichloroacetyl)piperazin-1-yl]-4-OMe; 4-CH₂CONH₂; 4-SPh; 4[S-(4-CO₂H-Ph)]; and 4-OCH₂CO₂H.

When R¹ and R³ together with the nitrogen atom to which they are attached form indolinyl, suitable substituents include C₁₋₆alkyl, perfluoroC₁₋₆ alkyl, C₁₋₆alkylSO₂NH-hydroxyC₁₋₆alkyl, carboxy, C₁₋₆alkoxycarbonyl, C₁₋₆alkoxy, halo, t-butoxycarbonylpiperazin-1-yl, 4-(C₁₋₆alkyl)piperazinyl, piperazinyl, amido, and nitro.

When R¹ and R³ together with the nitrogen atom to which they are attached form piperazinyl, suitable substituents include alkylcarbonyl, alkyl, or aryl.

When R¹ and R³ together with the nitrogen atom to which they are attached form tetrahydroquinolinyl, suitable substituents include perfluoroC₁₋₆alkyl.

When R¹ and R³ together with the nitrogen atom to which they are attached form a pyridinium ring, suitable substituents include amino.

When R¹ and R³ together with the nitrogen atom to which they are attached form pyrrolidinyl, suitable substituents include hydroxy.

When R¹ and R³ together with the nitrogen atom to which they are attached form piperidinyl, suitable substituents include benzyl, hydroxyC₁₋₆alkyl, C₁₋₆alkyl, hydroxy, carbamoyl, and C₁₋₆alkoxycarbonyl.

When R¹ and R³ together with the nitrogen atom to which they are attached form oxindolyl, suitable substituents include C₁₋₆alkyl.

As disclosed in WO 00/21927, there is a sub-group of compounds, falling wholly within formula (I), and being of formula (IA), wherein R, R¹, R² and R³ are as defined in relation to formula (I), with the proviso that formula (IA) does not include the following compounds, hereinafter referred to as List A:
3-phenyl-4-(4-methylpiperazino)-pyrrole-2,5-dione;
3-[4-(diphenylmethyl)-1-piperazinyl]-4-(1H-indol-3-yl)-1-methyl-1H-pyrrole-2,5-dione;
3-phenyl-4-(4-phenylpiperazino)-pyrrole-2,5-dione;
1-methyl-3-phenyl-4-(4-phenylpiperazino)-pyrrole-2,5-dione;
1-ethyl-3-phenyl-4-(4-chlorophenylpiperazino)-pyrrole-2,5-dione;
1-allyl-3-phenyl-4-(4-methylpiperazino)-pyrrole-2,5-dione;
3-indol-1-yl-4-(1-methyl-1H-indol-3-yl)-pyrrole-2,5-dione;
1-(1-methyl-2,5-dioxo-4-phenylamino-2,5-dihydro-1H-pyrrol-3-yl)pyridinium chloride;
1-[1-(4-methyl-pentyl)-2,5-dioxo-4-phenylamino-2,5-dihydro-1H-pyrrol-3-yl]pyridinium chloride;
1-(1-dodecyl-2,5-dioxo-4-phenylamino-2,5-dihydro-1H-pyrrol-3-yl)-pyridinium chloride;
3-[2-benzo[b]thien-2-yl-3-[4-(dimethylamino)-2,5-dihydro-2,5-dioxo-1H-pyrrol-3-yl]-1H-indol-1-yl]-carbamimidothioic acid, propyl ester;
3-(dimethylamino)-4-(1H-indol-3-yl)-1-methyl-1H-pyrrole-2,5-dione;
3-(1H-indol-3-yl)-1-methyl-4-(phenylamino)-1H-pyrrole-2,5-dione;
3-(1H-indol-3-yl)-1-methyl-4-[[4-(trifluoromethyl)phenyl]amino]-1H-pyrrole-2,5-dione;
3-(1H-indol-3-yl)-1-methyl-4-(methylamino)-1H-pyrrole-2,5-dione;
3-(1H-imidazo[4,5-b]pyridin-1-yl)-4-(1H-indol-3-yl)-1-methyl-1H-pyrrole-2,5-dione;
3-(6-chloro-9H-purin-9-yl)-4-(1H-indol-3-yl)-1-methyl-1H-pyrrole-2,5-dione;
3-(6-amino-9H-purin-9-yl)-4-(1H-indol-3-yl)-1-methyl-1H-pyrrole-2,5-dione;
3-(1H-indol-3-yl)-1-methyl-4-(1H-pyrrolo[2,3-b]pyridin-1-yl)-1H-pyrrole-2,5-dione;
3-(1H-indol-3-yl)-1-methyl-4-(1-piperidinyl)-1H-pyrrole-2,5-dione;
1-acetyl-3-[2,5-dihydro-1-methyl-2,5-dioxo-4-[[4-(trifluoromethyl)phenyl]amino]-1H-pyrrol-3-yl]-1H-indole;
3-(1H-benzimidazol-1-yl)-4-(1H-indol-3-yl)-1-methyl-1H-pyrrole-2,5-dione;
3-(1H-benzotriazol-1-yl)-4-(1H-indol-3-yl)-1-methyl-1H-pyrrole-2,5-dione;
3-(1H-imidazol-1-yl)-4-(1H-indol-3-yl)-1-methyl-1H-pyrrole-2,5-dione;
3-(1H-indol-1-yl)-4-(1H-indol-3-yl)-1-methyl-1H-pyrrole-2,5-dione;
3-(1H-indazol-1-yl)-4-(1H-indol-3-yl)-1-methyl-1H-pyrrole-2,5-dione;
3-[3-[(dimethylamino)methyl]-1H-indol-1-yl]-4-(1H-indol-3-yl)-1-methyl-1H-pyrrole-2,5-dione;
3-(1H-benzimidazol-1-yl)-4-(1H-indol-3-yl)-1H-pyrrole-2,5-dione;
3-(1H-indol-1-yl)-4-(1-methyl-1H-indol-3-yl)-1H-pyrrole-2,5-dione;
3-amino-4-(1H-indol-3-yl)-1H-pyrrole-2,5-dione;
3-amino-4-(5-methoxy-1H-indol-3-yl)-1H-pyrrole-2,5-dione;
1H-indole-1-carboxylic acid, 3-(4-amino-2,5-dihydro-1-methyl-2,5-dioxo-1H-pyrrol-3-yl)-1,1-dimethylethyl ester;
3-(1H-indol-3-yl)-1-methyl-4-[(phenylmethyl)amino]-1H-pyrrole-2,5-dione;
Glycine, N-[2,5-dihydro-4-(1H-indol-3-yl)-1-methyl-2,5-dioxo-1H-pyrrol-3-yl]-, ethyl ester;
3-amino-4-(1H-indol-3-yl)-1-methyl-1H-pyrrole-2,5-dione;
3-[[3-[(3-aminopropyl)amino)propyl]amino]-4-(1H-indol-3-yl)-1H-pyrrole-2,5-dione;
[[3-[4-(3-aminopropyl)-1-piperazinyl]propyl]amino]-4-(1H-indol-3-yl)-1H-pyrrole-2,5-dione;
3-(1H-indol-3-yl)-4-[[3-(4-methyl-1-piperazinyl)propyl]amino]-1H-pyrrole-2,5-dione;
1-[3-[(3-aminopropyl)amino]propyl]-3-[[3-[(3-aminopropyl)amino]propyl]amino]-4-(1H-indol-3-yl)-1H-pyrrole-2,5-dione;
1-[3-[4-(3-aminopropyl)-1-piperazinyl]propyl]-3-[[3-[4-(3-aminopropyl)-1-piperazinyl]propyl]amino]-4-(1H-indol-3-yl)-1H-pyrrole-2,5-dione;
3-(1H-indol-3-yl)-1-[3-(4-methyl-1-piperazinyl)propyl]-4-[[3-(4-methyl-1-piperazinyl)propyl]amino]-1H-pyrrole-2,5-dione;
3,3′-[iminobis(3,1-propanediylimino)]bis[4-(1H-indol-3-yl)-1H-pyrrole-2,5-dione;
3,3′-[1,4-piperazinediylbis(3,1-propanediylimino)]bis[4-(1H-indol-3-yl)-1H-pyrrole-2,5-dione;
3-[(5-aminopentyl)amino]-4-(1H-indol-3-yl)-1H-pyrrole-2,5-dione;
3-[[5-[(2-aminoethyl)amino]penty]amino]-4-(1H-indol-3-yl)-1-H-pyrrole-2,5-dione;
3-[(2-aminoethyl)amino]-4-(1H-indol-3-yl)-1H-pyrrole-2,5-dione;
3-[(6-aminohexyl)amino]-4-(1H-indol-3-yl)-1H-pyrrole-2,5-dione;
3-[(7-aminoheptyl)amino]-4-(1H-indol-3-yl)-1H-pyrrole-2,5-dione;
3-[[2-[(2-aminoethyl)amino]ethyl]amino]-4-(1H-indol-3-yl)-1H-pyrrole-2,5-dione;
Benzenepropanamide, α-amino-N-[5-[[2,5-dihydro-4-(1H-indol-3-yl)-2,5-dioxo-1H-pyrrol-3-yl]amino]pentyl]-, (S)—;
Pentanoic acid, 4-amino-5-[[5-[[2,5-dihydro-4-(1H-indol-3-yl)-2,5-dioxo-1H-pyrrol-3-yl]amino]pentyl]amino]-5-oxo-, (S)—;
Pentanamide, 2-amino-5-[(aminoiminomethyl)amino]-N-[2-[[5-[[2,5-dihydro-4-(1H-indol-3-yl)-2,5-dioxo-1H-pyrrol-3-yl]amino]pentyl]amino]ethyl, (S)—;
Benzenepropanamide, α-amino-N-[2-[[5-[[2,5-dihydro-4-(1H-indol-3-yl)-2,5-dioxo-1H-pyrrol-3-yl]amino]penty]amino]ethyl-, (S)-butanamide, 4-(aminoiminomethyl)amino-N-[5-[[2,5-dihydro-4-(1H-indol-3-yl)-2,5-dioxo-1H-pyrrol-3-yl]amino]pentyl]-, (S)—;
3-phenyl-4-(diethylamino)-pyrrole-2,5-dione;
3-phenyl-4-(benzylamino)-pyrrole-2,5-dione;
1-methyl-3-phenyl-4-(2-diethylaminoethylamino)-pyrrole-2,5-dione;
1-allyl-3-phenyl-4-(2-dimethlyaminoethylamino)-pyrrole-2,5-dione; and,
1,3-diphenyl-4-piperidino-pyrrole-2,5-dione.

As disclosed in WO 00/21927, there is a further sub-group of compounds, falling wholly within formula (I), and being of formula (IB), wherein R, R¹, R² and R³ are as defined in relation to formula (I), with the proviso that formula (IB) does not include the following compounds, hereinafter referred to as List B:
3-(4-methylpiperazin-1-yl)-4-phenyl-pyrrole-2,5-dione;
3-(4-ethylpiperazin-1-yl)-4-phenyl-pyrrole-2,5-dione;
3-(4-chlorophenyl)-4-(4-methyl-piperazin-1-yl)-pyrrole-2,5-dione;
3-[4-(diphenylmethyl)-1-piperazinyl]-4-(1H-indol-3-yl)-1-methyl-1H-pyrrole-2,5-dione;
3-phenyl-4-(4-methylpiperazino)-pyrrole-2,5-dione;
3-phenyl-4-(4-phenylpiperazino)-pyrrole-2,5-dione;
1-methyl-3-phenyl-4-(4-phenylpiperazino)-pyrrole-2,5-dione;
1-ethyl-3-phenyl-4-(4-chlorophenylpiperazino)-pyrrole-2,5-dione;
1-allyl-3-phenyl-4-(4-methylpiperazino)-pyrrole-2,5-dione;
3-phenylamino-4-phenyl-1H-pyrrole-2,5-dione;
3-phenyl-4-piperidin-1-yl-pyrrole-2,5-dione;
3-(3,5-dimethyl-1-phenyl-1H-pyrazol-4-yl)-4-morpholin-4-yl-pyrrole-2,5-dione;
3-indol-1-yl-4-(1-methyl-1H-indol-3-yl)-pyrrole-2,5-dione;
1-(1-methyl-2,5-dioxo-4-phenylamino-2,5-dihydro-1H-pyrrol-3-yl)-pyridinium chloride;
1-1-(4-methyl-pentyl)-2,5-dioxo-4-phenylamino-2,5-dihydro-1H-pyrrol-3-yl)-pyridinium chloride;
1-(1-dodecyl-2,5-dioxo-4-phenylamino-2,5-dihydro-1H-pyrrol-3-yl)-pyridinium chloride;
3-[2,5-dihydro-4-(1H-imidazol-1-yl)-1-methyl-2,5-dioxo-1H-pyrrol-3-yl]-1H-indole-1-carboxylic acid, 1,1-dimethylethyl ester;
3-[2-benzo[b]thien-2-yl-3-[4-(dimethylamino)-2,5-dihydro-2,5-dioxo-1H-pyrrol-3-yl]-1H-indol-1-yl]-carbamimidothioic acid, propyl ester;
3-(dimethylamino)-4-(1H-indol-3-yl)-1-methyl-1H-pyrrole-2,5-dione;
3-(1H-indol-3-yl)-1-methyl-4-(phenylamino)-1H-pyrrole-2,5-dione;
3-(1H-indol-3-yl)-1-methyl-4-[[4-(trifluoromethyl)phenyl]amino]-1H-pyrrole-2,5-dione;
3-(1H-indol-3-yl)-1-methyl-4-(methylamino)-1H-pyrrole-2,5-dione;
3-(1H-imidazo[4,5-b]pyridin-1-yl)-4-(1H-indol-3-yl)-1-methyl-1H-pyrrole-2,5-dione;
3-(6-chloro-9H-purin-9-yl)-4-(1H-indol-3-yl)-1-methyl-1H-pyrrole-2,5-dione;
3-(6-amino-9H-purin-9-yl)-4-(1H-indol-3-yl)-1-methyl-1H-pyrrole-2,5-dione;
3-(1H-indol-3-yl)-1-methyl-4-(1H-pyrrolo[2,3-b]pyridin-1-yl)-1H-pyrrole-2,5-dione;
3-(1H-indol-3-yl)-1-methyl-4-(1-piperidinyl)-1H-pyrrole-2,5-dione;
1-acetyl-3-[2,5-dihydro-1-methyl-2,5-dioxo-4-[[4-(trifluoromethyl)phenyl]amino]-1H-pyrrol-3-yl]-1H-indole;
3-(1H-benzimidazol-1-yl)-4-(1H-indol-3-yl)-1-methyl-1H-pyrrole-2,5-dione;
3-(1H-benzotriazol-1-yl)-4-(1H-indol-3-yl)-1-methyl-1H-pyrrole-2,5-dione;
3-(1H-imidazol-1-yl)-4-(1H-indol-3-yl)-1-methyl-1H-pyrrole-2,5-dione;
3-(1H-indol-1-yl)-4-(1H-indol-3-yl)-1-methyl-1H-pyrrole-2,5-dione;
3-(1H-indazol-1-yl)-4-(1H-indol-3-yl)-1-methyl-1H-pyrrole-2,5-dione;
3-[3-[(dimethylamino)methyl]-1H-indol-1-yl]-4-(1H-indol-3-yl)-1-methyl-1H-pyrrole 2,5-dione;
3-(1H-benzimidazol-1-yl)-4-(1H-indol-3-yl)-1H-pyrrole-2,5-dione;
3-(1H-indol-1-yl)-4-(1-methyl-1H-indol-3-yl)-1H-pyrrole-2,5-dione;
3-(3,5-dimethyl-1-phenyl-1H-pyrazol-4-yl)-4-(4-morpholinyl)-1H-pyrrole-2,5-dione;
3-amino-4-(1H-indol-3-yl)-1H-pyrrole-2,5-dione;
3-amino-4-(5-methoxy-1H-indol-3-yl)-1H-pyrrole-2,5-dione;
1H-Indole-1-carboxylic acid, 3-(4-amino-2,5-dihydro-1-methyl-2,5-dioxo-1H-pyrrol-3-yl)-,1,1-dimethylethyl ester;
3-(1H-indol-3-yl)-1-methyl-4-[(phenylmethyl)amino]-1H-pyrrole-2,5-dione;
Glycine, N-[2,5-dihydro-4-(1H-indol-3-yl)-1-methyl-2,5-dioxo-1H-pyrrol-3-yl]-, ethyl ester;
3-amino-4-(1H-indol-3-yl)-1-methyl-1H-pyrrole-2,5-dione;
1-(4-methylphenyl)-3-[(4-methylphenyl)amino]-4-phenyl-1H-pyrrole-2,5-dione;
3-[[3-[(3-aminopropyl)amino]propyl]amino]-4-(1H-indol-3-yl)-1H-pyrrole-2,5-dione;
3-[[3-[4-(3-aminopropyl)-1-piperazinyl]propyl]amino]-4-(1H-indol-3-yl)-1H-pyrrole-2,5-dione;
3-(1H-indol-3-yl)-4-[[3-(4-methyl-1-piperazinyl)propyl]amino]-1H-pyrrole-2,5-dione;
1-[3-[(3-aminopropyl)amino]propyl]-3-[[3-[(3-aminopropyl)amino]propyl]amino]-4-(1H-indol-3-yl)-1H-pyrrole-2,5-dione;
1-[3-[4-(3-aminopropyl)-1-piperazinyl]propyl]-3-[[3-[4-(3-aminopropyl)-1-piperazinyl)propyl][amino]-4-(1H-indol-3-yl)-1H-pyrrole-2,5-dione;
3-(1H-indol-3-yl)-1-[3-(4-methyl-1-piperazinyl)propyl]-4-[[3-(4-methyl-1-piperazinyl)propyl]amino]-1H-pyrrole-2,5-dione;
3,3′-[iminobis(3,1-propanediylimino)]bis[4-(1H-indol-3-yl)-1H-pyrrole-2,5-dione;
3,3′-[1,4-piperazinediylbis(3,1-propanediylimino)]bis[4-(1H-indol-3-yl)-1H-pyrrole-2,5-dione;
3-amino-4-(3,4-dimethoxyphenyl)-1H-pyrrole-2,5-dione;
3-[(5-aminopentyl)amino]-4-(1H-indol-3-yl)-1H-pyrrole-2,5-dione;
3-[[5-[(2-aminoethyl)amino]pentyl]amino]-4-(1H-indol-3-yl)-1H-pyrrole-2,5-dione;
3-[(2-aminoethyl)amino]-4-(1H-indol-3-yl)-1H-pyrrole-2,5-dione;
3-[(6-aminohexyl)amino]-4-(1H-indol-3-yl)-1H-pyrrole-2,5-dione;
3-[(7-aminoheptyl)amino]-4-(1H-indol-3-yl)-1H-pyrrole-2,5-dione;
3-[[2-[(2-aminoethyl)amino]ethyl]amino]-4-(1H-indol-3-yl)-1H-pyrrole-2,5-dione;
Benzenepropanamide, α-amino-N-[5-[[2,5-dihydro-4-(1H-indol-3-yl)-2,5-dioxo-1H-pyrrol-3-yl)amino]pentyl]-, (S)—;
Pentanoic acid, 4-amino-5-[[5-[[2,5-dihydro-4-(1H-indol-3-yl)-2,5-dioxo-1H-pyrrol-3-yl]amino]pentyl]amino]-5-oxo-, (S)—;
Pentanamide, 2-amino-5-[(aminoiminomethyl)amino]-N-[2-[[5-[[2,5-dihydro-4-(1H-indol-3-yl)-2,5-dioxo-1H-pyrrol-3-yl]amino]pentyl]amino]ethyl]-, (S)—;
Benzenepropanamide, α-amino-N-[2-[[5-[[2,5-dihydro-4-(1H-indol-3-yl)-2,5-dioxo-1H-pyrrol-3-yl]amino]pentyl]amino]ethyl]-, (S)—;
Butanamide, 4-[(aminoiminomethyl)amino]-N-[5-[[2,5-dihydro-4-(1H-indol-3-yl)-2,5-dioxo-1H-pyrrol-3-yl]amino]pentyl]-, (S)—;
3-(4-methylphenyl)-1-phenyl-4-(phenylamino)-1H-pyrrole-2,5-dione;
1,3-bis(4-methylphenyl)-4-[(4-methylphenyl)amino]-1H-pyrrole-2,5-dione;
3-amino-1,4-diphenyl-1H-pyrrole-2,5-dione;
3-(4-methylphenyl)-4-(4-morpholinyl)-1-phenyl-1H-pyrrole-2,5-dione;
3-(4-methylphenyl)-1-phenyl-4-[(phenylmethyl)amino]-1H-pyrrole-2,5-dione;
3-amino-4-(4-methylphenyl)-1-phenyl-1H-pyrrole-2,5-dione;
3-(3,5-dimethyl-1-phenyl-1H-pyrazol-4-yl)-4-(4-morpholinyl)-1H-pyrrole-2,5-dione;
3-(4-nitrophenyl)-1-phenyl-4-phenylamino-1H-pyrrole-2,5-dione;
3-amino-1-methyl-4-p-tolyl-1H-pyrrole-2,5-dione;
3-(2-diethylamino-ethylamino)-4-phenyl-pyrrole-2,5-dione;
3-[butyl-(2-diethylamino-ethyl)-amino]-4-phenyl-pyrrole-2,5-dione;
3-[benzyl-(2-dimethyl amino-ethyl)-amino]-4-phenyl-pyrrole-2,5-dione;
3-[benzyl-(2-dimethylamino-ethyl)-amino]-1-methyl-4-phenyl-pyrrole-2,5-dione;
3-[benzyl-(2-dimethylamino-ethyl)-amino]-4-(4-chloro-phenyl)-pyrrole-2,5-dione;
3-[benzyl-(2-diethylamino-ethyl)-amino]-4-phenyl-pyrrole-2,5-dione;
3-[benzyl-(2-dimethylamino-ethyl)-amino]-4-(3-methoxy-phenyl)-pyrrole-2,5-dione;
3-(4-chloro-phenyl)-4-[2-(4-methyl-piperazin-1-yl)-ethylamino]-pyrrole-2,5-dione;
3-[2-(4-methyl-piperazin-1-yl)-ethylamino]-4-phenyl-pyrrole-2,5-dione;
3-phenyl-4-(diethylamino)-pyrrole-2,5-dione;
3-phenyl-4-(benzylamino)-pyrrole-2,5-dione;
1-methyl-3-phenyl-4-(2-diethylaminoethylamino)-pyrrole-2,5-dione;
1-allyl-3-phenyl-4-(2-dimethylaminoethylamino)-pyrrole-2,5-dione; and
1,3-diphenyl-4-piperidino-pyrrole-2,5-dione.

As disclosed in WO 00/21927, there is a subgroup of compounds falling wholly within formula (I) of formula (IC): 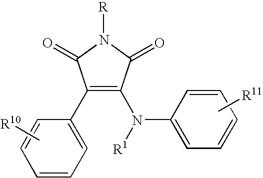
wherein;

R and R¹ are as defined in relation to formula (I);

R¹⁰ represents hydrogen or one or more substituents, suitably up to three, selected from the list consisting of: alkoxycarbonyl, alkoxyalkyl, perfluoroalkyl, perfluoroalkylS—, perfluoroalkylO—, phenyl(di-C₁₋₆alkoxy)C—, benzoyl, C₁₋₆alkylSO₂—, —[(CH═CH)₂]—, phenyl, nitro, —OCH₂O—, benzyloxy, phenoxy, halo, hydroxy, alkyl, alkoxy, amino, mono- or di-alkyl amino or thioalkyl;

R¹¹ represents hydrogen or one or more substituents, suitably up to three, selected from the list consisting of substituted or unsubstituted C₁₋₆alkyl, phenyl, benzyl, substituted or unsubstituted C₁₋₆alkylS—, halo, hydroxy, substituted or unsubstituted C₁₋₆alkoxy, substituted or unsubstituted phenoxy, indolyl, naphthyl, carboxy, C₁₋₆alkoxycarbonyl, benzyloxy, phenoxy, pentafluorophenoxy, nitro, substituted or unsubstituted carbamoyl, substituted or unsubstituted C₁₋₆alkylcarbonyl, benzoyl, cyano, perfluoroC₁₋₆alkylSO₂—, C₁₋₆alkylNHSO₂—, oxazolyl, substituted or unsubstituted phenylS—, C₁₋₆alkylpiperazinyl-, C₁₋₆alkylcarbonylpiperazinyl-, 1,2,3-thiadiazolyl, pyrimidin-2-yloxy, N-[pyrimidin-2-yl]-N-methylamino, phenylamino, C₁₋₆alkylsulphonylamino, N-morpholinylcarbonyl, cyclohexyl, adamantyl, trityl, substituted or unsubstituted C₁₋₆alkenyl, perfluoroC₁₋₆alkyl, perfluoroC₁₋₆alkoxy, perfluoroC₁₋₆alkylS—, aminosulphonyl, morpholino, (diC₁₋₆alkyl)amino, C₁₋₆alkylCONH—, (diC₁₋₆alkoxy)phenyl(CH₂)ₙNHC(O)CH(phenyl)S—, where n is 1 to 6, and C₁₋₆alkylCON(C₁₋₆alkyl)-, thiazolidinedionylC₁₋₆alkyl, phenylCH(OH)—, substituted or unsubstituted piperazinylC₁₋₆alkoxy, substituted or unsubstituted benzoylamino; or —(CH₂)ₓ—, —SCH═N—, —SC(C₁₋₆alkyl)═N—, —OCF₂O—, —[CH═CHC(O)O]—, —[N═CH—CHCH]—, —CH═N—NH—, —CH—CH—NH—, —OC(NHC₁₋₆alkyl)═N—, —OC(O)NH—, —C(O)NMeC(O)—, C(O)NHC(O)—, (CH₂)ₓC(O), —N═N—NH—, —N═C(C₁₋₆alkyl)O—, —O(CH₂)ₓO, (CH₂)ₓSO₂(CH₂)_{y}—, and —N(C₁₋₆alkylcarbonyl)(CH₂)ₓ—, where x and y are independently 1 to 4.

As disclosed in WO 00/21927, there is a subgroup of compounds within formula (IC) of formula (IC′) wherein R, R¹, R¹⁰ and R¹¹ are as defined in relation to formula (IC) with the proviso that formula (IC′) does not include:
3-phenylamino-4-phenyl-1H-pyrrole-2,5-dione;
1-(4-methylphenyl)-3-[(4-methylphenyl)amino]-4-phenyl-1H-pyrrole-2,5-dione;
3-(4-methylphenyl)-1-phenyl-4-(phenylamino)-1H-pyrrole-2,5-dione;
1,3-bis(4-methylphenyl)-4-[(4-methylphenyl)amino]-1H-pyrrole-2,5-dione; or
3-(4-nitrophenyl)-1-phenyl-4-phenylamino-1H-pyrrole-2,5-dione.

Suitably, R is hydrogen.

Suitably, R¹ is hydrogen.

Suitably, R¹⁰ represents hydrogen or one or more substituents selected from the list consisting of: halo, hydroxy, alkyl, alkylthio, alkoxy, amino or methylenedioxy, especially one or more halo and alkyl groups.

Favourably, R¹⁰ represents hydrogen or the substituents selected from the list consisting of: 2-Br, 2-Cl, 2-F, 2-OMe, 3-Cl, 3-F, 3-Me, 3-NH₂, 3-OMe, 4-Br, 4-Cl, 4-1,4-Me, 4-OH, 4-OMe, 4-SMe, 2,3-di-F, 2,5-di-F, 2,6-di-F, 3,4-di-F, 3,5-di-F, 2,3,5-tri-F, 2,4-di-Cl, 2,4-di-OMe, 3,4-(OCH₂O) and 3,5-di-Me.

More favourably, R¹⁰ represents the substituents selected from the list consisting of: 2-Br, 2-Cl, 2-F, 2-OMe, 3-Cl, 3-F, 3-Me, 4-Br, 4-Cl, 4-1,2,3-di-F, 2,5-di-F, 2,6-di-F, 3,4-di-F, 3,5-di-F, 2,3,5-tri-F, 2,4-di-Cl and 3,5-di-Me.

Preferably, R¹⁰ represents the substituents selected from the list consisting of: 2-F, 2-OMe, 3-F, 4-Cl and 2,3-di-F.

Suitably, R¹¹ represents hydrogen or one or more substituents selected from the list consisting of: 2-F, 2-Me, 3-Br, 3-Cl, 3-F, 3-1,3-OH, 3-OMe, 3-OPh, 3-SMe, 3-CO₂H, 3-CH₂CO₂H, 3-CH₂CO₂Me, 3-CH₂CONH₂, 3-CH₂CONHMe, 3-CH₂OH, 4-Cl, 4-F, 4-Me, 4-NHCOMe, 4-NHPh, 4-NHSO₂Me, 4-NMe₂, 4-OMe, 4-COPh, 4-SMe, 4-CH₂CN, 4-SO₂NH_{2,4}-(CH₂)₂OH, 4-CH(OH)Ph, 4-CH₂SO₂NHMe, 4-CH₂CO₂H, 4-(CH₂)₂CO₂H, 4-(CH₂)₂CO₂Me, 4-(CH₂)₂CONH₂, 4-(CH₂)₃CO₂H, 4-(CH₂)₃CONH₂, 4-CH═CHCO₂H, 4-CH═CHCONH₂, 4-OCH₂CO₂H, 4-SCH₂CO₂H, 4-S-[2-CO₂H-Ph], 4-S-[3-CO₂H-Ph], 4-CH₂(1,3-thiazolidin-2,4-dion-5-yl), 2,3-di-F, 2,4-di-F, 3,4-di-F, 3,5-di-F, 3-Cl-4-Br, 3-Cl-4-Me, 3-Br-4-Me, 3-Cl-4-OH, 3-Cl-4-OMe, 3,5-di-Me, 3,5-di-OMe, 3,4-OC(O)NH—, 3,4-OCF₂O—, 3,5-di-Br-4-OH, 3,5-di-Cl-4-Me, 3,5-di-Cl-4-OH, 3-CO₂H-4-[S-(2-CO₂H)-Ph], 3-CO₂H-4-[S-(2-CONHMe)-Ph], 3-CO₂H-4-Cl, 3-F-4-Me, 3-F-4-OMe, 3,4-[(CH═N—NH)]—, 3,4—[(N═N—NH)]—, 3,4-[(NH—N═CH)]—, 3,4-[(CH₂)₃]—, 3,4-[(O(CH₂)₃O)]—, 3,4-[O—C(NHMe)=N]—, 3,4-[OCH₂O—, 3,4-[S—C(NHMe)=N]— and 3,4-[S—CH═N]—.

Favourably, R¹¹ represents hydrogen or the substituents selected from the list consisting of: 2-F, 2-Me, 3-Cl, 3-F, 3-I, 3-OMe, 3-OPh, 3-SMe, 3-CH₂CO₂H, 3-CH₂CO₂Me, 3-CH₂CONH₂, 3-CH₂CONHMe, 3-CH₂OH, 4-Cl, 4-F, 4-Me, 4-NHCOMe, 4-NHPh, 4-NHSO₂Me, 4-NMe₂, 4-OMe, 4-COPh, 4-SMe, 4-CH₂CN, 4-SO₂NH₂, 4-(CH₂)₂OH, 4-CH(OH)Ph, 4-CH₂SO₂NHMe, 4-CH₂CO₂H, 4-(CH₂)₂CO₂H, 4-(CH₂)₂CO₂Me, 4-(CH₂)₂CONH₂, 4-(CH₂)₃CO₂H, 4-(CH₂)₃CONH₂, 4-CH═CHCONH₂, 4-OCH₂CO₂H, 4-SCH₂CO₂H, 4-S-[2-CO₂H-Ph], 4-S-[3-CO₂H-Ph], 4-CH₂(1,3-thiazolidin-2,4-dion-5-yl), 2,3-di-F, 2,4-di-F, 3,4-di-F, 3,5-di-F, 3-C₁₋₄-Br, 3-Cl-4-Me, 3-Br-4-Me, 3-Cl-4-OH, 3-Cl-4-OMe, 3,5-di-Me, 3,5-di-OMe, 3,4-[OC(O)NH], 3,4-[OCF₂O] 3,5-di-Cl-4-Me, 3-CO₂H-4-[S-(2-C(NHMe)-Ph], 3-F-4-Me, 3-F-4-OMe, 3,4-[(CH═N—NH)], 3,4-[(N═N—NH)], 3,4-[(NH—N═CH)], 3,4-[(CH₂)₃], 3,4-[O(CH₂)₃O], 3,4-[O—C(NHMe)=N], 3,4-[OCH₂O], 3,4-[S—C(NHMe)=N] and 3,4-[S—CH═N].

More favourably, R¹¹ represents the substituents selected from the list consisting of: 3-Cl, 3-Br, 4-OMe, 3,5-di-F, 4-CH₂SO₂NHMe, 4-(CH₂)₃CO₂H and 4-S-[3-CO₂H-Ph].

A particular compound of formula (IC) is that wherein R and R¹ each represent hydrogen and R¹⁰ and R¹¹ each have the following respective values:

| R¹⁰ | R¹¹ |
|---|---|
| 4-Cl | 3-Cl |
| 4-Cl | 3-Br |
| 2-OMe | 4-OMe |
| 4-Cl | 4-CH₂SO₂NHMe |
| 2-OMe | 3,5-di-F |
| 2-F | 3,5-di-F |
| 3-F | 4-(CH₂)₃CO₂H |
| 2,3-di-F-Ph | 3,5-di-F |

As disclosed in WO 00/21927, there is a subgroup of compounds falling wholly within formula (I) being of formula (ID): 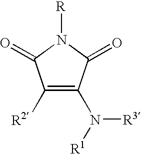
wherein R and R¹ are as defined in relation to formula (I);

R^{2′} is phenyl, substituted phenyl or indolyl;

R^{3′} is hydrogen, alkyl, cycloalkyl, phenyl, substituted phenyl, C₁₋₆ alkylphenyl wherein the phenyl group is optionally substituted, alkoxyalkyl, substituted or unsubstituted heterocyclyl.

In one aspect, there is provided a compound of formula (I) as hereinbefore defined, which excludes compounds of formula (ID).

There is a subgroup of compounds within formula (ID) of formula (ID′) wherein R, R¹, R^{2′} and R^{3′} are as defined in relation to formula (ID) with the proviso that formula (ID′) does not include the following compounds, hereinafter referred to as List D′:
3-[2-benzo[b]thien-2-yl-3-[4-(dimethylamino)-2,5-dihydro-2,5-dioxo-1H-pyrrol-3-yl]-1H-indol-1-yl]-carbamimidothioic acid, propyl ester;
3-(dimethylamino)-4-(1-indol-3-yl)-1-methyl-1H-pyrrole-2,5-dione;
3-(1H-indol-3-yl)-1-methyl-4-(phenylamino)-1H-pyrrole-2,5-dione;
3-(1H-indol-3-yl)-1-methyl-4-[[4-(trifluoromethyl)phenyl]amino]-1H-pyrrole-2,5-dione;
3-(1H-indol-3-yl)-1-methyl-4-(methylamino)-1H-pyrrole-2,5-dione;
3-(6-chloro-9H-purin-9-yl)-4-(1H-indol-3-yl)-1-methyl-1H-pyrrole-2,5-dione;
3-(6-amino-9H-purin-9-yl)-4-(1H-indol-3-yl)-1-methyl-1H-pyrrole-2,5-dione;
1-acetyl-3-[2,5-dihydro-1-methyl-2,5-dioxo-4-[[4-(trifluoromethyl)phenyl]amino]-1H-pyrrol-3-yl]-1H-indole;
3-amino-4-(1H-indol-3-yl)-1H-pyrrole-2,5-dione;
3-amino-4-(5-methoxy-1H-indol-3-yl)-1H-pyrrole-2,5-dione;
1H-indole-1-carboxylic acid, 3-(4-amino-2,5-dihydro-1-methyl-2,5-dioxo-1H-pyrrol-3-yl)-, 1,1-dimethylethyl ester;
3-(1H-indol-3-yl)-1-methyl-4-[(phenylmethyl)amino]-1H-pyrrole-2,5-dione;
Glycine, N-[2,5-dihydro-4-(1H-indol-3-yl)-1-methyl-2,5-dioxo-1H-pyrrol-3-yl]-, ethyl ester;
3-amino-4-(1H-indol-3-yl)-1-methyl-1H-pyrrole-2,5-dione;
3-[[3-[(3-aminopropyl)amino]propyl]amino]-4-(1H-indol-3-yl)-1H-pyrrole-2,5-dione;
3-[[3-[4-(3-aminopropyl)-1-piperazinyl [propyl]amino]-4-(1H-indol-3-yl)-1H-pyrrole-2,5-dione;
3-(1H-indol-3-yl)-4-[[3-(4-methyl-1-piperazinyl)propyl]amino]-1H-pyrrole-2,5-dione;
1-[3-[(3-aminopropyl)amino]propyl]-3-[[3-[(3-aminopropyl)amino]propyl]amino]-4-(1H-indol-3-yl)-1H-pyrrole-2,5-dione;
1-(3-[4-(3-aminopropyl)-1-piperazinyl]propyl]-3-[[3-[4-(3-aminopropyl)-1-piperazinyl]propyl]amino]-4-(1H-indol-3-yl)-1H-pyrrole-2,5-dione;
3-(1H-indol-3-yl)-1-[3-(4-methyl-1-piperazinyl)propyl]-4-[[3-(4-methyl-1-piperazinyl)propyl]amino]-1H-pyrrole-2,5-dione;
3,3′-[iminobis(3,1-propanediylimino)]bis[4-(1H-indol-3-yl)-1H-pyrrole-2,5-dione;
3,3′-[1,4-piperazinediylbis(3,1-propanediylimino)]bis[4-(1H-indol-3-yl)-1H-pyrrole-2,5-dione;
3-amino-4-(3,4-dimethoxyphenyl)-1H-pyrrole-2,5-dione;
3-[(5-aminopentyl)amino]-4-(1H-indol-3-yl)-1H-pyrrole-2,5-dione;
3-[[5-[(2-aminoethyl)amino]pentyl]amino]-4-(1H-indol-3-yl)-1H-pyrrole-2,5-dione;
3-[(2-aminoethyl)amino]-4-(1H-indol-3-yl)-1H-pyrrole-2,5-dione;
3-[(6-aminohexyl)amino]-4-(1H-indol-3-yl)-1H-pyrrole-2,5-dione;
3-[(7-aminoheptyl)amino]-4-(1H-indol-3-yl)-1H-pyrrole-2,5-dione;
3-[[2-[(2-aminoethyl)amino]ethyl]amino]-4-(1H-indol-3-yl)-1H-pyrrole-2,5-dione;
Benzenepropanamide, α-amino-N-[5-[[2,5-dihydro-4-(1H-indol-3-yl)-2,5-dioxo-1H-pyrrol-3-yl]amino]pentyl]-, (S)—;
Pentanoic acid, 4-amino-5-[[5-[[2,5-dihydro-4-(1H-indol-3-yl)-2,5-dioxo-1H-pyrrol-3-yl]amino]pentyl]amino]-5-oxo-, (S)—;
Pentanamide, 2-amino-5-[(aminoiminomethyl)amino]-N-[2-[[5-[[2,S-dihydro-4-(1H-indol-3-yl)-2,5-dioxo-1H-pyrrol-3-yl]amino]pentyl]amino]ethyl]-, (S)—;
Benzenepropanamide, α-amino-N-[2-[[5-[[2,5-dihydro-4-(1H-indol-3-yl)-2,5-dioxo-1H-pyrrol-3-yl]amino]pentyl]amino]ethyl]-, (S)—;
Butanamide, 4-[(aminoiminomethyl)amino]-N-[5-[[2,5-dihydro-4-(1H-indol-3-yl)-2,5-dioxo-1H-pyrrol-3-yl]amino]pentyl]-, (S)—;
3-amino-1,4-diphenyl-1H-pyrrole-2,5-dione;
3-(4-methylphenyl)-1-phenyl-4-[(phenylmethyl)amino]-1H-pyrrole-2,5-dione;
3-amino-4-(4-methylphenyl)-1-phenyl-1H-pyrrole-2,5-dione;
3-amino-1-methyl-4-p-tolyl-1H-pyrrole-2,5-dione;
3-(2-diethylamino-ethylamino)-4-phenyl-pyrrole-2,5-dione;
3-[butyl-(2-diethylamino-ethyl)-amino]-4-phenyl-pyrrole-2,5-dione;
3-[benzyl-(2-dimethylamino-ethyl)-amino]-4-phenyl-pyrrole-2,5-dione;
3-[benzyl-(2-dimethylamino-ethyl)-amino]-1-methyl-4-phenyl-pyrrole-2,5-dione;
3-[benzyl-(2-dimethylamino-ethyl)-amino]-4-(4-chloro-phenyl)-pyrrole-2,5-dione;
3-[benzyl-(2-diethylamino-ethyl)-amino]-4-phenyl-pyrrole-2,5-dione;
3-[benzyl-(2-dimethylamino-ethyl)-amino]-4-(3-methoxy-phenyl)-pyrrole-2,5-dione;
3-(4-chloro-phenyl)-4-[2-(4-methyl-piperazin-1-yl)-ethylamino]-pyrrole-2,5-dione;
3-[2-(4-methyl-piperazin-1-yl)-ethylamino]-4-phenyl-pyrrole-2,5-dione;
3-phenyl-4-(diethylamino)-pyrrole-2,5-dione;
3-phenyl-4-(benzylamino)-pyrrole-2,5-dione;
1-methyl-3-phenyl-(2-diethylaminoethylamino)-pyrrole-2,5-dione; and
1-allyl-3-phenyl-4-(2-dimethylaminoethylamino)-pyrrole-2,5-dione.

Suitably R^{2′} is indolyl, phenyl or phenyl substituted with one or more, suitably up to three, substituents selected from the list consisting of: halo, haloalkyl, alkoxy, nitro, alkyl and alkoxy.

Examples of R^{2′} include phenyl, indol-3-yl, 2-methoxyphenyl, 3-fluorophenyl, 3-nitrophenyl, 4-chlorophenyl, 4-iodophenyl, 4-(trifluoromethyl)phenyl, and 2,3-difluorophenyl.

Suitably R^{3′} represents hydrogen, C₁₋₆ alkyl, cyclohexyl, phenyl, fluorenyl, C₁₋₂ alkylphenyl, C₁₋₆alkoxyC₁₋₂alkyl or a substituted or unsubstituted single or a single or fused ring heterocyclyl group having 5 or 6 ring atoms and up to 3 hetero atoms in each ring, such as oxazolyl, benzofuranyl, dibenzofuranyl, pyridinyl, quinolinyl, and pyrimidinyl.

Examples of R^{3′} include hydrogen, ethyl, cyclohexyl, phenyl, fluoren-2-yl, benzyl, phenyl(CH₂)₂—, MeO(CH₂)₂—, 4-methyloxazol-2-yl, 2-acetylbenzofuran-5-yl, dibenzofuran-2-yl, dibenzofuran-3-yl, 2-methylpyridin-3-yl, 2,6-dimethylpyridin-3-yl, 2-chloropyridin-5-yl, quinolin-3-yl, pyrimidin-2-yl.

As disclosed in WO 00/21927, there is a subgroup of compounds falling wholly within formula (I) being of formula (IE): 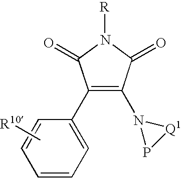
wherein R is as defined in relation to formula (I);

R^{10′} represents hydrogen or one or more, suitably up to three, substituents selected from the list consisting of alkoxy, halo, and nitro;

P′-Q′ represents (CH₂)ₐO(CH₂)_{b}—, (CH₂)ₐS(CH₂)_{b}—, —(CH₂)_{c}—, —(CH₂)_{d}CH(G)(CH₂)ₑ—, —(CH₂)ₐN(ZZ)(CH₂)_{b}—, where a, b, d, and e are independently 1 to 4, c is 1 to 6, ZZ is hydrogen, alkyl, aryl, or alkylcarbonyl, and G is alkyl, amido, hydroxyalkyl, aralkyl, or hydroxy.

There is a subgroup of compounds within formula (IE) of formula (IE′) wherein R, R^{10′}, and P′-Q′ are as defined in relation to formula (E) with the proviso that formula (IE′) does not include;
3-phenyl-4-piperidin-1-yl-pyrrole-2,5-dione;
3-(4-methylpiperazin-1-yl)-4-phenyl-pyrrole-2,5-dione;
3-(4-ethylpiperazin-1-yl)-4-phenyl-pyrrole-2,5-dione;
3-(4-chlorophenyl)-4-(4-methyl-piperazin-1-yl)-pyrrole-2,5-dione;
3-(4-methylphenyl)-4-(4-morpholinyl)-1-phenyl-1H-pyrrole-2,5-dione;
3-phenyl-4-(4-methylpiperazino)-pyrrole-2,5-dione;
3-phenyl-4-(4-phenylpiperazino)-pyrrole-2,5-dione;
1-methyl-3-phenyl-4-(4-phenylpiperazino)-pyrrole-2,5-dione;
1-ethyl-3-phenyl-4-(4-chlorophenylpiperazino)-pyrrole-2,5-dione;
1-allyl-3-phenyl-4-(4-methylpiperazino)-pyrrole-2,5-dione; and
1,3-diphenyl-4-piperidino-pyrrole-2,5-dione.

Suitably, R^{10′} is methoxy, chloro, or nitro.

Examples of R^{10′} include 4-methoxy, 4-chloro, 2,4-dichloro, and 3-nitro.

Examples of —P′-Q′- include —(CH₂)₄—, —(CH₂)₂O(CH₂)₂—, —(CH₂)₃CH(Me)CH₂—, —(CH₂)₃CH(CONH₂,)CH₂—, —(CH₂)₃CH(CH₂OH)CH₂—, —(CH₂)₂CH(CH₂Ph)(CH₂)₂—, —(CH₂)₂CH(OH)(CH₂)₂—, —(CH₂)₅—, and —(CH₂)S(CH₂)₂—.

As disclosed in WO 00/21927, there is a subgroup of compounds falling wholly within formula (I) being of formula (IF): 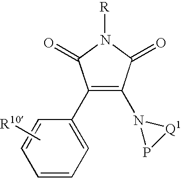
wherein R is as defined in relation to formula (I);

R^{10″} is one or more, suitably up to three, substituents selected from the list consisting of perfluoroalkyl, halo, nitro, alkoxy, arylcarbonyl, alkyl;

Z is a bond or an alkylene chain;

—X—Y— is —CH═N, —(CH₂)ₜ—, —(CH₂)ᵤCH(U)—, —(U)CH(CH₂)ᵤ—, —CH═CH—, —(CH₂)ᵥC(alkyl)₂—, —C(O)C(alkyl)₂—, —C(O)O—, where t, u, and v are independently 1 to 4, and U is alkyl, carboxy, alkoxycarbonyl, hydroxyalkyl, and amido;

R^{12a′}, R^{12b′}, and R^{12c′} are each independently hydrogen, nitro, alkoxy, 4-ethylpiperazin-1-yl, 4-BOC-piperazin-1-yl, 4-methyl-piperazin-1-yl, 4-methylpiperazin-1-yl, halo, alkyl, piperazin-1-yl, perfluoroalkyl, and alkylsulphonylamino. Suitably, Z is a bond or a C₁₋₂ alkylene chain.

Examples of Z include a bond, methylene or ethylene.

Examples of —X—Y— are —CH—N—, —(CH₂)₂—, —CH(Me)CH₂—, —CH═CH—, —CH(CO₂H)CH₂—, —CH(CO₂Me)CH₂—, —(CH₂)₃—, —CH(CH₂OH)CH₂—, —CH₂CH(CH₂OH)—, —CH₂CH(Me)-, —CH₂C(Me)₂—, —CH(CONH₂)CH₂—, —C(O)C(Me)₂—, and —C(O)O—.

Examples of R^{12a′}, R^{12b′}, and R^{12c′} include hydrogen, nitro, fluoro, methoxy, 4-ethylpiperazin-1-yl, 4-BOC-piperazin-1-yl, 4-methyl-piperazin-1-yl, 4-methylpiperazin-1-yl, chloro, bromo, trifluoromethyl, and methanesulphonylamino.

Preferably, Z is a bond.

Preferably, —X—Y— is —(CH₂)₂— or —CH(CH₂OH)CH₂—, —CH(Me)CH₂—, —CH₂CH(Me)-, or —CH₂C(Me)₂—.

Preferably, R^{12b′} is fluorine.

Preferably, R^{12a′} is fluorine.

Most preferably, R^{10″} is 2-Br, 2-Cl, 2-F, 2-OMe, 3-Cl, 3-F, 3-Me, 4-Br, 4-Cl, 4-1,2,3-di-F, 2,5-di-F, 2,6-di-F, 3,4-di-F, 3,5-di-F, 2,3,5-tri-F, 2,4-di-Cl, 3,5-di-Me;

Z is a bond;

—X—Y— is —(CH₂)₂—, —CH(CH₂OH)CH₂—, —CH(Me)CH₂—, —CH₂CH(Me)-, or —CH₂C(Me)₂—;

R^{12b′} is fluorine; and

R^{12a′} is fluorine.

As disclosed in WO 00/21927, there is a subgroup of compounds falling wholly within formula (I) being of formula (IG): 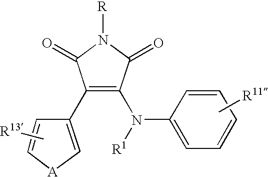
wherein R and R′ are as defined in relation to formula (I);

A is N(alkyl), oxygen, or sulphur.

Examples of A are N(methyl), oxygen, and sulphur.

Preferably, A is sulphur.

R^{11″} is one or more, suitably up to three, substituents selected from the group consisting of hydrogen, halo, alkyl, alkylthio, —S—CH═N—, phenoxy, —(CH₂)_{w}—, hydroxy, carboxy, —O(CH₂)ₓO—, hydroxyalkyl, and alkylaminosulphonylalkyl, where w and x are independently 1 to 4.

Examples of R^{11″} are hydrogen, bromo, methyl, methylthio, chloro, —S—CH═N—, phenoxy, —(CH₂)₃—, hydroxy, carboxy, —O(CH₂)O—, fluoro, hydroxymethyl, and MeNHSO₂CH₂—.

Preferably, R^{11″} is 3-Br, 4-Me, 4-SMe, 3-Br-4-Me, 3-Cl, 3,4-[S—CH═N]—, 3-OPh, 3,4-[(CH₂)₃]—, 3-SMe, hydrogen, 3,5-diBr-4-OH, 3,5-diCl-4-OH, 3-CO₂H-4-Cl, 3,4-[-OCH₂O]—, 3-Cl-4-OH, 3,5-diF, 3-CH₂OH, 3-OH, or 4-CH₂SO₂NHMe.

R^{13′} is one or more, suitably up to two, substituents selected from the group consisting of —(CH═CH)₂— and hydrogen.

Examples of R^{13′} include 4,5-[(CH═CH)₂]- and hydrogen.

Preferably, R^{13′} is hydrogen.

As disclosed in WO 00/21927, there is a subgroup of compounds falling wholly within formula (1) being of formula (IH): 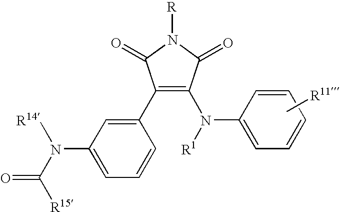
wherein R and R¹ are as defined in relation to formula (I);

R^{11′″} is —[(CH₂)ₐₐ] where aa is 1 to 4;

R^{14′} is hydrogen;

R^{15′} is alkyl, unsubstituted or substituted phenylamino, unsubstituted or substituted phenylalkylamino, cyclohexylamino, alkenylamino, phenyl, benzyl, styryl, or alkylamino.

Examples of R^{11′″} include 3,4-[(CH₂)₃].

Suitably, R^{15′} is C₁₋₆alkyl, (halophenyl)amino, phenylalkylamino, cyclohexylamino, propenylamino, phenyl, benzyl, styryl, propyl, ethylamino, or (methoxyphenyl)amino.

Examples of R^{15′} include methyl, (3-fluorophenyl)amino, phenylethylamino, cyclohexylamino, propenylamino, phenyl, benzyl, trans-styryl, n-propyl, ethylamino, and (3-methoxyphenyl)amino.

As disclosed in WO 00/21927, there is a subgroup of compounds falling wholly within formula (I) being of formula (IJ): 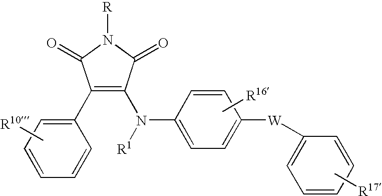
wherein R and R¹ are as defined in relation to formula (I);

R^{10′″} represents one or more, suitably up to three, substituents independently selected from alkoxy or halo;

R^{16′} represents one or more, suitably up to three, substituents independently selected from hydrogen, carboxy, alkoxycarbonyl, or alkylaminocarbonyl;

R^{17′} represents one or more, suitably up to three, substituents independently selected from carboxy, alkoxycarbonyl, halo, alkylaminocarbonyl, nitro, or hydrogen;

W is sulphur, oxygen, or substituted or unsubstituted NH.

Suitably, W is sulphur or oxygen. Favourably, W is sulphur.

Suitably, R^{10′″} is C₁₋₆alkoxy, chloro, or fluoro.

Examples of R^{10′″} are methoxy, 4-chloro, 2-chloro, and 2,3-difluoro.

Favourably, R^{10′″} is 2,3-difluoro.

Suitably, R^{16′} is hydrogen, carboxy, C₁₋₆alkoxycarbonyl, or C₁₋₆alkylaminocarbonyl.

Examples of R^{16′} are carboxy, hydrogen, ethoxycarbonyl, methoxycarbonyl, and methylaminocarbonyl.

Favourably, R^{16′} is hydrogen.

Suitably, R^{17′} is carboxy, C₁₋₆alkoxycarbonyl, halo, C₁₋₆alkylaminocarbonyl, nitro, or hydrogen;

Examples of R^{17′} are 2-carboxy, 3-carboxy, 4-carboxy, 4-chloro, 2-methylaminocarbonyl, 4-nitro, hydrogen, and 2-ethoxycarbonyl.

Favourably, R^{17′} is 3-carboxy.

As disclosed in WO 00/21927, there is a subgroup of compounds falling wholly within formula (I) being of formula (IK): 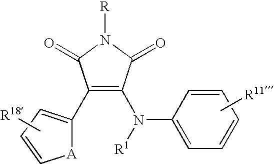
wherein R and R¹ are as defined in relation to formula (I);

R^{11″″} represents one or more, suitably up to three, substituents independently selected from halo and hydroxy;

R^{18′} represents one or more, suitably up to three, substituents independently selected from hydrogen, alkyl, and —(CH═CH)₂—;

A is sulphur.

Suitably, R^{11″″} is chloro or hydroxy.

Examples of R^{11″″} are 3-chloro and 3,5-dichloro-4-hydroxy.

Suitably, R^{18′} is hydrogen, C₁₋₆alkyl, or —(CH═CH)₂—.

Examples of R^{18′} include hydrogen, methyl, and 3-methyl-4,5-[(CH═CH)₂]—.

As disclosed in WO 00/21927, there is a subgroup of compounds falling wholly within formula (I) being of formula (IL): 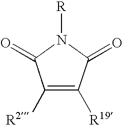
wherein R is as defined in relation to formula (I);

R^{2′″} is unsubstituted or substituted heterocyclyl or unsubstituted or substituted aryl;

R^{19′} is unsubstituted or substituted heterocyclyl, or a quaternized salt thereof.

There is a subgroup of compounds within formula (IL) of formula (IL′) wherein R, R^{2′″}, and R^{19′} are as defined in relation to formula (IL) with the proviso that (IL′) does not include the following compounds, hereinafter referred to as List L′:
3-indol-1-yl-4-(1-methyl-1H-indol-3-yl)-pyrrole-2,5-dione;
1-(1-methyl-2,5-dioxo-4-phenylamino-2,5-dihydro-1H-pyrrol-3-yl)-pyridinium chloride;
1-1-(4-methyl-pentyl)-2,5-dioxo-4-phenylamino-2,5-dihydro-1H-pyrrol-3-yl)-pyridinium chloride;
1-(1-dodecyl-2,5-dioxo-4-phenylamino-2,5-dihydro-1H-pyrrol-3-yl)-pyridinium chloride;
3-[2,5-dihydro-4-(1H-imidazol-1-yl)-1-methyl-2,5-dioxo-1H-pyrrol-3-yl]-1H-indole-1-carboxylic acid, 1,1-dimethylethyl ester;
3-(1H-imidazo[4,5-b]pyridin-1-yl)-4-(1H-indol-3-yl)-1-methyl-1H-pyrrole-2,5-dione;
3-(1H-indol-3-yl)-1-methyl-4-(1H-pyrrolo[2,3-b]pyridin-1-yl)-1H-pyrrole-2,5-dione;
3-(1H-indol-3-yl)-1-methyl-4-(1-piperidinyl)-1H-pyrrole-2,5-dione;
3-[4-(diphenylmethyl)-1-piperazinyl]-4-(1H-indol-3-yl)-1-methyl-1H-pyrrole-2,5-dione;
3-(1H-benzimidazol-1-yl)-4-(1H-indol-3-yl)-1-methyl-1H-pyrrole-2,5-dione;
3-(1H-benzotriazol-1-yl)-4-(1H-indol-3-yl)-1-methyl-1H-pyrrole-2,5-dione;
3-(1H-imidazol-1-yl)-4-(1H-indol-3-yl)-1-methyl-1H-pyrrole-2,5-dione;
3-(1H-indol-1-yl)-4-(1H-indol-3-yl)-1-methyl-1H-pyrrole-2,5-dione;
3-(1H-indazol-1-yl)-4-(1H-indol-3-yl)-1-methyl-1H-pyrrole-2,5-dione;
3-[3-[(dimethylamino)methyl]-1H-indol-1-yl]-4-(1H-indol-3-yl)-1-methyl-1H-pyrrole-2,5-dione;
3-(1H-benzimidazol-1-yl)-4-(1H-indol-3-yl)-1H-pyrrole-2,5-dione;
3-(1H-indol-1-yl)-4-(1-methyl-1H-indol-3-yl)-1H-pyrrole-2,5-dione; and
3-(3,5-dimethyl-1-phenyl-1H-pyrazol-4-yl)-4-(4-morpholinyl)-1H-pyrrole-2,5-dione.

Suitably, R^{2′″} is thienyl, phenyl, or phenyl substituted with one or more halogen groups.

Examples of R^{2′″} include phenyl, 3-thienyl, 2-thienyl, 4-chlorophenyl, and 2,4-dichlorophenyl.

Favourably, R^{2′″} is phenyl, 3-thienyl, 4-chlorophenyl, or 2,4-dichlorophenyl.

Suitably, R^{19′} is indolinyl, pyridinium halide, azabicyclooctanyl, or triazaspirodecanonyl.

Examples of R^{19′} include indolin-1-yl, 3-amino-1-pyridinium chloride, 2-methylindolin-1-yl, 1,3,3-trimethyl-6-azabicyclo[3,2,1]octan-6-yl, and 1-phenyl-1,3,8-triazaspiro-[4,5]-decan-4-one-8-yl.

Favourably, R^{19′} is indolin-1-yl, or 2-methylindolin-1-yl.

Certain of the compounds of formula (I) may contain at least one chiral carbon, and hence they may exist in one or more stereoisomeric forms. The present invention encompasses all of the isomeric forms of the compounds of formula (I) whether as individual isomers or as mixtures of isomers, including racemates.

Particularly preferred compounds of the subject invention include 3-(2,4-dichlorophenyl)-4-(1-methyl-1H-indol-3-yl)-1H-pyrrole-2,5-dione and 3-(3-chloro-4-hydroxyphenylamino)-4-(2-nitrophenyl)-1H-pyrrole-2,5-dione. These maleimides inhibit GSK-3α in vitro with Kᵢs of 9 nM and 31 nM, respectively (Coghlan et al., Chem. & Biol. 7(10): 793-803 (2000)). Both compounds inhibited the beta isoform of GSK-3 with similar potency.

Additional maleimide inhibitors (i.e., 3-anilino-4-arylmaleimide) of GSK-3 have been identified using automated array methodology (Smith et al., Bioorg. Med. Chem. Lett. 11(5): 635-9 (2001)).

Also contemplated herein is the use of maleimide compounds that are protein kinase C (PKC) inhibitors. Such maleimides include RO-31-8220, a bisindolylmaleimide, indolocarbozole K-252a, perylenequinone, calphostin C, calphostin C, Go 6976, Go 6983 and isoquinolinesulfonamide H7. See Debais et al., J. Cell. Biochem. 81(1): 68-81 (2001) and Yang et al., Mol. Pharm. 61(5): 1163-73 (2002) for activity of these maleimides. Preferable agents are those that are PKC selective, such as RO-31-8220, which has predominant specificity for the PKC alpha isoform (Schwaller et al., Br. J. Cancer 76(12): 1554-7 (1997))

Two maleimides that inhibit GSK-3 are SB-216763 and SB-415286. These maleimides inhibit GSK-3α in vitro with Kᵢs of 9 nM and 31 nM respectively (Coghlan et al., Chem. & Biol. 7(10): 793-803 (2000)). Both compounds inhibited the beta isoform of GSK-3 with similar potency.

Another group of maleimides are bisindolylmaleimide I and IX which have been shown to be potent inhibitors of GSK-3 (Hers et al., FEBS Lett. 460(3): 433-6 (1999)). Additional maleimide inhibitors (i.e., 3-anilino-4-arylmaleimide) of GSK-3 have been identified using automated array methodology (Smith et al., Bioorg. Med. Chem. Lett. 11(5): 635-9 (2001)).

Another group of compounds that can modulate GSK-3 are Akt-3 (also known as protein kinase B or RAC-PK) modulatory compounds. For example, the Akt-3 inhibitors RO 31-8220, staurosporine (Masure et al., Eur. J. Biochem. 265(1): 353-60 (1999)) and topotecan (Nakashio et al., Cancer Res. 60: 5303-09 (2000)) can be used to modulate GSK-3. Although RO 31-8220 is a PKC inhibitor and staurosporine is a broad spectrum kinase inhibitor, both work to suppress Akt-3 activity.

A group of protein kinase C inhibitors may also be effective. Preferred inhibitors are selective inhibitors such as RO 31-7549, RO 31-8220, calphostin C and ilmofosine (Amon et al., Agents & Actions 39(1-2): 13-9 (1993)).

Additional GSK-3 inhibitors and modulators can be determined using the following assays as would be known to one skilled in the art. Agents identified using such assays can then be further assessed using the in vivo and in vitro assays disclosed herein for assessing enhancement of bone mineralization.

One assay for assessing a GSK-3 modulatory compound uses a GSK-3 peptide. The GSK-3 specific peptide used in this assay was derived from the phosphorylation site of glycogen synthase and its sequence is: YRRAAVPPSPSLSRHSSPHQ(S)EDEEE. The serine (S) is pre-phosphorylated.

The buffer used to make up the glycogen synthase peptide and [γ-³³P] ATP consists of 25 mM MOPS, 0.2 mM EDTA, 10 mM magnesium acetate, 0.01% Tween-20, and 7.5 mM mercaptoethanol at pH 7. The compounds are dissolved in dimethyl sulphoxide (DMSO) to a final concentration of 100 mM. Various concentrations are prepared in DMSO and mixed with the substrate (i.e., GSK-3 peptide) solution (to a final concentration 20 μM) along with rabbit or human GSK-3α and GSK-3β (final concentration 0.5 U/mL enzyme). The reactions are initiated with the 11 addition of [γ-³³P] ATP (500 cpm/pmole) spiked into a mixture of ATP (final concentration of 10 μM). After 30 min at room temperature, the reaction is terminated by the addition of 10 μL of H₃PO₄/0.01% Tween-20 (2.5%). A volume (10 μL) of the mixture is spotted onto P-30 phosphocellulose paper. The paper is washed four times in H₃PO₄ (0.5%), 2 mins for each wash, air dried and the radioactive phosphate incorporated into the synthetic glycogen synthase peptide, which binds to the P-30 phosphocellulose paper and counted using a scintillation counter.

Another method for screening GSK-3 inhibitory compounds is based on the ability of the kinase to phosphorylate a biotinylated peptide, the sequence of which is derived from the phosphorylation site of glycogen synthase and its sequence is: Biot-KYRRAAVPPSPSLSRHSSPHQ(S)EDEEE, wherein “Biot” refers to the biotin moiety. The serine (S) is a pre-phosphorylated serine, as is glycogen synthase in vivo. The phosphorylated, biotinylated peptide is then captured, onto streptavidin coated SPA beads (Amersham Technology), where the signal from the ³³P can be amplified via the scintillant contained in the beads.

The kinase is assayed at a concentration of 10 nM final in 25 mM MOPS buffer, pH 7.0 containing 0.01% Tween-20, 7.5 mM 2-mercaptoethanol, 10 mM magnesium acetate, and 10 μM [γ-³³P]-ATP. After 60 minutes incubation at room temperature, the reaction is stopped by the addition of 50 mM EDTA solution containing the Streptavidin coated SPA beads to give a final 0.5 mg of beads per assay well in a 384 microtiter plate. Other plates can be utilized as appropriate.

10 mM stock solutions of the compounds of the invention in 100% DMSO are generated as a first step in the screening process. The second step involves the creation of dose-response plates where these compounds are diluted across the plate and where the final low and high concentrations are 0.008 and 10 μM in the kinase assay. The third step involves the creation of the assay plates. This can be achieved by transferring the compounds from four 96 dose response plates to a 384 assay plate. The fourth step is to perform the assay as described and count the resulting plates using a microbeta liquid scintillation and luminescence counter. The final step is data acquisition and analysis where IC50 values are generated for each compound.

Preferably, the most potent compounds of the present invention demonstrate IC50 values in the range of from between about 1 to 10 nM.

In yet another assay, a protein kinase C (PKC) peptide is utilized. The PKC peptide can be a fragment of bovine myelin basic protein (residues 4-14). This sequence is a specific substrate for PKC. The buffer used to make up the myelin basic protein and [γ-³³P]-ATP consisted of 10 mM Tris, 0.9 mM EGTA, 200 μM calcium chloride, 10 mM magnesium chloride and a final concentration of 40 μg/mL of L-a-phosphatidyl-L-serine and 1 μg/mL of 1,3 diolein at pH 7.50.

A candidate compound or other reagent is dissolved in dimethyl sulphoxide (DMSO) to a final concentration of 100 mM. Various concentrations are made up in DMSO and mixed with the substrate (i.e., myelin basic protein) solution (to a final concentration of 0.1 mg/mL) described above, along with the relevant human recombinant PKC isoform (final concentration of 88 mU/mL). The reactions is initiated with the addition of [γ-³³P]-ATP (500 cpm/pmole) spiked into a mixture of ATP (final concentration of 10 μM). After 20 min at room temperature 15 μL of the reaction was spotted onto P-30 phosphocellulose paper. The paper is washed four times in 0.5% H₃PO₄, for 2 mins for each wash, air dried and the radioactive phosphate incorporated into the myelin basic protein, which binds to the P-30 phosphocellulose paper, is counted in a microbeta scintillation counter. These assays can be modified for use in identifying compounds that modulate any of the other proteins discussed herein as being involved in bone remodeling.

7.1.2 PKA Inhibitors

As discussed above for GSK-3 inhibitors, PKA inhibitors would have similar uses. Preferred PKA inhibitors include but are not limited to H89 (Calbiochem). Additional PKA inhibitors include but are not limited to protein kinase A inhibitor 5-24, inhibitor 6-22 Amide and inhibitor 14-22 Amide (Calbiochem).

7.1.3 PKC Inhibitors

As discussed above for GSK-3 inhibitors, PKC inhibitors would have similar uses. Contemplated PKC inhibitors include but are not limited to PKC inhibitor 20-28 myristoylated, EGF-R fragment 651-658 myristoylated, Ro 31-8425, Ro32-0432 and the like (Calbiochem).

7.1.4 MEK1/2 Inhibitors

As discussed above for GSK-3 inhibitors, MEK 1/2 inhibitors would have similar uses. MEK1/2 inhibitors include but are not limited to U0126 (Calbiochem) and PD98059 (Calbiochem).

7.1.5 MAPK Inhibitors

As discussed above for GSK-3 inhibitors, MAPK inhibitors would have similar uses. P38 MAPK inhibitors contemplated include but are not limited to SB203580 (Ishizuka et al., J. Immunol. 167(4): 2298-304 (2001) and which can be obtained from Calbiochem), SB202190 (Karahashi et al., Biochim. Biophys. Acta 1502(2): 207-23 (2000)), PD169316 (Paine et al., J. Biol. Chem. 275(15): 11284-290 (2000)), fr-167653 (Matsuoka et al., Am. J. Physiol. Lung Cell Mol. Phsiol. 283: L103-12 (2002)), [trans-1-(4-hydroxycyclohexyl)-4-(4-fluorophenyl)-5-(2-methoxypyridimidin-4-yl)imidazole) (Underwood et al., Am. J. Physiol. Lung Cell Mol. Physiol. 279(5): L895-902 (2000)), and 2-(4-Chlorophenyl)-4-)4-fluorophenyl)-5-pyridin-4-yl-1,2-dihydropyrazol-3-one (Calbiochem).

7.1.6 JNK Inhibitors

As discussed above for GSK-3 inhibitors, c-Jun amino kinase (JNK) pathway inhibitors would have similar uses. JNK inhibitors contemplated for used include but are not limited to SP-600125 (Calbiochem), the indolocarbazole of the K252a family CEP-1347/KT-7515 (Saporito et al., Prog. Med. Chem. 40: 23-62 (2002); and Maroney et al., J. Neurochem. 73(5): 1901-12)), and JNK-interacting protein-1 (JIP-1) peptides that bind to JNK (Barr et al., J. Biol. Chem. 277(13): 10987-97 (2002)).

7.1.7 Calcium Mobilization Inhibitors

As discussed above for GSK-3 inhibitors, calcium mobilization inhibitors would have similar uses in modulating bone mineralization and the Wnt pathway and study thereof. One preferred calcium mobilization inhibitor is [8-(diethylamino)octyl-3,4,5-trimethoxybenzoate HCl (TMB-8) produced by Calbiochem.

7.1.8 MAPKAPK2 Inhibitors

Mitogen-activated protein kinase activated protein kinase-2 (MAPKAPK2) inhibitors can also be utilized for the same purposes as discussed for GSK-3 inhibitors. MAPKAPK2 is a downstream substrate of MAPK, discussed above. Therefore, inhibitors of MAPK will also inhibit MAPKAPK2. MAPKAPK2 inhibitors include but are not limited to Hsp25 kinase inhibitor (Calbiochem, Cat. No. 385880) and SB203580 (Ishizuka et al., J. Immunol. 167(4): 2298-304 (2001)).

7.1.9 G-protein Coupled Signaling Inhibitors

G-protein coupled signaling inhibitors, such as pertussis toxin (Sigma), can be used in the assays as discussed herein for GSK-3 inhibitors. Other G-protein coupled signaling inhibitors can also be utilized.

7.1.10 Nitric Oxide Synthase Inhibitors

Nitric oxide synthase (NOS) inhibitors are also contemplated for use in manners similar to the uses discussed herein for GSK-3 inhibitors. NOS inhibitors contemplated include but are not limited to N(G)-nitro-L-arginine (L-NNA) (Clark et al., Resuscitation 57(1): 101-8 (2003)) and L-NAME (Sigma).

7.1.11 COX-2 Inhibitors

COX-2 inhibitors are also contemplated for similar uses to those described herein for GSK-3 inhibitors. COX-2 inhibitors include but are not limited to indomethacin (Sigma), VIOXX (rofecoxib, Merck & Co.), CELEBREX (celecoxib, G. D. Searle & Co.), 2-aminosulfonylphenyl-3-phenyl-indole 5a (Hu et al., Bioorg. Med. Chem. 11(7): 1153-60 (2003)), and SC-560 (Pinheiro et al., Inflamm. Res. 51(12): 603-10 (2002)).

7.2. Nucleic Acids and Polypeptides

Also contemplated herein are nucleic acids that modulate (and preferably activate) the Wnt pathway or any of the proteins/genes listed as being up- or down-regulated in response to bone load alone or in combination with other agents. Preferably these nucleic acids enhance bone remodeling to allow for greater bone density. The nucleic acids contemplated herein include antisense compounds that bind to either the sense or antisense strand of a gene or to a transcript of a gene. Contemplated nucleic acids also include small inhibitory RNAs (siRNAs) that promote RNA interference. Suitable targets for antisense and siRNA molecules include GSK and catenin, LRP5, LRP5, axin, and any other members of the Wnt pathway.

Polypeptides that modulate the Wnt pathway are also contemplated. Such polypeptides include immunoglobulins, peptide aptamers, blocking compounds and the like which are discussed further below.

7.2.1. RNA Interference

Proteins in the Wnt pathway that are involved with bone mineralization can also be analyzed or modulated for treatment purposes using RNA interference (RNAi). This is a technique for post-transcriptional gene silencing, in which target gene activity is specifically abolished with cognate double-stranded RNA (dsRNA). RNAi resembles in many aspects PTGS in plants and has been detected in many invertebrates including trypanosome, hydra, planaria, nematode and fruit fly (Drosophila melanogaster). RNA interference may be involved in the modulation of transposable element mobilization and antiviral state formation. RNA interference in mammalian systems is disclosed in PCT application WO 00/63364, which is incorporated by reference herein in its entirety. Basically, dsRNA, homologous to the target (e.g., GSK-3 or β-catenin or homologous to any gene's RNA of any of the tables herein which discuss up- and down-regulated genes in response to bone load alone or in combination with other agents) is introduced into the cell and a sequence specific reduction in gene activity is observed. Small interfering RNAs (siRNAs) and short hairpin RNAs (shRNAs) are contemplated for such use. See for example Yu et al., Proc. Natl. Acad. Sci. USA, 99: 6047-6052 (2002); Paddison et al., Genes & Dev., 16: 948-58 (2002); Brummelkamp et al., Science 296: 550-53 (2002); Tuschl, (2002) Nature Biotechnology 20: 446-8 (2002); and the references cited therein. These moieties can be used as research tools to further characterize bone remodeling, as well as reagents to modulate bone remodeling in a subject.

One particular gene of interest in the Wnt pathway for study using RNAi techniques is β-catenin. β-catenin is an essential component of the canonical Wnt pathway. Upon activation of this pathway, β-catenin is no longer phosphorylated and therefore accumulates in the cytoplasm and translocates into the nucleus. Once in the nucleus, β-catenin relieves inhibitors of targeted transcription factors, including TCF and LEF, and in turn, activates transcription.

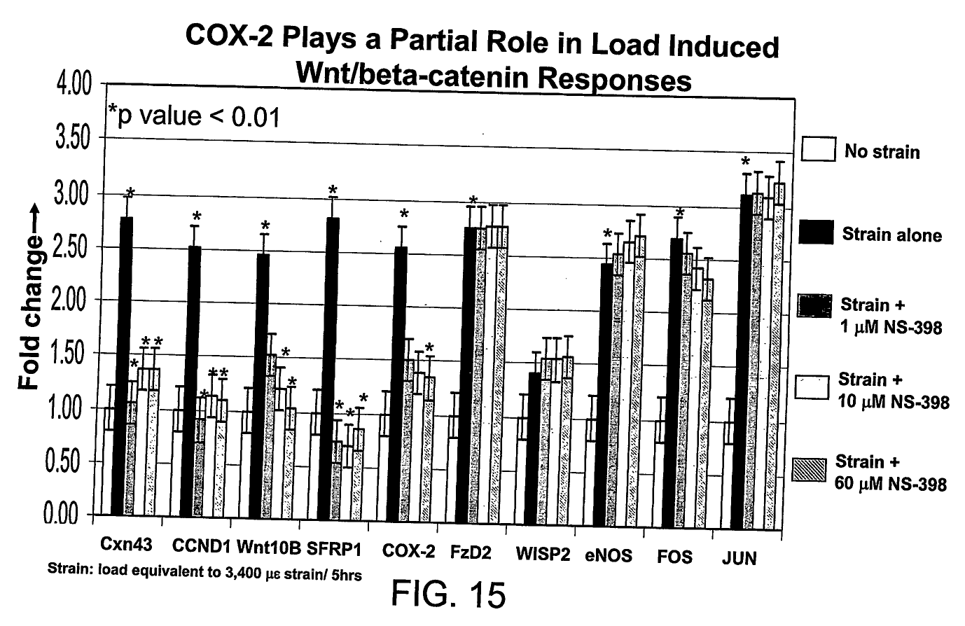

These experiments can be utilized with any of the genes in the pathways depicted in FIG. 15 or listed in any of the tables of up- and down-regulated genes can be used. For example, β-catenin RNAi can be transfected into MC3T3 cells (or other suitable bone cell line). The cells are then subjected to load for 5 hrs as previously described above. Real-time PCR can then be performed (or other means of analyzing RNA) on the genes. Gene expression is assessed for such genes as connexin 43, osteonectin, OPG, eNOS, COX-2, PTGS, IL-6, cyclin D1, Frizzled 2, Wnt 10B, SFRP1 and SFRP4 or any of the genes discussed herein as modulated in response to bone load and/or Wnt pathway modulation.

To specifically identify which load responsive genes are dependent upon LRP5 expression MC3T3 cells can be transfected with LRP5 RNAi. Similar to the experiments with the β-catenin RNAi, the responses in gene expression between the cells that were loaded in the presence and absence of the LRP5 RNAi are assessed. If LRP5 expression is confirmed to be blocked and no differences are seen with the LRP5 RNAi treated samples, it is possible that LRP6 (a close family member of LRP5) could be compensating for LRP5 function. To address this and to access whether there is LRP6 contributions in the loading responses observed, MC3T3 cells can be transfected with LRP6 RNAi alone, as well as LRP6 and LRP5 RNAi combined. Thus, in this instance, RNAi is being used to further characterize LRP5 and LRP6 activity relative to each other and bone remodeling.

More specifically, RNA interference experiments can be carried out as follows. Bone cells, such as MC3T3 cells, are cultured in a bioflex 6-well plates for 3 days in growth media until 80% confluent. The media is then removed, and the cells are washed with 2 mL OptiMEM (Invitrogen). The DNA/Lipofectamine 2000 mix is prepared by pre-diluting 10 μL Lipofectamine 2000 (per well) in 250 μL OptiMEM. This mixture is then combined with 4 μg double stranded RNAi in 250 μL OptiMEM. The OptiMEM is removed from the cells, and the combined DNA/lipofectamine mixture (500 μL total) is added to the cells and incubated for 4 hr at 37° C. The media then is changed to either growth media or serum free media containing 0.25% BSA and incubated for 24 hr. The cells are then subsequently subjected to 50 to 5,000 με of mechanical load (e.g., 3,400 με) as previously herein.

RNA is then harvested. RNA can be harvested immediately following administration of mechanical load, as well as at any time point thereafter (e.g., 24 hours post load). The RNA is then analyzed using any of the methods described herein, such as real-time PCR.

7.2.2 Antisense Compounds

In another aspect of the invention, proteins involved in Wnt pathway modulation (preferably Wnt pathway activation and thereby bone mineralization), can be altered using antisense compounds for diagnostic, research, and treatment purposes.

As an example, preparing antisense oligonucleotides can be performed as follows. Studies have been undertaken using antisense technology in the osteoblast-like murine cell line, MC3T3. These cells can be triggered to develop along the bone differentiation sequence. An initial proliferation period is characterized by minimal expression of differentiation markers and initial synthesis of collagenous extracellular matrix. Collagen matrix synthesis is required for subsequent induction of differentiation markers. Once the matrix synthesis begins, osteoblast marker genes are activated in a clear temporal sequence: alkaline phosphatase is induced at early times, while bone sialoprotein and osteocalcin appear later in the differentiation process. This temporal sequence of gene expression is useful in monitoring the maturation and mineralization process. Matrix mineralization, which does not begin until several days after maturation has started, involves deposition of mineral on and within collagen fibrils deep within the matrix near the cell layer-culture plate interface. The collagen fibril-associated mineral formed by cultured osteoblasts resembles that found in woven bone in vivo and therefore is used frequently as a study reagent.

MC3T3 cells (or other suitable bone cell line) are transfected with antisense oligonucleotides for the first week of the differentiation, according to the manufacturer's specifications (U.S. Pat. No. 5,849,902). Typically, the antisense oligonucleotides are transfected into bone cells, such as MC3T3. RNA is then isolated from the cells according to manufacturer instructions or other procedures known in the art. Northern analysis, real-time PCR or alternative RNA assay, is performed to analyze the effect of the antisense polynucleotide. Additionally, transcription profiling can be performed to study the impact on the Wnt pathway of an antisense compound against a gene that encodes a protein involved in Wnt signaling.

7.3 Polypeptides

In addition to nucleic acids that modulate, and preferably up-regulate, the Wnt pathway (thereby enhancing bone mineralization), polypeptides and biologically active fragments thereof as well as aptamers are also contemplated. Suitable proteins and biologically active fragments include polypeptides and aptamers (which modulate proteins of the pathways depicted in FIG. 16, e.g. GSK-3 and β-catenin. Also contemplated are any type of immunoglobulin (e.g., antibody) that can modulate activity (e.g., monoclonal, polyclonal, lambda phage antibodies (Cat technology) and fragments thereof).

The in vitro loading experiments discussed above can also be used to investigate the gene responses of the load responsive genes and the proteins they encode (i.e., bone load gene profile) to other known synthetic Wnt pathway agonists (e.g., other GSK-3 inhibitor-like compounds), natural Wnt pathway ligands and synthetic ligands.

The level of Wnt pathway activation can be assessed in MC3T3 cells (or other suitable bone cell lines) with known Wnt pathway activators include but are not limited to Wnt 1 and Wnt 3A, small molecule Wnt mimetics as well as peptide aptamers (e.g., aptamer 262) that interact with LRP5 and activate Wnt signaling. Such assays can also be used to study Wnt antagonists.

Wnt antagonists include but not limited to Dkk1 and small molecule Dkk1 antagonists. Similarly, gene activity and modulation to Wnt antagonists can be assessed using, for example, the TCF-luciferase reporter construct. The TCF-luciferase reporter can be used to measure the effects of mechanical loading itself on Wnt pathway activity.

For example, MC3T3 cells can be plated as previously described above and cultured for three days until confluence. The media is changed to either serum free containing BSA or low serum (1% FBS) containing αMEM and then incubated for 24 hrs. One hour prior to loading, one set of plates is pretreated with a dose range of a Wnt agonist (e.g., GSK-3 inhibitor or Dkk1 antagonist) while a similar control set is not be pretreated. For experiments involving Wnt1, Wnt 3A and Dkk1, conditioned media from 293 cells transiently transfected with these specific cDNA constructs (or control cector) can be used as a source of these proteins. For preparation of Wnt1, Wnt 3A and Dkk1 conditioned media's, 293 cells can be trasfected using Lipofectamine 2000 (Invitrogen) as described by the manufacturer using 10 μg plasmid DNA per 100 mm culture dish. Forty-eight hours following the 293 cell transfection, the conditioned media is collected (10 mL total), centrifuged to remove cell debris, aliquoted and frozen at −70° C. for subsequent MC3T3 cell FlexerCell experiments. Therefore, following pretreatment of the MC3T3 cells with any Wnt mimetic ligands, small molecules, or other Wnt pathway modulator, the MC3T3 bone cells are then subjected to mechanical load as discussed herein. RNA is harvested from the loaded and the non-loaded control samples immediately following load and at time-points post-load using the Qiagen Rneasy mini kit or other means. Real-time PCR is performed on the load signature set genes at desired time points to observe changes in gene expression with treatment.

For experiments that involve measuring the activation of the Wnt pathway, transient transfections with, for example, a TCF-luciferase reporter system can be performed. More specifically, 80% confluent bone cells are transfected with about 2.5 μg 16×-TCF(TK)-Luciferase and 0.5 μg TK-Renilla-luciferase per well using the TransFast transfection Reagent (Promega, Madison Wis.) as described by the manufacturer. The prediluted DNA (in 1 mL basal αMEM) is then mixed with 8 μL of the TransFast reagent and incubated for 30 min. At this time, the growth media from the cells is removed and 1 mL basal αMEM is added to each well and incubated for 30 min. Following the 30 min incubation, the media is aspirated from the cells and the TransFast/DNA mixture is then added to the cells and incubated for 1 hr at 37° C. For one group of samples, serum free media containing 0.25% BSA is added (2 mL). In a separate group, 2 mL of growth media is added. The cultures are then incubated overnight, and the media removed and replaced with 1 mL of BSA containing serum free αMEM. The cells are subjected to mechanical load and incubated for 24 hrs or other suitable time period for subsequent luciferase measurements. Luciferase activity is measured following cell lysis with 300-500 μL of passive lysis buffer (Promega, Madison, Wis.) using a Dual Luciferase Reporter Assay system (Promega).

7.4 Immunoglobulins

In another aspect, immunoglobulins are used either alone or in combination for therapy, diagnostics, screening, in combination therapies and the like. If used in the form of protein arrays, immunoglobulins or binding fragments thereof (e.g., Fab) can be used to bind to a suitable substrate to screen for proteins that respond to bone load/stress, augmentation of bone load/stress and the like. Suitable immunoglobulins are any of those which bind to proteins or protein fragments listed herein as responding to mechanical load or enhancement of mechanical load. Commercial producers of antibodies, including monoclonal antibodies, include Abcam, Bethyl Laboratories Inc., BioSource International Inc., Boston Biologicals Inc., Calbiochem-Novabiochem Corp., ICN Biomedicals Inc., MoBiTec, Oxford Biomedical Research, Promega Corp., Research Diagnostics Inc., Rockland Immunochemicals Inc., Santa Cruz Biotechnology, Sigma-Aldrich, Sigma-RBI, Stratagene, United States Biological, Upstate, and Zymed Laboratories Inc. Other manufacturers are also known to produce antibodies and can be used.

8. Combination Therapies

It is also contemplated that combinations of therapies be utilized to optimize bone mineralization in a subject in need thereof. This includes using the agents disclosed herein with such existing therapies as hormone replacement therapy (HRT), selective estrogen-receptor modulators (SERMS), calcitonin, bisphosphonates, raloxifene, calcitonin, and vitamin D or any reagent discussed below. Modulators of the Wnt pathway and bone profile genes are also contemplated for use with any of the agents below, alone (e.g., a GSK-3 inhibitor and a bisphosphonate) or in combination (e.g., alendronate, HRT and a GSK-3 inhibitor). The amounts of these additional agents would vary by patient, but would likely be less than the amount typically administered if the drug was being used as a single agent.

8.1 Hormone Replacement Therapy

Hormone replacement therapy (HRT) usually consists of estrogen and progesterone in postmenopausal women with an intact uterus and estrogen-only in women who have had a hysterectomy. Typical estrogens and their replacement dosages include oral conjugated equine estrogens (0.625 mg/day), oral ethinyl estradiol (0.2 mg/day) and transdermal estradiol (0.05 mg/day usually in the form of one patch twice per week). Oral preparations are more commonly used, however transdermal estrogen replacement may be more effective for individuals who smoke because of their increased hepatic metabolism of oral estrogens. Progesterone may be given cyclically (as medroxyprogesterone, 10 mg/day for 10 to 12 days each month) or continuously (2.5 mg/day). The required doses are greater for estrogen-deficient women (e.g., 20 mg/day of medroxyprogesterone acetate or 5 mg/day of norethindrone). The amount of hormone being replaced likely may be less when used in combination with reagents that modulate proteins involved in bone mineralization. For available approved drug formulations, see Table 6 below.

Hormone replacement therapy, as well as vitamin D and calcium supplementation are also utilized in male subjects suffering from bone loss. In hypogonadal men, testosterone replacement has been shown to increase bone mass. Accordingly, in one aspect, combinations of these agents with the reagents disclosed herein that modulate bone mineralization would be co-administered to male subjects in need thereof.

8.2 Selective Estrogen-Receptor Modulators

Selective estrogen-receptor modulators (SERMs) include but are not limited to raloxifene (Evista®), tamoxifen, torimifene, bazedoxifene acetate (1H-indol-5-ol, 1-[[4-[2-(hexahydro-1H-azepin-1-yl)ethoxy]phenyl]methyl]-2-(4-hydroxyphenyl)3-3-methyl-monoacetate or 1-[p-[2-(hexahydro-1H-azepin-1-yl)ethoxy]benzyl]-2-(p-hycroxyphenyl)-3-methylindol-5-ol monoacetate), tibolone and pharmaceutically acceptable salts thereof. Raloxifene (a nonsteroidal benzothiphene) is the most commonly administered SERM, with the other agents having other indications for which they are FDA approved. Raloxifene is typically administered at a dosage of 60 mg/day.

8.3 Calcitonin

Calcitonin is a peptide with antiresorptive properties. The biologically active form comprises 32 amino acids with an N-terminal disulfide bridge between residues 1 and 7. Salmon calcitonin is an FDA-approved form of calcitonin and is approved as an alternative to estrogen for the treatment but not the prevention of osteoporosis. Salmon calcitonin is the most potent and ironically human calcitonin is the least potent of the available calcitonins.

Salmon calcitonin is typically administered intranasally at 200 U/day with a single administration per day. However, for Paget's disease, salmon calcitonin is administered s.c. or i.m. at a dose of about 50 to about 100 IU, 3-7 times per week. Human calcitonin can be used at about 100 IU (0.5 mg) per day. The nasal dosage is higher, e.g., about 400 IU. For osteoporosis, salmon calcitonin is administered at a rate of 100 IU via injection or 200 IU via intranasal administration. For additional information regarding the administration of calcitonin, see M. Zaidi et al., Molecular and Clinical Pharmacology of Calcitonin in PRINCIPLES OF BONE BIOLOGY 1423-40 (2^{nd} ed., John P. Bilezildan et al., eds., 2002). Other forms of calcitonin are also contemplated for use in combination drug therapies.

8.4 Bisphosphonates

Although bisphosphonates are potent inhibitors of bone remodeling, for yet an unknown reason these agents have been demonstrated to prevent bone loss. Bisphosphonates include but are not limited to alendronate, clodronate, EB-1053, etidronate, ibandronate, incadronate, minodronate, neridronate, olpadronate, pamidronate, risedronate, tiludronate and zoledronate. Bisphosphonates are compounds characterized by two C—P bonds. When the two C—P bonds are on a single carbon atom (i.e., P—C—P), they are analogs of pyrophosphate (i.e., P—O—P).

Alendronate is the most comprehensively studied bisphosphonate currently approved for the treatment of osteoporosis. It is a bisphosphonate or pyrophosphate derivative, which has antiresorptive effects on the skeleton. Alendronate is typically administered in amount of about 5 mg/day for osteoporosis prevention, 10 mg/day for osteoporosis treatment and 40 mg/day to treat Paget's disease (see Table 6 below). Alendronate is also commonly coadministered with HRT (B. Dawson-Hughes, Pharmacologic Treatment of Postmenopausal Osteoporosis in PRIMER ON THE METABOLIC BONE DISEASES AND DISORDERS OF MINERAL METABOLISM 283-288 (4^{th} ed., Lippincott Williams & Wilks, 1999). For additional information on bisphosphonates, see H. Fleisch et al., Bisphosphonates: Mechanisms of Action in PRINCIPLES OF BONE BIOLOGY 1361-85 (2^{nd} ed., John P. Bilezikian et al., eds., 2002) and Table 6 below which provides the bisphosphonates and dosages currently available.

8.5 Vitamin D and Vitamin D Analogs

Currently only the compounds representing the main pathway of vitamin D activation are synthesized for use as drugs. This includes vitamin D₃, also referred to as 25-hydroxyvitamin D₃ or 25-OH-D₃ (calcidiol), and 1α,25-(OH)₂D₃ (calcitriol). The one exception is 24(R),25-(OH)₂D₃ (Secalciferol). Thus, natural prodrugs and metabolites of vitamin D can also be administered. Administration of vitamin D is age dependent. For example, typical oral administration of vitamin D is 200 IU up to age 50, 400 IU up to age 70 and 600 to 800 IU over age 70. For additional information on vitamin D and its analogs, see G. Jones, Vitamin D and Analogs in PRINCIPLES OF BONE BIOLOGY 1407-22 (2^{nd} ed., John P. Bilezikian et al., eds., 2002). For additional Vitamin D preparations, see Table 6 below.

8.6 Calcium Supplementation

Wnt pathway modulators can also be combined with any of the above methodologies and/or with calcium supplements. Calcium supplementation can be provided in the form of calcium carbonate, calcium citrate, calcium bionate, calcium gluconate, calcium lactate, calcium phosphate and tricalcium phosphate. Common dosages include but are not limited to those provided in Table 6 or in smaller dosages.

8.7 Other Drugs

Certain additional drugs have shown that they may aid to prevent bone loss or enhance bone mineralization. Progestins, such as tibolone, may be used to treat osteoporosis and other bone loss disorders. Another alternative is the anti-estrogen, tamoxifen. Tamoxifen is typically administered at about 20 to about 30 mg/day to women who are at risk for breast cancer. These drugs are not currently approved for use in treating bone mineralization disorders.

Other reagents such as omeprazole, amiloride and N-ethyl maleimide have also been shown to be effective at inhibiting bone resorption. The combination of amiloride and N-ethyl maleimide was inhibited more greatly when the reagents were combined than when the reagents were administered individually. Matsuda, J. Osaka City Medical Ctr. 41(2): 653-61 (1992).

TABLE 6

| | | |
|---|---|---|
| | Application in | |
| | Treatment of Bone and | |
| Drug | Mineral Disorders | Dosage (adult) |

Hormones and Analogs

| | | |
|---|---|---|
| Calicitonin | | |
| Human (Cibacalcin) | Paget's Disease | 0.25-0.5 mg i.m or s.c.; q24 h |
| Salmon (Calcimar, | Paget's Disease, | 50-100 IU, i.m. or s.c.; q.o.d. or |
| Miacalcin) | osteoporosis, | q.d. for Paget's disease or |
| | hypercalcemia | osteoporosis; 4-6 IU/kg i.m. or |
| | | s.c.; q.i.d. for hypercalcemia |
| Calcitonin Nasal Spray | Osteoporosis | 200 IU nasal q.d. |
| Estrogens | | |
| Estinyl estradiol | Postmenopausal | 0.02-0.05 mg; q.d. 3/4 wk |
| | osteoporosis | |
| 17β estradiol | | 0.5 mg q.d. |
| (Estrace) | | |
| Transderm Patch | | 0.05-0.1 mg 2x/wk |
| (Estraderm) | | |
| Conjugated equine | | 0.625-1.25 mg q.d. 3/4 wk |
| estrogens | | |
| (Premarin) | | |
| Esterified estrogens | | 0.3-1.25 mg q.d. |
| (Estratab) | | |
| Estropipate | | 0.75 mg q.d. |
| (Ortho-Est .625) | | |
| Conjugated equine | | 0.625 mg estrogen q.d. on days |
| estrogen with | | 1-14 and 0.625 mg estrogen with |
| medroxyprogesterone | | 5 mg MPA q.d. on days 15-28 |
| acetate (MPA) | | |
| (Premphase) | | |
| Prempro | | 0.625 mg estrogen with 2.5 or 5 mg |
| | | MPA q.d. |
| Selective estrogen-receptor | Postmenopausal | 60 mg q.d. |
| modulators (SERMs) | osteoporosis | |
| Raloxifene (Evista ®) | (prevention) | |
| Glucocorticoids | Hypercalcemia due to | 10-60 mg; q.d. |
| Prednisone | sarcoidosis, vitamin D | |
| (Deltasone) | intoxication, and certain | |
| | malignancies such as | |
| | multiple myeloma and | |
| | related | |
| | lymphoproliferative | |
| | disorders | |
| Parathyroid Hormone | Diagnosis of | 200 U; over 10 min infusion |
| Human 1-34 | pseudohypoparathyroidism | |
| (Parathor) | | |
| Testosterone | | |
| Testosterone cypionate | Male hypogonadism | 200-300 mg i.m. q2-3 wk |
| Testosterone enanthate | | 200-300 mg i.m. q2-3 wk |
| Transdermal patch | | |
| Testoderm | | 4-6 mg scrotal patch q24 hr |
| Testoderm TTS | | 5 mg body patch |
| Androderm | | Two 2.5 mg patches q24 hr |
| Vitamin D Preparations | | |
| Cholecalciferol or D₃ | Nutritional vitamin D | 400-1000 U; as dietary |
| | deficiency, osteoporosis, | supplement |
| | malabsorption, | |
| | hypoparathyroidism, | |
| | refractory rickets | |
| Ergocalciferol or D₂ | | 25,000-100,000 U; 3X/wk to |
| (Calciferol) | | q.d. |
| Calcifediol or 25 (OH) D₃ | Malabsorption; renal | 20-50 μg; 3X/wk to q.d. |
| (Calderol) | osteodystrophy | |
| Calcitriol or 1,25 (OH)₂ D₃ | Renal osteodystrophy, | 0.25-1.0 μg; q.d. to b.i.d. |
| (Rocaltrol) or | hypoparathyroidism, | |
| (Calcijex) | refractory rickets. | |
| Dihydrotachysterol (DHT) | Renal osteodystrophy, | 0.2-1.0 mg; q.d. |
| | hypoparathyroidism | |
| Bisphosphonates | | |
| Etidronate | Paget's disease, | p.o., 5 mg/kg, q.d. for 6/12 mo |
| | heterotopic ossification, | for Paget's disease; 20 mg/kg, |
| | hypercalcemia of | q.d. 1 mo before to 3 mo after |
| | malignancy | total hip replacement; 10/20 mg/kg, |
| | | q.d. for 3 mo after spinal |
| | | cord injury for heterotopic |
| | | ossification. |
| | | i.v., 7.5 mg/kg, q.d. for 3 d, |
| | | given in 250-500 mL normal |
| | | saline for hypercalcemia of |
| | | malignancy; 5 mg q.d. for |
| | | osteoporosis prevention. |
| Alendronate | Osteoporosis prevention | 5 mg q.d. for osteoporosis |
| (Fosamax) | and treatment, Paget's | prevention; 10 mg q.d. for |
| | disease | osteoporosis treatment; 40 mg |
| | | q.d. for Paget's disease |
| Pamidronate | Hypercalcemia of | 60-90 mg given as a single i.v. |
| (Aredia) | malignancy, Paget's | infusion over 24 h for |
| | disease | hypercalcemia of malignancy; 4-hr |
| | | infusions also effective for |
| | | 30- or 60-mg doses. 30-mg |
| | | doses over 4 hr on 3 consecutive |
| | | days for a total of 90 mg for |
| | | Paget's disease |
| Risedronate | Paget's disease | 30 mg q.d. for 2 mo. |
| (Actonal) | | |
| Tiludronate | Paget's disease | 400 mg q.d. for 3 mo. |
| (Skelid) | | |

Minerals

| | | |
|---|---|---|
| Bicarbonate, sodium | Chronic metabolic | Must be titrated for each patient |
| | acidosis leading to bone | |
| | disease | |
| Calcium preparations | Hypocalcemia (if | |
| | symptomatic should be | |
| | treated i.v.), | |
| | osteoporosis, rickets, | |
| | osteomalacia, chronic | |
| | renal failure, | |
| | hypoparathyroidism, | |
| | malabsorption, enteric | |
| | oxaluria | |
| Calcium carbonate | | p.o. 400-2000 mg elemental |
| (40% Ca) | | Ca in divided doses; q.d. |
| Calcium citrate | | |
| (21% Ca) | | |
| Calcium chloride | | |
| (36% Ca) | | |
| Calcium bionate | | |
| (6.5% Ca) | | |
| Calcium gluconate | | i.v., 2-20 mL 10% calcium |
| (9% Ca) | | gluconate over several hrs |
| Calcium lactate | | |
| (13% Ca) | | |
| Calcium phosphate, | | |
| dibasic (23% Ca) | | |
| Tricalcium phosphate | | |
| (39% Ca) | | |
| Magnesium | | |
| preparations | | |
| Magnesium oxide | Hypomagnesemia | 240-480 mg elemental Mg; |
| (Mag-Ox, Uro-Mag), | | q.d. |
| p.o. (84.5, 241.3 Mg) | | |
| Phosphate preparations | | |
| Neutra-Phos p.o. | Hypophosphatemia, | p.o., 1-3 g in divided doses; |
| (250 mg P, 278 mg K, | vitamin D-resistant | q.d. |
| 164 mg Na) | rickets, hypercalcemia, | |
| | hypercalciuria | |
| Neutra-Phos-K, p.o. | | |
| (250 mg P, 556 mg K) | | |
| Fleet Phospha-Soda, | | |
| p.o. | | |
| (815 mg P, 760 mg | | |
| Na in 5 mL) | | |
| In-Phos, i.v. | | i.v., 1.5 g over 6-8 hrs. |
| (1 g P in 40 mL) | | |
| Hyper-Phos-K, i.v. | | |
| (1 g P in 15 mL) | | |

Diuretics

| | | |
|---|---|---|
| Thiazides | | |
| Hydrochlorothiazide, | Hypercalciuria, | 25-50 mg; q.d. or b.i.d. |
| p.o. (25, 50, 100 mg) | nephrolithiasis | |
| Chlorthalidone, p.o. | | |
| (25, 50 mg) | | |
| Loop diuretics | | |
| Furosemide, | Hypercalcemia; if | p.o., 20-80 mg, g6 h as |
| p.o. (20, 40, 80 mg), | symptomatic, use i.v. | necessary |
| i.v. (10 mg/mL) | | i.v., 20-80 mg over several |
| | | minutes, repeat as necessary |

Miscellaneous

| | | |
|---|---|---|
| Mitramycine or | Hypercalcemia or | 25 μg/kg in 1 L D5W or |
| Plicamycin | malignancy | normal saline over 4-6 hr. |
| Mithracin, i.v. | | |
| (2.5 mg/vial) | | |

The above reagents can be combined with compounds and compositions that modulate and preferably activate the Wnt pathway (and thereby enhance bone remodeling) in any combination. Most often the existing therapeutic compounds, when administered with one of the Wnt pathway modulating compounds, will be administered in dosages less than those recommended if the existing therapeutic compound was administered alone.

9. Pharmaceutical Formulations

Pharmaceutical formulations of this invention include small compounds or immunoglobulins either alone or in combination. Combinations are contemplated to be both small compounds as well as small compounds and compositions combined with existing therapies.

9.1 Small Compound Formulations

When employed as pharmaceuticals, the compounds of the subject invention are usually administered in the form of pharmaceutical compositions. Pharmaceutical formulations of this invention include combinations of small compounds and combinations of small compounds and polypeptides (e.g., immunoglobulins) or nucleic acids as discussed herein

These compounds and combination therapies can be administered by a variety of routes including oral, parenteral, transdermal, topical, rectal, and intranasal. These compounds and combination therapies are effective as both injectable and oral compositions. Such compositions are prepared in a manner well known in the pharmaceutical art and comprise at least one active compound.

This invention also includes pharmaceutical compositions which contain, as the active ingredient, one or more of the compounds above associated with pharmaceutically acceptable carriers. In making the compositions of this invention, the active ingredient is usually mixed with an excipient, diluted by an excipient or enclosed within such a carrier which can be in the form of a capsule, sachet, paper or other container. The excipient employed is typically an excipient suitable for administration to human subjects or other mammals. When the excipient serves as a diluent, it can be a solid, semi-solid, or liquid material, which acts as a vehicle, carrier or medium for the active ingredient. Thus, the compositions can be in the form of tablets, pills, powders, lozenges, sachets; cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, it may be necessary to mill the active compound to provide the appropriate particle size prior to combining with the other ingredients. If the active compound is substantially insoluble, it ordinarily is milled to a particle size of less than 200 mesh. If the active compound is substantially water soluble, the particle size is normally adjusted by milling to provide a substantially uniform distribution in the formulation, e.g. about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The quantity of active component that is the compound according to the subject invention, in the pharmaceutical composition and unit dosage form thereof may be varied or adjusted widely depending upon the particular application, the potency of the particular compound and the desired concentration.

The compositions are preferably formulated in a unit dosage form, each dosage containing from about 5 to about 100 mg, more usually about 10 to about 30 mg, of the active ingredient. The term “unit dosage form” refers to physically discrete units suitable as unitary dosages for human subjects and other mammals, each unit containing a predetermined quantity of active material calculated to produce the desired therapeutic effect, in association with a suitable pharmaceutical excipient. Preferably, the compound of the subject invention above is employed at no more than about 20 weight percent of the pharmaceutical composition, more preferably no more than about 15 weight percent, with the balance being pharmaceutically inert carrier(s).

The active compound is effective over a wide dosage range and is generally administered in a pharmaceutically or therapeutically effective amount. It will be understood, however, that the amount of the compound actually administered will be determined by a physician, in the light of the relevant circumstances, including the condition to be treated, the severity of the bacterial infection being treated, the chosen route of administration, the actual compound administered, the age, weight, and response of the individual patient, the severity of the patient's symptoms, and the like.

In therapeutic use for treating, or combating, bacterial infections in warm-blooded animals, the compounds or pharmaceutical compositions thereof will be administered orally, topically, transdermally, and/or parenterally at a dosage to obtain and maintain a concentration, that is, an amount, or blood-level of active component in the animal undergoing treatment which will be antibacterially effective. Generally, such antibacterially or therapeutically effective amount of dosage of active component (i.e., an effective dosage) will be in the range of about 0.1 to about 100, more preferably about 1.0 to about 50 mg/kg of body weight/day.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, 0.1 to about 500 mg of the active ingredient of the present invention.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the novel compositions of the present invention may be incorporated for administration orally or by injection include aqueous solutions, suitably flavored syrups, aqueous or oil suspensions, and flavored emulsions with edible oils such as corn oil, cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner.

The following formulation examples illustrate representative pharmaceutical compositions of the present invention.

### FORMULATION EXAMPLE 1

Hard gelatin capsules containing the following ingredients are prepared:

| | Quantity |
|---|---|
| Ingredient | (mg/capsule) |
| Active Ingredient | 30.0 |
| Starch | 305.0 |
| Magnesium stearate | 5.0 |

The above ingredients are mixed and filled into hard gelatin capsules in 340 mg quantities.

### FORMULATION EXAMPLE 2

A tablet formula is prepared using the ingredients below:

| | Quantity |
|---|---|
| Ingredient | (mg/tablet) |
| Active Ingredient | 25.0 |
| Cellulose, microcrystalline | 200.0 |
| Colloidal silicon dioxide | 10.0 |
| Stearic acid | 5.0 |

The components are blended and compressed to form tablets, each weighing 240 mg.

### FORMULATION EXAMPLE 3

A dry powder inhaler formulation is prepared containing the following components:

| Ingredient | Weight % |
|---|---|
| Active Ingredient | 5 |
| Lactose | 95 |

The active ingredient is mixed with the lactose, and the mixture is added to a dry powder inhaling appliance.

### FORMULATION EXAMPLE 4

Tablets, each containing 30 mg of active ingredient, are prepared as follows

| | Quantity | |
|---|---|---|
| Ingredient | (mg/tablet) | |
| Active Ingredient | 30.0 | mg |
| Starch | 45.0 | mg |
| Microcrystalline cellulose | 35.0 | mg |
| Polyvinylpyrrolidone | 4.0 | mg |
| (as 10% solution in sterile water) | | |
| Sodium carboxymethyl starch | 4.5 | mg |
| Magnesium stearate | 0.5 | mg |
| Talc | 1.0 | mg |
| Total | 120 | mg |

The active ingredient, starch and cellulose are passed through a No. 20 mesh U.S. sieve and mixed thoroughly. The solution of polyvinylpyrrolidone is mixed with the resultant powders, which are then passed through a 16 mesh U.S. sieve. The granules so produced are dried at 50° C. to 60° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets each weighing 120 mg.

### FORMULATION EXAMPLE 5

Capsules, each containing 40 mg of medicament are made as follows:

| | Quantity |
|---|---|
| Ingredient | (mg/capsule) |
| Active Ingredient | 40.0 mg |
| Starch | 109.0 mg |
| Magnesium stearate | 1.0 mg |
| Total | 150.0 mg |

The active ingredient, starch and magnesium stearate are blended, passed through a No. 20 mesh U.S. sieve, and filled into hard gelatin capsules in 150 mg quantities.

### FORMULATION EXAMPLE 6

Suppositories, each containing 25 mg of active ingredient are made as follows:

| Ingredient | Amount |
|---|---|
| Active Ingredient | 25 mg |
| Saturated fatty acid glycerides to | 2,000 mg |

The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

### FORMULATION EXAMPLE 7

Suspensions, each containing 50 mg of medicament per 5.0 mL dose are made as follows:

| Ingredient | Amount | |
|---|---|---|
| Active Ingredient | 50.0 | mg |
| Xanthan gum | 4.0 | mg |
| Sodium carboxymethyl cellulose (11%) | 50.0 | mg |
| Microcrystalline cellulose (89%) | | |
| Sucrose | 1.75 | g |
| Sodium benzoate | 10.0 | mg |
| Flavor and Color | q.v. | |
| Purified water to | 5.0 | mL |

The active ingredient, sucrose and xanthan gum are blended, passed through a No. 10 mesh U.S. sieve, and then mixed with a previously made solution of the microcrystalline cellulose and sodium carboxymethyl cellulose in water. The sodium benzoate, flavor, and color are diluted with some of the water and added with stirring. Sufficient water is then added to produce the required volume.

### FORMULATION EXAMPLE 8

| | Quantity |
|---|---|
| Ingredient | (mg/capsule) |
| Active Ingredient | 15.0 mg |
| Starch | 407.0 mg |
| Magnesium stearate | 3.0 mg |
| Total | 425.0 mg |

The active ingredient, starch, and magnesium stearate are blended, passed through a No. 20 mesh U.S. sieve, and filled into hard gelatin capsules in 425.0 mg quantities.

### FORMULATION EXAMPLE 9

A subcutaneous formulation may be prepared as follows:

| Ingredient | Quantity | |
|---|---|---|
| Active Ingredient | 5.0 | mg |
| Corn Oil | 1.0 | mL |

### FORMULATION EXAMPLE 10

A topical formulation may be prepared as follows:

| Ingredient | Quantity | |
|---|---|---|
| Active Ingredient | 1-10 | g |
| Emulsifying Wax | 30 | g |
| Liquid Paraffin | 20 | g |
| White Soft Paraffin | to 100 | g |

The white soft paraffin is heated until molten. The liquid paraffin and emulsifying wax are incorporated and stirred until dissolved. The active ingredient is added and stirring is continued until dispersed. The mixture is then cooled until solid.

### FORMULATION EXAMPLE 11

An intravenous formulation may be prepared as follows:

| Ingredient | Quantity | |
|---|---|---|
| Active Ingredient | 250 | mg |
| Isotonic saline | 1000 | mL |

Another preferred formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. No. 5,023,252, herein incorporated by reference. Such patches may be constructed for continuous, pulsatile, or on demand delivery of pharmaceutical agents.

Other suitable formulations for use in the present invention can be found in REMINGTON'S PHARMACEUTICAL SCIENCES, Mace Publishing Company, Philadelphia, Pa., 17th ed. (1985).

As noted above, the compounds described herein are suitable for use in a variety of drug delivery systems described above. Additionally, in order to enhance the in vivo serum half-life of the administered compound, the compounds may be encapsulated, introduced into the lumen of liposomes, prepared as a colloid, or other conventional techniques may be employed which provide an extended serum half-life of the compounds. A variety of methods are available for preparing liposomes, as described in, e.g., Szoka, et al., U.S. Pat. Nos. 4,235,871, 4,501,728 and 4,837,028 each of which is incorporated herein by reference.

As noted above, the compounds administered to a patient are in the form of pharmaceutical compositions described above. These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the compound preparations typically will be between 3 and 11, more preferably from 5 to 9 and most preferably from 7 and 8. It will be understood that use of certain of the foregoing excipients, carriers, or stabilizers will result in the formation of pharmaceutical salts.

In general, the compounds of the subject invention will be administered in a therapeutically effective amount by any of the accepted modes of administration for agents that serve similar utilities. Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (the concentration of the test compound which achieves a half-maximal inhibition of symptoms) as determined in cell culture. Such information can be used to more accurately determine useful doses in humans. Levels in plasma may be measured, for example, by high performance liquid chromatography.

9.2 Immunoglobulin Formulations

One aspect of the invention contemplates the use of immunoglobulins that recognize and bind to proteins that are involved in bone mineralization, such as any of the proteins discussed herein. Preferably, the immunoglobulins modulate osteoblast-osteoclast homeostasis such that bone mineralization is enhanced. In certain diseases, compounds and compositions that decrease bone mineralization will be preferred.

Preferred immunoglobulins are antibodies or fragments thereof. Preferred antibodies are monoclonal antibodies, however embodiments utilizing polyclonal antibodies are also contemplated. Preferred monoclonal antibodies include human, humanized and primatized™ monoclonal antibodies.

The phrases “pharmaceutically or pharmacologically acceptable” refer to molecular entities and compositions that do not produce an adverse, allergic or other untoward reaction when administered to an animal, or a human, as appropriate. Veterinary uses are equally included herein and “pharmaceutically acceptable” formulations include formulations for both clinical and/or veterinary use. For example, compositions can be administered to certain agricultural animals, such as poultry, to increase bone mineralization to prevent bone breaks and fractures.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. For human administration, preparations should meet sterility, pyrogenicity, general safety and purity standards as required by FDA Office of Biologics standards. Supplementary active ingredients can also be incorporated into the compositions.

“Unit dosage” formulations are those containing a dose or sub-dose of the administered ingredient adapted for a particular timed delivery. For example, exemplary “unit dosage” formulations are those containing a daily dose or unit, or daily sub-dose or a weekly dose or unit, or weekly sub-dose and the like.

For example, a humanized antibody can be used as the active ingredient in a pharmaceutical composition to treat bone mineralization diseases. The pharmaceutical composition will more than likely be formulated for an intravenous, intramuscular or other form that can be administered locally. The composition can comprise inactive ingredients ordinarily used in pharmaceutical preparation such as diluents, fillers, disintegrants, sweeteners, lubricants and flavors. The pharmaceutical composition is preferably formulated for intravenous administration, either by bolus injection or sustained drip, or for release from an implanted capsule. A typical formulation for intravenous administration utilizes physiological saline as a diluent.

Also contemplated for use are fragments of immunoglobulins that modulate bone mineralization. Preferable fragments are those from monoclonal antibodies or which are synthesized recombinantly. Preparation of these antibody fragments is considered known in the art.

The dose of an immunoglobulin composition for a patient depends upon the specific antibody used, body weight, age, gender, state of health, diet, administration time and formulation of the composition, route of administration, and the disease to be treated. A typical dose is from 0.1 mg/kg/day to 100 mg/kg/day. More typically the dose is from 1 mg/kg/day to 50 mg/kg/day.

9.2.1 Diagnostic Immunoglobulins

The antibodies of the invention can also be used in a diagnostic assay. One preferred format for a diagnostic assay of the invention is quantitation of cells in a sample that express any of the proteins involved with bone mineralization on the cell surface. Methods for counting cells bearing particular surface markers are well-known in the art. For example, fluorescence activated cell sorting (FACS) can be used. Another format for a diagnostic assay of the invention is to quantify the amount of a bone mineralization protein of interest in a sample. There are many formats for performing such an assay known in the art, for example antigen-immobilized or sandwich format enzyme-linked immunosorbent assays.

9.2.2 Injectable Formulations

Antibodies, immunoglobulins or immunoconjugates which recognize and bind to proteins involved in bone mineralization will most often be formulated for parenteral administration, e.g., formulated for injection via the intravenous (i.v.), intramuscular (i.m.), subcutaneous (s.c.), transdermal, or other such routes, including peristaltic administration and direct instillation into a site (i.e., administration into regions of a long bone). The preparation of an aqueous composition that contains such an immunoglobulin as an active ingredient will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form should be sterile and fluid to the extent that syringability exists. It should be stable under the conditions of manufacture and storage and should be preserved against the contaminating action of microorganisms, such as bacteria and fungi.

The immunoglobulins that recognize and bind to proteins involved in bone mineralization can be formulated into a sterile aqueous composition in a neutral or salt form. Solutions as free base or pharmacologically acceptable salts can be prepared in water suitably mixed with a surfactant, such as hydroxypropylcellulose. Pharmaceutically acceptable salts, include the acid addition salts (formed with the free amino groups of the protein), and those that are formed with inorganic acids such as, for example, hydrochloric or phosphoric acids, or such organic acids as acetic, trifluoroacetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Suitable carriers to be used with immunoglobulins include solvents and dispersion media containing, for example, water, ethanol, polyol (e.g., glycerol, propylene glycol, and liquid polyethylene glycol, and the like), suitable mixtures thereof, and vegetable oils. In many cases, it will be preferable to include isotonic agents, for example, sugars or sodium chloride. The proper fluidity can be maintained, for example, by the use of a coating, such as lecithin, by the maintenance of the required particle size in the case of dispersion and/or by the use of surfactants.

Under ordinary conditions of storage and use, all such preparations should contain a preservative to prevent the growth of microorganisms. The prevention of microorganisms can be brought about by various antibacterial and antifungal agents, for example, parabens, chlorobutanol, phenol, sorbic acid, thimerosal, and the like. Prolonged absorption of the injectable compositions can be brought about by the use in the compositions of agents delaying absorption, for example, aluminum monostearate and gelatin.

Sterile injectable solutions are prepared by incorporating the active agents in the required amount in the appropriate solvent with various of the other ingredients enumerated above, as desired, followed by filtered sterilization. Generally, dispersions are prepared by incorporating the various sterilized active ingredients into a sterile vehicle that contains the basic dispersion medium and the required other ingredients from those discussed above.

In the case of sterile powders for the preparation of sterile injectable solutions, the preferred methods of preparation are vacuum-drying and freeze-drying techniques that yield a powder of the active ingredient, plus any additional desired ingredient from a previously sterile-filtered solution thereof.

9.2.3 Sustained Release Formulations

Formulations of immunoglobulins that recognize, bind to proteins thereby modulating bone mineralization are easily administered in a variety of dosage forms, such as the type of injectable solutions described above, but other pharmaceutically acceptable forms are also contemplated, e.g., tablets, pills, capsules or other solids for oral administration, suppositories, pessaries, nasal solutions or sprays, aerosols, inhalants, topical formulations, liposomal forms and the like. The type of form for administration will be matched to the disease or disorder to be treated.

Pharmaceutical “slow release” capsules or “sustained release” compositions or preparations may be used and are generally applicable. Slow release formulations are generally designed to give a constant drug level over an extended period. The slow release formulations are typically implanted in the vicinity of the disease site, for example, in a long bone.

Suitable examples of sustained-release preparations include semipermeable matrices of solid hydrophobic polymers containing the antibody or immunoconjugate, wherein the matrices are in the form of shaped articles, e.g., films or microcapsules. Examples of sustained-release matrices include polyesters; hydrogels, for example, poly(2-hydroxyethyl-methacrylate) or poly(vinylalcohol); polylactides; copolymers of L-glutamic acid and γ-ethyl-L-glutamate; non-degradable ethylene-vinyl acetate; degradable lactic acid-glycolic acid copolymers, such as the Lupron Depot™ (injectable microspheres composed of lactic acid-glycolic acid copolymer and leuprolide acetate); and poly-D-(−)-3-hydroxybutyric acid.

While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., thus reducing biological activity and/or changing immunogenicity. Rational strategies are available for stabilization depending on the mechanism involved. For example, if the aggregation mechanism involves intermolecular S—S bond formation through thio-disulfide interchange, stabilization is achieved by modifying sulfhydryl residues, lyophilizing from acidic solutions, controlling moisture content, using appropriate additives, developing specific polymer matrix compositions, and the like. Compositions comprising the desired immunoglobulins can also be formulated into liposome or nanoparticles.

### EXAMPLES

### Example 1

### TCF-Luci Assay with GSK Inhibitors

Certain GSK inhibitors are known. Lithium, typically administered in the form of lithium chloride (LiCl) is less specific and can inhibit GSK-3 only at high millimolar dosages (Stambolic et al., Curr. Biol. 6: 1664-68 (1996)). The more selective GSK inhibitor, 3-(3-chloro-4-hydroxyphenylamino)-4-(2-nitrophenyl)-1H-pyrrole-2,5-dione is more specific to the beta isoform of GSK-3. This compound, derivatives, homologs and analogs thereof can be used, amongst other things, to calibrate assays for identifying osteogenic molecule(s). For example, GSK-3 inhibitors can be used to calibrate bone or non-bone cell based TCF assays to identify LRP5/6 agonists, Wnt agonists, LRP5/6-Dkk1 antagonists and other cross talk pathway specific cis/trans element containing reporters. These compounds can also be used to study osteogenic gene activity, secondary assays on osteoblast/osteoclast function, proliferation, differentiation and apoptosis; osteoblast gene profiling assays with or without strain or mechanical loads, in vitro or in vivo; in vivo local effect assays using calvariae models; ex vivo calvaria or other bone derived bone-turnover assays, systemic effect evaluation assays using for example young rate models, or in vivo disuse/ovariectomy type assays can also utilize these compounds.

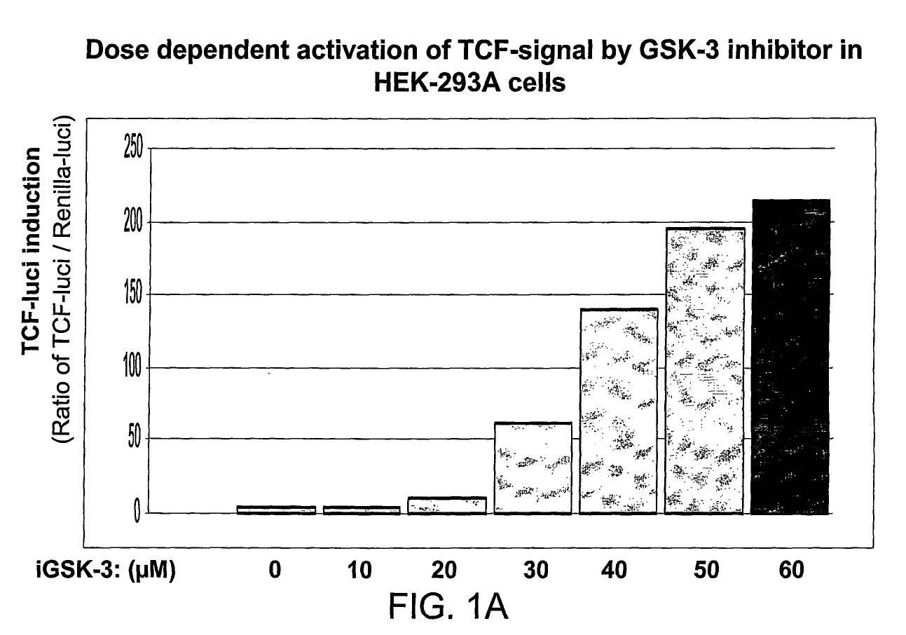

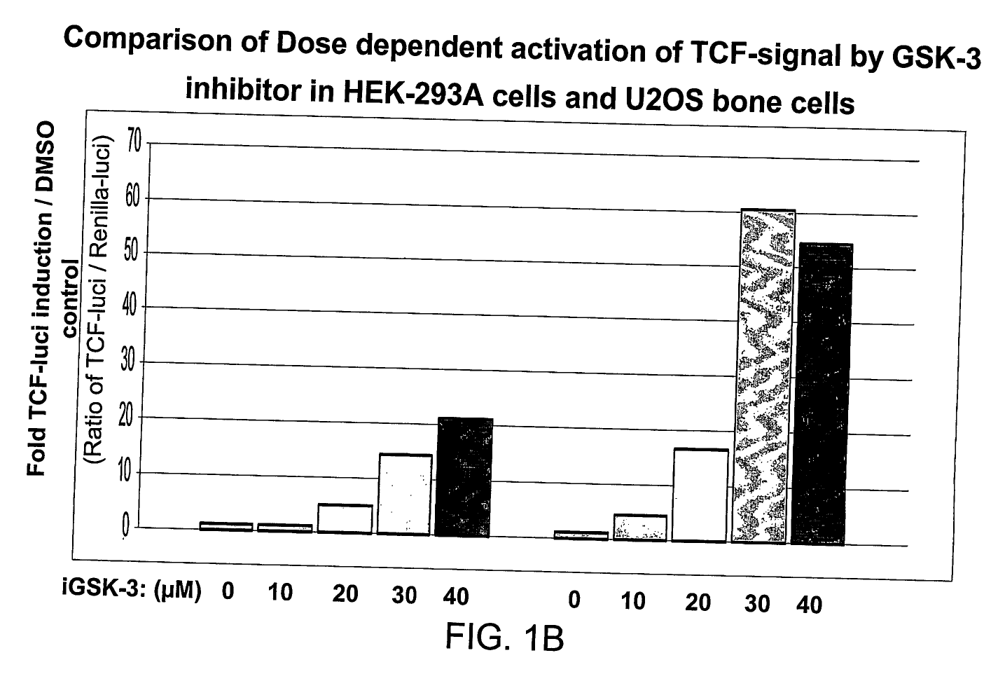

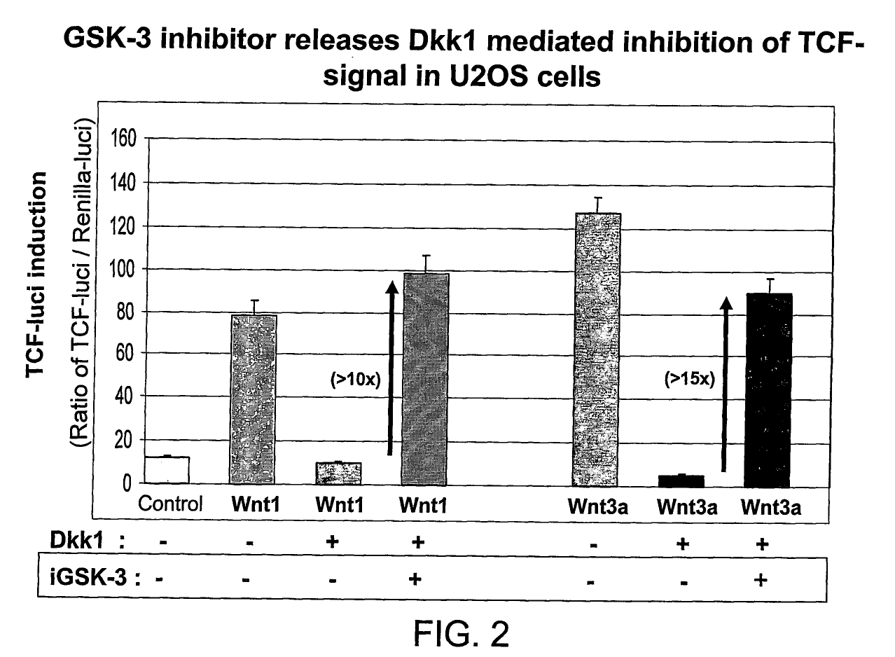

The TCF reporter assays involve a TCF reporter containing 16 copies (i.e., 16×) of Wnt-beta-catenin signal responsive TCF element, basal TK-promoter, and luciferase gene. Human embryonic kidney (HEK)-293A cells (ATCC) or other osteosarcoma derived bone cell line (e.g., U2OS) were cultured in Dulbecco's Minimum Essential Media (DMEM, Invitrogen) or in RPMI (Invitrogen) supplemented with 10% heat inactivated FBS, 1% glutamax (Invitrogen) and 1% penicillin-streptomycin (Invitrogen). HEK-293A cells (about 40,000 cells per well) or U2OS cells (25,000 cells per well) were plated. After 24 hours incubation (i.e., until 80-90% confluent), the media was replaced with either 100 μL of fresh serum free OPTIM (Gibco/BRL) or RPMI or DMEM media. Both cell types were transfected with 16×-TCF(TK)-firefly luciferase (0.3 μg/well) and TK-Renilla-luciferase (0.06 μg/well) using Lipofectamine 2000 transfection reagent (Promega, Madison, Wis.) as described by the manufacturer. The DNA mixture and the reagent are then incubated for 20 min at room temperature. 50 μl/well of the DNA-reagent mix is added per well to 100 μL of OPTIM and incubated for 4 hr at 37° C. The transfection medium was replaced with 140 μL fresh DMEM or RPMI media to the 293A or U2OS cells respectively. The GSK inhibitor (3-(3-chloro-4-hydroxyphenylamino)-4-(2-nitrophenyl)-1H-pyrrole-2,5-dione) was diluted in respective medium to get 10× stock of a final amount per well of 150 μL. 10 μL of the 10× stock was added per well along with appropriately diluted vehicle (i.e., DMSO) control. After 20-24 hr incubation at 37° C. in a CO₂ incubator, medium containing the compound was removed. Transfected and GSK-3 inhibitor treated cell monolayers were lysed by adding 150 μL of 1× lysis buffer of Dual Luci Reagent (Promega Corp., Madison, Wis.). After 10 min, 20 μL of the lysate was transferred into a 96 well white-plate (Packard/Costar). Cell lysates were mixed with 100 μL/well of LARII buffer (Dual Luci Reagent), and the relative luciferase units (RLUs) were measured. This was followed by the addition of 100 μL per well of “stop & glo” reagent (Dual Luci Reagent), and the internal control renilla luciferase was measured. The ratio of TCF-firefly-luci to renilla was calculated and is represented in FIGS. 1-2.

FIGS. 1A and 1B demonstrate that when TCF-reporter construct is transfected into HEK-293A and U2OS bone cells, iGSK-3 can transactivate the reporter in a dose dependent manner. The induction of TCF-luciferase signal and hence the Wnt-signal is more pronounced in U2OS bone cells than in HEK-293 cells. In addition, the FIG. 1B shows that in U2OS cells, a significant induction of the TCF-signal is observed at 10 μM concentration of iGSK-3 and at 30 μM it reached almost maximal unlike 293A cells. This indicates that U2OS bone cells are more sensitive to Wnt signal modulation than the HEK-293A cells.

### Example 2

### The GSK-3 Inhibitor Releases Dkk1 Mediated Inhibition of the TCF Signaling in U2OS Human Osteoblastic Cells

This example demonstrates that a GSK-3 inhibitor (3-(3-chloro-4-hydroxyphenylamino)-4-(2-nitrophenyl)-1H-pyrrole-2,5-dione) can be used to release Dkk1 mediated inhibition of TCF-signal in U2OS cells. As demonstrated in FIG. 2, Wnt1 and Wnt3A activates TCF-signal about 10-15× over control. Addition of Dkk1 inhibited Wnt mediated TCF signaling. However, the GSK-3 inhibitor can reverse the inhibition. Furthermore, these data demonstrate that iGSK-3 can be used as a small molecule tool to validate and calibrate another cell based TCF-assay that is designed to identify compounds which could block Dkk1 and LRP5 interaction in presence of a Wnt ligand (e.g., Wnt 3A). The final readout is activation of Dkk1 mediated suppressed TCF-signal. In the absence of a known small molecule that could block Dkk1-LRP5 interaction and in turn activate the TCF-signal, a iGSK-3 has been shown to activate the TCF-signal. This indicates that by modulating the pathway even internally, one can release the suppression exerted externally through LRP5 by Dkk1. The experiment represented in FIG. 2 involved U2OS (ATCC) bone cells and is based on the endogenous expression of LRP5/6 receptors. The cells are plated at 25,000 cells per well and after 24 hours incubation (i.e., until 80-90% confluent). The media was replaced with 100 μL of fresh serum free OPTIM (Gibco/BRL) or RPMI media. The cells were co-transfected with 16×-TCF(TK)-firefly luciferase (0.3 μg/well), TK-Renilla-luciferase (0.06 μg/well), Wnt1 or Wnt 3a (0.0025 μg/well) and Dkk1 (0.1 μg/well) using Lipofectamine 2000 transfection reagent (Promega, Madison, Wis.) as described by the manufacturer. The DNA mixture and the reagent are then incubated for 20 min at room temperature. 50 μl/well of the DNA-reagent mix is added per well to 100 μL of OPTIM and incubated for 4 hr at 37° C. The transfection medium was replaced with 140 μL fresh RPMI medium. The GSK-3 inhibitor was diluted in RPMI medium to get 15× stock of a final concentration (30 uM) per well of 150 μL. 10 μL of the 15× stock was added per well along with appropriately diluted vehicle (i.e., DMSO) control. After 20-24 hr incubation at 37° C. in a CO₂ incubator, medium containing the compound was removed. Transfected and GSK-3 inhibitor treated cell monolayers were lysed by adding 150 μL of 1× lysis buffer of Dual Luci Reagent (Promega Corp., Madison, Wis.). After 10 min, 20 μL of the lysate was transferred into a 96 well white-plate (Packard/Costar). Cell lysates were mixed with 100 μL/well of LARII buffer (Dual Luci Reagent), and the relative luciferase units (RLUs) were measured. This was followed by the addition of 100 μL per well of “stop & glo” reagent (Dual Luci Reagent), and the internal control renilla luciferase was measured. The ratio of TCF-firefly-luci to renilla was calculated and is represented in FIG. 2.

The results demonstrate that either with Wnt1 or Wnt 3A, there is about 10-15 fold increased TCF-signal respectively from the basal level. When Dkk1 was co-transfected with Wnt1 or Wnt 3A, the TCF-activity is suppressed almost completely. However, when iGSK-3 was added to Wnt1/Wnt 3A and Dkk1 transfected cells, the suppression is released almost completely in Wnt1 and about 75% with Wnt 3A. Even though this experiment was based on the endogenous expression of LRP5/6 receptors, such assays can be re-formatted by over expression of transfected LRP5/6 or suppression of endogenous LRP5/6 by specific siRNAs to address specific interaction of a molecule with LRP5/6.

### Example 3

### Effect of Glycogen Synthase Kinase-3 (GSK-3) Inhibitor on Osteogenesis in a Mouse Calvarial Model

To determine in vivo whether Wnt pathway activation through GSK-3 inhibition induces osteogenesis, the local administration of a GSK-3 inhibitor (iGSK-3) (i.e., 3-(3-chloro-4-hydroxyphenylamino)-4-(2-nitrophenyl)-1H-pyrrole-2,5-dione) on mouse calvariae were examined.

iGSK-3 at 1 mg/kg or vehicle only was injected s.c. daily for 7 and 18 days over the right side of the calvaria in 4 week-old male Swiss-Webster mice. The effect of the iGSK-3 on calvarial bone was assessed in histological sections by alkaline phosphatase (ALPase) enzyme histochemical staining, quantitative histomorphometry, and β-catenin expression by immunohistochemistry. Following sacrifice by CO₂ narcosis, calvariae were removed intact, soft tissues were gently dissected, and the bones were fixed in 70% ethanol for 24 h for further processing and analysis. Calvariae were then bisected perpendicular to the sagittal suture through the central portion of the parietal bones parallel to the lambdoidal and coronal sutures. The anterior portion of the calvaria was used for paraffin sections, and the posterior portion of the calvaria was used for frozen sections. Four to five 5 μm-thick representative, non-consecutive step sections were cut. The paraffin sections are routinely stained with hematoxylin and eosin (H&E) for the measurement of calvarial thickness. The frozen sections were used for alkaline phosphatase detection. To facilitate histomorphometric measurements, a standard length of 2 mm of each section from the edge of the sagittal suture to the muscle insertion at the lateral border of each bone was used. All measurements were made using the R&M Biometrics Inc. Bioquant Image Analysis System.

After fixation, the anterior portion of the calvaria was decalcified in Surgipath Decalcifier II (Richmond, Ill.) for 7-8 h and then dehydrated in graded alcohol. Four to five 5 μm-thick representative, non-consecutive coronal step paraffin sections were cut. Detection of non-phospho β-catenin in tissue sections utilized a mouse monoclonal antibody that was generated by Upstate Biotechnology (Lake Placid, N.Y.) using the synthetic peptide CGG-SYLDSGIHSGATTTAPSLSGK as immunogen. This monoclonal antibody recognizes the non-phosphorylated form of β-catenin (Cat. No. 06-734, Upstate Biotech). The binding of the antibody to the epitope was visualized (1 μg/1 mL) using an avidin-linked AP system (Vector Laboratories, Burlingame, Calif.). Controls comprised samples with the avidin-AP in the absence of primary antibody.

The activity of Alkaline Phosphatase (ALPase) was assessed with a histochemical stain using a Vector Red Alkaline Phosphatase Substrate Kit (Vector Laboratories, Inc. Burlingame, Calif.) in 6 μm frozen sections of the mouse parietal bone after fixing in 70% Ethanol.

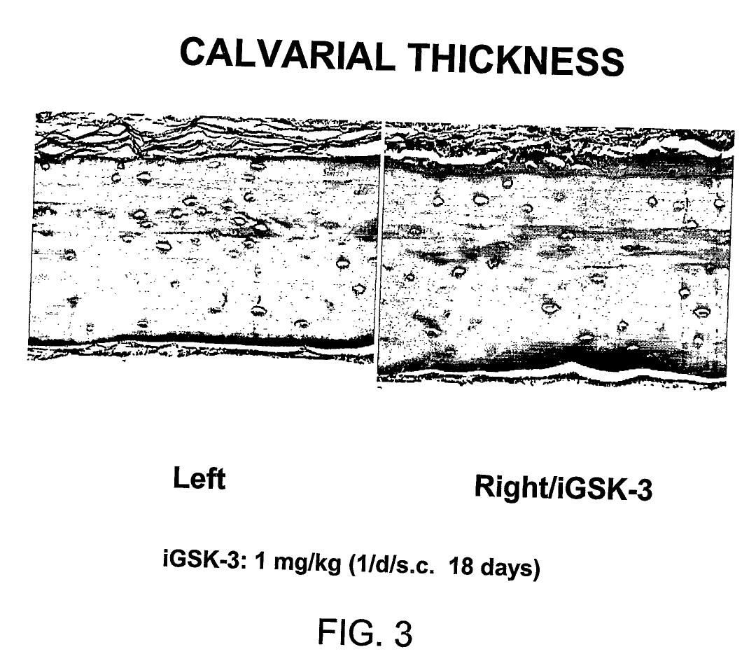

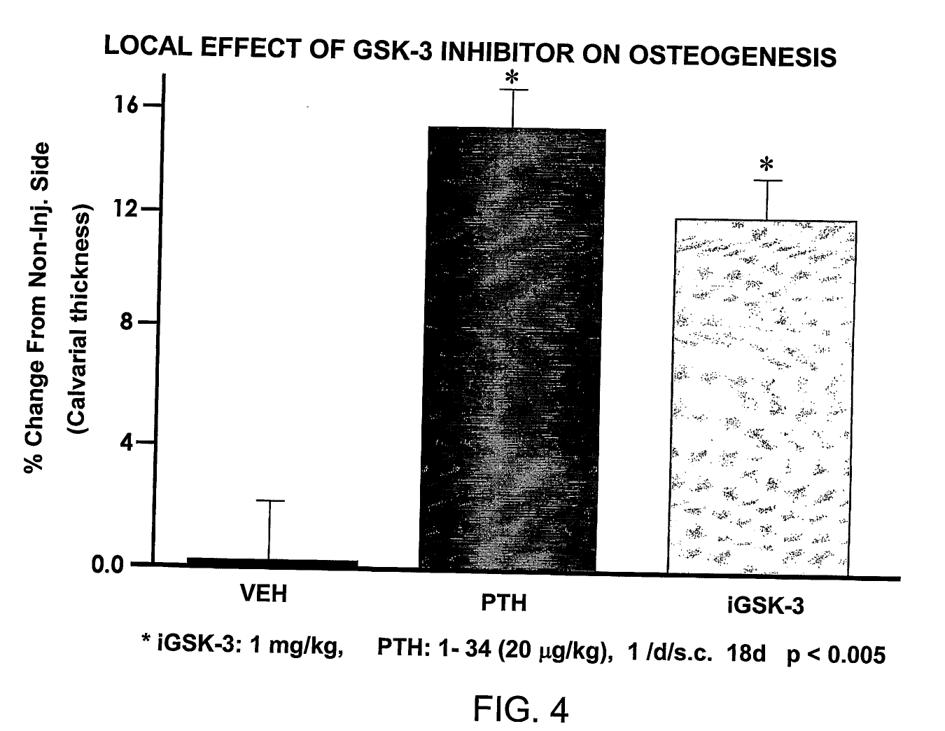

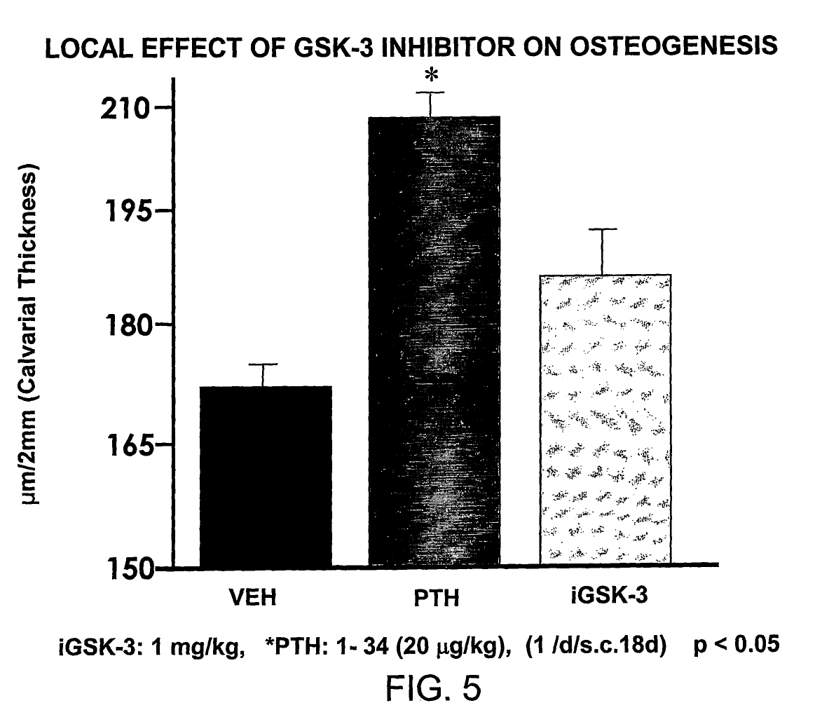

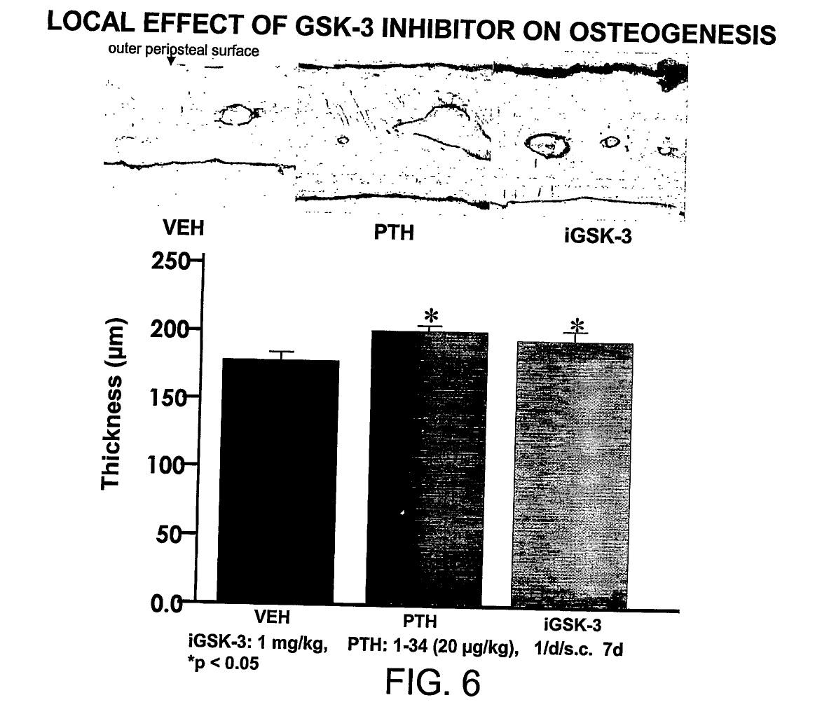

The experiments (FIGS. 3-4) demonstrate a statistical increase in calvarial thickness in the right hemicalvarium injected with GSK-3 inhibitor for 18 days as compared to the left non-injected hemicalvarium of the same animal (11.8%, p<0.005). However, when comparing the effects of the GSK3 inhibitor on the calvarial thickness to the mice treated with vehicle control only (vehicle being 50% DMSO containing 2% Tween 80 and 0.5% methylcellulose), there was only a 6% non-statistical increase in calvarial thickness (FIG. 5). Importantly, when the GSK-3 inhibitor was dissolved in a different vehicle containing 10% DMSO containing 2% Tween 80 with 0.5% methylcellulose, and injected 1 mg/kg/d/s.c. for 7 days there was a statistically significant 10% increase in calvarial thickness compared to vehicle control treated calvaria (FIG. 6).

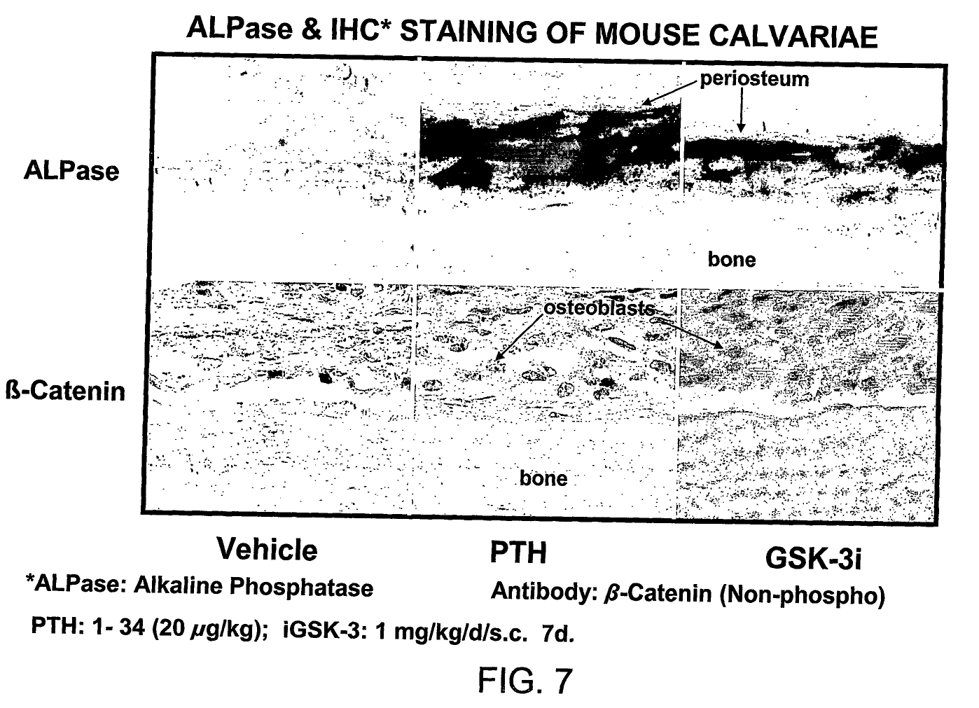

To determine mechanistically how the GSK3 inhibitor is eliciting its anabolic effect, histochemical staining was performed for alkaline phosphatase, an osteoblast differentiation and functional marker. A marked increase in alkaline phosphatase was observed in osteoblasts in the calvarium with local administrations of GSK-3 inhibitor for 7 days as compared to the vehicle controls (FIG. 7). Immunohistochemistry (IHC) of calvaria injected with GSK-3 inhibitor revealed strong β-catenin expression in pre-osteoblasts and osteoblastic cells lining the perisoteum (FIG. 7). Together, these findings demonstrate that inhibition of GSK-3 by local injection of an iGSK has a bone anabolic effect that is associated with an increase in the level of β-catenin leading to the induction of osteoblast activity.

### Example 4

### Flexercell® Loading and Gene Expression in Osteoblasts

The Flexercell® assay can be used with the following osteoblastic cell lines: U2OS (ATCC), MG-63 (ATCC), SAOS-2 (ATCC), HOS-TE85 (ATCC), HOBO3CE6 (Wyeth), HOB01C1 pre-osteocytes (Wyeth) and human primary osteoblasts. The assay can also be used with MC3T3 cells (ATCC) and mouse primary osteoblasts. Additionally, such rat cell lines as UMR-106 (ATCC), ROS17/2.8 and rat primary osteoblasts can similarly be used. Additional mammalian cell lines for use would be evident to the artisan of ordinary skill.

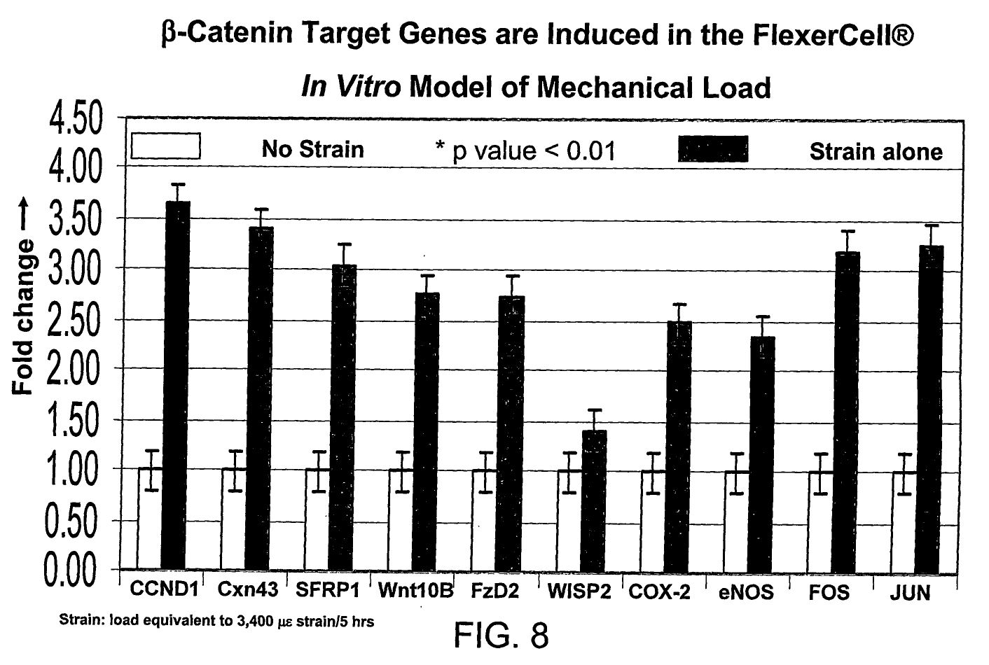

In this example, in vitro loading of cells and gene analysis was performed the mouse osteoblast MC3T3 cells. Application of mechanical strain (5 hr) on MC3T3 cells using the Flexercell® system discussed herein demonstrated an induction of COX-2 (2.5 fold), eNOS (2.5 fold), connexin 43 (3.5 fold), Jun (3.5 fold), cyclin D1 (3.5 fold), Wnt 10B (3 fold), SFRP1 (3 fold), c-Fos (3.5 fold) and Frizzled 2 (3 fold) immediately following load as compared to non-loaded controls (FIG. 8). There was minimal induction of WISP2 gene expression following administration of load.

For these experiments, the mouse MC3T3 osteoblastic cells were cultured in alpha minimum essential media (αMEM) (Invitrogen) supplemented with 10% heat inactivated FBS, 1% glutamax (Invitrogen) and 1% penicillin/streptomycin (Invitrogen). MC3T3 cells were plated at 80,000-100,000 cells per well in a collagen type I coated Bioflex 6 well plate (Flexcell International Corp., McKeesport, Pa.) and then cultured for 3-4 days or until confluent. Twenty-four hours prior to loading, the media was replaced with either 2 mL fresh growth media or serum free media containing αMEM, 0.25% BSA (Serologicals Proteins Inc., Kankakee, Ill.), glutamax and Penicillin/streptomycin as indicated. For those samples being pretreated in serum free media the cells were washed twice each with 2 mL of basal αMEM media prior to adding the BSA containing media. Immediately prior to mechanical loading, the media was removed (i.e., samples containing growth media were washed twice with basal αMEM media) and 1 mL of αMEM/BSA with or without compound (i.e., the GSK-3β inhibitor, 3-(3-chloro-4-hydroxyphenylamino)-4-(2-nitrophenyl)-1Hpyrrole-2,5-dione) added to each well. The cells were then subjected to mechanical distortion equivalent to 3,400 με (2 Hz, 7200 cycles/hr), for 5 hrs using a FX-3000 Flexercell® strain unit (Flexcell International Corp). RNA was then harvested (using the Qiagen Rneasy mini kit) immediately or 24 hr post loading from both the mechanical strained samples as well as the non-strained controls. All data shown in the examples that utilize the FlexerCell were derived from the RNA immediately harvested following loading. Although the magnitude of the regulation of Wnt/β-catenin target gene expression varied only modestly, the genes being regulated by load were the same when the RNA was harvested 24 hr post-load (data not shown).

Real-time PCR was then performed for the indicated genes using mouse gene specific primers and probes as discussed above. The primers and probes used are listed in Table 13.

### Example 5

### Enhancement of Bone Loading by Prior Activation of the Wnt Pathway

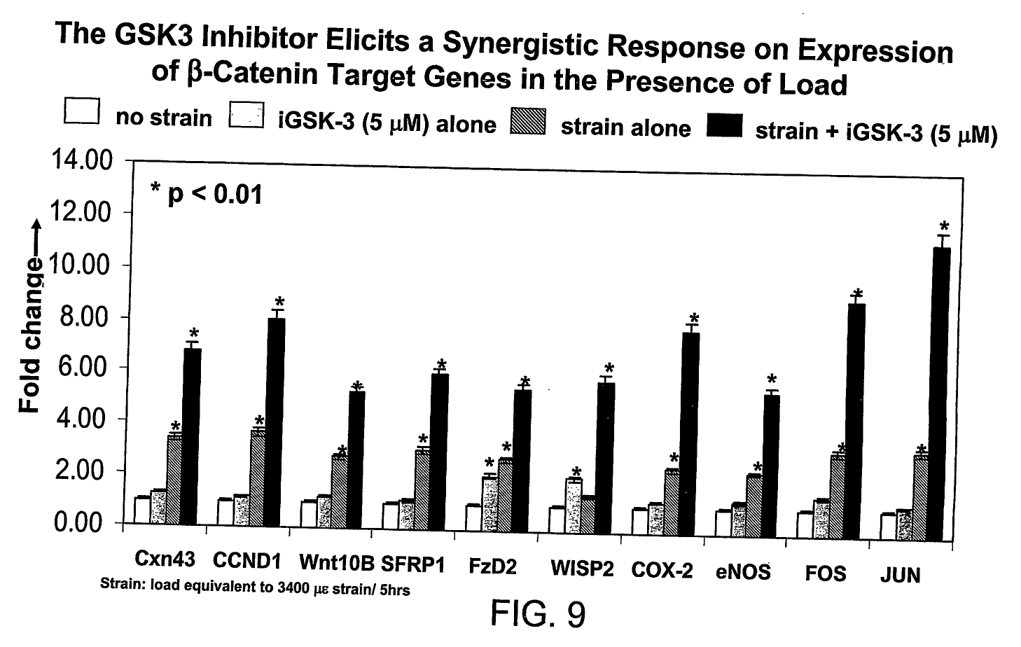

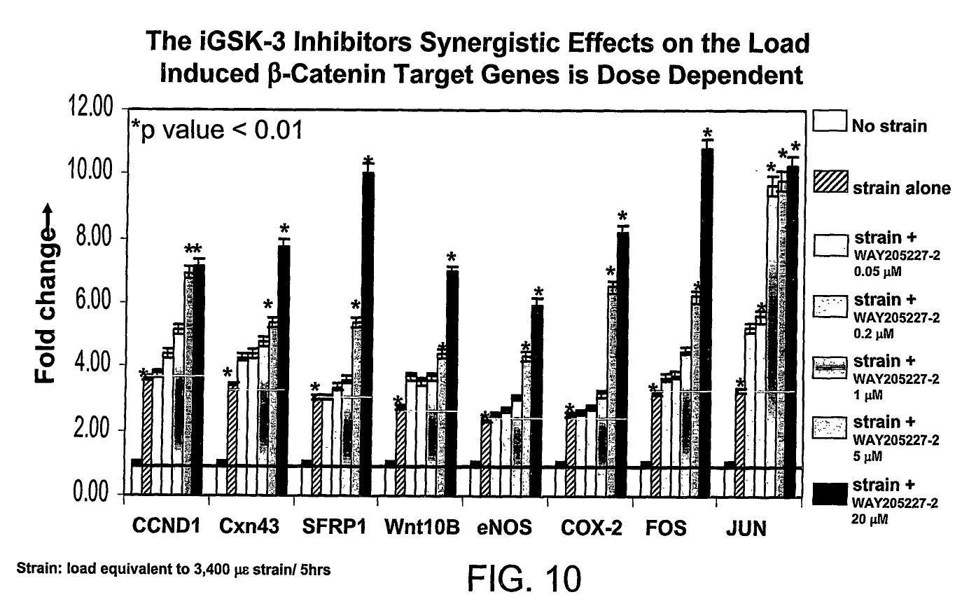

Based on the gene expression results observed in Example 4, the next step was to see whether prior activation of the Wnt pathway enhanced bone load response. Here, MC3T3 cells were treated with a glycogen synthase kinase-3 inhibitor (i.e., 3-(3-chloro-4-hydroxyphenylamino)-4-(2-nitrophenyl)-1Hpyrrole-2,5-dione) to increase β-catenin nuclear translocation and thereby activate the canonical Wnt/β-catenin pathway. Immediately following GSK-3 inhibitor administration, the cells were subject to load (3,400 με, 2 Hz, for 72,000 cycles/hr as discussed above in Example 4) for 5 hours. The GSK-3 inhibitor (5 μm) resulted in a synergistic induction in connexin 43, cyclin D1, Wnt 10B, SFRP1, FZD2, WISP2, COX-2, eNOS, FOS and JUN above the load response gene expression achieved in cells wherein the inhibitor was not administered (FIG. 9). Furthermore, we demonstrate that the synergistic induction of these Wnt/β-catenin target genes in the presence of load is dose dependent on the iGSK-3 concentration (see FIG. 10 and Table 4).

Based on this data, application of an agent that activates the Wnt pathway can enhance the gene expression response produced in response to a bone load stimuli. Such enhancement of bone load stimuli would be useful in identifying other agents that exhibit similar enhancement properties, as well as identifying agents that can be used to prevent bone loss or treat bone loss disorders.

### Example 6

### Activation of β-Catenin Mediated Signaling Pathway in Bone in Response to Mechanical Load In Vivo

Both increased and decreased bone mass are associated with mutations in the Wnt co-receptor, low-density lipoprotein receptor-related protein (LRP) 5. Following application of mechanical load using a tibia four-point (4-pt.) bending system on tibia bones from LRP5 G171V transgenic mice and their non-transgenic littermates, significant changes in the patterns of gene expression for several important components of bone cell signaling pathways was observed (Table 2). β-catenin mediated gene transcription, which is associated with increased osteoblast activity, was up-regulated in both non-transgenic and LRP5 G171V transgenic mice following loading, but with greater up-regulation observed in the LRP5 G171V transgenics (also known as HBM transgenics). The LRP5 G171V mutation also was observed to suppress RANKL/OPG signaling, which attenuates osteoclast recruitment and function. The results demonstrate that the HBM mutation (G171V) negatively affects catabolic activity in bone, thereby enhancing bone growth.

Application of cyclical mechanical load to bone, with devices such as the four-point bending system for rodent tibia, simulates the effect of weight bearing exercise and increases proliferation, differentiation and activity of periosteal osteoblasts (Tanner et al., J. Bone Miner. Res. 16: S203 (2001); Boppart et al., Bone 23(5): 409-415 (1998); Raab-Cullen et al., Calcif. Tissue Int. 55: 473-78 (1994); Cullen et al., J. Appl. Physiol. 88: 1943-48 (2002)). Although four-point bending stimulates gene expression for several growth factors, little is known about the precise molecular events that govern transformation of the mechanical signals into biochemical responses culminating in activation of bone modeling processes.

The low-density lipoprotein receptor-related proteins (LRP) are a family of cell-surface receptors involved in diverse biologic processes. LRP5 and 6, two members of this family, are putative Wnt co-receptors that help transduce signals through β-catenin to TCF/LEF activated promoters. Decreased bone mass are associated with inactivating mutations in the LRP5 gene. LRP5 knockout mice show reduced osteoblast proliferation and function resulting in low bone mineral density despite normal expression of the Runx2/CBFA1. On the other hand, increased bone mass is associated with other mutations in the same gene. One particular point mutation in the LRP5 gene, a glycine 171 to valine (G171V) mutation, results in a phenotype of high-bone mass (i.e., HBM) in all affected members of two independent human kindreds. Transgenic mice expressing the human LRP5 G171V gene (LRP5 G171V TG) faithfully replicate the phenotype of high bone mass. Thus, osteoblast biology, proliferation and differentiation appears to be linked to LRP5/Wnt mediated signaling.

The following data demonstrates that LRP5 G171 V transgenic (TG) mice show a greater bone formation and stress activated responses than non-TG mice following application of load. Further, β-catenin mediated gene transcription is induced in both non-transgenic (non-TG) and LRP5 G171V TG (HBM TG) mice following loading. The HBM TG mice, that have genotype dependent enhanced signaling via the β-catenin signaling pathway (even in the absence of load) respond to load by further up-regulating β-catenin mediated gene transcription. The HBM mutation in LRP5 (i.e., G171V) is also demonstrated herein to down-regulate genes involved in osteoclast proliferation and activity. We also discuss a hitherto unidentified role of the G171V mutation in LRP5 in down-regulating the expression of key genes involved in osteoclast proliferation and activity, thereby inhibiting resorption of bone.

For experiments involving in vivo loading of mouse bone, the heterozygous TIC-LRP5 G171V mice are described in Babij et. al., J. Bone Mineral Res. 18(6): 960-974 (2003) was utilized. These animals show a statistically significant increase (30-55%) in total volumetric bone density. Mechanical loads were delivered to the right tibiae with the mouse four-point bending device (Akhter et al., Calcif. Tissue Int. 63(5): 442-9 (1998). The device was characterized and calibrated for accurate, in vivo, external force application. Id.; Pedersen et al., Calcif. Tissue Int. 65(1): 41-6 (1999); Akhter et al., J. Clin. Densitom. 5(2): 207-16 (2002). The device applies force through four rounded pads composed of balsa wood and covered by 1 mm thick surgical tubing. The upper pads were 4.5 mm apart and centered between the lower pads that were 12 mm apart to create bending in the medial lateral direction. During loading, the animals were anesthetized with isoflurane to permit proper leg positioning.

In these experiments, the left legs served as the non-loaded controls and demonstrate size differences due to the mutation. Right tibiae were loaded in four-point bending for 5 days. Calcein was injected on days 5 and 12, and tissue collected on day 15. Females were loaded at 6 Newton (N) (i.e., non-TG 2,231±110 με; HBM TG 1,525±81 με) and males at 7 N (i.e., non-TG 2,740±157 με; HBM TG 1,841±131 με). Tibial cross sections were obtained from the loaded region of LRP5 G171V TG and non-TG mice. Mineral apposition rate (MAR) was calculated by measuring the distance between the resulting two calcein fronts in bone using fluorescence microscopy. Linear measurements of single label surface (SLS), double label surface (DLS) and bone surface (BS) were taken and the equation DLS+(½ SLS)/BS×100 was used to calculate percent MS/BS. Measurements were made on unstained 10 μm sections at 40× magnification using a 0.03 mm² image window and covering an area of approximately 1.67 mm². All measurements were made using the R&M Biometrics Inc. Bioquant Image Analysis System.

To obtain primary osteoblasts, tibia was dissected out from non-loaded ˜4 week old TIC-line 19 LRP5 G171V TG mice and their wild type non-TG littermates were cut into small chips and digested with collagenase (1 mg/mL) dissolved in DMEM at 37° C. for 30 min. in a shaking water bath. The digest supernatant was removed by centrifugation; collagenase digestion was repeated for two additional times. The chips obtained following the third digestion were transferred to fresh growth media (DMEM supplemented with 10% fetal bovine serum) and grown according to standard techniques until a confluent plate of cells was obtained. This plate was referred to as seeding 1. Chips were then re-seeded in culture and two further seedings were also collected.

RNA isolated from the first two bone chip seedings was used to generate cRNA (i.e., complementary RNA) for hybridization to the Affymetrix MGU74Av2 array. Total RNA was isolated from 80% confluent plates using the QIAGEN RNA kit as per manufacturer's instructions. Target complementary RNA (cRNA) preparation and hybridization to Affymetrix MGU74Av2 arrays were done essentially as described. Hill et. al., Science 290(5492): 809-12 (2000).

Data was normalized using a set of spike-in control and analyzed as described earlier. Hill et. al., Genome Biol. 2(12): RESEARCH0055 (2001).

For TaqMan® analysis, total RNA was isolated from the bones using the AMBION RNA kit as per manufacturer's instructions. The total RNA was subject to DNAse I treatment and then analyzed in TaqMan® reactions as per standard protocols as discussed below:

| | | |
|---|---|---|
| 1. | ABI 4322171 | High Capacity cDNA archive Kit |
| 2. | ABI 4313663 | Adhesive Cover Start Pack |
| 3 | ABI 4311971 | Adhesive Cover, 100/PK |
| 4 | ABI 4318157 | 2x Master Mix |
| 5. | ABI 450025 or 4316034 | Probe labeled as 6FAM or VIC |
| 6. | ABI 4304971 | Sequence Detection Primer, minimum |
| | | 40,000 pmol |
| 7 | Marsh AB-0626 | Adhesive PCR Foil Seals |
| 8 | Matrix 8095 25 mL | Reservoir w/Divider |
| 9 | Ambion 9937 | Nuclease Free Water |
| 10. | Ambion 2684 | Rnase Inhibitor |

Reverse Transcription (ABI 4322171 High Capacity cDNA Archive Kit)

Make cDNA Cocktail as Follows:

| Reagent | Volume per Reaction (per well) |
|---|---|
| 10X RT Buffer | 10 μL |
| 25X dNTP mix | 4 μL |
| Multiscribe RTase (50 U/μL) | 5 μL |
| 10X Random Primers | 10 μL |
| RNAse Inhibitor | 2 μL |
| H₂O | X (to 100 μL) |
| DNAsed RNA | Y (1 to 10 μg) |
| Total | 100 μL |

Mix well and incubate at room temperature for 10 min and 37° C. for 2 hours. The plate maybe stored at −80° C. for up to a year

### II. QC and PCR

Plate 50 ng/10 μl cDNA per well. The diluted cDNAs may be stored at −20° C. for a week. Make 50 μM primer mix. Use an aliquot of probe from ABI (100 μM)

Make PCR Cocktail as Follows:

| Reagent | Volume per reaction (per well) |
|---|---|
| 2X PCR master mix | 12.5 μL |
| 50 μM Primer Mix | 0.2 μL |
| 100 μM Probe | 0.05 μL |
| H₂O | 2.25 μL |
| cDNA | 10 μL |
| Total | 25 μL |

Briefly spin the plate and put into ABI 7000 and analyze the data according to ABI's instructions. Additional aspects to consider include:
1. The primer dilutions and PCR cocktail should be made at a pre-PCR hood and preferably are made same day of use.
2. Baseline may need to be adjusted for genes expressed at low levels.
3. Positive controls should be included on each plate, if possible. This helps normalize date form different plates and machines.
   (Use for In Vivo Loading Experiment)
   Dnase1 Digestion

Reagents from Ambion

| | Small Scale | |
|---|---|---|
| RNA | 10 | μg |
| 10× Dnase1 Buffer | 5 | μL |
| Rnase Inhibitor | 1 | μL |
| Dnased 1 | 2 | μL |
| DEPC H₂O | up to 50 | μL |
| Total | 50 | μL |

Incubate 37° C. for 45 min to 1 hr. Add 1× phenol CHCl3, exact (spin 15′ @ 14,000).

Precipitation:

| | EA | |
|---|---|---|
| DNAsed 1 Digestion | all | |
| DEPC H₂O | to 200 | μL |
| 5 M NaOAC | 5 | μL |
| Glycogen | 5 | μL |
| Cold 100% ETOH | 600 | μL |

Mix well. Keep at −80° C. for 3 hrs or in dry ice for 20 min. Spin at 4° C. for 15 min. Wash once with 75% ETOH. Resuspend in DEPC H₂O. To quantitate, take a 1:50 dilution and take OD. Other methods, such as those of Qiagen, can also be utilized. Arnold et al., BioTechniques 25(1):98-106 (1998). All probe-primer pairs were obtained from Applied Biosystems. A list of TaqMan® probe-primer pairs used in this study can be found in Table 13.

Bone size was observed to be increased in LRP5 G171V TG mice compared to non-TG mice. This result is directly associated with greater structural strength properties in the femurs and vertebra and that the actual strain per Newton (N) of external load is much lower in LRP5 G171V TG mice than in non-TG mice. Therefore, in contrast to their non-TG littermates, the LRP5 G171V TG mice perceive only ˜70% of the actual load applied as strain.

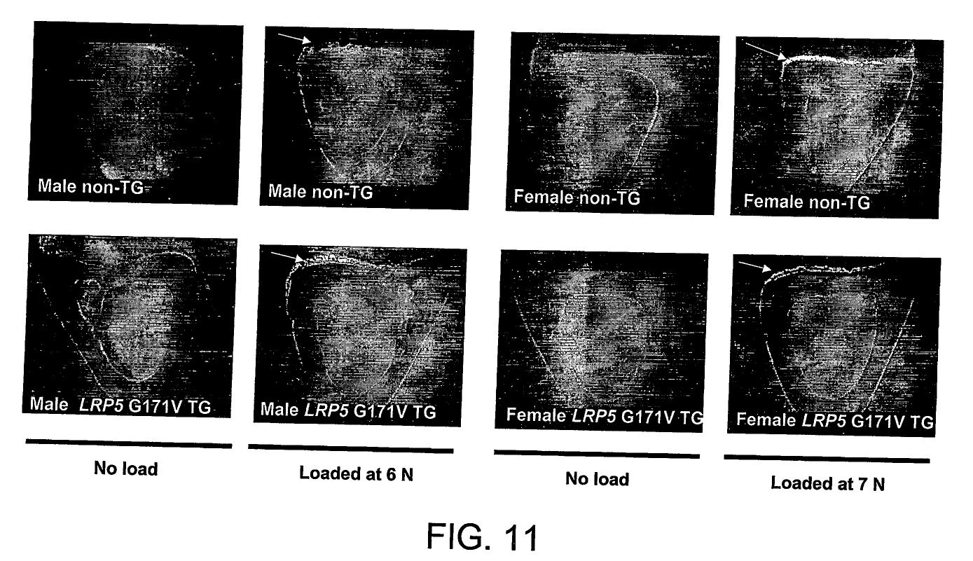

Bone formation in non-transgenic and LRP5 G171V transgenic male and female mice was evaluated using histomorphometric methods following loading on a 4-pt. bending system. Female mice were loaded with 6 N of load (equivalent strain in non-TG mice is 2,231±110 με and in LRP5 G171V TG mice is 1,525±81 με). The male mice were loaded at 7 N (equivalent strain in non-TG mice is 2,740±157 με and in LRP5 G171V TG mice is 1841±131 με). A robust bone formation response was observed in the tibia of both genotypes and sexes following loading compared to the non-loaded controls, as witnessed by the increased calcein labeled surface in the periosteum (FIG. 11). The increase in calcein labeling in loaded non-TG and loaded LRP5 G171V transgenic mice was not significantly different. However, taking into account the ˜30% lower strains perceived by LRP5 G171V TG mice than non-TG mice, the bone formation response in the LRP5 G171V TG mice is greater at the applied external load.

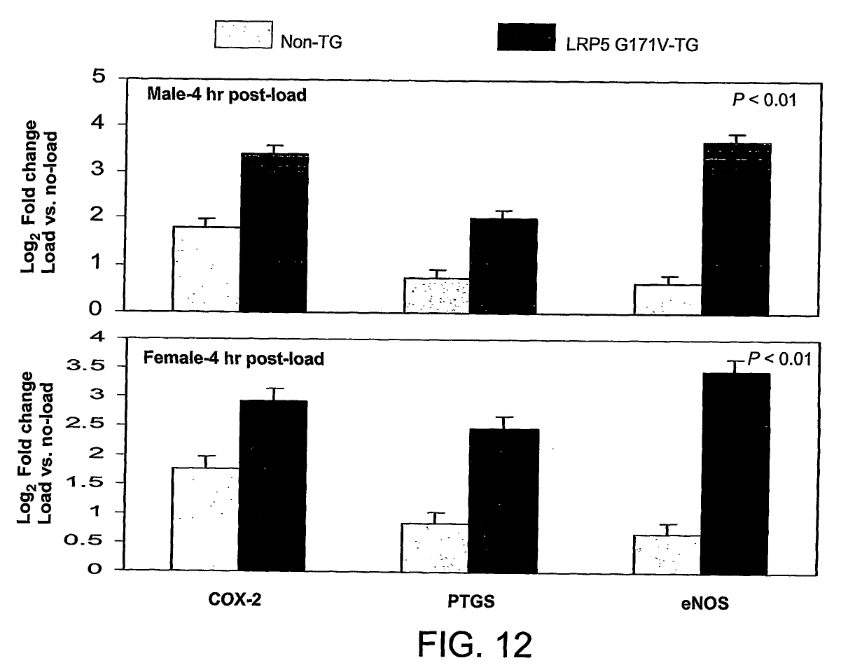

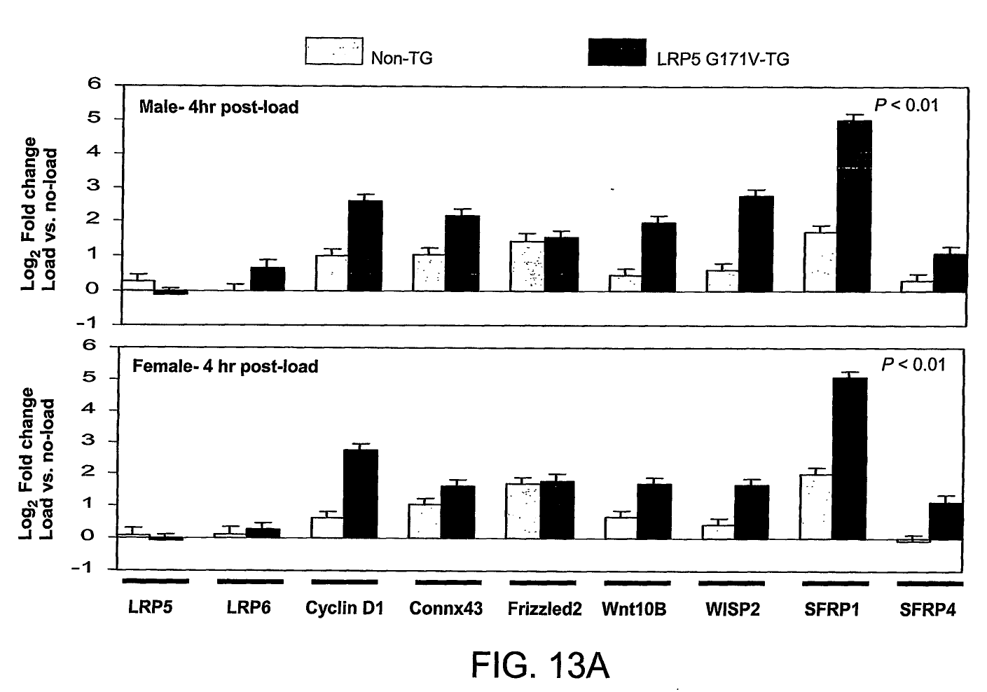

Based on our previous studies regarding the effect of mechanical load and anabolic bone growth, the LRP5 G171V mutation was tested for its ability to alter bone cell sensitivity to bone load, and thereby, increasing bone formation. See Boppart et al., Bone 23(5): 409-415 (1998); Cullen et al., Exercise: Basic and Applied Science 227-237 (Lippincott Williams & Wilkins, Baltimore, Md. 2000); Akhter et al., Calci. Tissue Int. 63(5): 442-9 (1998); and Akhter et al., J. Clin. Densitom. 5(2): 207-16 (2002). Cyclic mechanical loading in vitro induces the release of prostaglandin E (PGE) and expression of the prostaglandin synthase (COX-2), prostacyclin synthase (PTGIS) and endothelial nitric oxide synthase (eNOS) genes, which play important role in osteoblast function. Further analysis of genes transcribed in response to bone load was performed using real-time PCR (TAQMAN®) on RNA obtained from tibiae of 17 week old male and female LRP5 G171V TG and non-TG mice, 4 and 24 hr after application of load: 6 N for female and 7 N for male mice) in vivo. The transcription of all three genes (i.e., COX-2, eNOS and PTGIS) was up-regulated (P<0.01) in bones of all the mice (FIG. 12). However, this up-regulation was about 4 to 10 fold greater in the LRP5 G 71V TG mice than in their non-TG littermates.

The transcription of several bone cell marker genes such as osteonectin (SPARC), cathepsin K (CTSK) and tissue inhibitor of metalloproteinases (TIMP) were up-regulated in both non-TG and LRP5 G171V TG mice following loading. This was determined via TaqMan® using the primers and probes of Table 13. However, as in the case of the genes discussed above, the response is better in the LRP5 G171V TG mice, indicating increased osteogenic activity in these mice. Table 7 describes the genes up- and down-regulated in these mice in response to bone loading.

TABLE 7
Genotype and load induced transcription of HBM signature genes

| PATH- | GENE | GENOTYPE | |
|---|---|---|---|
| WAY | NAME | EFFECT | LOAD EFFECT |
| Wnt | Cyclin D1 | No significant change | Increased in both TG |
| | | | and non-TG animals. |
| | | | Greater in HBM TG |
| | | | animals. |
| | Connexin 43 | No significant change | Increased in both TG |
| | | | and non-TG animals. |
| | | | Greater in HBM TG |
| | | | animals. |
| | WISP2 | Increased in HBM | Increased in both TG |
| | | TG animals | and non-TG animals. |
| | | | Greater in HBM TG |
| | | | animals. |
| | Frizzled 2 | No significant change | Increased in both TG |
| | | | and non-TG animals. |
| | | | Greater in HBM TG |
| | | | animals. |
| | SFRP1 | Increased in HBM | Increased in both TG |
| | | TG animals | and non-TG animals. |
| | | | Greater in HBM TG |
| | | | animals. |
| | SFRP4 | Increased in HBM | Increased in both TG |
| | | TG animals | and non-TG animals. |
| | | | Greater in HBM TG |
| | | | animals. |
| | Wnt10B | Increased in HBM | Increased in both TG |
| | | TG animals | and non-TG animals. |
| | | | Greater in HBM TG |
| | | | animals. |
| HBM1 | IGFBP6 | Increased in HBM | Increased in both TG |
| Signature | | TG animals | and non-TG animals. |
| Genes | | | Greater in HBM TG |
| | | | animals. |
| | CTSK | Increased in HBM | Increased equally in |
| | | TG animals | both TG and non-TG |
| | | | animals. |
| | Osteonectin | No significant change | Increased in both TG |
| | | | and non-TG animals. |
| | | | Greater in HBM TG |
| | | | animals. |
| | IGF2 | Decreased in HBM | |
| | | TG animals | |
| | GADD45A | Decreased in HBM | |
| | | TG animals | |
| | Col1A1 | No significant change | |
| | TGFβ | Increased in HBM | |
| | | TG animals | |
| | TIMP3 | Increased in HBM | |
| | | TG animals | |
| | ACP5 | Increased in HBM | |
| | | TG animals | |
| Load | eNOS | No significant change | Increased in both TG |
| Sensor | | | and non-TG animals. |
| Genes | | | Greater in HBM TG |
| | | | animals. |
| | PTGS | No significant change | Increased in both TG |
| | | | and non-TG animals. |
| | | | Greater in HBM TG |
| | | | animals. |
| | IL-6 | Decreased in HBM | Increased in both TG |
| | | TG animals | and non-TG animals. |
| | | | Greater in HBM TG |
| | | | animals. |
| Stress & | IL-8 | Decreased in HBM | Increased in both TG |
| Osteoclast | | TG animals | and non-TG animals. |
| Function | | | Greater in HBM TG |
| Genes | | | animals. |
| | MK2 | Decreased in HBM | Increased only in |
| | | TG animals | non-TG animals. No |
| | | | significant change in |
| | | | HBM TGs. |
| | OPG | No significant change | Increased only in |
| | | | HBM TGs. No |
| | | | significant change in |
| | | | non-TGs. |
| | RANKL | No significant change | No significant change |
| | | | in either. |
| | LRP5 | Slightly increased in | No significant change |
| | | HBM TG | in either. |

By “genotype effect” is meant how the gene activity in the bones of either the HBM TG or non-TG littermates responded. Expression of the proteins monitored in bone has also been analyzed (column entitled “Load Effect”).

Statistically, the gene expression observed produced the following results, as displayed in Tables 8 to 10.

TABLE 8
Genotype dependent transcription of Wnt/β-catenin target
genes in non-TG and LRP5 G171V TG mice

| Wnt related and | Fold Change |
|---|---|
| Target Genes | HBM TG vs. non-TG animals |
| CCND1 | 1.17 |
| DKK3 | 0.33 |
| MT2 | 3.00 |
| NOTCH1 | 1.67 |
| SFRP1 | 7.00 |
| SFRP2 | 2.36 |
| WISP2 | 2.33 |
| WNT10B | 1.55 |

TABLE 9
Genotype dependent transcription of NF-kB and JNK signaling
pathway genes in non-TG and LRP5 G171V TG mice

| NF-kB/JNK | Fold Change |
|---|---|
| Signaling Genes | HBM TG vs. non-TG animals |
| GRO1 | 0.63 |
| Jun B | 0.38 |
| MAPKAPK5 | 0.50 |
| NFkB1 | 0.35 |

TABLE 10
Differences in transcription of bone cell function genes
in non-TG and LRP5 G171V TG mice

| Bone Function | Fold Change |
|---|---|
| Related Genes | HBM TG vs. non-TG animals |
| BGN | 1.67 |
| BMP1 | 0.52 |
| Col1A1 | 1.64 |
| Col3A1 | 3.14 |
| CSF1R | 0.42 |
| CSPG2 | 5.00 |
| CTSK | 2.42 |
| IGFBP5 | 0.48 |
| LUM | 4.20 |
| MMP-14 | 1.56 |
| MMP-9 | 5.29 |
| OGN | 3.00 |
| PCOLCE | 2.00 |
| PLAT | 0.45 |
| S100A10 | 1.89 |
| SDF1 | 6.80 |
| SERPINE1 | 3.09 |
| SPP1 | 2.16 |
| TOB1 | 0.66 |

All fold changes reported in the three tables above have associated P values of <0.05.

The greatest induction of gene transcription was observed for the Wnt antagonist, SRFP1. This may indicate a homeostatic response in the bone cells that prevents hyperproliferative effects of chronically activated β-catenin signaling. Wnt 10B RNA was also observed to be up-regulated in the bones of HBM mice. The role of Wnt/β-catenin signaling in early development is well studied, and has a demonstrated role in tumors. Thus, it is interesting that a mutation in a Wnt-coreceptor (i.e., LRP5) results in high bone mass with no malignant phenotype in the affected individuals or animals. Additionally, although β-catenin was extensively described as being involved in development, this is the first time that the β-catenin signaling pathway has been shown to be active in normal adult bone and involved in bone density regulation in response to mechanosensory signals.

Expression of β-catenin target genes was demonstrated to be up-regulated in bone cells of HBM TG mice (i.e., mice containing the G171V mutation) in the absence of load. To understand genotype specific differences in the transcriptional profile of bone cells from LRP5 G171V TG or non-TG mice that could contribute to the differences in bone formation, RNA from bone chip seedings of the tibiae (as described in material and methods above) was analyzed. Transcription of many genes affecting osteoblast activity was observed including: procollagen C-proteinase enhancer protein (PCOLCE), collagen 1 and 3, bone specific biglycan (BGN), osteoglycin (OGN), matrix metalloproteinase 9 and 14 (−9 and MMP-14, respectively), chondroitin sulphate proteoglycan (CSPG2), colony stimulating factor 1 receptor (CSF1R), transducer of ErbB-2.1 (TOB1) and lumican (LUM). These listed genes were induced in the bones of G 171V LRP5 TG mice indicating increased osteogenic activity in the bones of these mice. Transcriptional activity of some of these genes is discussed in Tables 8-10.

In addition to these bone specific genes, a preponderance of Wnt/β-catenin signaling related genes were observed to be differentially transcribed in the LRP5 G171V TG mice. Transcription of Wnt signaling component genes (e.g., Wnt 10B, SFRP1, SFRP2 and DKK3) and β-catenin target genes (i.e., metallothionien 2 (MT2), cyclin D1 (CCND1) and WNT1 inducible signaling pathway protein 2 (WISP2)) were induced in the bones from the LRP5 G171V TG mice (Tables 8-11). These observations are in accordance with studies performed regarding the role of the LRP5 G171V mutation in the Wnt signaling pathway. We also noticed transcription down-regulation of several signaling components and target genes of the NF-kB and JNK pathways (i.e., NF-kB1, GRO1, JUN B) in these mice. Thus, the LRP5 G171V mutation may affect bone density by modulating signaling in several pathways, but most significantly the β-catenin signaling pathway.

β-catenin mediated signaling was demonstrated to be activated following application of mechanical load on bone in both non-TG and HBM TG mice. Up-regulation of LRP5 dependent Wnt/β-catenin signaling is associated with increased osteoblast proliferation and function. To evaluate the role of the β-catenin pathway on osteogenic activity following application of mechanical load, RNA levels of several pathway components and target genes in non-loaded and loaded tibiae from HBM TG and non-TG mice were analyzed by TaqMan® as previously described.

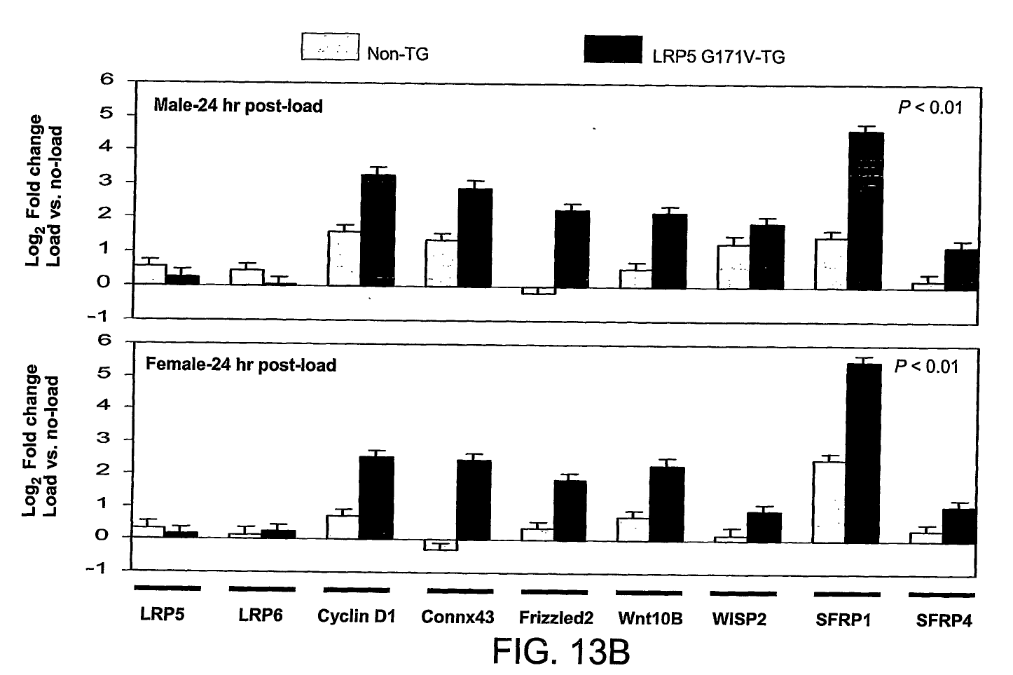

Levels of Wnt 10B, SFRP1, CCND1, Connexin 43 and WISP2 RNA, were significantly up-regulated at both 4 hr and 24 hr following application of mechanical load (an average increase of Log₂ 1-2) in the bones of non-TG mice (FIGS. 13A and 13B). Transcription of Frizzled 2 was induced 4 hr following load, but Frizzled 2 RNA levels returned to baseline by 24 hrs. Transcription of the SFRP4 gene was not significantly altered at either 4 hr or 24 hr following loading in non-TG mice. These data indicate that mechanical stress induces transcription of several signaling components and downstream target genes of the β-catenin signaling pathway in bone cells from non-TG, wild-type mice.

In LRP5 G171V TG mice, a more significant increase (i.e., Log₂ 1.5 to 5.0) was observed in the transcription of all Wnt-related and β-catenin target genes analyzed (including SFRP4). Frizzled 2 RNA levels were induced to approximately the same level in both non-TG and HBM TG mice at 4 hr following load. However, unlike non-TG mice, the HBM TG mice maintained this increase even at 24 hrs. These changes in gene transcription were statistically significant to P<0.01 at both time points (FIGS. 13A and 13B). These observations demonstrate that mechanical loading activates β-catenin mediated signaling and that the LRP5 G171V mutation acts as a gain-of-function mutation in the Wnt pathway.

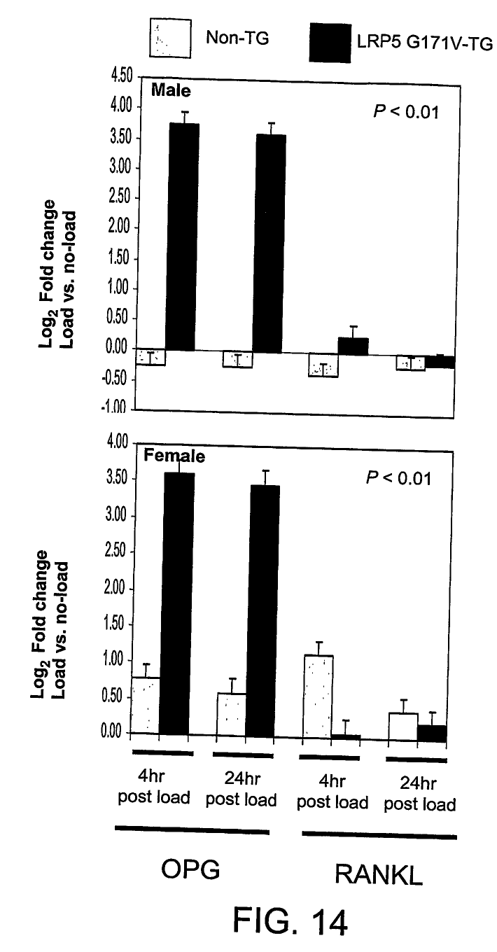

The effect of mechanical load on OPG/RANKL mediated signaling was also studied in the HBM TG and non-TG mice. Down-regulation was observed of genes involved in NF-kB and Jun/Fos mediated signaling in HBM TG mice bones. This observation could indicate alteration of expression of an upstream factor, such as the RANK ligand (RANKL) that stimulates both NF-kB and Jun/Fos driven pathways in osteoclasts (Romas et al., Bone 30(2): 340-6 (2002)). RANKL is the ligand for the Receptor activator of NF-kB (i.e., RANK). RANK/RANKL interactions drive osteoclast differentiation. This process is efficiently blocked by the decoy RANKL receptor, osteoprotegerin (OPG). The levels of OPG and RANKL in osteoblastic and stromal cells are often reciprocally regulated as observed both in vitro and in vivo. Given this reciprocal regulation, the levels of RANKL and OPG RNA in bones from non-TG and LRP5 G171V TG mice were analyzed. In the absence of load, no differences in RANKL and OPG RNA levels between non-TG and G171V LRP5-TG mice were observed. RANKL RNA levels were not affected by application of mechanical load in either genotype. While the level of OPG RNA was not observed to be significantly induced (0.9 Log₂ fold, P<0.01) in non-TG mice, the OPG RNA levels in the HBM TG mice were significantly increased (i.e., 3.5 log₂ fold, P<0.01) (FIG. 14). This significant increase in OPG levels in the absence of any simultaneous increase in RANKL in the HBM TG mice indicates that osteoclast differentiation and activity is suppressed by the LRP5 G171V mutation.

### Example 7

### Transcriptional Profiling of MC3T3 Cells Following Application of Gravitational Load

Gravitational load (i.e., 1 G, 6 G, 12 G, and 25 G) was applied to MC3T3 cells by centrifugation for 15 min. Cells were harvested 15 min following loading and processed for total RNA. The RNA was used to generate targets for hybridization to the Affymetrix MG U74Av2 arrays.

Under the conditions of the experiment, ERK (also known as p42/44 MAPK) is phosphorylated; the phosphorylation is maximal at 25 G. RNA levels of Fos, Jun and COX-2 were all evaluated and were determined that for all three genes maximum induction also occurred at 25 G. Additionally, most of the up-regulated genes were the Wnt/β-catenin pathway components. Table 1 provides the top genes identified as being either up-regulated or down-regulated in response to gravitational load.

TABLE 11
Transcription of several Wnt/β-catenin target genes is
induced in MC3T3 mouse osteoblast cells following
application of gravitational load

| Up- | | Down- | |
|---|---|---|---|
| Regulated | | Regulated | |
| Genes | Gene Category | Genes | Gene Category |
| AP1 | Wnt target gene | BMP4 | Wnt target gene |
| AXIN | Wnt signaling intermediate | BTG2 | Suppressor of |
| | | | growth |
| BMP1 | GSK inhibitor inducible | IDB2 | Wnt target gene |
| | gene | | |
| CBFA1 | Osteoblast function | IDB3 | Wnt target gene |
| CK1 | Wnt target gene | NRA1 | Wnt target gene |
| Connexin 31 | Wnt target gene | TOB1 | Suppressor of |
| | | | growth |
| Connexin 43 | Wnt target gene | | |
| CRABP2 | Osteoblast differentiation | | |
| CTGF | Growth factor | | |
| DVL | Wnt signaling intermediate | | |
| EPHB6 | Wnt signaling gene | | |
| FOS | Wnt target gene | | |
| GADD45B | Wnt target gene | | |
| GADD45G | Cell cycle regulator | | |
| HERPUD1 | Wnt target gene | | |
| IKK alpha | β-catenin nuclear | | |
| | translocation | | |
| IL1R1 | Inflammation | | |
| JUN | Stress signaling | | |
| LDLR | Lipoprotein receptor | | |
| MAPKAPK2 | Kinase-stress signaling | | |
| MSX-1 | Wnt target gene | | |
| MYC | Wnt target gene | | |
| NCAM1 | Wnt target gene | | |
| OPG | Wnt target gene | | |
| PTGS1 | Inflammation | | |
| PTGS2 | Wnt target gene | | |
| STAT3 | Cell growth & proliferation | | |
| TIMP1 | Matrix metalloproteinase | | |
| TIMP3 | Matrix metalloproteinase | | |
| WISP1 | Wnt target gene | | |

For a more detailed summary of the genes up and down regulated by load, see Table 12 below. By “Wnt target gene” is meant to include but is not limited to a gene whose transcription is induced in response to activation of Wnt/β-catenin signaling (FIG. 16). By “Wnt signaling intermediate” is meant to include but is not limited to a gene encodes a protein involved in cellular signaling downstream of activated Wnts. By “inflammation” as used in Table 11 is meant a gene that encodes a protein involved in inflammatory responses. By “Cell growth & proliferation” as used in Table 11 is meant to include but is not limited to a gene that encodes a protein involved in cell growth and proliferation. By “growth factor” as used Table 11 is meant to include but is not limited to a gene that encodes a protein required for cell growth. By “matrix metalloproteinase” as used Table 11 is meant to include but is not limited to a gene that encodes a proteinase involved in cleavage of matrix metalloproteins. By “kinase-stress signaling” as used Table 11 is meant to include but is not limited to a gene that encodes a kinase involved in a signaling cascade down stream of stress responses (for example the p38 MAPK pathway). By “lipoprotein receptor” as used Table 11 is meant to include but is not limited to a gene that encodes a receptor for lipoproteins. By “β-catenin nuclear translocation” as used Table 11 is meant to include but is not limited to a gene that encodes a protein involved in translocation of cytoplasmic β-catenin to nucleus. By “cell-cycle regulator” as used Table 11 is meant to include but is not limited to a gene that encodes a protein involved in the regulation of the cell-cycle. By “osteoblast function” as used Table 11 able is meant to include but is not limited to a gene that encodes a protein involved in osteoblast function and activity. By “osteoblast differentiation” as used Table 11 is meant to include but is not limited to a gene that encodes a protein involved in differentiation of osteoblastic lineage cells into mature osteoblasts and osteocytes. By “suppression of growth” as used Table 11 is meant to include but is not limited to a gene that encodes a protein that suppresses cell growth. By “induced by iGSK” as used Table 11 is meant to include but is not limited to a gene whose transcription has been observed to be induced by an iGSK.

TABLE 12
Differential transcriptional profile following application of gravitational load on MC3T3 mouse osteoblast cells

| | | | | | 6G | | avg | 12G/ | | 12G | | avg |
|---|---|---|---|---|---|---|---|---|---|---|---|---|
| QUALIFIER | avg 1G | avg 6G | 6G/1G | FC2 | score | avg 1G | 12G | 1G | FC2 | score | avg 1G | 25G |
| 102044_at | 18.98 | 22.25 | 1.17 | 1.17 | −3.00 | 18.98 | 134.47 | 7.08 | 7.08 | 15.73 | 18.98 | 184.53 |
| 103039_at | 1.19 | 1.84 | 1.55 | 1.55 | 6.31 | 1.19 | 9.15 | 7.70 | 7.70 | 9.17 | 1.19 | 25.46 |
| 92368_at | 1.19 | 2.84 | 2.39 | 2.39 | −1.61 | 1.19 | 15.66 | 13.16 | 13.16 | 15.79 | 1.19 | 18.56 |
| 93294_at | 17.82 | 26.66 | 1.50 | 1.50 | 7.25 | 17.82 | 183.10 | 10.28 | 10.28 | 15.92 | 17.82 | 238.20 |
| 160519_at | 38.36 | 49.86 | 1.30 | 1.30 | −1.00 | 38.36 | 191.87 | 5.00 | 5.00 | 15.87 | 38.36 | 248.46 |
| 102209_at | 2.55 | 2.68 | 1.05 | 1.05 | −9.00 | 2.55 | 31.58 | 12.36 | 12.36 | 15.80 | 2.55 | 50.67 |
| 102021_at | 9.45 | 12.07 | 1.28 | 1.28 | −7.00 | 9.45 | 101.60 | 10.76 | 10.76 | 15.81 | 9.45 | 104.49 |
| 99603_g_at | 2.66 | 3.58 | 1.35 | 1.35 | −9.00 | 2.66 | 16.02 | 6.03 | 6.03 | 15.05 | 2.66 | 20.24 |
| 104232_at | 5.32 | 6.16 | 1.16 | 1.16 | −9.00 | 5.32 | 38.39 | 7.21 | 7.21 | 15.33 | 5.32 | 47.19 |
| 94147_at | 62.32 | 104.83 | 1.68 | 1.68 | 8.63 | 62.32 | 207.98 | 3.34 | 3.34 | 15.30 | 62.32 | 245.17 |
| 100064_f_at | 55.07 | 57.32 | 1.04 | 1.04 | −3.00 | 55.07 | 133.29 | 2.42 | 2.42 | 9.42 | 55.07 | 222.29 |
| 104601_at | 44.37 | 56.90 | 1.28 | 1.28 | −3.00 | 44.37 | 135.61 | 3.06 | 3.06 | 13.19 | 44.37 | 159.78 |
| 92676_at | 7.76 | 7.92 | 1.02 | 1.02 | −9.00 | 7.76 | 24.15 | 3.11 | 3.11 | 15.36 | 7.76 | 26.85 |
| 100130_at | 8.13 | 12.15 | 1.49 | 1.49 | −0.04 | 8.13 | 40.63 | 5.00 | 5.00 | 12.53 | 8.13 | 47.19 |
| 160832_at | 12.31 | 16.87 | 1.37 | 1.37 | −3.00 | 12.31 | 45.82 | 3.72 | 3.72 | 15.44 | 12.31 | 95.71 |
| 161666_f_at | 3.83 | 5.29 | 1.38 | 1.38 | −9.00 | 3.83 | 32.10 | 8.37 | 8.37 | 13.91 | 3.83 | 27.28 |
| 161177_f_at | 38.98 | 40.69 | 1.04 | 1.04 | −3.00 | 38.98 | 55.16 | 1.42 | 1.42 | −3.00 | 38.98 | 87.84 |
| 101526_at | 3.40 | 14.08 | 4.14 | 4.14 | 11.00 | 3.40 | 18.45 | 5.43 | 5.43 | 13.96 | 3.40 | 21.18 |
| 98500_at | 9.66 | 11.56 | 1.20 | 1.20 | −1.84 | 9.66 | 22.33 | 2.31 | 2.31 | 9.31 | 9.66 | 41.00 |
| 102887_at | 6.52 | 6.37 | 0.98 | 1.02 | −9.00 | 6.52 | 28.05 | 4.30 | 4.30 | 15.39 | 6.52 | 48.38 |
| 92877_at | 18.14 | 19.18 | 1.06 | 1.06 | −3.00 | 18.14 | 49.18 | 2.71 | 2.71 | 12.65 | 18.14 | 67.91 |
| 98501_at | 41.72 | 47.42 | 1.14 | 1.14 | −1.00 | 41.72 | 163.79 | 3.93 | 3.93 | 15.63 | 41.72 | 208.34 |
| 92364_at | 7.02 | 10.58 | 1.51 | 1.51 | 5.96 | 7.02 | 10.64 | 1.52 | 1.52 | −1.91 | 7.02 | 18.88 |
| 94932_at | 11.61 | 19.40 | 1.67 | 1.67 | 5.02 | 11.61 | 63.00 | 5.43 | 5.43 | 15.38 | 11.61 | 90.48 |
| 95597_at | 5.17 | 7.10 | 1.37 | 1.37 | −9.00 | 5.17 | 11.45 | 2.22 | 2.22 | 5.22 | 5.17 | 15.19 |
| 99602_at | 5.74 | 6.42 | 1.12 | 1.12 | −9.00 | 5.74 | 24.64 | 4.30 | 4.30 | 12.22 | 5.74 | 30.38 |
| 103328_at | 7.20 | 12.43 | 1.73 | 1.73 | 1.36 | 7.20 | 13.92 | 1.93 | 1.93 | 2.60 | 7.20 | 28.12 |
| 100065_r_at | 13.95 | 10.52 | 0.75 | 1.33 | −7.00 | 13.95 | 34.44 | 2.47 | 2.47 | 12.03 | 13.95 | 42.27 |
| 100127_at | 42.07 | 44.53 | 1.06 | 1.06 | −1.00 | 42.07 | 83.88 | 1.99 | 1.99 | 8.99 | 42.07 | 117.86 |
| 93546_s_at | 34.86 | 40.83 | 1.17 | 1.17 | −3.00 | 34.86 | 64.06 | 1.84 | 1.84 | 8.03 | 34.86 | 106.90 |
| 96033_at | 8.55 | 9.08 | 1.06 | 1.06 | −9.00 | 8.55 | 18.68 | 2.19 | 2.19 | 9.19 | 8.55 | 24.30 |
| 99070_at | 9.12 | 10.15 | 1.11 | 1.11 | −7.00 | 9.12 | 17.39 | 1.91 | 1.91 | 6.44 | 9.12 | 27.50 |
| 93914_at | 16.86 | 21.65 | 1.28 | 1.28 | −3.00 | 16.86 | 42.12 | 2.50 | 2.50 | 9.50 | 16.86 | 53.40 |
| 92399_at | 3.38 | 2.47 | 0.73 | 1.37 | −9.00 | 3.38 | 10.45 | 3.09 | 3.09 | 9.44 | 3.38 | 15.52 |
| 162371_r_at | 3.47 | 9.72 | 2.80 | 2.80 | 1.80 | 3.47 | 8.63 | 2.49 | 2.49 | 1.49 | 3.47 | 23.30 |
| 93076_at | 44.37 | 49.28 | 1.11 | 1.11 | −3.00 | 44.37 | 71.31 | 1.61 | 1.61 | 2.64 | 44.37 | 110.55 |
| 102779_at | 1.24 | 1.40 | 1.13 | 1.13 | −9.00 | 1.24 | 23.06 | 18.63 | 18.63 | 15.13 | 1.24 | 12.48 |
| 160319_at | 9.21 | 12.56 | 1.36 | 1.36 | −5.00 | 9.21 | 15.26 | 1.66 | 1.66 | 2.94 | 9.21 | 19.30 |
| 103904_at | 56.45 | 86.00 | 1.52 | 1.52 | 8.10 | 56.45 | 91.28 | 1.62 | 1.62 | 2.70 | 56.45 | 124.06 |
| 95704_at | 1.33 | 4.81 | 3.61 | 3.61 | 2.00 | 1.33 | 1.26 | 0.95 | 1.06 | −9.00 | 1.33 | 10.30 |
| 101918_at | 1.25 | 2.33 | 1.87 | 1.87 | −0.30 | 1.25 | 5.34 | 4.28 | 4.28 | 4.00 | 1.25 | 7.93 |
| 95010_at | 6.69 | 7.70 | 1.15 | 1.15 | −9.00 | 6.69 | 10.17 | 1.52 | 1.52 | −1.88 | 6.69 | 18.54 |
| 98427_s_at | 18.20 | 22.39 | 1.23 | 1.23 | −3.00 | 18.20 | 41.13 | 2.26 | 2.26 | 9.26 | 18.20 | 50.30 |
| 93547_at | 32.35 | 36.22 | 1.12 | 1.12 | −1.00 | 32.35 | 56.86 | 1.76 | 1.76 | 7.55 | 32.35 | 87.52 |
| 160701_at | 10.08 | 15.90 | 1.58 | 1.58 | 2.47 | 10.08 | 15.96 | 1.58 | 1.58 | 2.50 | 10.08 | 27.07 |
| 95557_at | 10.82 | 12.21 | 1.13 | 1.13 | −7.00 | 10.82 | 18.24 | 1.69 | 1.69 | 3.12 | 10.82 | 28.59 |
| 95721_at | 14.59 | 21.14 | 1.45 | 1.45 | 1.99 | 14.59 | 29.03 | 1.99 | 1.99 | 8.99 | 14.59 | 38.34 |
| 103975_at | 1.37 | 1.75 | 1.28 | 1.28 | −14.00 | 1.37 | 4.37 | 3.20 | 3.20 | 9.29 | 1.37 | 9.33 |
| 98418_at | 7.19 | 9.09 | 1.26 | 1.26 | −1.65 | 7.19 | 11.40 | 1.59 | 1.59 | 0.51 | 7.19 | 17.97 |
| 101979_at | 14.76 | 20.79 | 1.41 | 1.41 | −3.00 | 14.76 | 33.81 | 2.29 | 2.29 | 9.29 | 14.76 | 33.55 |
| 92701_at | 8.04 | 8.16 | 1.01 | 1.01 | −9.00 | 8.04 | 10.36 | 1.29 | 1.29 | −7.00 | 8.04 | 17.70 |
| 101464_at | 267.45 | 308.38 | 1.15 | 1.15 | −3.00 | 267.45 | 451.79 | 1.69 | 1.69 | 8.01 | 267.45 | 587.13 |
| 99100_at | 19.86 | 27.06 | 1.36 | 1.36 | −3.00 | 19.86 | 34.24 | 1.72 | 1.72 | 3.35 | 19.86 | 43.36 |
| 95057_at | 8.47 | 7.95 | 0.94 | 1.07 | −9.00 | 8.47 | 22.90 | 2.71 | 2.71 | 9.71 | 8.47 | 18.10 |
| 99835_at | 58.37 | 72.72 | 1.25 | 1.25 | −1.00 | 58.37 | 53.64 | 0.92 | 1.09 | −3.00 | 58.37 | 116.08 |
| 100152_at | 2.28 | 3.76 | 1.65 | 1.65 | 3.71 | 2.28 | 3.41 | 1.50 | 1.50 | −4.51 | 2.28 | 8.79 |
| 93126_at | 3.15 | 3.79 | 1.20 | 1.20 | −9.00 | 3.15 | 6.30 | 2.00 | 2.00 | 5.82 | 3.15 | 6.52 |
| 102364_at | 68.18 | 93.10 | 1.37 | 1.37 | −1.00 | 68.18 | 86.33 | 1.27 | 1.27 | −3.00 | 68.18 | 128.57 |
| 104354_at | 1.78 | 2.54 | 1.43 | 1.43 | −12.00 | 1.78 | 6.32 | 3.55 | 3.55 | 6.43 | 1.78 | 10.49 |
| 104647_at | 73.47 | 76.91 | 1.05 | 1.05 | −3.00 | 73.47 | 88.97 | 1.21 | 1.21 | −3.00 | 73.47 | 129.55 |
| 94231_at | 9.85 | 9.49 | 0.96 | 1.04 | −9.50 | 9.85 | 11.24 | 1.14 | 1.14 | −7.50 | 9.85 | 17.92 |
| 160545_at | 6.70 | 13.85 | 2.07 | 2.07 | 10.83 | 6.70 | 11.95 | 1.78 | 1.78 | 3.69 | 6.70 | 14.13 |
| 102581_at | 3.02 | 3.26 | 1.08 | 1.08 | −9.00 | 3.02 | 4.18 | 1.38 | 1.38 | −9.50 | 3.02 | 7.86 |
| 162371_r_at | 3.47 | 9.72 | 2.80 | 2.80 | 1.80 | 3.47 | 8.63 | 2.49 | 2.49 | 1.49 | 3.47 | 23.30 |
| 99532_at | 33.19 | 37.56 | 1.13 | 1.13 | 2.03 | 33.19 | 15.81 | 0.48 | 2.10 | 9.10 | 33.19 | 34.25 |
| 102371_at | 154.66 | 172.23 | 1.11 | 1.11 | −3.00 | 154.66 | 9.31 | 0.06 | 16.62 | 15.71 | 154.66 | 36.71 |
| 92614_at | 64.65 | 92.80 | 1.44 | 1.44 | −1.00 | 64.65 | 3.01 | 0.05 | 21.46 | 15.98 | 64.65 | 2.88 |
| 93013_at | 27.81 | 31.19 | 1.12 | 1.12 | −3.00 | 27.81 | 3.11 | 0.11 | 8.95 | 16.00 | 27.81 | 3.27 |
| 160901_at | 224.53 | 258.08 | 1.15 | 1.15 | −3.00 | 224.53 | 12.27 | 0.05 | 18.30 | 16.00 | 224.53 | 35.45 |
| 94820_r_at | 22.51 | 6.05 | 0.27 | 3.72 | 10.00 | 22.51 | 13.15 | 0.58 | 1.71 | 5.28 | 22.51 | 5.85 |
| 93456_r_at | 20.57 | 20.29 | 0.99 | 1.01 | −3.00 | 20.57 | 7.90 | 0.38 | 2.61 | 12.51 | 20.57 | 7.14 |
| 101583_at | 140.08 | 182.43 | 1.30 | 1.30 | −3.00 | 140.08 | 18.54 | 0.13 | 7.56 | 15.87 | 140.08 | 49.79 |

| | 25G/ | | 25G | | |
|---|---|---|---|---|---|
| QUALIFIER | 1G | FC2 | score | NAME | GENE DESCRIPTION |
| 102044_at | 9.72 | 9.72 | 16.00 | WISP1-Wnt target | WNT1 inducible signaling pathway protein 1 |
| | | | | gene | |
| 103039_at | 21.42 | 21.42 | 16.00 | ITGA5 | integrin alpha 5 (fibronectin receptor alpha) |
| 92368_at | 15.60 | 15.60 | 16.00 | RAMP3 | receptor (calcitonin) activity modifying |
| | | | | | protein 3 |
| 93294_at | 13.37 | 13.37 | 16.00 | CTGF | connective tissue growth factor |
| 160519_at | 6.48 | 6.48 | 15.91 | TIMP3 | tissue inhibitor of metalloproteinase 3 |
| 102209_at | 19.84 | 19.84 | 15.89 | NFATC1 | nuclear factor of activated T-cells, |
| | | | | | cytoplasmic 1 |
| 102021_at | 11.06 | 11.06 | 15.89 | IL4RA | interleukin 4 receptor, alpha |
| 99603_g_at | 7.61 | 7.61 | 15.81 | TIEG | TGFB inducible early growth response |
| 104232_at | 8.87 | 8.87 | 15.63 | GJB3-Wnt target | gap junction membrane channel protein |
| | | | | gene | beta 3 |
| 94147_at | 3.93 | 3.93 | 15.49 | SERPINE1 | serine (or cysteine) proteinase inhibitor, |
| | | | | | clade E (nexin, plasminogen activator |
| | | | | | inhibitor type 1), member 1 |
| 100064_f_at | 4.04 | 4.04 | 15.48 | GJA1-Wnt target | gap junction membrane channel protein |
| | | | | gene | alpha 1 |
| 104601_at | 3.60 | 3.60 | 15.43 | THBD | thrombomodulin |
| 92676_at | 3.46 | 3.46 | 15.39 | RUNX2/CBFA1 | runt related transcription factor 2 |
| 100130_at | 5.80 | 5.80 | 15.36 | JUN-Wnt target | Jun oncogene |
| | | | | gene | |
| 160832_at | 7.77 | 7.77 | 15.29 | LDLR | low density lipoprotein receptor |
| 161666_f_at | 7.12 | 7.12 | 15.27 | GADD45B-Wnt | growth arrest and DNA-damage-inducible |
| | | | | target gene | 45 beta |
| 161177_f_at | 2.25 | 2.25 | 15.25 | LOX | lysyl oxidase |
| 101526_at | 6.23 | 6.23 | 15.18 | MSX1-Wnt target | homeo box, msh-like 1 |
| | | | | gene | |
| 98500_at | 4.25 | 4.25 | 15.15 | IL1RL1 | interleukin 1 receptor-like 1 |
| 102887_at | 7.42 | 7.42 | 15.13 | TNFRSF11B/ | tumor necrosis factor receptor superfamily, |
| | | | | OPG-Wnt target | member 11b (osteoprotegerin) |
| | | | | gene | |
| 92877_at | 3.74 | 3.74 | 15.09 | TGFBI | transforming growth factor, beta induced, |
| | | | | | 68 kDa |
| 98501_at | 4.99 | 4.99 | 15.08 | IL1RL1 | interleukin 1 receptor-like 1 |
| 92364_at | 2.69 | 2.69 | 14.96 | CELSR2 | cadherin EGF LAG seven-pass G-type |
| | | | | | receptor 2 |
| 94932_at | 7.79 | 7.79 | 13.99 | PDGFA | Cluster incl M29464: Platelet derived |
| | | | | | growth factor, alpha /cds = (62,652) |
| | | | | | /gb = M29464 /gi = 200272 /ug = Mm.2675 |
| | | | | | /len = 906 /STRA = for |
| 95597_at | 2.94 | 2.94 | 13.88 | PTGS1 | prostaglandin-endoperoxide synthase 1 |
| 99602_at | 5.30 | 5.30 | 13.58 | TIEG | TGFB inducible early growth response |
| 103328_at | 3.91 | 3.91 | 13.56 | TANK | TRAF family member-associated Nf-kappa |
| | | | | | B activator |
| 100065_r_at | 3.03 | 3.03 | 13.51 | GJA1-Wnt target | gap junction membrane channel protein |
| | | | | gene | alpha 1 |
| 100127_at | 2.80 | 2.80 | 13.18 | CRABP2 | cellular retinoic acid binding protein II |
| 93546_s_at | 3.07 | 3.07 | 13.08 | CBFB | core binding factor beta |
| 96033_at | 2.84 | 2.84 | 13.04 | SDC1 | syndecan 1 |
| 99070_at | 3.02 | 3.02 | 13.00 | CHUK/IKK | alpha-conserved helix-loop-helix ubiquitous |
| | | | | | facilitates beta- kinase |
| | | | | | catenin nuclear |
| | | | | | translocation |
| 93914_at | 3.17 | 3.17 | 12.89 | IL1R1 | interleukin 1 receptor, type I |
| 92399_at | 4.59 | 4.59 | 12.39 | RUNX1 | runt related transcription factor 1 |
| 162371_r_at | 6.72 | 6.72 | 12.19 | EPHB6-Wnt target | Eph receptor B6 |
| | | | | gene | |
| 93076_at | 2.49 | 2.49 | 11.99 | CK1 alpha-Wnt | casein kinase 1, alpha 1 |
| | | | | | pathway |
| | | | | | component |
| 102779_at | 10.08 | 10.08 | 11.81 | GADD45B-Wnt | growth arrest and DNA-damage-inducible |
| | | | | target gene | 45 beta |
| 160319_at | 2.10 | 2.10 | 11.57 | SPARCL1 | SPARC-like 1 (mast9, hevin) |
| 103904_at | 2.20 | 2.20 | 11.55 | IGFBP6 | insulin-like growth factor binding protein 6 |
| 95704_at | 7.72 | 7.72 | 11.49 | AP1B1-Wnt target | adaptor protein complex AP-1, beta 1 |
| | | | | gene | subunit |
| 101918_at | 6.35 | 6.35 | 10.00 | TGFB1 | transforming growth factor, beta 1 |
| 95010_at | 2.77 | 2.77 | 9.77 | TRAF3 | TNF receptor-associated factor 3 |
| 98427_s_at | 2.76 | 2.76 | 9.76 | NFKB1 | nuclear factor of kappa light chain gene |
| | | | | | enhancer in B-cells 1, p105 |
| 93547_at | 2.71 | 2.71 | 9.71 | CBFB | core binding factor beta |
| 160701_at | 2.69 | 2.69 | 9.69 | AXIN-Wnt | target axin |
| | | | | | gene(?); Wnt |
| | | | | | pathway |
| | | | | | component |
| 95557_at | 2.64 | 2.64 | 9.64 | BMP1-observed | bone morphogenetic protein 1 |
| | | | | | to be induced by |
| | | | | | iGSK |
| 95721_at | 2.63 | 2.63 | 9.63 | MAPKAPK2 | MAP kinase-activated protein kinase 2 |
| 103975_at | 6.83 | 6.83 | 9.50 | PRDC-PENDING | protein related to DAC and cerberus |
| 98418_at | 2.50 | 2.50 | 9.50 | DVL1-Wnt | disheveled, dsh homolog (Drosophila) |
| | | | | | pathway |
| | | | | | component |
| 101979_at | 2.27 | 2.27 | 9.27 | GADD45G | growth arrest and DNA-damage-inducible |
| | | | | | 45 gamma |
| 92701_at | 2.20 | 2.20 | 9.20 | BMP1-observed | bone morphogenetic protein 1 |
| | | | | | to be induced by |
| | | | | | IGSK-3 |
| 101464_at | 2.20 | 2.20 | 9.20 | TIMP1 | tissue inhibitor of metalloproteinase |
| 99100_at | 2.18 | 2.18 | 9.18 | STAT3 | RIKEN cDNA 1110034C02 gene |
| 95057_at | 2.14 | 2.14 | 9.14 | HERPUD1-Wnt | homocysteine-inducible, endoplasmic |
| | | | | target gene | reticulum stress-inducible, ubiquitin-like |
| | | | | | domain member 1 |
| 99835_at | 1.99 | 1.99 | 8.99 | FOSL1 | fos-like antigen 1 |
| 100152_at | 3.86 | 3.86 | 8.85 | NCAM1-Wnt | neural cell adhesion molecule |
| | | | | target gene | |
| 93126_at | 2.07 | 2.07 | 8.70 | CKB | creatine kinase, brain |
| 102364_at | 1.89 | 1.89 | 8.31 | JUND1 | Jun proto-oncogene related gene d1 |
| 104354_at | 5.89 | 5.89 | 8.02 | CSF1R | colony stimulating factor 1 receptor |
| 104647_at | 1.76 | 1.76 | 7.58 | PTGS2-Wnt target | prostaglandin-endoperoxide synthase 2 |
| | | | | gene | |
| 94231_at | 1.82 | 1.82 | 5.41 | CCND1-Wnt target | cyclin D1 |
| | | | | gene | |
| 160545_at | 2.11 | 2.11 | 5.11 | CCND3 | cyclin D3 |
| 102581_at | 2.60 | 2.60 | 6.45 | MYCS-Wnt target | myc-like oncogene, s-myc protein |
| | | | | gene | |
| 162371_r_at | 6.72 | 6.72 | 12.19 | EPHB6-Wnt target | Eph receptor B6 |
| | | | | gene | |
| 99532_at | 1.03 | 1.03 | −3.00 | TOB1 | transducer of ErbB-2.1 |
| 102371_at | 0.24 | 4.21 | 16.00 | NR4A1-Wnt target | nuclear receptor subfamily 4, group A, |
| | | | | gene; up/down | member 1 |
| | | | | depending on cell | |
| | | | | type | |
| 92614_at | 0.04 | 22.45 | 16.00 | IDB3 | inhibitor of DNA binding 3 |
| 93013_at | 0.12 | 8.49 | 15.73 | IDB2-Wnt target | inhibitor of DNA binding 2 |
| | | | | gene; up/down | |
| | | | | depending on cell | |
| | | | | type | |
| 160901_at | 0.16 | 6.33 | 15.64 | FOS | FBJ osteosarcoma oncogene |
| 94820_r_at | 0.26 | 3.85 | 12.40 | CCNI | cyclin I |
| 93456_r_at | 0.35 | 2.88 | 9.88 | BMP4-Wnt target | bone morphogenetic protein 4 |
| | | | | gene; up/down | |
| | | | | depending on cell | |
| | | | | type | |
| 101583_at | 0.36 | 2.81 | 9.81 | BTG2 | B-cell translocation gene 2, anti- |
| | | | | | proliferative |

Other assays which can be used to perform transcriptional profiling (i.e., assess the up- and down-regulation of genes in response to bone load) would be the use of other oligonucleotide arrays prepared by Metrigenix and others, the use of cDNA arrays (e.g., Incyte, Becton Dickinson, Clontech and the like), or arrays as discussed herein, protein and antibody arrays (e.g. Becton Dickenson, Clontech and other vendor arrays), polymerase chain reaction using traditional methods (see e.g., PCR PROTOCOLS: A GUIDE TO METHODS AND APPLICATIONS (Michael Innis et al., ed., 1990)) or using TaqMan® (i.e., Real-time PCR of ABI), eTAG (ACLARA Biosciences), Northern blot analysis, S1 nuclease analysis, RNase protection assays and Western blot. Methods for doing these assays are known in the art. See for example, USING ANTIBODIES: A LABORATORY MANUAL, Harlow, Ed and Lane, David (Cold Spring Harbor Press, 1999); Sambrook et al., MOLECULAR CLONING: A LABORATORY MANUAL (2nd Ed. Cold Spring Harbor Laboratory Press, 1989); and Maniatis et al., MOLECULAR CLONING, A LABORATORY MANUAL, (Cold Spring Harbor Laboratory, Cold Spring Harbor, N.Y. 1982).

The primers and probes used for analyzing the genes are provided in Table 13.

TABLE 13
List of probe-primers used in TaqMan ® analysis

| GENE | Symbols | Accessions | Forward Primer | Reverse Primer | Probe |
|---|---|---|---|---|---|
| 100064_f_at | GJA1 | M63801 | F1:TGAAGGGAAGAAGCGATCCTT | R1:GGAGATCCGCAGTCTTTGGA | ACGCCACCACCGGCCCACT |
| Connexin43 | | X61576 | F1:GGCCGGAAGCACCATCT | R1:TGGCTGTCGTCAGGGAAATC | CAACTCCCACGCCCAGC CGTT |
| 101918_at | TGFB1 | AJ009862 | F1:GGAGCCTGGACACACAGTACAG | R1:GCTTGCGACCCACGTAGTAGA | ACCAACACAACCCGGGCGCTT |
| 102801_at | BGLAP-RS1 | L24430 | F1:TGCATGTTGAAAGGTTCCTGAA | R1:CACCCTCCTGTTGCCTCTGA | AGTGTCGTCGTTTCTTTCTGCTGGTCAGA |
| 103709_at | COL1A1 | AA763466 | F1:AGTTCCTGGGCCTATCTGATCTC | R1:CCTGATGCAGGACAGACCAA | TCCCCTCTTGCTGCTGCTCCCTC |
| 103904_at | IGFBP6 | X81584 | F1:CGGCCCAATCCTGTTCAA | R1:CGCCTCGGAAGACCTCAGT | CCCCTGCCGCAGACACTTGGA |
| 103991_at | AKP5 | M61704 | F1:TGTTGGCAGGGAAAATGTTGA | R1:GGCACTGAACAAGCCAACAA | TTCAGCCGCCGCCATCAGC |
| 160406_at | cathepsin K | AJ006033 | F1:GGTGCAAGATATTGGTGGCTTT | R1:TCGCTGCGTCCCTCTCA | AGCGCCATGCCCACTCCCTTC |
| 160519_at | TIMP3 | U26437 | F1:AGTCGGCTGTTTGGGTTGAG | R1:ACAGCTGGCTTGCTAGAGGAA | CCCGAGGAAATGACCATGCTCTGG |
| 161515_i_at | TIMP2 | AV156389 | F1:TTCCCGCGATGAGTGCTT | R1:ATTTGGCCTGGTGCTCATTAA | TGCTCTTCTCTGTGACCCAGTCCATCC |
| 161666_f_at | GADD45B | AV136783 | F1:TGCGGAACAGTGAAATGTGTATAA | R1:AGATTTGCTGTAGCTGCGAAGTC | ACGACCCTTGCCGCGGGAC |
| 92469_at | SFRP4 | AF117709 | F1:TGGAGCCACCCTTACAGGAT | R1:GCAAGTGGTATGTGGCCTTCTG | AGGCTGTCCCAGGCAGCACCA |
| 94231_at | CCND1 | M64403 | F1:AGAAATGTACTCTGCTTTGCTGAA | R1:GGGCTGTAGGCACTGAGCAA | AGGCCCTCAGCCTCACTCCCTGG |
| 94704_at | WISP2 | AF100778 | F1:GGTGACCTTGTAAGTGTGCCTTT | R1:TCCATCTCTTCATGTTCCCAGAA | TCTGAGAACACCCTGCCCGGCT |
| 97997_at | SFRP1 | U88566 | F1:CCCTCCAAGGCTTGAGTAAAAG | R1:AGCACATGCATAGGCGGTGTA | TCGTTGACTGCCCAAGGCTGCC |
| 98623_g_at | IGF2 | X71922 | F1:CCTCCCTTTGTCATCATGTGAA | R1:GGACAGTGGCACAGGTGACA | TTCCCACGCGTCGAACGCC |
| 98859_at | ACP5 | M99054 | F1:TCATATATGTGGAAGCCTCTGGAA | R1:GCAGGACTCTCGTGGTGTTCA | CCAGCCTCCCAAGGAGACCCAGA |
| Frizzled2 | Frizzled 2 | af363723 | F1:GCCCGACTTCACAGTCTACATG | R1:GCCGGACCAGATCCAGAA | CCGACGTGATGCCCACGATGA |
| | Catnbip1 | NM_023465 | F1:GGAGCAGGACAGTGGAGAATC | R1:CCTGAGAGGAGAGCGTCATTG | CCAACCCCAGCCTGACCAGCAA |
| | Osteonectin | M20692 | F1:CGGGCGTTTCTTTCCATGT | R1:GCCCAATTGCAGTTGAGTGA | TTCTGGCCCACCCATGGCTCA |
| | beta2 M | | F1:GAGAATGGGAAGCCGAACATAC | R1:TTTCCCGTTCTTCAGCATTTG | CACAGTTCCACCCGCCTCACATTG |
| mLPR5 | Lrp5 | AF064984 | F1:CCCCTCTATGACCGGAATCAC | R1:CGGATATAGTGTGGCCTTTGTG | CATCCAGCAGCTCGT(MGB) |
| 104647 | PTGIS | M88242 | F1:AGGCTGTTGGAATTTACGCATAA | R1:CATGCTTGGGTCAGTCAATATTG | AGCAGACTGCATAGAT(MGB) |
| 104538 | PTGS2 | AB001607 | F1:TGGCTTCGGTCTGATGCA | R1:CCCAGGTGAGTCTGCTCCAT | CCAGAGGAAGACGTGCCCATCCG |
| | eNOS | nm_008713 | F1:TCTGCGGCGATGTCACTATG | R1:GCCCTCTGTTGCCAGAATTC | AGCGTCCTGCAAACCGTGCA |
| Wnt10B | Wnt10B | AF029307 | F1:CCTCGGGCTCAGGTTCCTA | R1:AAGAGGAGTGGCCAAAAGATAGACT | CCCTATCCAAAGGAAG(MGB) |
| | MK2 | XM_129464 | F1:CATTTCATGCATCTCCCCTGAT C | R1:GCGAAGACTGTCCCATCCA | CACGTGGTCCTGCCCTTGTCGA |
| 95348_at | GRO1 | J04596_ | F1:CCCCAAGTAACGGAGAAAGAAGA | R1:GTTGTCAGAAGCCAGCGTTCA | CAGACTGCTCTGATGGCACCGTCTG |
| | OPG | NM_008764 | F1:CCACTCGAACCTCACCACAGA | R1:CAATCTCTTCTGGGCTGATCTTC | CAGGCAGGCTCTCCATCAAGGCA |
| 93416_at | RANKL | | F1:GACTTGTCAAAACTATGCAAGCAA | R1:TGGCTATGTCAGCTCCTAAAGTCA | TGTTGGTCACCAGGTGCCTTTCAAATTT |
| | H_mk2 | | F1:ACCAGCCCGTCTTCTCTCTCT | R1:CAGCACCAGGAAGGGTACAGA | TGCCGCCTCACCTGCCCTTGT |
| 99333_at | E_SELECTIN | M80778 | F1:TGTTCTGTGTCCTGGCACTGA | R1:TTTGACCCTTGAGCTGACATAAGAA | CCAGCATGAGATCCA(MGB) |
| 102802_at | mIL18 | D49949 | F1:GGACACTTTCTTGCTTGCCAA | R1:CAGATTTATCCCCATTTTCATCCT | TGAAAGCATCATCTTC(MGB) |
| 96574_at | mIL9 | M30136 | F1:AAGCCATGCAACCAGACCAT | R1:GTCCCCAGGAGACTCTTCAGAA | AGGCAACACACTGTCA(MGB) |
| 97497_at | mNOTCH1 | Z11886 | F1:TCCGAACCAGTAGCTCCTAA | R1:ACTTGGTGGGCAGCAGATG | AGCACAACCCAGGATG(MGB) |
| 92560_at | mVCAM | U12884 | F1:CCCTCCACAAGGCTTCAAGA | R1:GGTAGACCCTCGCTGGAACA | TGCTGTGACAATGAC(MGB) |

| | | |
|---|---|---|
| mIL6 | IL6 | PDAR_#4329592F from ABI - no sequence info. |
| hIL6 | hIL6 | PDAR: #4327040F from ABI - no sequence info. |
| mFOS | mFOS | PDAR from ABI - no sequence info. |
| mJUN | mJUN | PDAR from ABI - no sequence info. |

### Example 8

### COX-2 Inhibitor Induced Modulation of Wnt Pathway Activity and Impact on Bone Load

As discussed above, COX-2 gene transcription is induced by application of mechanical bone load both in vitro and in vivo. COX-2 expression can be induced by Wnt 1 (Howe et al., J. Biol. Chem. 276(23): 20108-15 (2001)). It is further known that the promoter for COX-2 has TCF-4 binding sites (Araki et al., Cancer Res. 63(3): 728-34 (2003)). Therefore, it was questioned whether COX-2 activity was necessary for load induced transcription of Wnt/β-catenin target genes. The following experiment and associated data answered this question.

MC3T3 cells were either left untreated or treated with 1, 10 and 60 μM of the COX-2 inhibitor, NS-398 ([N-(2-cyclohexyloxy-4-nitrophenyl)methanesulfonamide]) 1 hr prior to loading via Flexercell® as discussed previously.

RNA was isolated immediately after application of load and processed for TaqMan® analysis. The transcripts analyzed were COX-2, eNOS, Connexin 43, SFRP1, Wnt10B, cyclin D1, Frizzled 2, WISP2, Fos and Jun (FIG. 15).

The results demonstrated that load induced transcription of FzD2, eNOS FOS, JUN, COX-2, Connexin 43, cyclin D1, SFRP1 and Wnt10B. The latter four genes are all dependent on COX-2 activity, because in the presence of the COX-2 inhibitor, load does not induce transcription in these genes. Load induced transcription of Frizzled 2, eNOS, Fos and Jun were independent of COX-2 activity (FIG. 15). WISP2 gene expression was not load inducible in MC3T3 cells. These experiments utilizing the COX-2 inhibitor and the resulting conclusions that can drawn from these experiments are just one example of how this and other signaling pathway modulators can be used to identify essential elements/factors required for the bone anabolic effect of loading and the contribution of activating the Wnt/β-catenin pathway.

### Example 9

### Wnt 3A Synergistically Induces β-Catenin Target Gene Expression

Like the experiments above involving the treatment of MC3T3 cells (see Example 1) with the GSK3-β inhibitor in the presence of load and its effects on β-catenin target gene expression, the following loading experiment was performed to see if another compound could enhance bone load. This experiment was performed in the presence of the natural Wnt ligand, Wnt 3A. The aim was to determine whether the activation of the Wnt/β-catenin pathway at the level of LRP5/6/Frizzled co-receptors would have similar synergistic induction of β-catenin target gene expression in the presence of load as was observed with the GSK-3 inhibitor (examples above).

Here, Wnt 3A conditioned media was obtained from mouse L-cells transfected with murine Wnt 3A. The MC3T3 cells were seeded and cultured for 3 days in growth media until confluence, as described above. The media for the MC3T3 cells was then changed to BSA containing media, and the cells were incubated for 24 hours. The BSA-containing media was then removed and replaced with fresh BSA media at a final volume of 1 mL containing various amounts of L-cell Wnt 3A conditioned media or control conditioned media from untransfected L-cells. The amount of Wnt 3A conditioned media varied from 0.5, 2.0, 5.0, 10.0, 20.0 and 100 μL in a final volume of 1 ml serum free BSA media. The MC3T3 cells were then subjected to 3,400 με at 2 Hz, 7200 cycles/hr for 5 hours as described in the prior examples.

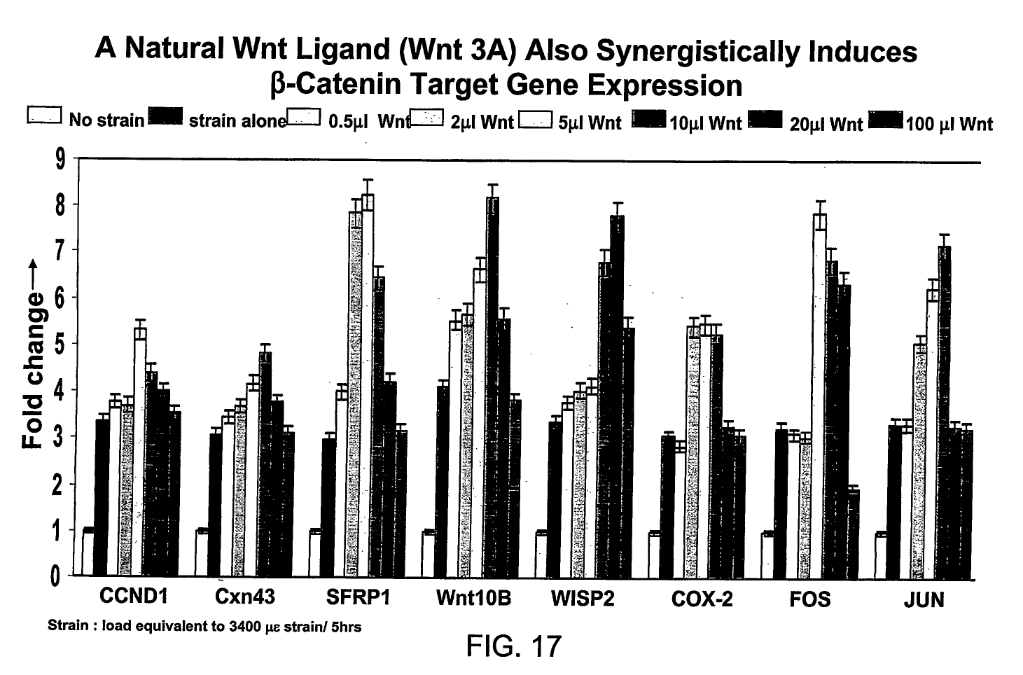

The cells were harvested and processed as discussed in the examples above. The results depicted in FIG. 17 demonstrate that Wnt 3A alone (i.e., no load) had no effect on cyclin D1, connexin 43, SFRP1, Wnt 10B, WISP2, COX-2, FOS and JUN gene expression. However, in the presence of load, Wnt 3A dose dependently (i.e., in a biphasic and fashion) and synergistically induced the expression of cyclin D1, connexin 43, SFRP1, Wnt 10B, WISP2, COX-2, FOS and JUN. The fold induction above load alone ranged from 1.8 fold to 2.6 fold induction. The amount of Wnt 3A conditioned media most effective at enhancing load ranged from about 2 μL to 20 μL and more preferably between 2 to 10 μL. The control L-cell conditioned media that did not contain Wnt 3A had no additional effect on β-catenin target gene expression in the presence of load.

These data further support the concept that activation of the Wnt/β-catenin pathway with a natural Wnt ligand causes bone cells to be more responsive to mechanical loading. Thus, Wnt 3A and its mimetics can be used for the same purposes as proposed for the GSK inhibitors discussed herein. For example, enhancement of Wnt3A expression or use of Wnt 3A mimetics or functional variants can be used to enhance bone load in order to increase bone mass in a patient.

### Example 10

### Effect of Systemic GSK Inhibitor Administration on In Vivo Response to Mechanical Load

A hypothesis was developed that systemic treatment with a GSK inhibitor would activate Wnt signaling, thereby mimicking the bone response to mechanical load. The response was expected to be similar to what is observed with the high bone mass (“HBM”) transgenic animal model, i.e. bones experience the anabolic load effect in the HBM animals (activated Wnt signaling) at lower amounts of strain on the bone than in wild-type animals (see FIGS. 12, 13a and 13b; Example 6). The hypothesis was tested using the following materials and methods.

Materials and Methods. Wild type 17-week old female mice were injected with 10 μg/mL/kg (low), 50 μg/mL/kg (high), or vehicle (control) respectively. The injections were administered subcutaneously, twice daily for a period of 14 days. There were a total of 20 animals in each cohort. The GSK inhibitor used was 3-(3-chloro-4-hydroxyphenylamino)-4-(2-nitrophenyl)-1H-pyrrole-2,5-dione.

The right tibiae of the animals were loaded at 6 N for 36 cycles at 2 Hz. The left tibiae of the animals were unloaded controls. After this procedure, animals were sacrificed at 4 hours post-load. Tissue was processed at that time and flash frozen in liquid nitrogen. Tibiae were pooled into 4 groups of 5 for each cohort, loaded (left) and unloaded (right). mRNA was purified from tibiae (loaded and unloaded), liver, spleen, kidney, brain, colon, and skin. Transcriptional analyses were performed by Taqman® real-time RT-PCR on samples from the tibiae on selected load- and Wnt-response genes (described in FIGS. 12, 13a and 13b; Example 6). More global profiling was performed using Affymetrix® gene chips using manufacturer's instructions for the gene chips.

All the animals completed the full protocol. Expression of the following genes was monitored in the tibiae: Cox2, eNos, Wnt10B, SFRP1, Cxn43, CCND1, Fzd2, and WISP2. Robust transcriptional changes were observed in the GSK inhibitor treated animals, with a dose-dependent trend between those animals administered with low dose versus high dose of the GSK inhibitor. At high dose GSKi treatment, all of the monitored genes were significantly induced in the loaded tibiae versus the unloaded tibiae. In this comparison, Cox2 was induced approximately 27-fold, eNos 5-fold, Wnt10B 7-fold, SFRP1 2.5-fold, Cx43 5-fold, CCND1 4-fold, Fzd2 7-fold, and WISP2 3-fold. In the presence of load, treatment with high dose GSKi synergistically induced gene expression of the following genes compared to vehicle treatment: Cox2, eNos, Wnt10B, SFRP1, Cx43, CCND1, and Fzd2. Together this data confirms the previous observations from the HBM transgenics (Example 6) and in vitro Flexer cell studies (Example 9) and confirm that activation of the Wnt signaling pathway enhances the normal bone response to mechanical load resulting in bones experiencing or perceiving lower strain at equivalent loads.

Although the present invention has been described in detail with reference to the examples above, it is understood that various modifications can be made without departing from the spirit of the invention, and would be readily known to the skilled artisan.

1. A gene expression profile of bone cells subjected to bone load, and wherein bone load has been modulated by a Wnt pathway modulator.

2. The gene expression profile of claim 1, wherein the gene expression profile comprises COX-2, Jun, Fos, SFRP1, Connexin 43, and eNOS genes.

3. The gene expression profile of claim 1, wherein the gene expression profile comprises two or more genes of Tables 1-5, 11, or 12.

4. The gene expression profile of claim 1, wherein the Wnt pathway modulator is an agonist.

5. The gene expression profile of claim 4, wherein the agonist is a GSK-3 inhibitor.

6. The gene expression profile of claim 4, wherein the agonist is a Wnt 3A, a Wnt 3A variant, a Wnt 3A mimetic, or Wnt 3A agonist.

7. The gene expression profile of claim 5, wherein the GSK-3 inhibitor is a selective GSK-3 inhibitor.

8. The gene expression profile of claim 5, wherein the GSK-3 inhibitor is lithium chloride or a pharmaceutically acceptable salt thereof, a maleimide, a muscarinic agonist, an aloisine, a hymeninidisine, or an inidirubin.

9. The gene expression profile of claim 8, wherein the maleimide is 3-(2,4-dichlorophenyl)-4-(1-methyl-1H-indol-3-yl)-1H-pyrrole-2,5-dione or 3-(3-chloro-4-hydroxyphenylamino)-4-(2-nitrophenyl)-1Hpyrrole-2,5-dione.

10. The gene expression profile of claim 4, wherein the gene expression profile is derived from cultured cells or cells obtained from animal tissue.

11. The gene expression profile of claim 1, wherein the bone cells are preosteoblasts, osteoprogenitor cells, osteoblasts, osteoclasts, osteocytes, or mesenchymal stem cells, or combinations thereof.

12. A method of identifying Wnt pathway modulating agents and thereby modulate bone remodeling comprising the steps of:
(A) obtaining a gene expression profile of bone cells exposed to a candidate agent; and
(B) comparing the gene expression profile of step (A) with the gene expression profile of claim 1 thereby determining whether the Wnt pathway was modulated.

13. The method of claim 12, wherein the mechanical load is applied to an animal and the bone cells are obtained from the animal, or wherein mechanical load is applied to cultured bone cells.

14. A gene expression profile of HBM cells subjected to mechanical stress and a Wnt pathway modulator.

15. A method of preparing a bone loading gene expression profile comprising the steps of:
(A) obtaining a first gene expression profile of bone cells which are not exposed to bone load, a second gene expression profile of bone cells which are exposed to bone load, and a third gene expression profile of bone cells which are exposed to bone load and a Wnt pathway modulator; and
(B) comparing the first, second and third gene expression profiles to thereby obtain a bone loading gene expression profile of Wnt pathway modulator regulated genes.

16. The method of claim 15, wherein the bone cells are osteoclasts, osteoblasts, osteocytes, or a combination of said bone cells.

17. The method of claim 16, wherein the Wnt pathway modulator is a Wnt pathway agonist.

18. The method of claim 17, wherein the Wnt pathway agonist is a GSK-3 inhibitor, a Wnt 3A, a Wnt 3A mimetic, a Wnt 3A agonist, a LRP5 agonist, a LRP6 agonist, a β-catenin agonist, or a Dkk1 antagonist.

19. A bone loading gene expression profile comprising genes regulated by a Wnt pathway modulator obtained by the method of claim 15.

20. A method of screening an agent which enhances bone load associated remodeling comprising the steps of:
(A) obtaining a gene expression profile of bone cells cultured with the agent and exposed to bone load; and
(B) comparing the gene expression profile of step (A) with the bone loading gene expression profiles of claim 19, and wherein the Wnt pathway modulator is a reference Wnt pathway modulator.

21. The method of claim 20, wherein the reference Wnt pathway modulator is a GSK-3 inhibitor or Wnt 3A.

22. The method of claim 20, wherein when the cultured bone cell assessed in the absence of a candidate agent is an HBM bone cell.

23. The method of claim 20, wherein the bone cells are osteoblasts, preosteoblasts, osteoprogenitor cells, mesenchymal stem cells, or combinations thereof.

24. The method of claim 23, wherein the bone cells are osteoblasts, and wherein the effect of the agent on osteoblast number and/or proliferation is measured by [³H]-thymidine incorporation, 5-bromo-2′-deoxyuridine (BrdU) incorporation, 3-(4,5-dimethylthiazol-2-yl)-5-(3-carboxymethoxyphenyl)-2(4-sulfophenyl)-2H-tetrazolium salt (MTS) assay, or an apoptosis assay.

25. The method of claim 20, wherein the bone load administered in steps (A) and (B) of claim 20 is mechanical load in the amount of about 50 to about 5,000 με.

26. A candidate agent for treating a low bone mass condition identified by the method of claim 20.

27. A method of treating a bone mineralization disease or disorder comprising administering in a therapeutic effective amount the candidate agent of claim 26.

28. The method of claim 27, wherein the bone disease or disorder is osteoporosis, a bone fracture, chondrodystrophies, a drug-induced bone disorder, high bone turnover, hypercalcemia, hyperostosis, osteoarthritis, osteomyelitis, and Paget's disease.

29. The method of claim 28, wherein the bone fracture is a hip fracture, Colle's fracture, or a vertebral crush fracture.

30. The method of claim 28, wherein the drug-induced disorder is glucocorticoid induced osteoporosis, heparin-induced osteoporosis, an aluminum hydroxide induced osteomalacia, anticonvulsant induced osteomalacia, or glutethimide induced osteomalacia.

31. The candidate agent of claim 25, wherein the candidate agent is a GSK-3 antagonist, a Wnt 3A, a Wnt 3A mimetic, a Wnt 3A agonist, a Dkk1 antagonist, an LRP5 agonist, a β-catenin agonist, or a LRP6 agonist.

32. A composition comprising a plurality of probes, wherein the probes comprise nucleic acid sequences that anneal to nucleic acids of the bone loading gene expression profile of claim 19.

33. The composition of claim 32, wherein the plurality of probes are attached to a solid substrate.

34. The composition of claim 33, wherein the solid substrate is a bead, a plate, or a slide.

35. The composition of claim 32, wherein the plurality of probes comprise nucleic acid sequences which anneal to nucleic acids sequences encoding connexin 43, COX-2, eNOS, SFRP1, Jun, and Fos proteins.

36. The composition of claim 32, wherein the plurality of probes comprise nucleic acid sequences that anneal to nucleic acid sequences of genes or gene transcripts of Tables 1-5, 11, or 12.

37. The composition of claim 35 further comprising probes that anneal to nucleic acid sequences of PDGFRA, MET, OSMR, ITGBL1, CTGF, WNT6, TIMP3, GJA1, GAS6, LOX, MYBL1, THBS1, ITGB5, CTSK, COL1A1, FBLN1, CCND1, TIMP2, COL6A3, GADD45A, WISP2, FZD2, SFRP4, IGFBP6, LRP5, LRP6, LSP1, CX3CR1, TGFBR2, VCAM1, IL6, FGF2, FGF7, STAT1, TNFRSF10B, IFG2R, IGF2, SPARC, MAPKAPK2, TNF, TNFRSF11b, TNFSF11, ACP5, FAP, MCC, DELTEX, EPHB2, CNK1, ERBB3, GRO1, MYC, COX-2, eNOS and WNT10B.

38. A method of modulating bone mineralization in a cell comprising administering an agent which produces a bone load expression profile of any of claims 1 or 14.

39. The method of claim 38, wherein the agent is a Wnt agonist, a Wnt 3A, a Wnt 3A mimetic, a Wnt 3A variant, a Wnt 3A agonist, a Dkk antagonist, a COX-2 antagonist, a LRP5 agonist, a LRP6 agonist, a GSK-3 antagonist, or a β-catenin agonist.

40. The method of claim 39, wherein the GSK-3 antagonist is a maleimide, a muscarinic agonist, an aloisine, a hymeninidisine or an inidirubin.

41. The method of claim 40, wherein the maleimide is administered in combination with a second bone remodeling modulating agent.

42. The method of claim 41, wherein the second bone remodeling modulating agent is parathyroid hormone, estrogen, vitamin D, a vitamin D analog, a selective estrogen receptor modulator, a glucocorticoid, a calcium preparation or a bisphosphonate.

43. A method of modulating bone mineralization and/or bone remodeling in a subject in need thereof comprising administering a compound which produces a bone load expression profile of claim 19.

44. A composition comprising a substrate and a plurality of immunoglobulins adhered to the substrate, wherein said immunoglobulins recognize and bind to two or more proteins of Tables 1-5, 11, or 12.

45. The composition of claim 44, wherein the plurality of immunoglobulins comprise two or more immunoglobulins that recognize and bind to said two or more proteins of Tables 1-5, 11, or 12.

46. The composition of claim 45, wherein the two or more proteins are eNOS, connexin 43, SFRP1, cyclin D1, Wnt10B, Jun, Fos, or COX-2.

47. The composition of claim 44, wherein the substrate is a microchip, a bead, a plate, a slide, or a tube.

48. A composition for studying bone load modulation comprising:
(A) a substrate; and
(B) a plurality of two bone cell lysates or more cell lysates adhered to said substrate, wherein the lysate is from (i) cells without mechanical stress, (ii) cells exposed to mechanical stress, (iii) HBM cells without mechanical stress, (iv) HBM cells exposed to mechanical stress, and (v) any of the prior cells exposed to a Wnt pathway modulator.

49. The composition of claim 48, wherein the substrate is a microchip, a bead, a plate, a slide or a tube.

50. A method of screening reagents that bind to proteins that modulate bone remodeling and/or bone mineralization comprising the steps of:
(A) exposing a candidate reagent to a composition of claim 48 under suitable conditions for binding of the candidate reagent to the composition of claim 48; and
(B) determining whether said candidate reagent bound to the composition of claim 48 and further determining which protein of the composition of claim 48 bound said candidate reagent.

51. A method of determining whether a compound or a composition enhances the effect of bone load on bone cell activity/function and/or mineralization comprising the steps of:
(A) administering the compound or the composition to a cell line;
(B) administering thereafter a mechanical stimulus to the cell line;
(C) obtaining a cell lysate from the cell line;
(D) contacting the cell lysate to the composition of claim 44 under suitable conditions to allow binding of proteins in the cell lysate to the composition of claim 44; and
(E) determining whether the compound or the composition enhances the effect of bone load on bone cell activity/function and/or mineralization by comparing the pattern obtained from step (D) with an expression pattern obtained from a cell lysate of cells to which mechanical load stimulus only was administered.

52. The gene expression profile of claim 3, wherein the gene expression profile comprises COX-2, Jun, FOS, SFRP1, Connexin 43, eNOS, Wnt10B, cyclin D1, Frizzled2, and WISP2.

53. The composition of claim 32, wherein the probes that anneal to nucleic acids of the bone expression profile comprising COX-2, Jun, FOS, SFRP1, Connexin 43, eNOS, Wnt10B, cyclin D1, Frizzled2, and WISP2.
